(19) United States
(12) Patent Application Publication
(10) Pub. No.: US 2026/0055110 A1
(43) Pub. Date: Feb. 26, 2026

(54) COMPOUNDS AND METHODS FOR THE TREATMENT OF DERMAL AND OCULAR DISORDERS

(71) Applicant: Azura Ophthalmics Ltd., Tel Aviv (IL)

(72) Inventors: Ian HOLMES, Melbourne, Victoria (AU); Yair ALSTER, Tel Aviv (IL); Hila BARASH, Shoham (IL); Charles BOSWORTH, Las Vegas, NV (US); Omer RAFAELI, Udim (IL); Robert M. BURK, Tel Aviv (IL); Marc GLEESON, Longueville (AU); Mark Richard STEWART, Cambridge (GB); Jonathan DUNN, Cambridge (GB); Soultana KATSINA, London (GB); Elisa PILEGGI, Cambridge (GB); Alexander James NICHOLLS, Cambridge (GB)

(21) Appl. No.: 18/702,759

(22) PCT Filed: Oct. 19, 2022
(86) PCT No.: PCT/IB2022/000609

§ 371 Date: Apr. 18, 2024

Related U.S. Application Data
(60) Provisional application No. 63/257,855, filed on Oct. 20, 2021.

(51) Int. Cl. C07D 487/04; A61K 31/277; A61K 31/385; A61K 31/439; A61K 31/519; A61K 31/573; A61K 31/58; A61P 27/02; C07C 69/42; C07C 255/46; C07D 339/04; C07D 491/18; C07J 3/00; C07J 33/00; C07J 41/00; C07J 43/00

(52) U.S. Cl. CPC C07D 487/04; A61K 31/277; A61K 31/385; A61K 31/439; A61K 31/519; A61K 31/573; A61K 31/58; A61P 27/02; C07C 69/42; C07C 255/46; C07D 339/04; C07D 491/18; C07J 3/005; C07J 33/002; C07J 41/0038; C07J 41/0094; C07J 43/003

(57) ABSTRACT

Described herein are compositions and methods for the treatment or prevention of dermal or ocular surface disorders. Ocular surface disorders include ocular allergy, dry eye disease, ocular manifestation of graft versus host disease and other inflammatory and/or infectious diseases of the (e.g., anterior) surface of the eye(s). Said compositions and methods comprise keratolytic conjugates which demonstrate immunological, keratolytic, anti-inflammatory, and/or other desirable activities. Topical administration of said compositions to the eyelid margin or surrounding areas provides therapeutic benefit to patients suffering from ocular surface disorders.

### CROSS-REFERENCE

This application claims the benefit of priority to U.S. Provisional Application No. 63/257,855, filed on Oct. 20, 2021, the entirety of which is incorporated herein by reference.

### BACKGROUND OF THE DISCLOSURE

Dry eye disease (DED) is reported to have a global prevalence of 5 to 50%. While there are some treatments available for treating DED, DED is generally a symptom of a variety of underlying diseases, so effective treatments for individual patients can remain elusive.

### SUMMARY OF THE DISCLOSURE

Provided in certain embodiments herein are compounds, such as compounds suitable for (e.g., simultaneously) targeting multiple underlying etiologies of symptomatic disease, such as dry eye disease (DED). In some instances, such compounds are suitable for treating multiple underlying etiologies of disease, which can be useful in providing (1) better outcomes for individuals suffering from disease as a result of multiple etiologies, and (2) improving disease response in a class of patients who may have different causes of disease. Also provided in certain embodiments herein are pharmaceutical (e.g., dermal and/or ophthalmic) compositions comprising such compounds, and methods of treating disease by administering a compound or composition provided herein to an individual (e.g., an individual suffering from such a disease). In specific embodiments, the disease treated by any method provided herein is an ocular, periocular, or dermal disorder, such as dry eye disease (DED).

In some instances, provided herein is a compound that delivers a therapeutically effective amount of (e.g., a free form of) an immunomodulator, such as an immunomodulator described herein (e.g., which treats a dysregulated immunoreaction), and/or (e.g., a free form of) at least one keratolytic agent, such as a keratolytic agent described herein. In some instances, the free form of the immunomodulator is selected from the group consisting of cilomilast, ruxolitinib, ritlecitinib, tofacitinib, oclacitinib, methotrexate, loteprednol, and tacrolimus.

Provided in some embodiments herein is a compound, or a pharmaceutically acceptable salt or solvate thereof, having a structure represented by Formula (A):

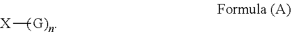

In some embodiments, X is an immunomodulator radical (e.g., an immunostimulant radical or an immunosuppressant radical). In some embodiments, n is 1-3. In some embodiments, each G independently comprises at least one radical of a keratolytic agent. In some embodiments, each G independently comprises at least one linker and at least one radical of a keratolytic agent.

In some embodiments, X is an immunomodulator radical.

In some embodiments, X is an immunosuppressant radical.

In some embodiments, X is an immunostimulant radical.

In some embodiments, X (e.g., in its free form) is an anti-inflammatory agent.

In some embodiments, X (e.g., in its free form) is an immunomodulator (e.g., an immunosuppressant or an immunostimulant) and an anti-inflammatory agent.

In some embodiments, X (e.g., in its free form) modulates an immune response (e.g., a dysregulated immunoreaction) in an individual (e.g., in need thereof), such as, in an individual suffering from any ocular or dermal disease or disorder described elsewhere herein. In some embodiments, X (e.g., in its free form) reduces inflammation in an individual (e.g., in need thereof), such as, in an individual suffering from any ocular or dermal disease or disorder described elsewhere herein. In some embodiments, X (e.g., in its free form) modulates an immune response (e.g., a dysregulated immunoreaction) and reduces inflammation in an individual (e.g., in need thereof), such as, in an individual suffering from any ocular or dermal disease or disorder described elsewhere herein.

In some embodiments, each linker is the same.

In some embodiments, each linker is different.

In some embodiments, each linker is independently a bond, substituted or unsubstituted (e.g., branched or straight) alkyl (alkylenyl), or substituted or unsubstituted (e.g., branched or straight) heteroalkyl (heteroalkylenyl).

In some embodiments, each linker is a bond.

In some embodiments, each linker is independently substituted or unsubstituted (e.g., branched or straight) alkyl (alkylenyl).

In some embodiments, each linker is independently a bond, —CH(CH₃)—, or —CH₂—. In some embodiments, each linker is independently —CH(CH₃)— or —CH₂—. In some embodiments, each linker is —CH(CH₃)—. In some embodiments, each linker is —CH₂—.

In some embodiments, each linker is independently substituted or unsubstituted (e.g., branched or straight) heteroalkyl (heteroalkylenyl).

In some embodiments, each radical of a keratolytic agent is a radical of the same keratolytic agent.

In some embodiments, each radical of a keratolytic agent is a radical of a different keratolytic agent.

In some embodiments, each radical of a keratolytic agent comprises one or more keratolytic group.

Provided in some embodiments herein is a compound, or a pharmaceutically acceptable salt or solvate thereof, having a structure represented by Formula (A-I):

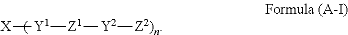

In some embodiments, X is an immunomodulator radical (e.g., an immunostimulant radical or an immunosuppressant radical). In some embodiments, n is 1-3. In some embodiments, each Y¹ and Y² is independently a linker. In some embodiments, each Z¹ and Z² is independently a radical of a keratolytic agent.

In some embodiments, each Y¹ is the same. In some embodiments, each Y² is the same. In some embodiments, each Y¹ and Y² are the same.

In some embodiments, each Y¹ is different. In some embodiments, each Y² is different. In some embodiments, each Y¹ and Y² are different.

In some embodiments, each Y¹ is independently a bond, substituted or unsubstituted (e.g., branched or straight) alkyl (alkylenyl), or substituted or unsubstituted (e.g., branched or straight) heteroalkyl (heteroalkylenyl).

In some embodiments, each Y¹ is a bond.

In some embodiments, each Y¹ is independently substituted or unsubstituted (e.g., branched or straight) alkyl (alkylenyl).

In some embodiments, each Y¹ is independently a bond, —CH(CH₃)—, or —CH₂—. In some embodiments, each Y¹ is independently —CH(CH₃)— or —CH₂—. In some embodiments, each Y¹ is —CH(CH₃)—. In some embodiments, each Y¹ is —CH₂—.

In some embodiments, each Y¹ is independently substituted or unsubstituted (e.g., branched or straight) heteroalkyl (heteroalkylenyl).

In some embodiments, each Y² is independently a bond, substituted or unsubstituted (e.g., branched or straight) alkyl (alkylenyl), or substituted or unsubstituted (e.g., branched or straight) heteroalkyl (heteroalkylenyl).

In some embodiments, each Y² is a bond.

In some embodiments, each Y² is independently substituted or unsubstituted (e.g., branched or straight) alkyl (alkylenyl).

In some embodiments, each Y² is independently a bond, —CH(CH₃)—, or —CH₂—. In some embodiments, each Y² is independently —CH(CH₃)— or —CH₂—. In some embodiments, each Y² is —CH(CH₃)—. In some embodiments, each Y² is —CH₂—.

In some embodiments, each Y² is independently substituted or unsubstituted (e.g., branched or straight) heteroalkyl (heteroalkylenyl).

In some embodiments, each Y¹ is a bond and each Y² is independently —CH(CH₃)— or —CH₂—. In some embodiments, Y¹ is a bond and each Y² is —CH(CH₃)—. In some embodiments, Y¹ is a bond and each Y² is —CH₂—.

In some embodiments, each Z¹ is a radical of the same keratolytic agent. In some embodiments, each Z² is a radical of the same keratolytic agent. In some embodiments, each Z¹ and Z² are a radical of the same keratolytic agent.

In some embodiments, each Z¹ is a radical of a different keratolytic agent. In some embodiments, each Z² is a radical of a different keratolytic agent. In some embodiments, each Z¹ and Z² are a radical of a different keratolytic agent.

In some embodiments, each Z¹ comprises one or more keratolytic group.

In some embodiments, each Z² comprises one or more keratolytic group.

Provided in some embodiments herein is a compound, or a pharmaceutically acceptable salt or solvate thereof, having a structure represented by Formula (A-II):

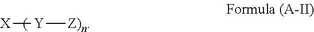

In some embodiments, X is an immunomodulator radical (e.g., an immunostimulant radical or an immunosuppressant radical). In some embodiments, n is 1-3. In some embodiments, each Y is independently a linker. In some embodiments, each Z is independently a radical of a keratolytic agent.

In some embodiments, each Y is the same.

In some embodiments, each Y is different.

In some embodiments, each Y is independently a bond, substituted or unsubstituted (e.g., branched or straight) alkyl (alkylenyl), or substituted or unsubstituted (e.g., branched or straight) heteroalkyl (heteroalkylenyl).

In some embodiments, each Y is a bond.

In some embodiments, each Y is independently substituted or unsubstituted (e.g., branched or straight) alkyl (alkylenyl).

In some embodiments, each Y is independently a bond, —CH(CH₃)—, or —CH₂—. In some embodiments, each Y is independently —CH(CH₃)— or —CH₂—. In some embodiments, each Y is —CH(CH₃)—. In some embodiments, each Y is —CH₂—.

In some embodiments, each Y is independently substituted or unsubstituted (e.g., branched or straight) heteroalkyl (heteroalkylenyl).

In some embodiments, each Z is a radical of the same keratolytic agent.

In some embodiments, each Z is a radical of a different keratolytic agent.

In some embodiments, each Z comprises one or more keratolytic group.

In some embodiments, each radical of a keratolytic agent (e.g., Z, Z¹, or Z²) comprises one or more group, each group being independently selected from the group consisting of —O—, oxo, substituted or unsubstituted (e.g., branched or straight) alkyl (alkylenyl), substituted or unsubstituted (e.g., branched or straight) heteroalkyl (heteroalkylenyl), substituted or unsubstituted alkoxyl, substituted or unsubstituted cycloalkyl, and substituted or unsubstituted heterocyclyl.

In some embodiments, each radical of a keratolytic agent (e.g., Z, Z¹, or Z²) is (e.g., branched or straight) alkyl (alkylenyl) substituted with one or more substituent, each substituent being independently selected from the group consisting of oxo, hydroxy, alkyl, alkoxy, and substituted or unsubstituted heterocyclyl.

In some embodiments, each radical of a keratolytic agent (e.g., Z, Z¹, or Z²) is (e.g., branched or straight) heteroalkyl (heteroalkylenyl) substituted with one or more substituent, each substituent being independently selected from the group consisting of oxo, alkyl, thioalkyl, and substituted or unsubstituted heterocyclyl.

In some embodiments, Z¹ is straight alkyl (alkylenyl) substituted with one or more substituent, each substituent being independently selected from the group consisting of oxo and hydroxy and Z² is straight alkyl (alkylenyl) substituted with one or more substituent, each substituent being independently selected from the group consisting of oxo and substituted or unsubstituted heterocyclyl.

In some embodiments, n is 1-3.

In some embodiments, n is 1.

In some embodiments, n is 2.

In some embodiments, n is 3.

In some embodiments, Y¹ is a bond, Y² is —CH(CH₃)— or —CH₂—, Z¹ is

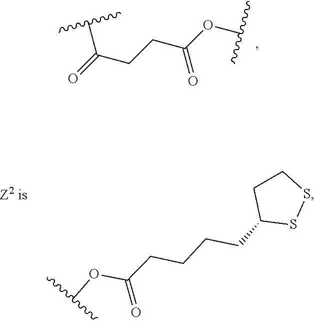 [SMILES: *OC(=O)CCC(*)=O ; *OC(=O)CCCC[C@@H]1CCSS1]

and n is 1.

In some embodiments, X is selected from the group consisting of a radical of a phosphodiesterase (PDE) inhibitor, a radical of a Janus kinase (JAK) inhibitor, a radical of a folate reductase inhibitor, a radical of a steroid, and a radical of a calcineurin inhibitor.

In some embodiments, X is a radical of a PDE inhibitor. In some embodiments, X is a radical of a selective PDE inhibitor. In some embodiments, X is a radical of a PDE4 inhibitor. In some embodiments, X is a radical of a selective PDE4 inhibitor. In some embodiments, X is a cilomilast, or a derivative thereof, radical. In some embodiments, X is a cilomilast radical.

In some embodiments, X is a radical of a JAK inhibitor. In some embodiments, X is a radical of a selective JAK inhibitor. In some embodiments, X is a radical of a JAK1, JAK2, and/or JAK3 inhibitor. In some embodiments, X is a radical of a JAK1 and/or JAK2 inhibitor. In some embodiments, X is a radical of a JAK1 and/or JAK3 inhibitor. In some embodiments, X is a radical of a JAK2 and/or JAK3 inhibitor. In some embodiments, X is a radical of a JAK1 inhibitor. In some embodiments, X is a radical of a JAK2 inhibitor. In some embodiments, X is a radical of a JAK3 inhibitor. In some embodiments, X is a radical of a selective JAK1 inhibitor. In some embodiments, X is a radical of a selective JAK2 inhibitor. In some embodiments, X is a radical of a selective JAK3 inhibitor. In some embodiments, X is selected from the group consisting of a ruxolitinib radical, a tofacitinib radical, an oclacitinib radical, and a ritlecitinib radical. In some embodiments, X is a ruxolitinib, or a derivative thereof, radical. In some embodiments, X is a ruxolitinib radical. In some embodiments, X is a tofacitinib, or a derivative thereof, radical. In some embodiments, X is a tofacitinib radical. In some embodiments, X is an oclacitinib, or a derivative thereof, radical. In some embodiments, X is an oclacitinib radical. In some embodiments, X is a ritlecitinib, or a derivative thereof, radical. In some embodiments, X is a ritlecitinib radical.

In some embodiments, X is a radical of a folate reductase inhibitor. In some embodiments, X is a methotrexate, or a derivative thereof, radical. In some embodiments, X is a methotrexate radical.

In some embodiments, X is a radical of a steroid. In some embodiments, X is a corticosteroid radical. In some embodiments, X is a glucocorticoid radical. In some embodiments, X is a loteprednol, or a derivative thereof, radical. In some embodiments, X is a loteprednol radical.

In some embodiments, X is a radical of a calcineurin inhibitor. In some embodiments, X is a tacrolimus, or a derivative thereof, radical. In some embodiments, X is a tacrolimus radical.

In some embodiments, X has a structure represented by Formula (I-A):

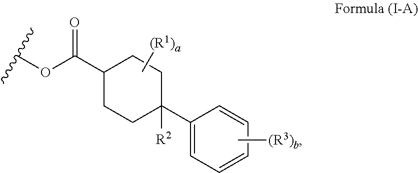

In some embodiments, each R¹ is independently halogen, alkyl, or —CN.

In some embodiments, a is 0.

In some embodiments, R² is hydrogen, —CN, halogen, or alkyl. In some embodiments, R² is hydrogen. In some embodiments, R² is —CN. In some embodiments, R² is halogen. In some embodiments, R² is alkyl.

In some embodiments, b is 0-9. In some embodiments, b is 1-4. In some embodiments, b is 2.

In some embodiments, each R³ is independently —OR^{a}, alkyl, heteroalkyl, cycloalkyl, or heterocyclyl. In some embodiments, each R³ is independently alkyl. In some embodiments, each R³ is independently heteroalkyl. In some embodiments, each R³ is independently cycloalkyl. In some embodiments, each R³ is independently heterocyclyl. In some embodiments, each R³ is independently —OR^{a}.

In some embodiments, b is 2 and each R³ is independently —OR^{a}.

In some embodiments, each R^{a} is independently hydrogen, alkyl, cycloalkyl, or heterocyclyl. In some embodiments, each R^{a} is independently alkyl or cycloalkyl. In some embodiments, each R^{a} is hydrogen. In some embodiments, each R^{a} is independently alkyl. In some embodiments, each R^{a} is independently cycloalkyl. In some embodiments, each R^{a} is independently heterocyclyl.

In some embodiments, each R³ is independently —OMe or —OC₃-C₅ cycloalkyl. In some embodiments, each R³ is independently —OMe or —O-cyclopentyl.

In some embodiments, b is 2 and each R³ is independently —OMe or —OC₃-C₅ cycloalkyl.

In some embodiments, b is 2 and each R³ is independently —OMe or —O-cyclopentyl.

In some embodiments, a is 0, R² is —CN, b is 2, and each R³ is independently —OMe or —OC₃-C₅ cycloalkyl.

In some embodiments, X has a structure represented by Formula (I-AA):

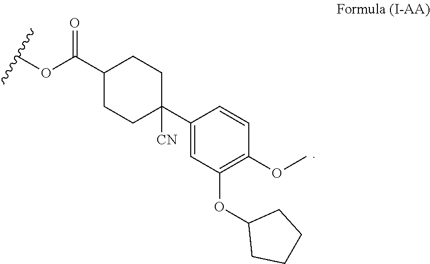 [SMILES: *OC(=O)C1CCC(C#N)(c2ccc(OC)c(OC3CCCC3)c2)CC1]

In some embodiments, X has a structure represented by Formula (I-B):

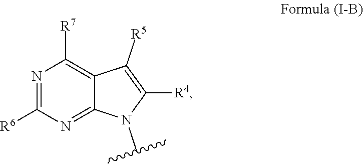

In some embodiments, R⁴ is hydrogen, halogen, or alkyl. In some embodiments, R⁴ is hydrogen. In some embodiments, R⁴ is halogen. In some embodiments, R⁴ is alkyl.

In some embodiments, R⁵ is hydrogen, halogen, or alkyl. In some embodiments, R⁵ is hydrogen. In some embodiments, R⁵ is halogen. In some embodiments, R⁵ is alkyl.

In some embodiments, R⁴ and R⁵ are hydrogen.

In some embodiments, R⁶ is hydrogen, halogen, or alkyl. In some embodiments, R⁶ is hydrogen. In some embodiments, R⁶ is halogen. In some embodiments, R⁶ is alkyl.

In some embodiments, R⁴—R⁶ are hydrogen.

In some embodiments, R⁷ is —NR^{b}R^{c} or optionally substituted heterocyclyl.

In some embodiments, R⁷ is optionally substituted heterocyclyl. In some embodiments, R⁷ is heterocyclyl substituted with alkyl substituted with CN and cycloalkyl.

In some embodiments, X has a structure represented by Formula (I-BA):

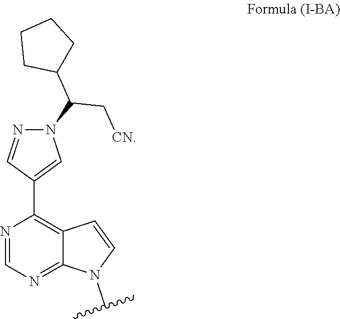

In some embodiments, R⁷ is —NR^{b}R^{c}.

In some embodiments, R^{b} and R^{c} are each independently hydrogen, alkyl, optionally substituted cycloalkyl, or optionally substituted heterocyclyl. In some embodiments, R^{b} and R^{c} are hydrogen. In some embodiments, R^{b} and R^{c} are each independently alkyl. In some embodiments, R^{b} and R^{c} are each independently optionally substituted cycloalkyl. In some embodiments, R^{b} and R^{c} are each independently optionally substituted heterocyclyl.

In some embodiments, R^{b} is hydrogen or C₁-C₃ alkyl.

In some embodiments, R^{b} is hydrogen.

In some embodiments, R^{b} is C₁-C₃ alkyl. In some embodiments, R^{b} is CH₃.

In some embodiments, R^{c} is substituted cycloalkyl or substituted heterocyclyl. In some embodiments, R^{c} is substituted cycloalkyl. In some embodiments, R^{c} is substituted heterocyclyl.

In some embodiments, R^{c} is cycloalkyl or heterocyclyl substituted with one or more substituent, each substituent being independently optionally substituted alkyl.

In some embodiments, R^{c} is cycloalkyl substituted with one or more substituent, each substituent being independently optionally substituted alkyl.

In some embodiments, R^{c} is cycloalkyl substituted with C₁-C₃ alkyl substituted with —SO₂NHCH₃.

In some embodiments, R^{c} is heterocyclyl substituted with one or more substituent, each substituent being independently optionally substituted alkyl.

In some embodiments, R^{c} is heterocyclyl substituted with unsubstituted C₁-C₃ alkyl and (unsaturated) C₁-C₃ alkyl substituted with oxo.

In some embodiments, R^{c} is heterocyclyl substituted with unsubstituted C₁-C₃ alkyl and C₁-C₃ alkyl substituted with oxo and —CN.

In some embodiments, R⁷ is —NR^{b}R^{c}, R^{b} is hydrogen, and R^{c} is heterocyclyl substituted with unsubstituted C₁-C₃ alkyl and (unsaturated) C₁-C₃ alkyl substituted with oxo.

In some embodiments, X has a structure represented by Formula (I-BB):

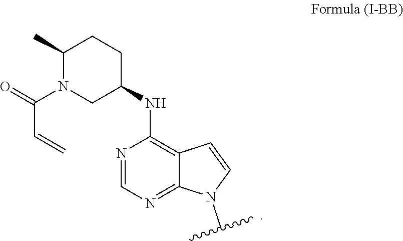 [SMILES: *n1ccc2c(N[C@@H]3CC[C@H](C)N(C(=O)C=C)C3)ncnc21]

In some embodiments, R⁷ is —NR^{b}R^{c}, R^{b} is CH₃, and R^{c} is heterocyclyl substituted with unsubstituted C₁-C₃ alkyl and C₁-C₃ alkyl substituted with oxo and —CN.

In some embodiments, X has a structure represented by Formula (I-BC):

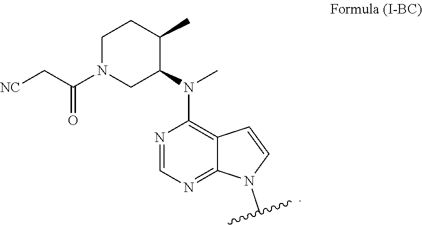 [SMILES: *n1ccc2c(N(C)[C@H]3CN(C(=O)CC#N)CC[C@H]3C)ncnc21]

In some embodiments, R⁷ is —NR^{b}R^{c}, R^{b} is CH₃, and R^{c} is cycloalkyl substituted with C₁-C₃ alkyl substituted with —SO₂NHCH₃.

In some embodiments, X has a structure represented by Formula (I-BD):

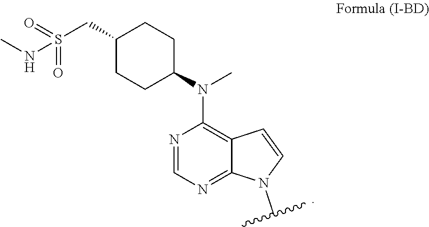 [SMILES: *n1ccc2c(N(C)[C@H]3CC[C@H](CS(=O)(=O)NC)CC3)ncnc21]

In some embodiments, X has a structure represented by Formula (I-C1):

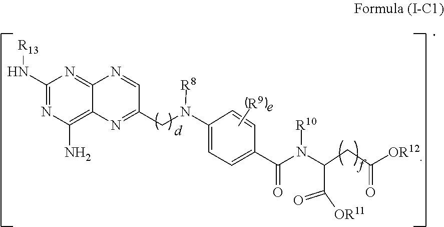

In some embodiments, X has a structure represented by Formula (I-C2):

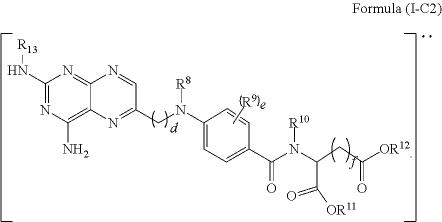

In some embodiments, X has a structure represented by Formula (I-C3):

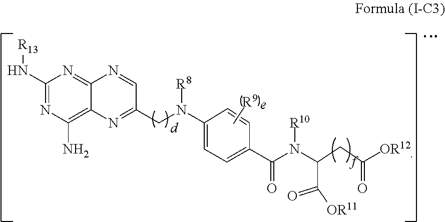

In some embodiments, R⁸ is hydrogen or alkyl. In some embodiments, R⁸ is hydrogen. In some embodiments, R⁸ is alkyl. In some embodiments, R⁸ is —CH₃.

In some embodiments, each R⁹ is independently halogen or alkyl. In some embodiments, each R⁹ is hydrogen. In some embodiments, each R⁹ is independently alkyl.

In some embodiments, R¹⁰ is hydrogen or alkyl. In some embodiments, R¹⁰ is hydrogen.

In some embodiments, R¹⁰ is alkyl.

In some embodiments, R¹¹ is a radical, hydrogen, or alkyl. In some embodiments, R¹¹ is a radical. In some embodiments, R¹¹ is hydrogen. In some embodiments, R¹¹ is alkyl.

In some embodiments, R¹² is a radical, hydrogen, or alkyl. In some embodiments, R¹² is a radical. In some embodiments, R¹² is hydrogen. In some embodiments, R¹² is alkyl.

In some embodiments, R¹¹ is a radical and R¹² is a radical.

In some embodiments, R¹³ is a radical or hydrogen. In some embodiments, R¹³ is a radical.

In some embodiments, R¹³ is hydrogen.

In some embodiments, R¹¹ and R¹² are a radical and R¹³ is hydrogen.

In some embodiments, R¹³ is a radical and R¹¹ and R¹² are hydrogen.

In some embodiments, d is 1-3. In some embodiments, d is 1. In some embodiments, d is 2. In some embodiments, d is 3.

In some embodiments, e is 0-4. In some embodiments, e is 0. In some embodiments, e is 1. In some embodiments, e is 2. In some embodiments, e is 3. In some embodiments, e is 4.

In some embodiments, f is 1-4. In some embodiments, f is 1. In some embodiments, f is 2.

In some embodiments, f is 3. In some embodiments, f is 4.

In some embodiments, R⁸ is —CH₃, R¹⁰ is hydrogen, R¹¹ and R¹² are a radical, R¹³ is hydrogen, e is 0, d is 1, and f is 2.

In some embodiments, X has a structure represented by Formula (I-CA):

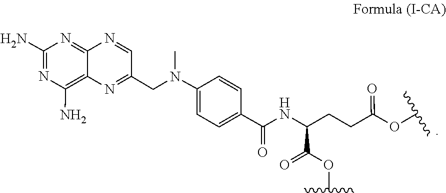 [SMILES: *OC(=O)CC[C@H](NC(=O)c1ccc(N(C)Cc2cnc3nc(N)nc(N)c3n2)cc1)C(=O)O*]

In some embodiments, R⁸ is —CH₃, R¹⁰ is hydrogen, R¹¹ and R¹² are hydrogen, R¹³ is a radical, e is 0, d is 1, and f is 2.

In some embodiments, X has a structure represented by Formula (I-CB):

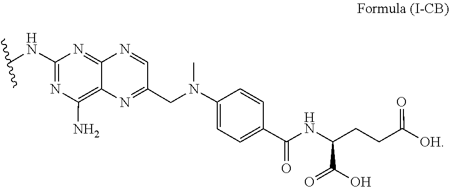 [SMILES: *Nc1nc(N)c2nc(CN(C)c3ccc(C(=O)N[C@@H](CCC(=O)O)C(=O)O)cc3)cnc2n1]

In some embodiments, X has a structure represented by Formula (I-D′):

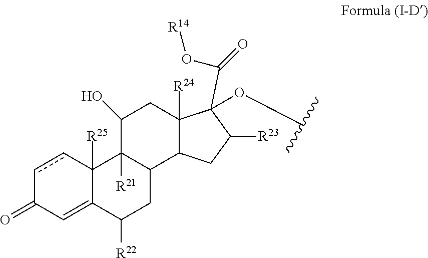

In some embodiments, [symbol] is a single bond or a double bond.

In some embodiments, R¹⁴ is hydrogen or optionally substituted alkyl (e.g., alkyl substituted with halo (e.g., chloro) or cyano).

In some embodiments, R²¹ is hydrogen, halogen, or alkyl. In some embodiments, R²¹ is hydrogen or halogen (e.g., fluoro).

In some embodiments, R²² is hydrogen, halogen, or alkyl. In some embodiments, R²² is hydrogen or halogen (e.g., fluoro).

In some embodiments, R²³ is hydrogen or alkyl. In some embodiments, R²³ is alkyl (e.g., methyl).

In some embodiments, R²⁴ is hydrogen or alkyl. In some embodiments, R²⁴ is alkyl (e.g., methyl).

In some embodiments, R²⁵ is hydrogen or alkyl. In some embodiments, R²⁵ is alkyl (e.g., methyl).

In some embodiments, X has a structure represented by Formula (I-D):

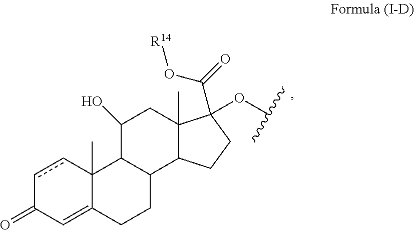

In some embodiments, [symbol] is a single bond or a double bond. In some embodiments, [symbol] is a single bond. In some embodiments, [symbol] is a double bond.

In some embodiments, R¹⁴ is hydrogen or optionally substituted alkyl.

In some embodiments, R¹⁴ is hydrogen.

In some embodiments, [symbol] is a double bond and R¹⁴ is hydrogen.

In some embodiments, R¹⁴ is optionally substituted alkyl. In some embodiments, R¹⁴ is alkyl substituted with halogen.

In some embodiments, R¹⁴ is hydrogen or alkyl substituted with halogen or cyano. In some embodiments, R¹⁴ is alkyl substituted with halogen. In some embodiments, R¹⁴ is alkyl substituted with chloro.

In some embodiments, [symbol] is a double bond and R¹⁴ is alkyl substituted with halogen. In some embodiments, [symbol] is a double bond and R¹⁴ is alkyl substituted with chloro.

In some embodiments, X has a structure represented by Formula (I-DA):

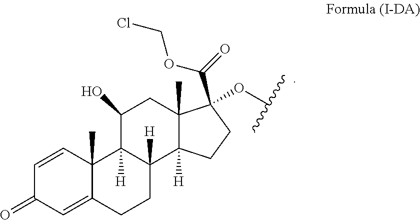

In some embodiments, X has a structure represented by Formula (I-DB):

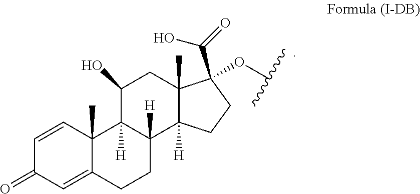

In some embodiments, R¹⁴ is alkyl substituted with cyano. In some embodiments, [symbol] is a double bond and R¹⁴ is alkyl substituted with cyano.

In some embodiments, X has a structure represented by Formula (I-DC):

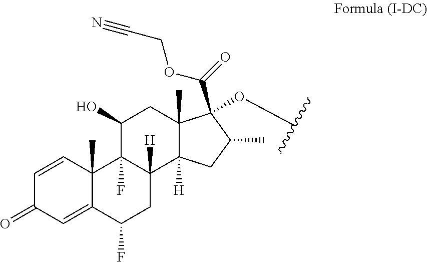

In some embodiments, X has a structure represented by Formula (I-E):

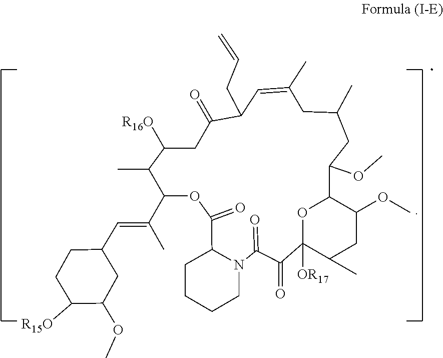

In some embodiments, X has a structure represented by Formula (I-E′):

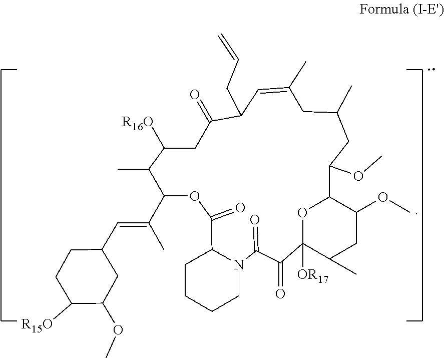

In some embodiments, R¹⁵, R¹⁶, and R¹⁷ are each independently selected from a radical, hydrogen, or alkyl. In some embodiments, R¹⁵, R¹⁶, and R¹⁷ are each independently a radical or hydrogen. In some embodiments, R¹⁵, R¹⁶, and R¹⁷ are each independently a radical or alkyl.

In some embodiments, R¹⁵ is a radical.

In some embodiments, R¹⁶ is a radical.

In some embodiments, R¹⁵ and R¹⁶ are a radical.

In some embodiments, R¹⁷ is a radical.

In some embodiments, R¹⁷ is hydrogen.

In some embodiments, R¹⁵ is a radical, R¹⁶ is hydrogen, and R¹⁷ is hydrogen.

In some embodiments, X has a structure represented by Formula (I-EA):

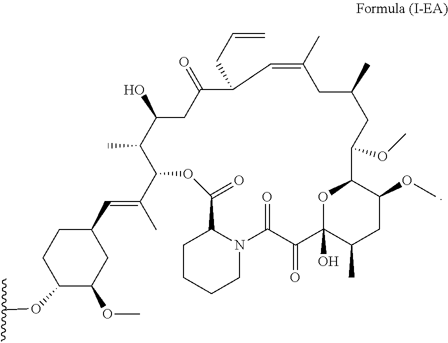

In some embodiments, R¹⁵ is hydrogen, R¹⁶ is a radical, and R¹⁷ is hydrogen.

In some embodiments, X has a structure represented by Formula (I-EB):

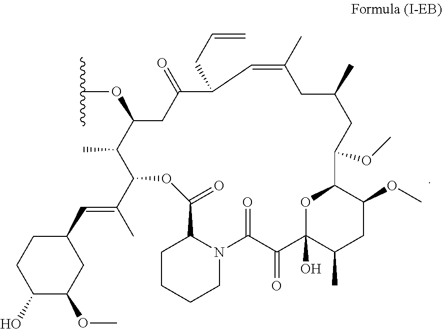

In some embodiments, R¹⁵ is a radical, R¹⁶ is a radical, and R¹⁷ is hydrogen.

In some embodiments, X has a structure represented by Formula (I-EC):

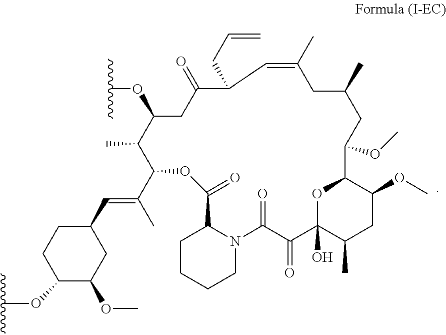

In some embodiments, each radical of a keratolytic agent (e.g., Z, Z¹, or Z²) has a structure represented by:

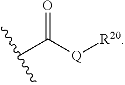

In some embodiments, Q is —O— or —(CR¹⁸R¹⁹)ₘ—.

In some embodiments, Q is —(CR¹⁹R¹⁹)ₘ—.

In some embodiments, Q is —O—.

In some embodiments, m is 1-6. In some embodiments, m is 1-4. In some embodiments, m is 1. In some embodiments, m is 2. In some embodiments, m is 3. In some embodiments, m is 4.

In some embodiments, each R¹⁸ and R¹⁹ is independently H, halo, alkyl, alkoxy, haloalkyl, or thioalkyl, or an adjacent R¹⁸ and R¹⁹ combine to the atoms to which they are attached to form an oxo.

In some embodiments, each R¹⁸ and R¹⁹ is independently H, halo, alkyl, alkoxy, haloalkyl, or thioalkyl. In some embodiments, each R¹⁸ and R¹⁹ is H. In some embodiments, each R¹⁸ and R¹⁹ is independently H or halo. In some embodiments, each R¹⁸ and R¹⁹ is independently H or alkyl. In some embodiments, each R¹⁸ and R¹⁹ is independently H or alkoxy. In some embodiments, each R¹⁸ and R¹⁹ is independently H or haloalkyl. In some embodiments, each R¹⁸ and R¹⁹ is independently H or thioalkyl.

In some embodiments, each R¹⁸ and R¹⁹ is independently H, C₁-C₆ alkyl, or C₁-C₃ thioalkyl. In some embodiments, each R¹⁸ and R¹⁹ is independently H or C₁-C₆ alkyl. In some embodiments, each R¹⁸ and R¹⁹ is independently H or C₁-C₃ thioalkyl.

In some embodiments, each R¹⁸ and R¹⁹ is independently H, CH₃, or CH₂SH. In some embodiments, each R¹⁸ and R¹⁹ is independently H or CH₃. In some embodiments, each R¹⁸ and R¹⁹ is independently H or CH₂SH.

In some embodiments, each R¹⁸ and R¹⁹ is H.

In some embodiments, an adjacent R¹⁸ and R¹⁹ combine to the atoms to which they are attached to form an oxo.

In some embodiments, Q is —CH₂—, —CH(CH₃)—, —(CH₂)₂C(═O)—, —CH₂C(CH₃)₂CH₂—, or —CH(CH₂SH)—. In some embodiments, Q is —CH₂—. In some embodiments, Q is —CH(CH₃)—. In some embodiments, Q is —(CH₂)₂C(═O)—. In some embodiments, Q is —CH₂C(CH₃)₂CH₂—. In some embodiments, Q is —CH(CH₂SH)—.

In some embodiments, R²⁰ is alkyl, heteroalkyl, heterocyclyl, alkoxy, or hydroxy, the alkyl, heteroalkyl, heterocyclyl, or alkoxy each independently being optionally substituted. In some embodiments, R²⁰ is optionally substituted alkyl. In some embodiments, R²⁰ is optionally substituted heteroalkyl. In some embodiments, R²⁰ is optionally substituted heterocyclyl. In some embodiments, R²⁰ is optionally substituted alkoxy. In some embodiments, R²⁰ is hydroxy.

In some embodiments, R²⁰ is dithiolanyl or dithiolanyl oxide. In some embodiments, R²⁰ is dithiolanyl. In some embodiments, R²⁰ is dithiolanyl oxide.

In some embodiments, R²⁰ is:

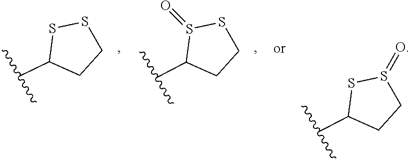 [SMILES: *C1CCSS1 ; *C1CCSS1=O ; *C1CCS(=O)S1]

In some embodiments, R²⁰ is:

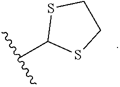 [SMILES: *C1SCCS1]

In some embodiments, R²⁰ is:

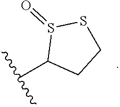 [SMILES: *C1CCSS1=O]

In some embodiments, R²⁰ is:

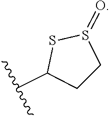 [SMILES: *C1CCS(=O)S1]

In some embodiments, R²⁰ is hydroxy, optionally substituted C₁-C₆ alkyl, optionally substituted C₁-C₆ alkoxy, or optionally substituted C₁-C₆ heteroalkyl. In some embodiments, R²⁰ is hydroxy. In some embodiments, R²⁰ is optionally substituted C₁-C₆ alkyl. In some embodiments, R²⁰ is optionally substituted C₁-C₆ alkoxy. In some embodiments, R²⁰ is optionally substituted C₁-C₆ heteroalkyl.

In some embodiments, R²⁰ is methyl, ethyl, propyl, isopropyl, butyl, or tert-butyl. In some embodiments, R²⁰ is methyl, ethyl, propyl, or isopropyl. In some embodiments, R²⁰ is methyl. In some embodiments, R²⁰ is ethyl. In some embodiments, R²⁰ is propyl. In some embodiments, R²⁰ is isopropyl.

In some embodiments, R²⁰ is CH₃, hydroxy, —O(C₁-C₃ alkoxy), or substituted C₁-C₆ heteroalkyl. In some embodiments, R²⁰ is CH₃. In some embodiments, R²⁰ is hydroxy. In some embodiments, R²⁰ is —O(C₁-C₃ alkoxy). In some embodiments, R²⁰ is substituted C₁-C₆ heteroalkyl. In some embodiments, R²⁰ is C₁-C₆ heteroalkyl substituted with CH₃, oxo, and dithiolanyl or dithiolanyl oxide. In some embodiments, R²⁰ is C₁-C₆ heteroalkyl substituted with CH₃, oxo, and dithiolanyl. In some embodiments, R²⁰ is C₁-C₆ heteroalkyl substituted with CH₃, oxo, and dithiolanyl oxide.

In some embodiments, R²⁰ is —OH, —CH₃, —OCH₃, —OCH₂CH₃, —NH(C═O)CH₃,

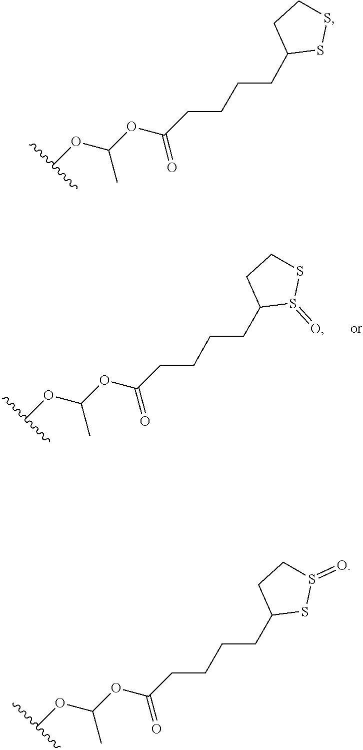 [SMILES: *OC(C)OC(=O)CCCCC1CCSS1 ; *OC(C)OC(=O)CCCCC1CCSS1=O ; *OC(C)OC(=O)CCCCC1CCS(=O)S1]

In some embodiments, Q is —(CR¹⁸R¹⁹)ₘ—, m is 1-4, R¹⁸ and R¹⁹ are each independently H or C₁-C₆ alkyl, and R²⁰ is optionally substituted heterocyclyl.

In some embodiments, Q is —CH₂—, —CH(CH₃)—, —(CH₂)₂C(═O)—, —CH₂C(CH₃)₂CH₂—, or —CH(CH₂SH)— and R²⁰ is hydroxy, optionally substituted C₁-C₆ alkyl, optionally substituted C₁-C₆ alkoxy, or optionally substituted C₁-C₆ heteroalkyl.

In some embodiments, Q is —O— and R²⁰ is optionally substituted C₁-C₆ alkyl.

In some embodiments, a compound, or a pharmaceutically acceptable salt or solvate thereof, is provided elsewhere herein, such as, for example, in Table 1, Table 2, Table 3, Table 4, Table 5, Table 6, Table 7, Table 8, or Table 9.

In some embodiments, provided herein is a pharmaceutical composition comprising any compound provided herein, such as a compound represented by any structure herein, such as, for example, Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, or a pharmaceutically acceptable salt thereof, and at least one pharmaceutically acceptable excipient.

In some embodiments, the pharmaceutical composition is suitable for topical administration (e.g., to the skin and/or (e.g., in or around) the eye). In some embodiments, the pharmaceutical composition is suitable for ophthalmic administration. In some embodiments, the pharmaceutical composition is suitable for topical ophthalmic administration. In some embodiments, (e.g., topical) ophthalmic administration is administration in and/or around the eye, such as to the eyelid margin. In some embodiments, topical ophthalmic administration is administration to the ocular surface and the inner surface to the eyelid. In some embodiments, (e.g., topical) ophthalmic administration is administration to the eyelid margin.

In some embodiments, a compound or a pharmaceutical composition comprising any compound provided herein, such as a compound of any one of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, or a pharmaceutically acceptable salt thereof, is substantially hydrolytically stable (e.g., stable in an aqueous composition (e.g., solution), such as a buffer solution or ophthalmically-acceptable (aqueous) composition). In some embodiments, the compound or the pharmaceutical composition is formulated in an aqueous vehicle. In some embodiments, the compound or the pharmaceutical composition is formulated and stored in an aqueous vehicle. In some instances, compositions or formulations provided herein are chemically and/or physically stable in an aqueous composition.

In some embodiments, a compound provided herein, such as a compound of any one of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, or a pharmaceutically acceptable salt thereof, is reduced to one or more keratolytic agent (e.g., a free form of a radical of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, such as wherein R is a negative charge or H) and/or hydrolyzed to an active pharmaceutical agent (e.g., a free form of a radical of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, such as wherein R is a negative charge or H). In some embodiments, the compound or pharmaceutical composition is reduced to one or more keratolytic agent in an ocular space. In some embodiments, the compound or pharmaceutical composition is reduced to one or more keratolytic agent by a reductase in an ocular space.

In some embodiments, a compound provided herein, such as a compound of any one of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, or a pharmaceutically acceptable salt thereof, is hydrolyzed to an active pharmaceutical agent (e.g., a free form of a radical of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, such as wherein R is a negative charge or H) and a keratolytic agent. In some embodiments, the compound or pharmaceutical composition is hydrolyzed to an active pharmaceutical agent and a keratolytic agent in an ocular space. In some embodiments, the compound or pharmaceutical composition is hydrolyzed to an active pharmaceutical agent and a keratolytic agent by an esterase in an ocular space.

In some embodiments, the active pharmaceutical agent is an immunomodulator (e.g., immunostimulant or immunosuppressant) and/or an anti-inflammatory agent. In some embodiments, the active pharmaceutical agent is an immunomodulator (e.g., immunostimulant or immunosuppressant). In some embodiments, the active pharmaceutical agent is an anti-inflammatory agent. In some embodiments the immunomodulator and/or anti-inflammatory agent is cilomilast, ruxolitinib, tofacitinib, oclacitinib, ritlecitinib, methotrexate (or a derivative thereof), loteprednol (or a derivative thereof), or tacrolimus. In some embodiments, the active keratolytic agent comprises a carboxylic acid, a thiol (—SH), and/or a thioether (S-S). In some embodiments, the keratolytic agent is a carboxylic acid. In some embodiments, the carboxylic acid is selected from the group consisting of acetic acid, glycolic acid, lactic acid, lipoic acid, pivalic acid, isobutryic acid, butyric acid, propionic acid, formic acid, and carbonic acid. In some embodiments, the active keratolytic agent is a thiol (—SH). In some embodiments, the active keratolytic agent is a thioether (S-S).

In some embodiments, a compound or a pharmaceutical composition comprising any compound provided herein, such as a compound of any one of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, or a pharmaceutically acceptable salt thereof. In certain embodiments, the composition further comprises an amount of a free form of a radical of any of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, or the like (such as wherein the free form is the radical, wherein R is a negative charge or an H). In some embodiments, a composition provided herein comprises a (e.g., weight or molar) ratio of a compound provided herein to a free form of a radical of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, or a pharmaceutically acceptable salt thereof (e.g., wherein R is a negative charge or an H) is about 1:99 to about 100:0 (e.g., the amount of the free form of the radical relative to the overall amount of free form of the radical plus the conjugate is between 0% (weight or molar) and 99%). In some embodiments, the relative amount of the free form of the radical is 0% to about 50%, such 0% to about 20%, 0% to about 10%, about 0.1% to about 10%, about 0.1% to about 5%, less than 5%, less than 2.5%, less than 2%, or the like (percentages being weight/weight or mole/mole percentages). In some instances, such aqueous compositions are pre-manufactured or are manufactured at the time of application in order to maintain high concentrations of the compound relative to the free form of a radical thereof. In some embodiments, such concentrations of the compound are present in the composition for at least 45 minutes in an aqueous composition (such as in an aqueous composition, e.g., a HEPES buffer, such as under the conditions described herein, such as in Tables 11 and 12). Tables 11 and 12 of the Examples illustrate good stability of the compositions provided herein and such recitations are incorporated in the disclosure hereof. Further, in some instances, compounds provided herein release free form of a radical of a compound of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9 (e.g., wherein R is a negative charge or H), such as when administered to an individual (e.g., ocular (e.g., peri-ocular) or dermatological administration). In more specific instances, when administered to an individual at a location with esterases and/or reductases present, rapid release of active (free) forms of a radical of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, (e.g., wherein R is a negative charge or H) (and, a keratolytic agent and/or agent that further produces active keratolytic agent(s) (e.g., by further hydrolysis and/or reduction thereof)).

In some embodiments, provided herein a compound or a pharmaceutical composition comprising any compound provided herein, such as a compound of any one of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, or a pharmaceutically acceptable salt thereof, has keratolytic effects (e.g., reduces disulfide (S—S) bonds) (e.g., in any environment provided herein).

In some embodiments, the dermal, ocular, and/or periocular disorders treated by administering a composition or compound provided herein are disorders that have multifactorial etiologies and/or interactions, such as an immunological disorder. In some instances, a disorder treated according to any method provided herein is a disorder caused by a dysfunction of the immune system, such as involving one or more dysregulated immunological pathway in an individual. In some embodiments, provided herein are compounds (and compositions comprising such compounds) that have multifunctional efficacies, such as when administered to the skin and/or in or around the eye (e.g., to the ocular surface, the eyelid, such as the eyelid margin or the inner surface of the eyelid).

In some embodiments, provided herein is a method of treating an immunological disease or disorder (e.g., an autoimmune and/or an inflammatory disease or disorder) by administering (e.g., ocular, periocular, dermal) (e.g., therapeutically effective amount of) a compound or composition provided herein to an individual (e.g., in need thereof). In some instances, the immunological disease or disorder is, at least partially, mediated by an immunoreaction in the individual (e.g., in need thereof). In some instances, the immunoreaction is an immunological reaction in the individual (e.g., in need thereof) between an antigen and an antibody and/or a T cell sensitized for cell-mediated immunity. In some instances, the immunoreaction includes dysregulation of one or more immunological reaction and/or response in the individual (e.g., in need thereof). In some embodiments, provided herein is a method of treating a (e.g., dysregulated) immunoreaction, inflammation, and/or hyperkeratosis (e.g., of the eye or skin). In some embodiments, provided herein is a method of treating a dysregulated immunoreaction and/or hyperkeratosis (e.g., of the eye or skin). In some embodiments, provided herein is a method of treating inflammation and/or hyperkeratosis (e.g., of the eye or skin).

In certain embodiments, provided herein are methods of treating ocular (or dermatological) disorders associated with keratosis (e.g., lid keratosis, surface ocular keratosis, and/or gland blockage—such as in MGD, an ocular allergy (e.g., keratoconjunctivitis (e.g., atopic keratoconjunctivitis (AKC) or vernal keratoconjunctivitis (VKC))), dry eye disease, or the like), microbial infiltration/infection (e.g., bacterial infiltration/infection), improper immunomodulation (e.g., dysregulated immunoreaction(s)), and/or inflammation (such as inflammation associated keratosis or not associated with keratosis, such as an ocular manifestation of graft versus host disease). In certain instances, disorders of the skin and/or eye (and/or surround tissue/skin) are difficult to differentially diagnose and/or have multiple etiologies. For example, in some instances, it can be difficult to distinguish between ocular disorders that involve (1) inflammation only, (2) improper immunomodulation only, (3) inflammation and/or improper immunomodulation associated with keratolytic activity, (4) inflammation and/or improper immunomodulation associated with both keratolytic activity (e.g., inducing keratosis) and microbial infiltration, (5) keratolytic activity, but not inflammation, improper immunomodulation, and/or microbial infiltration, or various other combinations. In some instances, compounds and compositions provided herein can be used in such ocular and/or dermatological indications without the need for differential diagnosis (which can be difficult, e.g., because of similar symptom scores, etc.).

Further, many ocular and/or dermatological disorders involve multiple etiologies, such as improper immunomodulation, inflammation, microbial infiltration, keratolytic activity, or various combinations thereof. As a result, therapeutic agents, such as those described herein, that target multiple etiologies are beneficial in providing therapeutic efficacy, such as by targeting both an underlying condition (e.g., a dysregulated immunoreaction, keratolytic activity and/or microbial infiltration) and a symptom, such as inflammation or dry eye.

As such, provided herein are compounds, compositions, methods, and formulations for the treatment of ocular (e.g., periocular) or dermatological disorders, such as those having multifactorial etiologies (e.g., dysregulated immunoreaction(s), inflammation, keratosis, microbial infiltration/infection, or the like).

In specific embodiments, ocular disorders include, by way of non-limiting example, surface disorders, such as MGD, dry eye and associated inflammatory and bacterial disease, an (e.g., severe) ocular allergy (e.g., keratoconjunctivitis (e.g., atopic keratoconjunctivitis (AKC) or vernal keratoconjunctivitis (VKC))), a (e.g., inflammatory and/or aqueous) dry eye disease, and an ocular manifestation of graft versus host disease (ocular GVHD).

In some embodiments, the ocular disorder is a periocular disorder. In some embodiments, periocular disorders include, by way of non-limiting example, sties, blepharitis, chalazion, and dacryoadenitis.

In some embodiments, dermal disorders include, by way of non-limiting example, comedonal acne, hyperkeratosis, scleroderma, seborrheic dermatitis, atopic dermatitis, psoriasis, lichen planus, insect bites, intertrigo, pemphigus, and pityriasis rubra pilaris.

Provided in some embodiments herein is a method of treating a (e.g., dysregulated) immunoreaction, inflammation, and/or hyperkeratosis, the method comprising administering to an individual (e.g., in need thereof) any compound provided herein (e.g., of any Formula or Table provided herein) (e.g., in a therapeutically effective amount). In specific embodiments, the (e.g., dysregulated) immunoreaction, inflammation, and/or hyperkeratosis is a (e.g., dysregulated) immunoreaction, inflammation, and/or hyperkeratosis of the eye, periocular structures (e.g., eyelid), and/or skin.

Provided in some embodiments herein is a method of treating a dermatological or an ophthalmic disease or disorder in an individual in need of thereof, comprising administering to the individual in need thereof a composition comprising any compound provided herein, such as a compound represented by any structure herein, such as, for example, Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9, or a pharmaceutically acceptable salt thereof. In some embodiments, the dermatological or ophthalmic disease or disorder is a (e.g., dysregulated) immunoreaction, inflammation, and/or hyperkeratosis of the eyes or skin (e.g., the ocular surface).

In some embodiments, the dermatological or ophthalmic disease or disorder is selected from the group consisting of meibomian gland dysfunction (MGD), (e.g., inflammatory and/or aqueous) dry eye disease (DED), ocular manifestations of graft versus host disease (ocular GVHD), Cornelia de Lange Syndrome, evaporative eye disease, aqueous deficiency dry eye, blepharitis, seborrheic blepharitis, and an ocular allergy (e.g., a severe ocular allergy), such as, vernal keratoconjunctivitis, atopic keratoconjunctivitis. In some embodiments, the dermatological or ophthalmic disease or disorder is inflammation or hyperkeratosis (e.g., of the eyes or skin), such as, for example, meibomian gland dysfunction (MGD), dry eye disease (DED), ocular manifestations of graft versus host disease, vernal keratoconjunctivitis, atopic keratoconjunctivitis, Cornelia de Lange Syndrome, evaporative eye disease, aqueous deficiency dry eye, blepharitis, seborrheic blepharitis, or any combination thereof.

In some embodiments, the dermatological or ophthalmic disease or disorder is selected from the group consisting of an ocular allergy, a dry eye disease, or an ocular manifestation of graft versus host disease (ocular GVHD).

In some embodiments, the dermatological or ophthalmic disease or disorder is an ocular allergy. In some embodiments, the dermatological or ophthalmic disease or disorder is a severe ocular allergy. In some embodiments, the dermatological or ophthalmic disease or disorder is keratoconjunctivitis. In some embodiments, the dermatological or ophthalmic disease or disorder is atopic keratoconjunctivitis (AKC) or vernal keratoconjunctivitis (VKC). In some embodiments, the dermatological or ophthalmic disease or disorder is atopic keratoconjunctivitis (AKC). In some embodiments, the dermatological or ophthalmic disease or disorder is vernal keratoconjunctivitis (VKC).

In some embodiments, the dermatological or ophthalmic disease or disorder is a dry eye disease. In some embodiments, the dermatological or ophthalmic disease or disorder is an inflammatory and/or aqueous dry eye disease. In some embodiments, the dermatological or ophthalmic disease or disorder is an inflammatory dry eye disease. In some embodiments, the dermatological or ophthalmic disease or disorder is an aqueous dry eye disease.

In some embodiments, the dermatological or ophthalmic disease or disorder is an ocular manifestation of graft versus host disease (ocular GVHD).

In some embodiments, the ophthalmic disease or disorder is selected from dry eye, lid wiper epitheliopathy (LWE), contact lens discomfort (CLD), contact lens discomfort, dry eye syndrome, evaporative dry eye syndrome, aqueous deficiency dry eye syndrome, blepharitis, keratitis, meibomian gland dysfunction, conjunctivitis, lacrimal gland disorder, inflammation of the anterior surface of the eye, infection of the anterior surface of the eye, infection of the lid, demodex lid infestation, lid wiper epitheliopathy and autoimmune disorder of the anterior surface of the eye.

In some embodiments, provided herein is a method of treating an ocular (e.g., peri-ocular) or dermatological indication (e.g., associated with a (dysregulated) immunoreaction, keratolytic activity, inflammation, and/or microbial infiltration), the method comprising administering a therapeutically effective amount of a compound or composition provided herein. In some embodiments, a composition provided herein (e.g., used in a method provided herein) comprises a compound provided herein in a therapeutically effective amount (e.g., at a concentration effective to treat a (dysregulated) immunoreaction, keratosis/keratolytic activity, inflammation, and/or microbial infiltration), in the eye, surrounding tissue, or skin. In some embodiments, a (e.g., pharmaceutical and/or ophthalmic) composition provided herein comprises about 0.1 wt. % to about 10 wt. % of a compound provided herein.

In some embodiments, ocular and/or dermatological disorders include, for example, inflammatory conditions of the eyelids (e.g., hordeolum (stye), blepharitis, and chalazion), ocular surface (e.g., dry eye disease and anterior uveitis) and posterior eye (e.g., posterior and pan-uveitis), conditions of the peri-ocular glands (e.g., meibomian gland dysfunction (MGD)), allergic-type conditions, (e.g., eczema, atopic dermatitis, atopic keratoconjunctivitis refractory to topical steroid treatment, and vernal keratoconjunctivitis), surgical complications (e.g., corneal transplant rejection, post-corneal transplant glaucoma, cataracts secondary to phakic corneal transplant, fungal infections in keratoplasty patients, and post-LASIK dry eye and/or poor refractive outcomes), corneal conditions (e.g., inflammatory corneal ulceration, rheumatoid corneal ulcers, and Thygeson's superficial punctate keratitis), conjunctival conditions (e.g., iridocyclitis, ligneous conjunctivitis), ocular complications from systemic treatments and/or autoimmune diseases (e.g., pauciarticular juvenile rheumatoid arthritis, graft versus host disease, and sjogren's syndrome) and/or infectious disease of the anterior surface of the eye. In some embodiments, provided herein are compositions and methods for the treatment of ocular and periocular conditions that have multifactorial etiologies and interactions.

### INCORPORATION BY REFERENCE

All publications, patents, and patent applications mentioned in this specification are herein incorporated by reference for the specific purpose identified herein.

### DETAILED DESCRIPTION OF THE DISCLOSURE

### Certain Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

The terms “treat,” “treating,” or “treatment” as used herein, include reducing, alleviating, abating, ameliorating, managing, relieving, or lessening the symptoms associated with a disease, disease state, condition, or indication (e.g., provided herein) in either a chronic or acute therapeutic scenario. Also, treatment of a disease or disease state described herein includes the disclosure of use of such compound or composition for the treatment of such disease, disease state, disorder, or indication.

“Amino” refers to the —NH₂ radical.

“Cyano” refers to the —CN radical.

“Nitro” refers to the —NO₂ radical.

“Oxo” refers to the ═O radical.

“Hydroxyl” refers to the —OH radical.

“Alkyl” generally refers to a straight or branched hydrocarbon chain radical consisting solely of carbon and hydrogen atoms, such as having from one to fifteen carbon atoms (e.g., C₁-C₁₅ alkyl). Unless otherwise stated, alkyl is saturated or unsaturated (e.g., an alkenyl, which comprises at least one carbon-carbon double bond). Disclosures provided herein of an “alkyl” are intended to include independent recitations of a saturated “alkyl,” unless otherwise stated. Alkyl groups described herein are generally monovalent, but may also be divalent (which may also be described herein as “alkylene” or “alkylenyl” groups). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted.

Each recitation of “alkyl” provided herein, unless otherwise stated, includes a specific and explicit recitation of an unsaturated “alkyl” group. Similarly, unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)tOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is optionally substituted as described for “alkyl” groups.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like. Unless stated otherwise specifically in the specification, an alkylene chain is optionally substituted as described for alkyl groups herein.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” or “aryl-alkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkenyl” refers to a radical of the formula —R^{c}-carbocyclyl where R^{c} is an alkenylene chain as defined above. The alkenylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to fluoro, bromo, chloro, or iodo substituents.

“Haloalkyl” refers to an alkyl radical, as defined above, that is substituted by one or more halogen radicals, as defined above, for example, trihalomethyl, dihalomethyl, halomethyl, and the like. In some embodiments, the haloalkyl is a fluoroalkyl, such as, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, sulfur, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C18 heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁—C₄ heteroalkyl. Representative heteroalkyl groups include, but are not limited to —OCH₂OMe, or —CH₂CH₂OMe. In some embodiments, heteroalkyl includes alkoxy, alkoxyalkyl, alkylamino, alkylaminoalkyl, aminoalkyl, heterocycloalkyl, heterocycloalkyl, and heterocycloalkylalkyl, as defined herein. Unless stated otherwise specifically in the specification, a heteroalkyl group is optionally substituted as defined above for an alkyl group.

“Heteroalkylene” refers to a divalent heteroalkyl group defined above which links one part of the molecule to another part of the molecule. Unless stated specifically otherwise, a heteroalkylene is optionally substituted, as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl radical is saturated (i.e., containing single C—C bonds only) or unsaturated (e.g., containing one or more double bonds or triple bonds in the ring system). In some instances, the heterocyclyl radical is saturated (e.g., dithiolanyl or dithiolanyl oxide). In some instances, the heterocyclyl radical is saturated and substituted (e.g., dithiolanyl oxide). In some instances, the heterocyclyl radical is unsaturated. The heterocyclyl is attached to the rest of the molecule through any atom of the ring(s). Examples of such heterocyclyl radicals include, but are not limited to, dithiolanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals.

Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals.

Examples of such C-heterocyclyl radicals include, but are not limited to, 2-morpholinyl, 2- or 3-or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s).

Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

In general, optionally substituted groups are each independently substituted or unsubstituted. Each recitation of an optionally substituted group provided herein, unless otherwise stated, includes an independent and explicit recitation of both an unsubstituted group and a substituted group (e.g., substituted in certain embodiments, and unsubstituted in certain other embodiments). Unless otherwise stated, a substituted group provided herein (e.g., substituted alkyl) is substituted by one or more substituent, each substituent being independently selected from the group consisting of halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2), where each R^{a} is independently hydrogen, alkyl (e.g., optionally substituted with halogen, hydroxy, methoxy, or trifluoromethyl), fluoroalkyl, carbocyclyl (e.g., optionally substituted with halogen, hydroxy, methoxy, or trifluoromethyl), carbocyclylalkyl (e.g., optionally substituted with halogen, hydroxy, methoxy, or trifluoromethyl), aryl (e.g., optionally substituted with halogen, hydroxy, methoxy, or trifluoromethyl), aralkyl (e.g., optionally substituted with halogen, hydroxy, methoxy, or trifluoromethyl), heterocyclyl (e.g., optionally substituted with halogen, hydroxy, methoxy, or trifluoromethyl), heterocyclylalkyl (e.g., optionally substituted with halogen, hydroxy, methoxy, or trifluoromethyl), heteroaryl (e.g., optionally substituted with halogen, hydroxy, methoxy, or trifluoromethyl), or heteroarylalkyl (e.g., optionally substituted with halogen, hydroxy, methoxy, or trifluoromethyl).

“Pharmaceutically acceptable salt” includes both acid and base addition salts. A pharmaceutically acceptable salt of any one of the pharmacological agents described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like.

Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The term “keratinized obstruction,” as used herein, generally refers to a blockage of the meibomian gland, regardless of the location of the blockage. In some embodiments, the blockage is complete, whereas in other embodiments, the blockage is partial. Regardless of the degree of blockage, such keratinized obstruction leads to meibomian gland dysfunction. In some embodiments, the keratinized obstruction is composed of keratinized material and lipids. In some embodiments, the keratinized obstruction is a blockage at the meibomian gland orifice and excretory duct. In some embodiments, the keratinized obstruction is caused by keratinization of the epithelium at the lid margin and meibomian gland. In certain instances, the keratin obstruction is influenced by the migration or aberrant differentiation of stem cells. In some embodiments, the keratinized obstruction results in reduced delivery of oil to the lid margin and tear film, and stasis inside the meibomian gland that causes increased pressure, resultant dilation, acinar atrophy, and low secretion. In certain instances, keratinization of the meibomian gland causes degenerative gland dilation and atrophy.

The term, “meibomian gland dysfunction,” as used herein, refers to chronic, diffuse abnormality of the meibomian glands, that is characterized by terminal duct obstruction or qualitative or quantitative changes in the glandular secretion, or both. MGD may result in alteration of the tear film, eye irritation symptoms, inflammation, or ocular surface disease. The most prominent aspects of MGD are obstruction of the meibomian gland orifices and terminal ducts and changes in the meibomian gland secretions.

The meibomian glands are large sebaceous glands located in the eyelids, and unlike skin, are unassociated with hair. The meibomian glands produce the lipid layer of the tear film that protects it against evaporation of the aqueous phase. The meibomian gland orifice is located on the epithelial side of the lid margin, and can be a few hundred microns from the mucosal side. The glands are located on both upper and lower eyelids, with higher amounts of the glands on the upper eyelid. A single meibomian gland is composed of clusters of secretory acini that are arranged circularly around a long central duct and connected to it by short ductules. The terminal part of the central duct is lined by an ingrowth of the epidermis that covers the free lid margin and forms a short excretory duct that opens as an orifice at the posterior part of the lid margin just anterior to the mucocutaneous junction near the inner lid border. The oily secretion composed of lipids is synthesized within the secretory acini. The lipid secretion is a liquid at near body temperature and is delivered to the skin of the lid margin as a clear fluid, called “meibum.” It forms shallow reservoirs on the upper and lower lid margins, and consists of a complex mixture of cholesterol, wax, cholesteryl esters, phospholipids, with small amounts of triglycerides, triacylglycerols, and hydrocarbons. The separate meibomian glands are arranged in parallel, and in a single row throughout the length of the tarsal plates in the upper and lower lids. The extent of the glands corresponds roughly to the dimensions of the tarsal plates.

Ocular surface diseases are a group of diseases including, but not limited to, dry eye syndrome (including evaporative DES and/or aqueous deficiency DES), blepharitis, keratitis, meibomian gland dysfunction, conjunctivitis, lacrimal gland disorder, contact lens related conditions and inflammatory, infectious, or autoimmune diseases or disorders of the anterior surface of the eye.

In some instances, meibomian gland dysfunction (MGD) is a chronic, diffuse abnormality of the meibomian glands, which can be characterized by terminal duct obstruction and/or qualitative/quantitative changes in the glandular secretion. Terminal duct obstruction is caused by hyperkeratinization of the ductal epithelium (Nichols et al, Inv. Oph. & Vis. Sci. (2011); 52(4):1922-1929). These alterations in both meibum quality and expression may result in alteration of the tear film, symptoms of eye irritation, and ocular surface disease such as evaporative dry eye. The principal clinical consequence of MGD is evaporative dry eye syndrome and large population based studies (i.e., Bankok Study and the Shihpai Eye Study) estimate that over 60% of patients with dry eye symptoms also have MGD (Schaumberg et al, Investigative Ophthalmology and Visual Science. (2011); 52(4):1994-2005).

MGD is a leading contributor of dry eye syndrome. The occurrence of dry eye syndrome is widespread and affects about 20 million patients in the United States alone. Dry eye syndrome is a disorder of the ocular surface resulting from either inadequate tear production or excessive evaporation of moisture from the surface of the eye. Tears are important to corneal health because the cornea does not contain blood vessels, and relies on tears to supply oxygen and nutrients. Tears and the tear film are composed of lipids, water, and mucus, and disruption of any of these can cause dry eye. An inadequate amount of lipids flowing from the meibomian glands as caused by a keratinized obstruction, may cause excessive evaporation, thereby causing dry eye syndrome.

Currently there are no approved pharmacological agents useful for the treatment of MGD. The recognition that terminal duct obstruction from hyperkeratinization of the ductal epithelium on meibomian glands is a core mechanism behind meibomian gland dysfunction (MGD) is consistent with clinical experience demonstrating that effective treatments for MGD require resolution of ductal obstruction and evacuation of glandular contents (Nichols et al, 2011; Lane et al, 2012; Blackie et al, 2015). Warm compresses and thermal/mechanical devises (e.g., LipiFlow) are used in an attempt to raise the internal temperature of the meibomian glands over the normal melting point for meibum (i.e., 32° C. to 40° C.) in an attempt to resolve terminal duct obstruction (Lane et al, 2012). Unfortunately, warm compresses are unable to achieve this benefit for severely obstructed glands which can having a melting point >40° C. Current technology for removing keratinized obstruction of the meibomian gland also includes physical removal methods (e.g., debridement and gland probing), which are quite painful to patients.

Subsequent to a period of MGD, various stages of immune, inflammatory, and/or bacterial disease at the ocular surface are frequently observed because meibomian gland obstruction can cause a cascade of events that include further deterioration of the glands (Knop, IOVS, 2011) from stasis of the meibum in the secretory glands, mechanical pressure and stress from glandular obstruction, and increased bacterial growth that is associated with the downstream release of bacterial lipases, toxic mediators, and/or inflammatory mediators. All these factors reduce the quality and/or quantity of meibum the glands can release which in turn can cause chronic mechanical traumatization of the conjunctival, corneal and eyelid tissues which will lead to further tissue damage and the release of inflammatory mediators. Thus, many patients suffering from MGD also have inflammatory disease affecting their conjunctiva, cornea, larcrimal gland, lids or goblet cells causing comorbid conditions such as dry eye syndrome or blepharitis for which there is an unmet medical need.

For example, literature has used the terms posterior blepharitis and MGD as if they were synonymous, but these terms are not interchangeable. Posterior blepharitis describes inflammatory conditions of the posterior lid margin, of which MGD can be one possible cause. In its earliest stages, MGD may not be associated with clinical signs characteristic of posterior blepharitis. At this stage, affected individuals may be symptomatic, but alternatively, they may be asymptomatic and the condition regarded as subclinical. As MGD progresses, symptoms develop and lid margin signs, such as changes in meibum expressibility and quality and lid margin redness, may become more visible. At this point, an MGD-related posterior blepharitis is said to be present.

In some instances, altered meibomian gland secretion is detected by physically expressing the meibomian glands by applying digital pressure to the tarsal plates. In subjects without MGD, the meibum is a pool of clear oil. In MGD, both the quality and expressibility of the expressed material is altered. The altered meibum is also known as meibomian excreta and is made up of a mixture of altered secretions and keratinized epithelial material. In MGD, the quality of expressed lipid varies in appearance from a clear fluid, to a viscous fluid containing particulate matter and densely opaque, toothpaste-like material. The meibomian orifices may exhibit elevations above surface level of the lid, which is referred to as plugging or pouting, and is due to obstruction of the terminal ducts and extrusion of a mixture of meibomian lipid and keratinized material.

In some instances, obstructive MGD is characterized by all or some of the following: 1) chronic ocular discomfort, 2) anatomic abnormalities around the meibomian gland orifice (which is one or more of the following: vascular engorgement, anterior or posterior displacement of the mucocutaneous junction, irregularity of the lid margin) and 3) obstruction of the meibomian glands (obstructive findings of the gland orifices by slit lamp biomicroscopy (pouting, plugging or ridge), decreased meibum expression by moderate digital pressure).

Methods for assessing and monitoring MGD symptoms may include, but are not limited to patient questionnaires, meibomian gland expression, tear stability break up time, and determining the number of patent glands as seen by digital expression.

In some embodiments, the symptoms of a patient are assessed by asking the patient a series of questions. Questionnaires allow the assessment of a range of symptoms associated with ocular discomfort. In some embodiments, the questionnaire is the SPEED questionnaire. The SPEED questionnaire assesses frequency and severity of a patient's dry eye symptoms. It examines the occurrence of symptoms on the current day, past 72 hours and past three months. A SPEED score is tallied based on the patient's answers to the questions, to give a range of severity of the patient's symptoms. The SPEED questionnaire includes questions such as the following: 1) what dry eye symptoms are you experiencing, and when do they occur?2) how frequently do you experience dryness, grittiness, or scratchiness in your eyes?3) how often do you experience soreness or irritation of the eyes?4) how often do you experience burning or watering of the eyes?5) how often do you experience eye fatigue? and 6) how severe are the symptoms?

Meibomian gland expressibility is optionally determined to assess the meibomian gland function. In normal patients, meibum is a clear to light yellow oil. Meibum is excreted from the glands when digital pressure is placed on the glands. Changes in meibomian gland expressibility are one potential indicator of MGD. In some embodiments, during expression, quantifying the amount of physical force applied during expression is monitored in addition to assessing lipid volume and lipid quantity.

Tear stability break up time (TBUT) is a surrogate marker for tear stability. Tear film instability is a core mechanism in dry eye and MGD. Low TBUT implies a possibility of lipid layer compromise and MGD. TBUT is optionally measured by examining fluorescein breakup time, as defined as the time to initial breakup of the tear film after a blink. Fluorescein is optionally applied by wetting a commercially available fluorescein-impregnated strip with saline, and applied to the inferior fornix or bulbar conjuctiva. The patient is then asked to blink several times and move the eyes. The break up is then analyzed with a slit lamp, a cobalt blue filter, and a beam width of 4 mm. The patient is instructed to blink, and the time from upstroke of the last blink to the first tear film break or dry spot formation is recorded as a measurement.

Other methods for assessing MGD symptoms, include but are not limited to, Schirmer test, ocular surface staining, lid morphology analysis, meibography, meibometry, interferometry, evaporimetry, tear lipid composition analysis, fluorophotometry, meiscometry, osmolarity analysis, indices of tear film dynamics, evaporation and tear turnover.

Treatments for MGD can include lid warming, lid massage, lid hygiene, lid expression and meibomian gland probing. Pharmacological methods, prior to those described herein, have not been used.

Lid hygiene is considered the primary treatment for MGD and consists of three components: 1) application of heat, 2) mechanical massage of eyelids and 3) cleansing the eyelid. Eyelid warming procedures improve meibomian gland secretion by melting the pathologically altered meibomian lipids. Warming is achieved by warm compresses or devices. Mechanical lid hygiene includes the use of scrubs, mechanical expression and cleansing with various solutions of the eyelashes and lid margins. Lid margins are optionally also cleansed with hypoallergenic bar soap, dilute infant shampoo or commercial lid scrubs. Physical expression of meibomian glands is performed in a physician's office or is performed by the patient at home. The technique varies from gentle massage of the lids against the eyeball to forceful squeezing of the lids either against each other or between a rigid object on the inner lid surface and a finger, thumb, or rigid object (such as a glass rod, cotton swab, or metal paddle) on the outer lid surface. The rigid object on the inner lid surface protects the eyeball from forces transferred through the eyelid during expression and to offer a stable resistance, to increase the amount of force that is applied to the glands.

Eyelid warming is limited because the warming melts the lipids, but does not address movement of the keratinized material. Further, eyelid warming induces transient visual degradation due to corneal distortion. Mechanical lid hygiene is also limited because the force needed to remove an obstruction can be significant, resulting in significant pain to the patient. The effectiveness of mechanical lid hygiene is limited by the patient's ability to tolerate the associated pain during the procedure. Other treatments for MGD are limited.

Physical opening of meibomian glands obstruction by meibomian gland expression is an acceptable method to improve meibomian gland secretion and dry eye symptoms. In addition, probing of the meibomian gland canal has been used to open the obstructed canal. Both methods, expression and probing, are limited, however, by the pain induced by the procedure, the possible physical insult to the gland and canal structures and their short lived effect estimated at days and weeks. Therefore, methods are needed to improve patient comfort, which will not cause harm to the meibomian glands and canals, that will reduce the dependency on frequent office visits and improve secretion of meibum.

Patent U.S. Pat. No. 9,463,201 entitled, “Compositions and methods for the treatment of meibomian gland dysfunction” describes a method for treating meibomian gland dysfunction involving the topical administration of a therapeutically-effective amount of at least one keratolytic agent in an ophthalmically-acceptable carrier. The patent includes keratolytic agents that are inorganic selenium (Se) compounds such as selenium disulfide (SeS₂) or organoselenium compounds such as Ebselen (2-Phenyl-1,2-benzoselenazol-3-one). This agent would treat the underlying cause of MGD, but not a “plus” inflammatory disease as described by the DEWS report on MGD.

The role of inflammation in the etiology of MGD is controversial. The terms posterior blepharitis and MGD are not synonymous. Posterior blepharitis describes inflammatory conditions of the posterior lid margin and has various causes, of which MGD can be one possible cause (Nichols et al 2011). In its earliest stages, MGD is not associated with clinical signs characteristic of posterior blepharitis. As MGD progresses, an MGD-related posterior blepharitis is said to be present. MGD-related posterior blepharitis affects the meibomian glands and meibomian gland orifices. MGD-related posterior blepharitis is characterized by flora changes, esterase and lipase release, lipid changes, and eyelid inflammation. Hyperkeratinization of the meibomian gland epithelium (thickening of the lining of the glands) may lead to obstruction and a decrease in the quantity of meibomian gland secretions and may be responsible for MGD-related posterior blepharitis. Diagnosis of MGD-related posterior blepharitis includes meibomian gland expression with demonstration of an altered quality of expressed secretions, and/or by a loss of gland functionality (decreased or absent expressibility). The TFOS report on Meibomian Gland Disease specifically notes that anterior blepharitis and exacerbated inflammatory ocular surface disease are “plus” diseases to MGD which are managed by topical, ocular steroids (Nichols et al 2011).

Since these “plus” conditions can be present in various levels of severity from early to late MGD there is a need for treatments and/or combinations of treatments that can target both the underlying non-inflammatory pathophysiology of MGD and inflammation associated with these comorbid conditions.

MGD-related inflammatory eye disease may comprise a different mechanism than blepharitis-related MGD. MGD-related inflammatory eye disease is characterized by an inflammatory cascade involving activation and migration of T lymphocytes to the inflamed tissue. T lymphocyte infiltration may result in lacrimal gland stimulation and upregulation of cytokines. Exemplary cytokines that may be involved in MGD-related inflammatory eye disease include, but are not limited to, interleukin-1, interleukin-4, interleukin-6, inteleukin-8, interferon gamma, macrophage inflammatory protein 1 alpha, and tumor necrosis factor alpha. Kinase pathways including the mitogen activated protein kinase (MAPK) pathway are also activated in the inflammatory cascade. The inflammatory process results in loss of mucin-producing goblet cells and destruction of the ocular surface that can lead to further damage.

Dry eye syndrome, also known as keratoconjunctivitis sicca (KCS), is considered a self-sustaining disease that is progressively disconnected from its initial cause. Dry eye syndrome is associated with inflammation at the ocular surface and periocular tissue. Inflammation is characterized by the activation and migration of T lymphocytes to the inflamed tissue including in the conjunctiva and lacrimal glands. Inflammatory cytokines, chemokines, and matrix metalloproteinase have also been identified as being increased.

Animal models of dry eye disease have been established and reviewed (Barabino, et al, (Invest. Ophthalmol. Vis. Sci. 2004, 45:1641-1646)). Barabino, et al, (Invest. Ophthalmol. Vis. Sci. 2005, 46:2766-2771) described a model wherein exposure of normal mice to a low-humidity environment in a controlled-environment chamber leads to significant alterations in tear secretion, goblet cell density, and acquisition of dry eye-related ocular surface signs. However, no single animal model adequately accounts for the immune, endocrine, neuronal and environmental factors which contribute to dry eye pathogenesis.

Anti-inflammatory agents may be used to treat ocular surface diseases or disorders including dry eye syndrome. Corticosteroids are an effective anti-inflammatory therapy in dry eye disease. For example, in a 4-week, double-masked, randomized study in 64 patients with dry eye and delayed tear clearance, loteprednol etabonate 0.5% ophthalmic suspension (Lotemax [Bausch and Lomb, Rochester, NY]), QID, was found to be more effective than its vehicle in improving some signs and symptoms (Pflugfelder et al, Am J Ophthalmol (2004); 138:444-57). The TFOS 2007 report on dry eye disease went so far as to conclude that, “In the US Federal Regulations, ocular corticosteroids receiving “class labeling” are indicated for the treatment “ . . . of steroid responsive inflammatory conditions of the palpebral and bulbar conjunctiva, cornea and anterior segment of the globe such as allergic conjunctivitis, acne rosacea, superficial punctate keratitis, herpes zoster keratitis, iritis, cyclitis, selected infective conjunctivitis, when the inherent hazard of steroid use is accepted to obtain an advisable diminution in edema and inflammation.” KCS, in some instances, is included in this list of steroid-responsive inflammatory conditions (Therapy Subcommittee of the International Dry Eye WorkShop, 2007. Management and Therapy of Dry Eye Disease: Report of the Management and Therapy Subcommittee of the International Dry Eye WorkShop (2007). 2007; 5: 163-178).” While the US FDA does not agree with this conclusion, short courses of steroids, especially Lotemax, can be commonly used to treat inflammation associated with dry eye disease.

Other anti-inflammatory agents include nonsteroidal anti-inflammatory drugs (NSAIDs).

NSAIDs inhibit the activity of cyclooxygenases including cyclooxygenase-1 (COX-1) and cyclooxygenase-2 (COX-2), which are enzymes involved in the synthesis of prostaglandins and thromboxanes from arachidonic acid. Prostaglandin and thromboxane signaling are involved in inflammation and immune modulation. In some cases, NSAIDs are used for treating dry eye disease by treating the inflammation at the ocular surface.

In some instances, provided herein is a compound that provides a therapeutically effective amount of (e.g., a free form of) an immunomodulator, such as an immunomodulator described herein, and/or (e.g., a free form of) a keratolytic agent, such as a keratolytic agent described herein.

In some instances, the free form of the immunomodulator is selected from the group consisting of cilomilast, ruxolitinib, ritlecitinib, tofacitinib, oclacitinib, methotrexate, loteprednol, and tacrolimus.

The chemical name for cilomilast is cis-4-cyano-4-[3-(cyclopentyloxy)-4-methoxyphenyl]cyclohexanecarboxylic acid having a molecular formula of C₂₀H₂₅NO₄ and a molecular weight of 343.4 g/mol. The structural formula of cilomilast is:

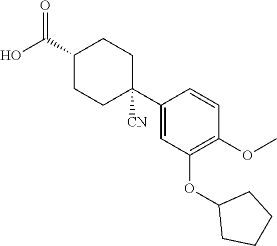 [SMILES: COc1ccc([C@]2(C#N)CC[C@@H](C(=O)O)CC2)cc1OC1CCCC1]

Cilomilast is a potent and selective phosphodiesterase-4 (PDE4) inhibitor that has demonstrated improvements in objective signs of dry eye in a murine model (Sadrai et al, Invest Ophthalmol Vis Sci. (2012) 53 (7), 3584-3591). The potent and selective phosphodiesterase-4 (PDE4) inhibitor was found to act locally at the level of the ocular surface, by suppressing the generation of IL-17-associated immunity.

The chemical name for ruxolitinib is (3R)-3-Cyclopentyl-3-[4-(7H-pyrrolo[2,3-d]pyrimidin-4-yl)pyrazol-1-yl]propanenitrile having a molecular formula of C₁₇H₁₈N₆ and a molecular weight of 306.4 g/mol. The structural formula of ruxolitinib is:

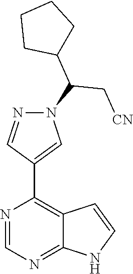

Ruxolitinib is a potent and selective janus kinase (JAK) 1 and JAK2 inhibitor that has demonstrated improvements in objective signs (in murine models) of psoriasis (Mesa, IDrugs. (2010) 13 (6), 394-403), alopecia (Falto-Aizpurua et al, Expert Opinion on Emerging Drugs. (2014) 19 (4), 545-556), and uveitis (caused by Salmonella typhiurium endotoxin) (Lin et al, Microorganisms (2021) 9 (7), 1-12). The potent and selective JAK1 and JAK2 inhibitor was found to act locally at the level of the ocular surface, possibly by suppressing the expression of mediators of proinflammatory cytokine pathways (e.g., of the JAK2-signal transducers and activators of transcription 3 (STAT3) pathway) in the ciliary body and iris.

The chemical name for ritlecitinib is 1-((2S,5R)-5-((7H-pyrrolo[2,3-d]pyrimidin-4-yl)amino)-2-methylpiperidin-1-yl)prop-2-en-1-one having a molecular formula of C₁₅H₁₉N₅O and a molecular weight of 285.3 g/mol. The structural formula of ritlecitinib is:

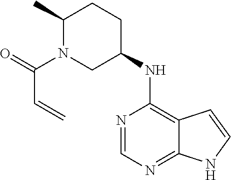 [SMILES: C=CC(=O)N1C[C@H](Nc2ncnc3[nH]ccc23)CC[C@@H]1C]

Ritlecitinib is an irreversible covalent JAK3 selective inhibitor that has demonstrated improvements in objective signs (in humans and murine models) of alopecia (NCT02974868). The irreversible covalent JAK3 selective inhibitor was found to inhibit the expression of mediators of proinflammatory cytokine pathways (such as IL-7, IL-9, IL-15 and IL-21), some of which being implicated in the pathophysiology of ocular indications, such as, dry eye disease and uveitis.

The chemical name for tofacitinib is 3-[(3R,4R)-4-Methyl-3-[methyl(7H-pyrrolo[2,3-d]pyrimidin-4-yl)amino]piperidin-1-yl]-3-oxopropanenitrile having a molecular formula of C₁₆H₂₀N₆O and a molecular weight of 312.4 g/mol. The structural formula of tofacitinib is:

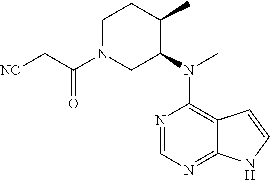 [SMILES: C[C@@H]1CCN(C(=O)CC#N)C[C@@H]1N(C)c1ncnc2[nH]ccc12]

Tofacitinib is a potent and selective JAK1 and JAK3 inhibitor that has demonstrated improvements in objective signs (in humans and/or murine models) of psoriasis (Di Lernia et al, Drug Design, Development and Therapy. (2016) 10, 533-539), alopecia (Kennedy Crispin et al, JCI Insight. (2016) 1 (15), e89776), dermatitis (Levy et al, Journal of the American Academy of Dermatology (2015) 73 (3), 395-399), and dry eye disease (Jing-Feng Huang, US Ophthalmic Review. (2014) 7(1), 12-15). The potent and selective JAK1 and JAK3 inhibitor was found to act locally at the level of the ocular surface, possibly by suppressing the expression of mediators of proinflammatory cytokine pathways (e.g., IFN-7, IL-12, IL-23, and IL-6).

The chemical name for oclacitinib is N-methyl{trans-4-[methyl(7H-pyrrolo[2,3-d]pyrimidin-4-yl)amino]cyclohexyl}methanesulfonamide having a molecular formula of C₁₅H₂₃N₅O₂S and a molecular weight of 337.4. The structural formula of oclacitinib is:

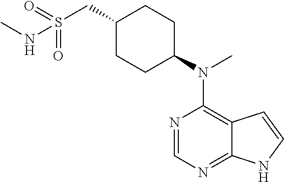 [SMILES: CNS(=O)(=O)C[C@H]1CC[C@H](N(C)c2ncnc3[nH]ccc23)CC1]

Oclacitinib is a potent and selective JAK1, JAK2, and JAK3 inhibitor that has demonstrated improvements in objective signs (in humans and/or dogs) of dermatitis (Gonzalez et al, Journal of Veterinary Pharmacology and Therapeutics (2014) 37 (4), 317-324), keratoconjunctivitis, and dry eye disease (Kravetz de Oliveira, Vet Ophthalmol. (2019)₂₂(5), 633-643). The potent and selective JAK1, JAK2, and JAK3 inhibitor was found to act locally at the level of the ocular surface, possibly by suppressing the expression of mediators of proinflammatory cytokine pathways (e.g., IL-2, IL-4, IL-6, IL-13, and IL-31).

The chemical name for methotrexate is (2S)-2-[(4-{[(2,4-Diaminopteridin-6-yl)methyl](methyl)amino}benzoyl)amino]pentanedioic acid having a molecular formula of C₂₀H₂₂N₈O₅ and a molecular weight of 454.4 g/mol. The structural formula of methotrexate is:

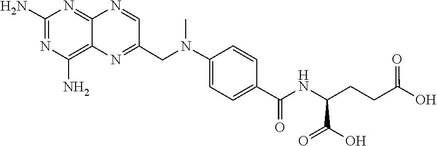 [SMILES: CN(Cc1cnc2nc(N)nc(N)c2n1)c1ccc(C(=O)N[C@@H](CCC(=O)O)C(=O)O)cc1]

While prophylactic methotrexate has been associated with ocular side effect, such as, eye irritation and dry eye, methotrexate has demonstrated improvements in objective signs (in humans and/or murine models) of uveitis (Gangaputra, Ophthalmology (2009) 116(11), 2188-2198). Methotrexate was found to act locally at the level of the ocular surface, possibly by suppressing and/or activating the expression of mediators of certain immunological pathways.

The chemical name for loteprednol is chloromethyl 17-ethoxycarbonyloxy-11-hydroxy-10,13-dimethyl-3-oxo-7,8,9,11,12,14,15,16-octahydro-6H-cyclopenta[a]phenanthrene-17-carboxylate having a molecular formula of C₂₄H₃₁ClO₇ and a molecular weight of 467.0 g/mol. The structural formula of loteprednol is:

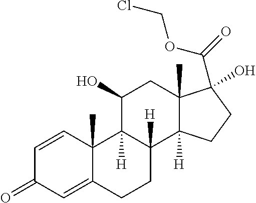

Loteprednol has demonstrated improvements in objective signs (in humans) of anterior segment inflammatory conditions of the eye, such as, for example, ocular inflammation, conjunctivitis, uveitis, keratitis, keratoconjunctivitis, and the like (Bartlett et al, US Ophthalmic Review. (2011) 4(1), 57-62). Loteprednol was found to act locally at the level of the ocular surface, possibly by suppressing and/or activating the expression of mediators of certain immunological pathways.

The chemical name for tacrolimus is (−)-(3S,4R,5S,8R,9E,12S,14S,15R,16S,18R,26aS)-8-allyl-5,6,8,11,12,13,14,15,16,17,18,19,24,25,26,26a-hexadecahydro-5,19-dihydroxy-3-{(E)-2-[(1R,3R,4R)-4-hydroxy-3-methylcyclohexyl]-1-methylvinyl}-14,16-dimethoxy-4,10,12,18-tetramethyl-15,19-epoxy-3H-pyrido[2,1-c][1,4]oxaazacyclotricosane-1,7,20,21(4H,23H)-tetrone having a molecular formula of C₄₄H₆₉NO₁₂ and a molecular weight of 804.0 g/mol. The structural formula of tacrolimus is:

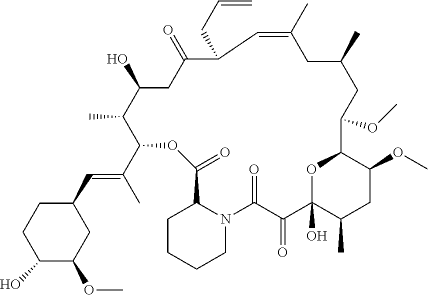

Tacrolimus is a calcineurin inhibitor that has demonstrated improvements in objective signs (in humans and/or dogs) of keratoconjunctivitis and dry eye disease (Kravetz de Oliveira, Vet Ophthalmol. (2019) 22(5), 633-643). The potent and selective JAK1, JAK2, and JAK3 inhibitor was found to act locally at the level of the ocular surface, possibly by interfering with interleukin-2 transcription, reducing lacrimal gland inflammation, and/or restoring corneal health.

### Compositions

Described herein are compounds (e.g., keratolytic conjugates and/or dual acting-agents) which address simultaneously the non-inflammatory keratosis (e.g., keratolytic blockage) component of dermal and/or ocular diseases or disorders described herein (e.g., MGD) and the improper immunomodulation and/or inflammation associated with the disease or disorder described herein (e.g., an ocular allergy, dry eye disease (including inflammatory DED and aqueous deficiency, ocular GVHD, or the like). In some embodiments, a compound provided herein is useful as either an acute therapy (e.g., by a trained specialist or physician) or as a chronic therapy (e.g., in the hands of a patient, or alternatively, by a trained specialist or physician). A compound provided herein is tested, in some embodiments, using the assays and methods described herein (e.g., as described in the examples). In some embodiments, a compound provided herein represents a significant advance in the art as the first-order metabolites obtained from metabolism of the agents are operative against both the keratolytic and the immunomodulation and/or inflammatory component of diseases, such as, for example, ocular allergies, dry eye disease, ocular GVHD, and the like.

Provided in some embodiments herein is a compound having a structure represented by Formula (A):

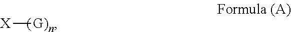

wherein,
X is an immunomodulator radical (e.g., an immunosuppressant radical or an immunostimulant radical);
each G independently comprises at least one linker and at least one radical of a keratolytic agent; and
n is 1-3,
   or a pharmaceutically acceptable salt or solvate thereof.

Provided in some embodiments herein is a compound having a structure represented by Formula (A-I):

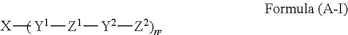

wherein,
X is an immunomodulator radical (e.g., an immunosuppressant radical or an immunostimulant radical);
each Y¹ and Y² is independently a linker;
each Z¹ and Z² is independently a radical of a keratolytic agent; and
n is 1-3,
   or a pharmaceutically acceptable salt or solvate thereof.

Provided in some embodiments herein is a compound having a structure represented by Formula (A-II):

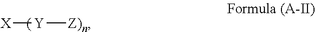

wherein,
X is an immunomodulator radical (e.g., an immunosuppressant radical or an immunostimulant radical);
each Y is independently a linker;
each Z is independently a radical of a keratolytic agent; and
n is 1-3,
or a pharmaceutically acceptable salt or solvate thereof.

In some embodiments, X is an immunomodulator radical. In some embodiments, X is an immunosuppressant radical. In some embodiments, X is an immunostimulant radical.

In some embodiments, X (e.g., in its free form) is an anti-inflammatory agent.

In some embodiments, X (e.g., in its free form) is an immunomodulator (e.g., an immunosuppressant or an immunostimulant) and an anti-inflammatory agent.

In some embodiments, X is selected from the group consisting of a radical of a (e.g., selective) phosphodiesterase (PDE) inhibitor (e.g., a radical of a (e.g., selective) phosphodiesterase-4 (PDE-4) inhibitor (e.g., a cilomilast radical)), a radical of a (e.g., selective) Janus kinase (JAK) inhibitor (e.g., a radical of a JAK1, JAK2, and/or JAK3 inhibitor (e.g., a ruxolitinib radical, a tofacitinib radical, a oclacitinib radical, or a ritlecitinib radical), a radical of a folate reductase inhibitor (e.g., a methotrexate (or a derivative thereof) radical), a radical of a steroid (e.g., a corticosteroid radical (e.g., a glucocorticoid radical (e.g., a loteprednol (or a derivative thereof) radical))), and a radical of a calcineurin inhibitor (e.g., a tacrolimus radical).

In some embodiments, X is selected from the group consisting of a cilomilast radical, a ruxolitinib radical, a tofacitinib radical, an oclacitinib radical, a ritlecitinib radical, a methotrexate (or a derivative thereof) radical), a loteprednol (or a derivative thereof) radical, and a tacrolimus radical.

In some embodiments, X is selected from the group consisting of a cilomilast radical.

In some embodiments, X is selected from the group consisting of a ruxolitinib radical.

In some embodiments, X is selected from the group consisting of a tofacitinib radical.

In some embodiments, X is selected from the group consisting of an oclacitinib radical.

In some embodiments, X is selected from the group consisting of a ritlecitinib radical.

In some embodiments, X is selected from the group consisting of a methotrexate radical.

In some embodiments, X is selected from the group consisting of a loteprednol radical.

In some embodiments, X is selected from the group consisting of a tacrolimus radical.

In some embodiments, X is not lifitegrast.

In some embodiments, X is not azithromycin.

In some embodiments, X is not (S)-3-(4-((4-carbamoylpiperidine-1-carbonyl)oxy)phenyl)-2-((S)-4-methyl-2-(2-(o-tolyloxy)acetamido)pentanamido)propanoic acid.

In some embodiments, the anti-inflammatory agent is not lifitegrast. In some embodiments, the anti-inflammatory agent is not azithromycin. In some embodiments, the anti-inflammatory agent is not (S)-3-(4-((4-carbamoylpiperidine-1-carbonyl)oxy)phenyl)-2-((S)-4-methyl-2-(2-(o-tolyloxy)acetamido)pentanamido)propanoic acid.

In some embodiments, X has a structure represented by Formula (I-A):

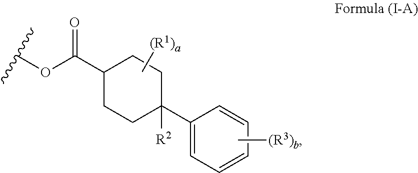

wherein,
each R¹ is independently halogen, alkyl, or —CN;
R² is hydrogen, —CN, halogen, or alkyl;
each R³ is independently —OR^{a}, alkyl, heteroalkyl, cycloalkyl, or heterocyclyl;
   each R^{a} is independently hydrogen, alkyl, cycloalkyl, or heterocyclyl;
a is 0-9; and
b is 0-5.

In some embodiments, a is 0. In some embodiments, R² is —CN. In some embodiments, b is 2. In some embodiments, each R³ is independently —OR^{a}. In some embodiments, each R^{a} is independently alkyl or cycloalkyl. In some embodiments, each R³ is independently —OMe or —OC₃-C₅ cycloalkyl.

In some embodiments, X has a structure represented by Formula (I-AA):

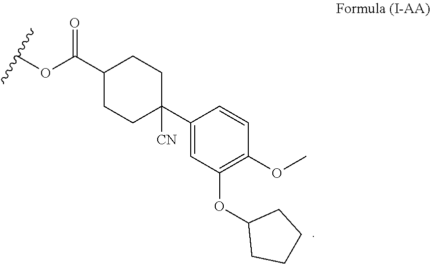 [SMILES: *OC(=O)C1CCC(C#N)(c2ccc(OC)c(OC3CCCC3)c2)CC1]

In some embodiments, X has a structure represented by Formula (I-B):

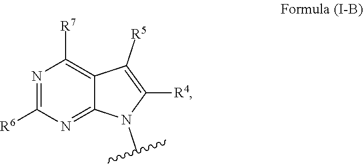

wherein,
R⁴ is hydrogen, halogen, or alkyl;
R⁵ is hydrogen, halogen, or alkyl;
R⁶ is hydrogen, halogen, or alkyl;
R⁷ is —NR^{b}R^{c} or optionally substituted heterocyclyl; and
   R^{b} and R^{c} are each independently hydrogen, alkyl, optionally substituted cycloalkyl, or optionally substituted heterocyclyl.

In some embodiments, R⁴ and R⁵ are hydrogen. In some embodiments, R⁶ is hydrogen. In some embodiments, R⁷ is substituted heterocyclyl. In some embodiments, R⁷ is heterocyclyl substituted with alkyl substituted with CN and cycloalkyl.

In some embodiments, X has a structure represented by Formula (I-BA):

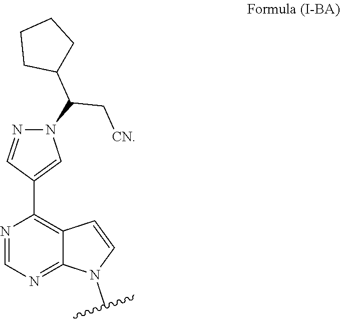

In some embodiments, R⁴ and R⁵ are hydrogen. In some embodiments, R⁶ is hydrogen. In some embodiments, R⁷ is —NR^{b}R^{c}. In some embodiments, R^{b} is hydrogen or C₁-C₃ alkyl. In some embodiments, R^{c} is substituted cycloalkyl or substituted heterocyclyl. In some embodiments, R^{b} is hydrogen. In some embodiments, R^{c} is heterocyclyl substituted with one or more substituent, each substituent being independently optionally substituted alkyl. In some embodiments, R^{c} is heterocyclyl substituted with unsubstituted C₁-C₃ alkyl and (unsaturated) C₁-C₃ alkyl substituted with oxo.

In some embodiments, X has a structure represented by Formula (I-BB):

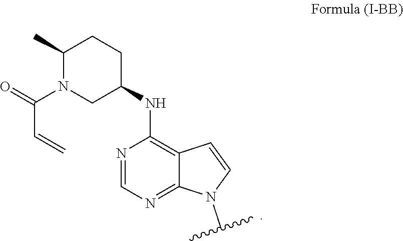 [SMILES: *n1ccc2c(N[C@@H]3CC[C@H](C)N(C(=O)C=C)C3)ncnc21]

In some embodiments, R⁴ and R⁵ are hydrogen. In some embodiments, R⁶ is hydrogen. In some embodiments, R⁷ is —NR^{b}R^{c}. In some embodiments, R^{b} is CH₃. In some embodiments, R^{c} is cycloalkyl or heterocyclyl substituted with one or more substituent, each substituent being independently optionally substituted alkyl. In some embodiments, R^{c} is heterocyclyl substituted with unsubstituted C₁-C₃ alkyl and C₁-C₃ alkyl substituted with oxo and —CN.

In some embodiments, X has a structure represented by Formula (I-BC):

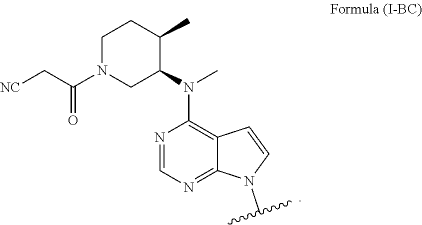 [SMILES: *n1ccc2c(N(C)[C@H]3CN(C(=O)CC#N)CC[C@H]3C)ncnc21]

In some embodiments, R⁴ and R⁵ are hydrogen. In some embodiments, R⁶ is hydrogen. In some embodiments, R⁷ is —NR^{b}R^{c}. In some embodiments, R^{b} is CH₃. In some embodiments, R^{c} is cycloalkyl or heterocyclyl substituted with one or more substituent, each substituent being independently optionally substituted alkyl. In some embodiments, R^{c} is cycloalkyl substituted with C₁-C₃ alkyl substituted with —SO₂NHCH₃.

In some embodiments, X has a structure represented by Formula (I-BD):

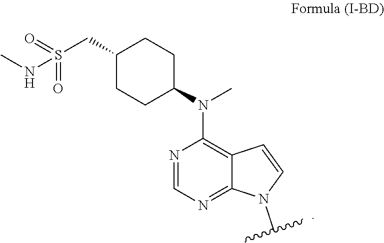 [SMILES: *n1ccc2c(N(C)[C@H]3CC[C@H](CS(=O)(=O)NC)CC3)ncnc21]

In some embodiments, X has a structure represented by Formula (I-C1), Formula (I-C2), or Formula (I-C3):

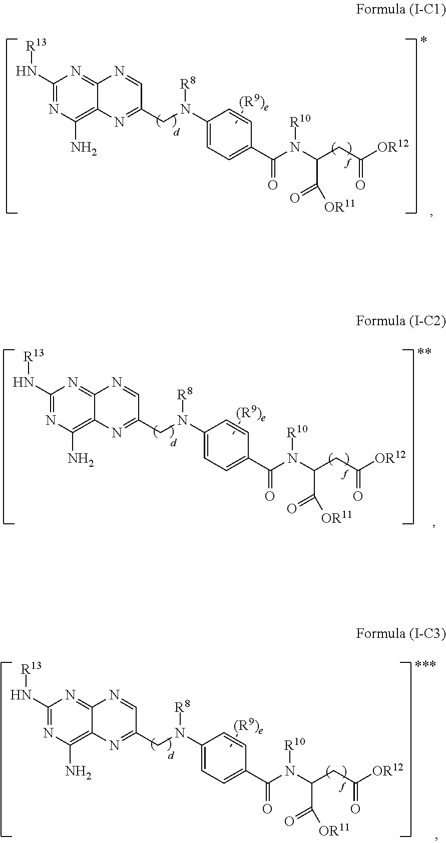

wherein,
R⁸ is hydrogen or alkyl;
each R⁹ is independently halogen or alkyl;
R¹⁰ is hydrogen or alkyl;
R¹¹ is a radical, hydrogen, or alkyl;
R¹² is a radical, hydrogen, or alkyl;
R¹³ is a radical or hydrogen;
d is 1-3;
e is 0-4; and
f is 1-4.

In some embodiments, R⁸ is —CH₃. In some embodiments, e is 0. In some embodiments, R¹⁰ is hydrogen. In some embodiments, d is 1. In some embodiments, f is 2. In some embodiments, R¹¹ is a radical. In some embodiments, R¹² is a radical. In some embodiments, R¹¹ is a radical and R¹² is a radical. In some embodiments, R¹³ is a radical. In some embodiments, R¹¹ is a radical, R¹² is a radical, and R¹³ is hydrogen.

In some embodiments, X has a structure represented by Formula (I-CA):

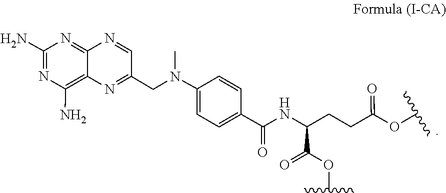 [SMILES: *OC(=O)CC[C@H](NC(=O)c1ccc(N(C)Cc2cnc3nc(N)nc(N)c3n2)cc1)C(=O)O*]

In some embodiments, R⁸ is —CH₃. In some embodiments, e is 0. In some embodiments, R¹⁰ is hydrogen. In some embodiments, d is 1. In some embodiments, f is 2. In some embodiments, R¹³ is a radical and R¹¹ and R¹² are hydrogen.

In some embodiments, X has a structure represented by Formula (I-CB):

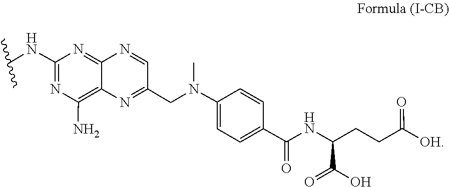 [SMILES: *Nc1nc(N)c2nc(CN(C)c3ccc(C(=O)N[C@@H](CCC(=O)O)C(=O)O)cc3)cnc2n1]

In some embodiments, X has a structure represented by Formula (I-D′):

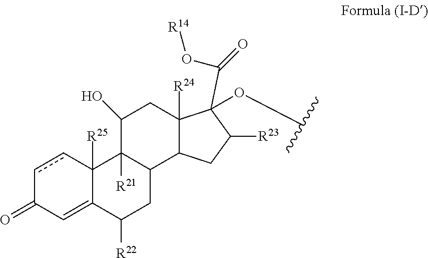

wherein,
[symbol] is a single bond or a double bond;
R¹⁴ is hydrogen, or optionally substituted alkyl;
R²¹ is hydrogen, halogen, or alkyl;
R²² is hydrogen, halogen, or alkyl;
R²³ is hydrogen or alkyl;
R²⁴ is hydrogen or alkyl; and
R²⁵ is hydrogen or alkyl.

In some embodiments, [symbol] is a single bond. In some embodiments, [symbol] is a double bond. In some embodiments, R¹⁴ is hydrogen or optionally substituted alkyl. In some embodiments, R¹⁴ is hydrogen. In some embodiments, R¹⁴ is optionally substituted alkyl. In some embodiments, R¹⁴ is alkyl substituted with halo or cyano. In some embodiments, R¹⁴ is alkyl substituted with halo. In some embodiments, R¹⁴ is alkyl substituted with chloro. In some embodiments, R¹⁴ is alkyl substituted with cyano. In some embodiments, R²¹ is hydrogen, halogen, or alkyl. In some embodiments, R²¹ is hydrogen. In some embodiments, R²¹ is halogen. In some embodiments, R²¹ is alkyl. In some embodiments, R²¹ is hydrogen or halogen. In some embodiments, R²¹ is fluorine. In some embodiments, R²² is hydrogen, halogen, or alkyl. In some embodiments, R²² is hydrogen. In some embodiments, R²² is halogen. In some embodiments, R²² is alkyl. In some embodiments, R²² is hydrogen or halogen. In some embodiments, R²² is fluorine. In some embodiments, R²³ is hydrogen or alkyl. In some embodiments, R²³ is hydrogen. In some embodiments, R²³ is alkyl. In some embodiments, R²³ is methyl. In some embodiments, R²⁴ is hydrogen or alkyl. In some embodiments, R²⁴ is hydrogen. In some embodiments, R²⁴ is alkyl. In some embodiments, R²⁴ is methyl. In some embodiments, R²⁵ is hydrogen or alkyl. In some embodiments, R²⁵ is hydrogen. In some embodiments, R²⁵ is alkyl. In some embodiments, R²⁵ is methyl. In some embodiments, R²⁴ and R²⁵ are methyl. In some embodiments, R²³, R²⁴, and R²⁵ are methyl. In some embodiments, R²¹ and R²² are fluorine.

In some embodiments, X has a structure represented by Formula (I-D):

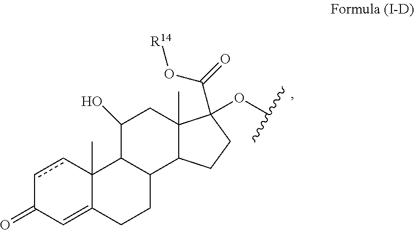

wherein,
[symbol] is a single bond or a double bond; and
R¹⁴ is hydrogen or optionally substituted alkyl.

In some embodiments, [symbol] is a double bond. In some embodiments, R¹⁴ is alkyl substituted with halogen. In some embodiments, R¹⁴ is hydrogen or alkyl substituted with halogen or cyano. In some embodiments, R¹⁴ is hydrogen. In some embodiments R¹⁴ is alkyl substituted with halo. In some embodiments R¹⁴ is alkyl substituted with chloro.

In some embodiments, X has a structure represented by Formula (I-DA):

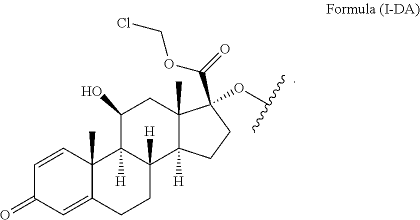

In some embodiments, [symbol] is a double bond. In some embodiments, R¹⁴ is hydrogen.

In some embodiments, X has a structure represented by Formula (I-DB):

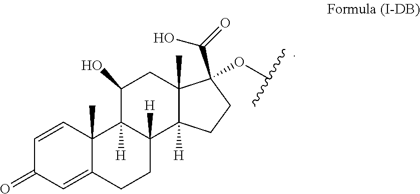

In some embodiments, R¹⁴ is alkyl substituted with cyano.

In some embodiments, X has a structure represented by Formula (I-DC):

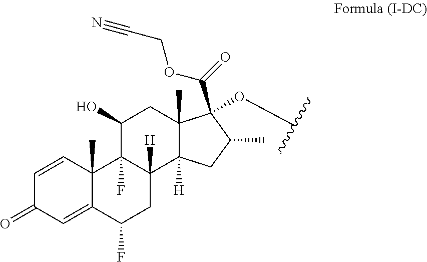

In some embodiments, X has a structure represented by Formula (I-E) or Formula (I-E′):

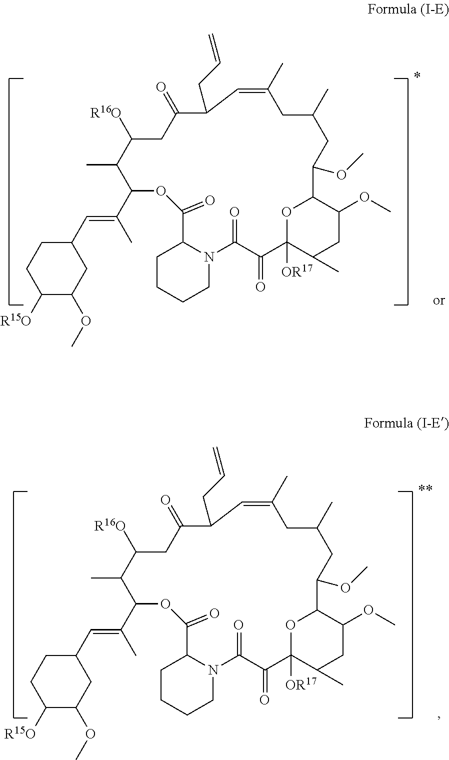

wherein,
   R¹⁵, R¹⁶, and R¹⁷ are each independently selected from a radical, hydrogen, or alkyl.

In some embodiments, R¹⁵ is a radical. In some embodiments, R¹⁶ is a radical. In some embodiments, R¹⁵ is a radical and R¹⁶ is a radical. In some embodiments, R¹⁷ is hydrogen.

In some embodiments, R¹⁵ is a radical, R¹⁶ is hydrogen, and R¹⁷ is hydrogen.

In some embodiments, X has a structure represented by Formula (I-EA):

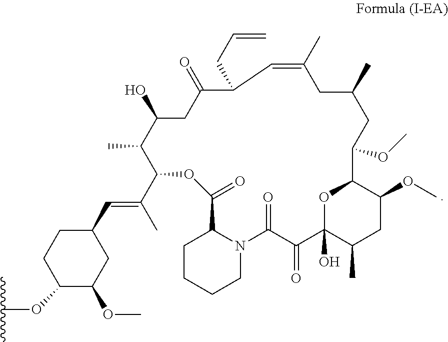

In some embodiments, R¹⁵ is hydrogen, R¹⁶ is a radical, and R¹⁷ is hydrogen.

In some embodiments, X has a structure represented by Formula (I-EB):

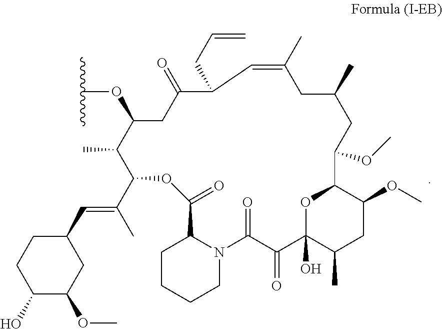

In some embodiments, R¹⁵ is a radical, R¹⁶ is a radical, and R¹⁷ is hydrogen.

In some embodiments, X has a structure represented by Formula (I-EC):

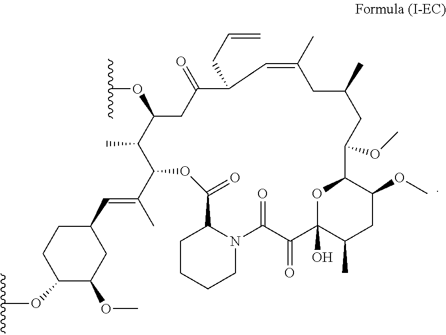

In some embodiments, n is 1. In some embodiments, n is 2.

In some embodiments, each linker (e.g., Y, Y¹, or Y²) is the same.

In some embodiments, each linker (e.g., Y, Y¹, or Y²) is different.

In some embodiments, each linker (e.g., Y, Y¹, or Y²) is independently a bond, substituted or unsubstituted (e.g., branched or straight) alkyl (alkylenyl), or substituted or unsubstituted (e.g., branched or straight) heteroalkyl (heteroalkylenyl). In some embodiments, each linker (e.g., Y, Y¹, or Y²) is independently substituted or unsubstituted (e.g., branched or straight) alkyl (alkylenyl) or substituted or unsubstituted (e.g., branched or straight) heteroalkyl (heteroalkylenyl). In some embodiments, each linker (e.g., Y, Y¹, or Y²) is independently a bond, —CH(CH₃)—, or —CH₂—. In some embodiments, each linker (e.g., Y, Y¹, or Y²) is a bond. In some embodiments, Y¹ is a bond and each Y² is independently —CH(CH₃)— or —CH₂—.

In some embodiments, each radical of a keratolytic agent (e.g., Z, Z¹, or Z²) is a radical of the same keratolytic agent.

In some embodiments, each radical of a keratolytic agent (e.g., Z, Z¹, or Z²) is a radical of a different keratolytic agent.

In some embodiments, each radical of a keratolytic agent (e.g., Z, Z¹, or Z²) comprises one or more keratolytic group. In some embodiments, each radical of a keratolytic agent (e.g., Z, Z¹, or Z²) comprises one or more group, each group being independently selected from the group consisting of —O—, oxo, substituted or unsubstituted (e.g., branched or straight) alkyl (alkylenyl), substituted or unsubstituted (e.g., branched or straight) heteroalkyl (heteroalkylenyl), substituted or unsubstituted alkoxyl, substituted or unsubstituted cycloalkyl, and substituted or unsubstituted heterocyclyl. In some embodiments, each radical of a keratolytic agent (e.g., Z, Z¹, or Z²) is (e.g., branched or straight) alkyl (alkylenyl) substituted with one or more substituent, each substituent being independently selected from the group consisting of oxo, hydroxy, alkyl, alkoxy, and substituted or unsubstituted heterocyclyl. In some embodiments, each radical of a keratolytic agent (e.g., Z, Z¹, or Z²) is (e.g., branched or straight) heteroalkyl (heteroalkylenyl) substituted with one or more substituent, each substituent being independently selected from the group consisting of oxo, alkyl, thioalkyl, and substituted or unsubstituted heterocyclyl. In some embodiments, Z¹ is straight alkyl (alkylenyl) substituted with one or more substituent, each substituent being independently selected from the group consisting of oxo and hydroxy and Z² is straight alkyl (alkylenyl) substituted with one or more substituent, each substituent being independently selected from the group consisting of oxo and substituted or unsubstituted heterocyclyl.

In some embodiments, Y¹ is a bond, Y² is —CH(CH₃)— or —CH₂—, Z¹ is

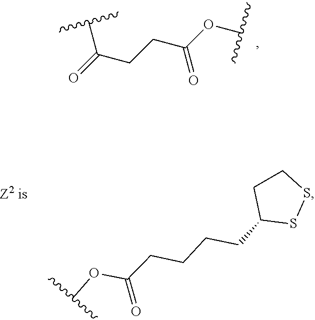 [SMILES: *OC(=O)CCC(*)=O ; *OC(=O)CCCC[C@@H]1CCSS1]

n is 1.

In some embodiments, each radical of a keratolytic agent (e.g., Z, Z¹, or Z²) has a structure represented by:

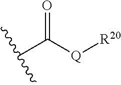

wherein:
Q is —O— or —(CR¹⁸R¹⁹)ₘ—;
m is 1-6;
each R¹⁸ and R¹⁹ is independently H, halo, alkyl, alkoxy, haloalkyl, or thioalkyl;
   or an adjacent R¹⁸ and R¹⁹ combine to the atoms to which they are attached to form an oxo; and
R²⁰ is alkyl, heteroalkyl, heterocyclyl, alkoxy, or hydroxy, the alkyl, heteroalkyl, heterocyclyl, or alkoxy each independently being optionally substituted.

In some embodiments, each R¹⁸ and R¹⁹ is independently H, C₁-C₆ alkyl, or C₁-C₃ thioalkyl. In some embodiments, each R¹⁸ and R¹⁹ is independently H, CH₃, or CH₂SH. In some embodiments, m is 1-4.

In some embodiments, Q is —(CR¹⁸R¹⁹)ₘ—, m is 1-4, R¹⁸ and R¹⁹ are each independently H or C₁-C₆ alkyl, and R²⁰ is optionally substituted heterocyclyl.

In some embodiments, R²⁰ is dithiolanyl or dithiolanyl oxide. In some embodiments, R²⁰ is:

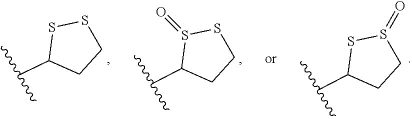 [SMILES: *C1CCSS1 ; *C1CCSS1=O ; *C1CCS(=O)S1]

In some embodiments, Q is —CH₂—, —CH(CH₃)—, —(CH₂)₂C(═O)—, —CH₂C(CH₃)₂CH₂—, or —CH(CH₂SH)— and R²⁰ is hydroxy, optionally substituted C₁-C₆ alkyl, optionally substituted C₁-C₆ alkoxy, or optionally substituted C₁-C₆ heteroalkyl.

In some embodiments, R²⁰ is CH₃, hydroxy, —O(C₁-C₃ alkoxy), or substituted C₁-C₆ heteroalkyl (e.g., heteroalkyl substituted with CH₃, oxo, and dithiolanyl or dithiolanyl oxide). In some embodiments, R²⁰ is —OH, —CH₃, —OCH₃, —OCH₂CH₃, —NH(C═O)CH₃,

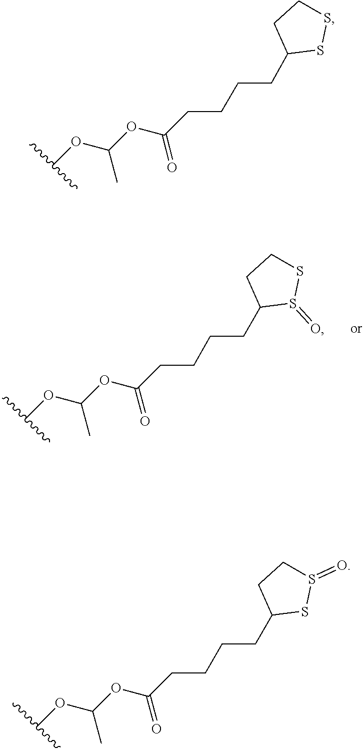 [SMILES: *OC(C)OC(=O)CCCCC1CCSS1 ; *OC(C)OC(=O)CCCCC1CCSS1=O ; *OC(C)OC(=O)CCCCC1CCS(=O)S1]

In some embodiments, Q is —O— and R²⁰ is optionally substituted C₁-C₆ alkyl.

In some embodiments, R²⁰ is methyl, ethyl, propyl, isopropyl, butyl, or tert-butyl.

Provided in some embodiments herein is a compound having a structure provided in Table 1, a stereoisomer thereof, or a pharmaceutically acceptable salt or solvate of the compound or the stereoisomer.

TABLE 1

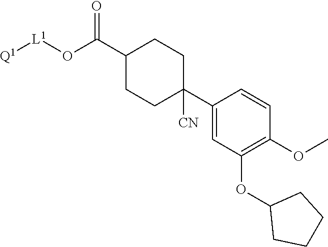

| Com- | | |
|---|---|---|
| pound | L¹ | Q¹ |
| 1 | —CH(CH₃)— | 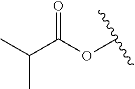 [SMILES: *OC(=O)C(C)C] |
| 2 | —CH(CH₃)— | 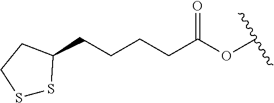 [SMILES: *OC(=O)CCCC[C@@H]1CCSS1] |
| 3 | —CH(CH₃)— | 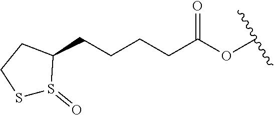 [SMILES: *OC(=O)CCCC[C@@H]1CCSS1=O] |
| 4 | —CH₂— | 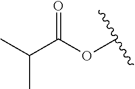 [SMILES: *OC(=O)C(C)C] |
| 5 | —CH₂— | 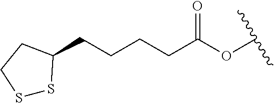 [SMILES: *OC(=O)CCCC[C@@H]1CCSS1] |
| 6 | —CH₂— | 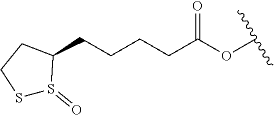 [SMILES: *OC(=O)CCCC[C@@H]1CCSS1=O] |

Provided in some embodiments herein is a compound having a structure provided in Table 2, a stereoisomer thereof, or a pharmaceutically acceptable salt or solvate of the compound or the stereoisomer.

TABLE 2

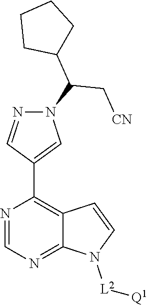

| Compound | L² | Q² |
|---|---|---|
| 7 | —CH(CH₃)— | 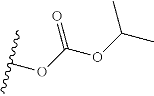 [SMILES: *OC(=O)OC(C)C] |
| 8 | bond | 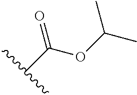 [SMILES: *C(=O)OC(C)C] |
| 9 | —CH₂— | 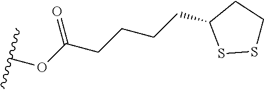 [SMILES: *OC(=O)CCCC[C@@H]1CCSS1] |
| 10 | —CH(CH₃)— | 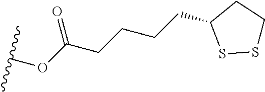 [SMILES: *OC(=O)CCCC[C@@H]1CCSS1] |
| 11 | —CH₂— | 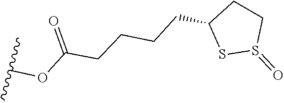 [SMILES: *OC(=O)CCCC[C@@H]1CCS(=O)S1] |
| 12 | bond | 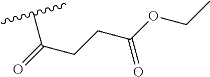 [SMILES: *C(=O)CCC(=O)OCC] |
| 13 | bond | 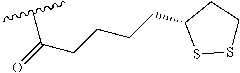 [SMILES: *C(=O)CCCC[C@@H]1CCSS1] |
| 14 | bond | 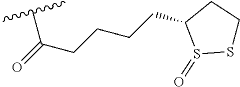 [SMILES: *C(=O)CCCC[C@@H]1CCSS1=O] |
| 15 | bond | 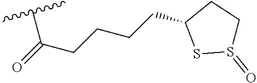 [SMILES: *C(=O)CCCC[C@@H]1CCS(=O)S1] |
| 16 | bond | 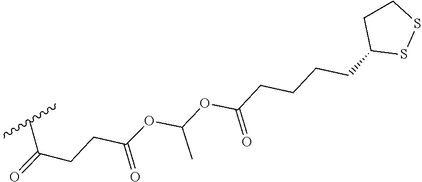 [SMILES: *C(=O)CCC(=O)OC(C)OC(=O)CCCC[C@@H]1CCSS1] |

Provided in some embodiments herein is a compound having a structure provided in Table 3, a stereoisomer thereof, or a pharmaceutically acceptable salt or solvate of the compound or the stereoisomer.

TABLE 3

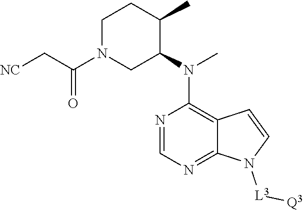

| Compound | L³ | Q³ |
|---|---|---|
| 17 | bond | 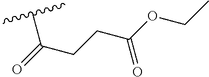 [SMILES: *C(=O)CCC(=O)OCC] |
| 18 | bond | 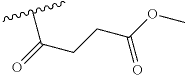 [SMILES: *C(=O)CCC(=O)OC] |
| 19 | —CH(CH₃)— | 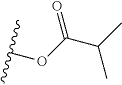 [SMILES: *OC(=O)C(C)C] |
| 20 | bond | 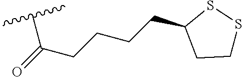 [SMILES: *C(=O)CCCC[C@@H]1CCSS1] |
| 21 | —CH₂— | 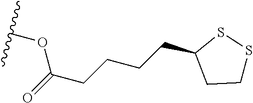 [SMILES: *OC(=O)CCCC[C@@H]1CCSS1] |
| 22 | bond | 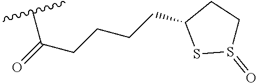 [SMILES: *C(=O)CCCC[C@@H]1CCS(=O)S1] |
| 23 | bond | 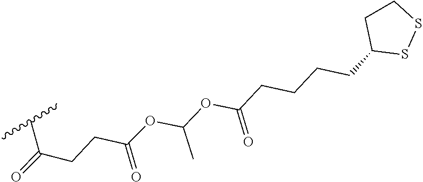 [SMILES: *C(=O)CCC(=O)OC(C)OC(=O)CCCC[C@@H]1CCSS1] |
| 24 | bond | 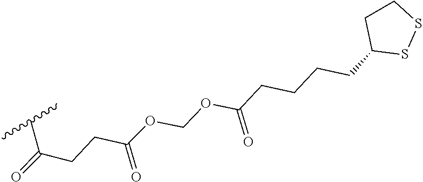 [SMILES: *C(=O)CCC(=O)OCOC(=O)CCCC[C@@H]1CCSS1] |

Provided in some embodiments herein is a compound having a structure provided in Table 4, a stereoisomer thereof, or a pharmaceutically acceptable salt or solvate of the compound or the stereoisomer.

TABLE 4

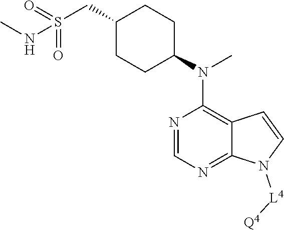

| Com- | | |
|---|---|---|
| pound | L⁴ | Q⁴ |
| 25 | —CH(CH₃)— | 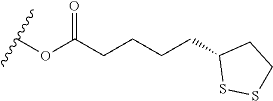 [SMILES: *OC(=O)CCCC[C@@H]1CCSS1] |
| 26 | —CH(CH₃)— | 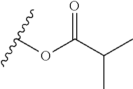 [SMILES: *OC(=O)C(C)C] |
| 27 | —CH₂— | 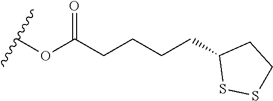 [SMILES: *OC(=O)CCCC[C@@H]1CCSS1] |
| 28 | —CH₂— | 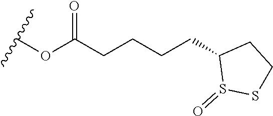 [SMILES: *OC(=O)CCCC[C@@H]1CCSS1=O] |
| 29 | —CH(CH₃)— | 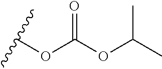 [SMILES: *OC(=O)OC(C)C] |
| 30 | bond | 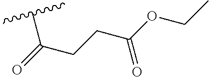 [SMILES: *C(=O)CCC(=O)OCC] |
| 31 | bond | 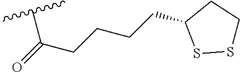 [SMILES: *C(=O)CCCC[C@@H]1CCSS1] |
| 32 | bond | 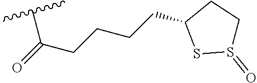 [SMILES: *C(=O)CCCC[C@@H]1CCS(=O)S1] |
| 33 | bond | 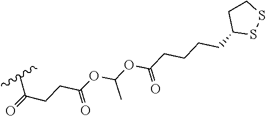 [SMILES: *C(=O)CCC(=O)OC(C)OC(=O)CCCC[C@@H]1CCSS1] |

Provided in some embodiments herein is a compound having a structure provided in Table 5, a stereoisomer thereof, or a pharmaceutically acceptable salt or solvate of the compound or the stereoisomer.

TABLE 5

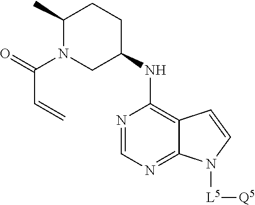

| Compound | L⁵ | Q⁵ |
|---|---|---|
| 34 | —CH(CH₃)— | 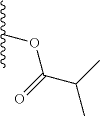 [SMILES: *OC(=O)C(C)C] |
| 35 | —CH(CH₃)— | 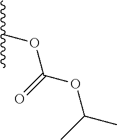 [SMILES: *OC(=O)OC(C)C] |
| 36 | bond | 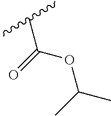 [SMILES: *C(=O)OC(C)C] |
| 37 | —CH₂— | 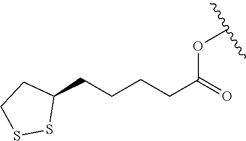 [SMILES: *OC(=O)CCCC[C@@H]1CCSS1] |
| 38 | —CH₂— | 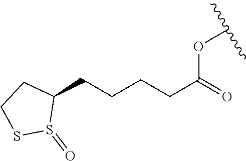 [SMILES: *OC(=O)CCCC[C@@H]1CCSS1=O] |
| 39 | bond | 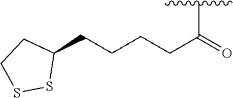 [SMILES: *C(=O)CCCC[C@@H]1CCSS1] |
| 40 | bond | 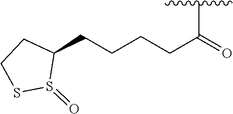 [SMILES: *C(=O)CCCC[C@@H]1CCSS1=O] |
| 41 | —CH(CH₃)— | 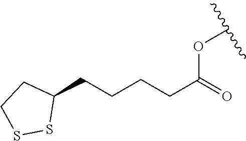 [SMILES: *OC(=O)CCCC[C@@H]1CCSS1] |
| 42 | —CH(CH₃)— | 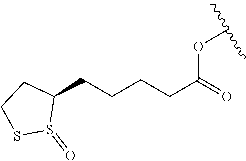 [SMILES: *OC(=O)CCCC[C@@H]1CCSS1=O] |
| 43 | bond | 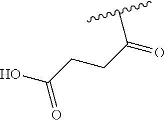 [SMILES: *C(=O)CCC(=O)O] |
| 44 | bond | 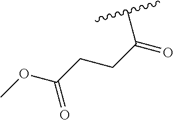 [SMILES: *C(=O)CCC(=O)OC] |
| 45 | bond | 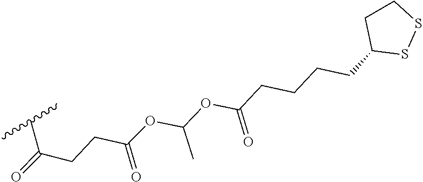 [SMILES: *C(=O)CCC(=O)OC(C)OC(=O)CCCC[C@@H]1CCSS1] |
| 46 | bond | 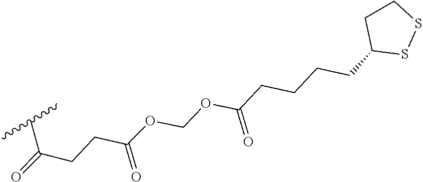 [SMILES: *C(=O)CCC(=O)OCOC(=O)CCCC[C@@H]1CCSS1] |

Provided in some embodiments herein is a compound having a structure provided in Table 6, a stereoisomer thereof, or a pharmaceutically acceptable salt or solvate of the compound or the stereoisomer.

TABLE 6

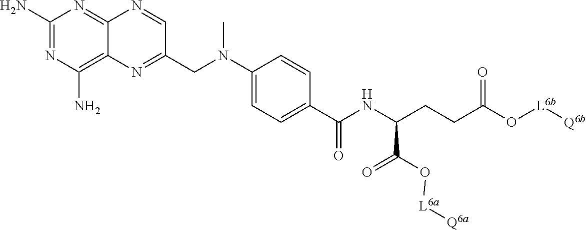

| Compound | L^{6a} | L^{6b} | Q^{6a} | Q^{6b} |
|---|---|---|---|---|
| 47 | —CH(CH₃)— | —CH(CH₃)— | 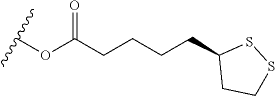 [SMILES: *OC(=O)CCCC[C@@H]1CCSS1] | 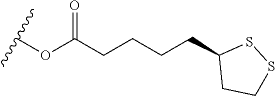 [SMILES: *OC(=O)CCCC[C@@H]1CCSS1] |
| 48 | —CH₂— | —CH₂— | 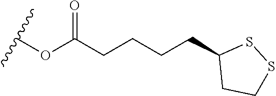 [SMILES: *OC(=O)CCCC[C@@H]1CCSS1] | 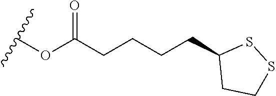 [SMILES: *OC(=O)CCCC[C@@H]1CCSS1] |
| 49 | —CH(CH₃)— | —CH₂— | 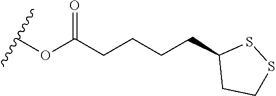 [SMILES: *OC(=O)CCCC[C@@H]1CCSS1] | 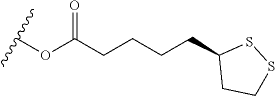 [SMILES: *OC(=O)CCCC[C@@H]1CCSS1] |
| 50 | —CH₂— | —CH(CH₃)— | 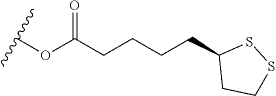 [SMILES: *OC(=O)CCCC[C@@H]1CCSS1] | 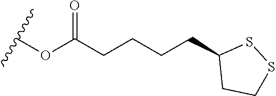 [SMILES: *OC(=O)CCCC[C@@H]1CCSS1] |
| 51 | —CH(CH₃)— | bond | 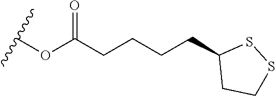 [SMILES: *OC(=O)CCCC[C@@H]1CCSS1] | —CH₂CH₃ |
| 52 | —CH₂— | bond | 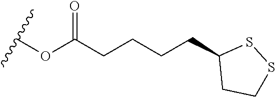 [SMILES: *OC(=O)CCCC[C@@H]1CCSS1] | —CH₂CH₃ |
| 53 | bond | —CH(CH₃)— | —CH₂CH₃ | 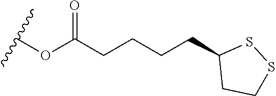 [SMILES: *OC(=O)CCCC[C@@H]1CCSS1] |
| 54 | bond | —CH₂— | —CH₂CH₃ | 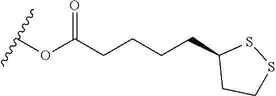 [SMILES: *OC(=O)CCCC[C@@H]1CCSS1] |
| 55 | —CH(CH₃)— | —CH(CH₃)— | 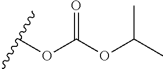 [SMILES: *OC(=O)OC(C)C] | 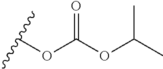 [SMILES: *OC(=O)OC(C)C] |
| 56 | bond | bond | —CH(CH₃)₂ | —CH(CH₃)₂ |
| 57 | —CH(CH₃)— | —CH(CH₃)— | 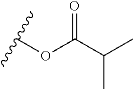 [SMILES: *OC(=O)C(C)C] | 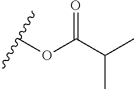 [SMILES: *OC(=O)C(C)C] |
| 58 | bond | bond | —CH₂CH₃ | —CH₂CH₃ |
| 59 | bond | bond | —CH₂CH₃ | H |
| 60 | bond | bond | H | —CH₂CH₃ |

Provided in some embodiments herein is a compound having a structure provided in Table 7, a stereoisomer thereof, or a pharmaceutically acceptable salt or solvate of the compound or the stereoisomer.

TABLE 7

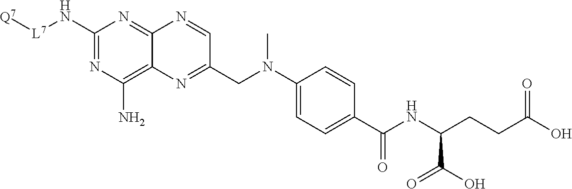

| Compound | L⁷ | Q⁷ |
|---|---|---|
| 61 | —CH(CH₃)— | 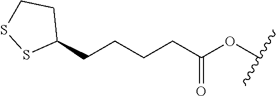 [SMILES: *OC(=O)CCCC[C@@H]1CCSS1] |
| 62 | —CH₂— | 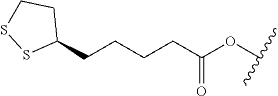 [SMILES: *OC(=O)CCCC[C@@H]1CCSS1] |
| 63 | bond | 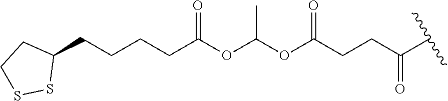 [SMILES: *C(=O)CCC(=O)OC(C)OC(=O)CCCC[C@@H]1CCSS1] |
| 64 | bond | 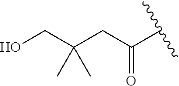 [SMILES: *C(=O)CC(C)(C)CO] |

Provided in some embodiments herein is a compound having a structure provided in Table 8, a stereoisomer thereof, or a pharmaceutically acceptable salt or solvate of the compound or the stereoisomer.

TABLE 8

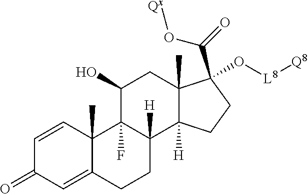

| Compound | Q^{x} | L⁸ | Q⁸ |
|---|---|---|---|
| 65 | ClCH₂— | bond | 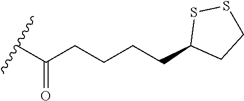 [SMILES: *C(=O)CCCC[C@@H]1CCSS1] |
| 66 | ClCH₂— | bond | 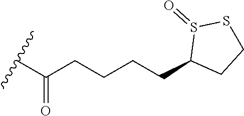 [SMILES: *C(=O)CCCC[C@@H]1CCSS1=O] |
| 67 | ClCH₂— | bond | 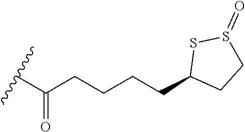 [SMILES: *C(=O)CCCC[C@@H]1CCS(=O)S1] |
| 68 | ClCH₂— | bond | 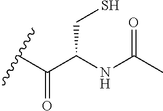 [SMILES: *C(=O)[C@H](CS)NC(C)=O] |
| 69 | H— | bond | 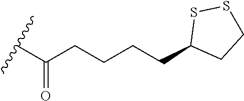 [SMILES: *C(=O)CCCC[C@@H]1CCSS1] |
| 70 | H— | bond | 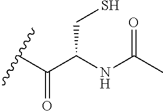 [SMILES: *C(=O)[C@H](CS)NC(C)=O] |
| 71 | ClCH₂— | bond | 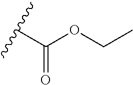 [SMILES: *C(=O)OCC] |
| 72 | H— | bond | 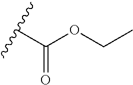 [SMILES: *C(=O)OCC] |
| 73 | ClCH₂— | bond | —H |

Provided in some embodiments herein is a compound having a structure provided in Table 8A, a stereoisomer thereof, or a pharmaceutically acceptable salt or solvate of the compound or the stereoisomer.

TABLE 8A

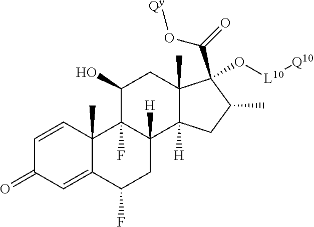

| Compound | Q^{y} | L^{8A} | Q^{8A} |
|---|---|---|---|
| 73A | NC(CH)₂— | bond | 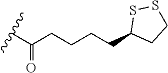 [SMILES: *C(=O)CCCC[C@@H]1CCSS1] |
| 73B | NC(CH)₂— | bond | 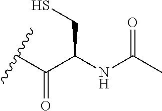 [SMILES: *C(=O)[C@@H](CS)NC(C)=O] |

In some instances, a free-acid is inactive in the glucocorticoid binding assay described in the Examples herein. In some instances, an ester is active in the glucocorticoid binding assay described in the Examples herein. In some instances, an ester retains activity, such as when attached to a keratolytic agent (or a radical thereof), in the glucocorticoid binding assay described in the Examples herein.

Provided in some embodiments herein is a compound having a structure provided in Table 9, a stereoisomer thereof, or a pharmaceutically acceptable salt or solvate of the compound or the stereoisomer.

TABLE 9

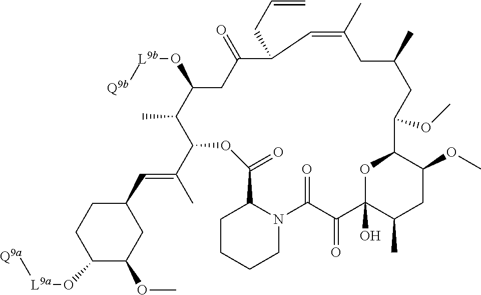

| Compound | L^{9a} | L^{9b} | Q^{9a} | Q^{9b} |
|---|---|---|---|---|
| 74 | bond | bond | 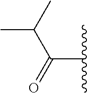 [SMILES: *C(=O)C(C)C] | —H |
| 75 | bond | bond | 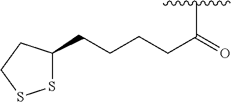 [SMILES: *C(=O)CCCC[C@@H]1CCSS1] | —H |
| 76 | bond | bond | 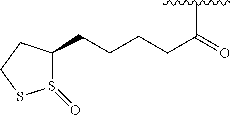 [SMILES: *C(=O)CCCC[C@@H]1CCSS1=O] | —H |
| 77 | bond | bond | —H | 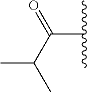 [SMILES: *C(=O)C(C)C] |
| 78 | bond | bond | —H | 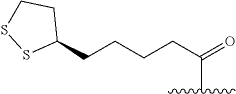 [SMILES: *C(=O)CCCC[C@@H]1CCSS1] |
| 79 | bond | bond | —H | 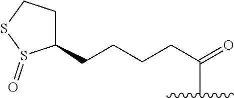 [SMILES: *C(=O)CCCC[C@@H]1CCSS1=O] |
| 80 | bond | bond | 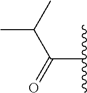 [SMILES: *C(=O)C(C)C] | 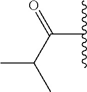 [SMILES: *C(=O)C(C)C] |
| 81 | bond | bond | 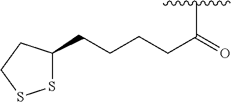 [SMILES: *C(=O)CCCC[C@@H]1CCSS1] | 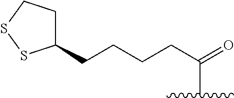 [SMILES: *C(=O)CCCC[C@@H]1CCSS1] |
| 82 | bond | bond | 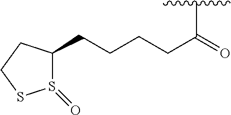 [SMILES: *C(=O)CCCC[C@@H]1CCSS1=O] | 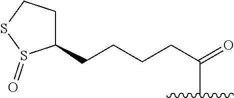 [SMILES: *C(=O)CCCC[C@@H]1CCSS1=O] |

Each recitation of

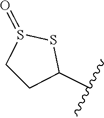 [SMILES: *C1CCS(=O)S1]

provided herein, unless otherwise stated, includes a specific and explicit recitation of:

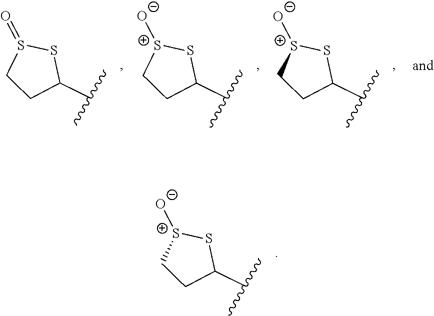

Each recitation of

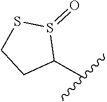 [SMILES: *C1CCSS1=O]

provided herein, unless otherwise stated, includes a specific

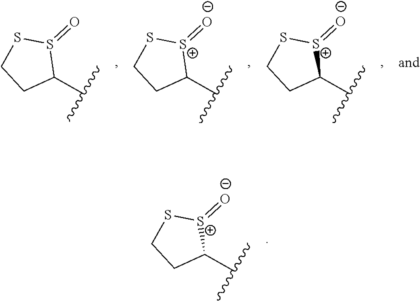

The compounds used in the reactions described herein are made according to organic synthesis techniques starting from commercially available chemicals and/or from compounds described in the chemical literature or provided herein. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen A G (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2^{nd} Ed., Academic Press, New York, 1983; H. 0. House, “Modern Synthetic Reactions”, 2^{nd} Ed., W. A. Benjamin, Inc. Menlo Park, Calif 1972; T. L. Gilchrist, “Heterocyclic Chemistry”, 2^{nd} Ed., John Wiley & Sons, New York, 1992; J. March, “Advanced Organic Chemistry: Reactions, Mechanisms and Structure”, 4^{th} Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R.V. “Organic Chemistry, An Intermediate Text” (1996) Oxford University Press, ISBN 0-19-509618-5; Larock, R. C. “Comprehensive Organic Transformations: A Guide to Functional Group Preparations” 2^{nd} Edition (1999) Wiley-VCH, ISBN: 0-471-19031-4; March, J. “Advanced Organic Chemistry: Reactions, Mechanisms, and Structure” 4^{th} Edition (1992) John Wiley & Sons, ISBN: 0-471-60180-2; Otera, J. (editor) “Modern Carbonyl Chemistry” (2000) Wiley-VCH, ISBN: 3-527-2987 1-1; Patai, S. “Patai's 1992 Guide to the Chemistry of Functional Groups” (1992) Interscience ISBN: 0-471-93022-9; Solomons, T. W. G. “Organic Chemistry” 7^{th} Edition (2000) John Wiley & Sons, ISBN: 0-471-19095-0; Stowell, J. C., “Intermediate Organic Chemistry” 2^{nd} Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of chemicals prepared by the Chemical Abstract Service of the American Chemical Society, which are available in most public and university libraries, as well as through on-line databases (contact the American Chemical Society, Washington, D.C. for more details). Chemicals not commercially available in catalogs are optionally prepared by custom chemical synthesis houses, where many of the standard chemical supply houses (e.g., those listed above) provide custom synthesis services. A reference for the preparation and selection of pharmaceutical salts of the keratolytic conjugate described herein is P. H. Stahl & C. G. Wermuth “Handbook of Pharmaceutical Salts”, Verlag Helvetica Chimica Acta, Zurich, 2002.

In some embodiments, a compound provided herein is represented by any one of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9. In some embodiments, a compound provided herein is administered as a pure chemical. In other embodiments, a compound provided herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided in some embodiments herein is a pharmaceutical composition comprising at least one keratolytic conjugate together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

In some embodiments, a compound provided herein (e.g., a compound having a structure represented by any one of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9) is substantially pure, in that it contains less than, for example, about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments provided herein is a pharmaceutical composition comprising a compound provided herein (e.g., a compound having a structure represented by any one of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9) and at least one pharmaceutically acceptable excipient. In some embodiments, the pharmaceutical composition is suitable for dermal administration. In some embodiments, the pharmaceutical composition is suitable for ophthalmic administration. In some embodiments, the pharmaceutical composition is suitable for topical ophthalmic administration. In some embodiments, topical ophthalmic administration is administration in and/or around the eye, such as to the eyelid margin. In some embodiments, the pharmaceutical composition is suitable for administration to the eyelid margin. In some embodiments, topical ophthalmic administration is administration to the ocular surface and the inner surface to the eyelid.

In some embodiments, a keratolytic conjugate provided herein (e.g., a compound having a structure represented any one of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9) is formulated as a solution or suspension for topical administration to the eye.

In some embodiments, a keratolytic conjugate provided herein (e.g., a compound having a structure represented any one of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9) is formulated as a solution or suspension for topical administration to the skin.

In some embodiments, a keratolytic conjugate provided herein (e.g., a compound having a structure represented by any one of Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9) is formulated for administration by injection. In some instances, the injection formulation is an aqueous formulation. In some instances, the injection formulation is a non-aqueous formulation. In some instances, the injection formulation is an oil-based formulation, such as sesame oil, or the like.

In some embodiments, the dose of the composition comprising at least one keratolytic conjugate as provided herein differ, depending upon the patient's (e.g., human) condition, that is, general health status, age, and other factors.

Pharmaceutical compositions provided in some embodiments herein are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. The optimal dose depends upon the body mass, weight, or blood volume of the patient.

In other embodiments, the topical compositions described herein are combined with a pharmaceutically suitable or acceptable carrier (e.g., a pharmaceutically suitable (or acceptable) excipient, physiologically suitable (or acceptable) excipient, or physiologically suitable (or acceptable) carrier). Exemplary excipients are described, for example, in Remington: The Science and Practice of Pharmacy (Gennaro, 21st Ed. Mack Pub. Co., Easton, PA (2005)).

### Methods of Treatment Utilizing Keratolytic Conjugates

In some embodiments provided herein is a method of treating a dermatological or ophthalmic disease or disorder in a patient in need of thereof, comprising administering to the patient any compound provided herein, or a pharmaceutically acceptable salt thereof, or a (e.g., pharmaceutical) composition comprising any compound provided herein, or a pharmaceutically acceptable salt thereof, such as a compound represented by any structure herein, such as, for example, Formula (A), Formula (A-I), Formula (A-II), Formula (I-A), Formula (I-AA), Formula (I-B), Formula (I-BA), Formula (I-BB), Formula (I-BC), Formula (I-BD), Formula (I-C1), Formula (I-C2), Formula (I-C3), Formula (I-CA), Formula (I-CB), Formula (I-D), Formula (I-DA), Formula (I-DB), Formula (I-E), Formula (I-E′), Formula (I-EA), Formula (I-EB), Formula (I-EC), Table 1, Table 2, Table 3, Table 4, Table 5, Table 6, Table 7, Table 8, or Table 9. In some embodiments provided herein the pharmaceutical composition is in the form of a solution or suspension suitable for (e.g., topical) ophthalmic administration. In some embodiments, (e.g., topical) ophthalmic administration is administration in and/or around the eye, such as to the eyelid margin. In some embodiments, (e.g., topical) ophthalmic administration is administration to the ocular surface and the inner surface to the eyelid. In some embodiments, the dermatological or ophthalmic disease or disorder is improper immunomodulation, inflammation, and/or hyperkeratosis (e.g., of the eyes or skin). In some embodiments, the dermatological or ophthalmic disease or disorder is improper immunomodulation, inflammation or hyperkeratosis of the eyes or skin (e.g., the ocular surface). In some embodiments, the dermatological or ophthalmic dermatological disease or disorder is selected from the group consisting of meibomian gland dysfunction (MGD), dry eye disease (DED), ocular manifestations of graft versus host disease, vernal keratoconjunctivitis, atopic keratoconjunctivitis, Cornelia de Lange Syndrome, evaporative eye disease, aqueous deficiency dry eye, blepharitis, and seborrheic blepharitis. In some embodiments, the dermatological or ophthalmic disease or disorder is inflammation or hyperkeratosis (e.g., of the eyes or skin), such as, for example, meibomian gland dysfunction (MGD), dry eye disease (DED), ocular manifestations of graft versus host disease, vernal keratoconjunctivitis, atopic keratoconjunctivitis, Cornelia de Lange Syndrome, evaporative eye disease, aqueous deficiency dry eye, blepharitis, seborrheic blepharitis, or any combination thereof.

In some embodiments, the ophthalmic disease or disorder is selected from the group consisting of dry eye, lid wiper epitheliopathy (LWE), contact lens discomfort (CLD), dry eye syndrome, evaporative dry eye syndrome, aqueous deficiency dry eye syndrome, blepharitis, keratitis, meibomian gland dysfunction, conjunctivitis, lacrimal gland disorder, contact lens related conditions and inflammation of the anterior surface of the eye, infection of the anterior surface of the eye, and autoimmune disorder of the anterior surface of the eye.

In certain embodiments, methods provided herein involve the method of treating meibomian gland dysfunction (MGD).

In some embodiments, the ocular disorder is a surface disorder, such as MGD, dry eye and associated inflammatory and bacterial disease, an (e.g., severe) ocular allergy (e.g., keratoconjunctivitis (e.g., atopic keratoconjunctivitis (AKC) or vernal keratoconjunctivitis (VKC))), a (e.g., inflammatory and/or aqueous) dry eye disease, or an ocular manifestation of graft versus host disease (ocular GVHD).

In some embodiments, the ocular disorder is a periocular disorder. In some embodiments, the periocular disorder is a sty, blepharitis, a chalazion, or dacryoadenitis.

In some embodiments, the dermal disorder is comedonal acne, hyperkeratosis, scleroderma, seborrheic dermatitis, atopic dermatitis, psoriasis, lichen planus, an insect bite, intertrigo, pemphigus, or pityriasis rubra pilaris.

Provided herein is a method for treating an ocular surface disorder in an individual in need thereof comprising topical administration of a keratolytic conjugate to the individual in need thereof. In some embodiments, administration occurs with the assistance of a health-care provider (e.g., this category includes both acute and maintenance uses of the keratolytic conjugate). An acute use, in some embodiments, requires a stronger keratolytic conjugate (either in terms of concentration of the agent or the inherent activity of the agent). A maintenance use, in some embodiments, allows for the use of lower concentrations of the agent, or agents with lower inherent activity. A maintenance use, in some embodiments, involves a patient at a routine visit to the health care provider. Both acute uses and maintenance uses optionally involve use of an eye-protecting device or apparatus. In some embodiments, the acute use is performed by the health care provider, and the maintenance use is performed by the patient or non-health care provider. In some embodiments, administration does not occur with the active assistance of a health care provider (e.g., but rather involves the patient applying the keratolytic conjugate to his/her own eyelid margin). In some embodiments, such administration occurs over an extended period of time (e.g., one way of describing this patient-administered multi-administration mode is as a chronic use). In some embodiments, different or second formulations of the keratolytic conjugate are used for chronic or patient-administered uses. In some embodiments the different or second formulation utilizes a lower concentration of the keratolytic conjugate. In some embodiments, the second or different formulation utilizes a keratolytic conjugate that has a lower activity than the first formulation.

It should be understood that the present methods also include the physical removal of an obstruction in an meibomian gland (e.g., followed by chronic and/or maintenance administration of a keratolytic conjugate provided herein).

In some embodiments provided herein is a method for treating meibomian gland dysfunction in a patient in need thereof, comprising topically administering to the patient a composition comprising a therapeutically-effective amount of at least one keratolytic conjugate in an ophthalmically-acceptable carrier. In some embodiments, the topical administration of the composition comprising a therapeutically-effective amount of at least one keratolytic conjugate in an ophthalmically-acceptable carrier results in enhanced meibum production.

In some embodiments, the topical administration of the composition comprising a therapeutically-effective amount of at least one keratolytic conjugate in an ophthalmically-acceptable carrier occurs until the keratinized obstruction is relieved. In some embodiments, the topical administration of the composition comprising a therapeutically-effective amount of at least one keratolytic conjugate in an ophthalmically-acceptable carrier occurs periodically after relieving of the keratinized obstruction. In some embodiments, the topical administration of the composition comprising a therapeutically-effective amount of at least one keratolytic conjugate in an ophthalmically-acceptable carrier is a single administration. In some embodiments, the topical administration of the composition comprising a therapeutically-effective amount of at least one keratolytic conjugate in an ophthalmically-acceptable carrier is a periodic administration. In some embodiments, the topical administration of the composition comprising a therapeutically-effective amount of at least one keratolytic conjugate in an ophthalmically-acceptable carrier occurs once a day. In some embodiments, the topical administration of the composition comprising a therapeutically-effective amount of at least one keratolytic conjugate in an ophthalmically-acceptable carrier occurs twice a day. In some embodiments, the topical administration of the composition comprising a therapeutically-effective amount of at least one keratolytic conjugate in an ophthalmically-acceptable carrier occurs more than twice a day.

In some embodiments, the composition for topical administration comprises a therapeutically-effective amount of at least one keratolytic conjugate in an ophthalmically-acceptable carrier is a solution. In some embodiments, the composition for topical administration comprises a therapeutically-effective amount of at least one keratolytic conjugate in an ophthalmically-acceptable carrier is a solution suitable for topical administration as eye drops. In some embodiments, the composition for topical administration comprises a therapeutically-effective amount of at least one keratolytic conjugate in an ophthalmically-acceptable carrier is a gel, ocular insert, spray, or other topical ocular delivery method. In some embodiments, the composition for topical administration comprises a therapeutically-effective amount of at least one keratolytic conjugate in an ophthalmically-acceptable carrier is a semi-solid. In some embodiments, the composition for topical administration comprises a therapeutically-effective amount of at least one keratolytic conjugate in an ophthalmically-acceptable carrier is homogenous. In some embodiments, the composition for topical administration comprises a therapeutically-effective amount of at least one keratolytic conjugate in an ophthalmically-acceptable carrier is a dispersion. In some embodiments, the composition for topical administration comprises a therapeutically-effective amount of at least one keratolytic conjugate in an ophthalmically-acceptable carrier is hydrophilic. In some embodiments, the composition for topical administration comprises a therapeutically-effective amount of at least one keratolytic conjugate in an ophthalmically-acceptable carrier and an oleaginous base. In some embodiments, the composition for topical administration comprises a therapeutically-effective amount of at least one keratolytic conjugate in an ophthalmically-acceptable carrier and at least one ophthalmically-acceptable excipient.

In some embodiments provided herein is a method for treating MGD in a patient in need thereof comprising topical administration of a composition comprising a keratolytic conjugate. In some embodiments, the topical administration of the composition comprising a keratolytic conjugate occurs once a week. In some embodiments, the topical administration of the composition comprising a keratolytic conjugate occurs twice a week. In some embodiments, the topical administration of the composition comprising a keratolytic conjugate occurs every other day. In some embodiments, the topical administration of the composition comprises a keratolytic conjugate occurs every day. In some embodiments, the topical administration of the composition comprises a keratolytic conjugate occurs several times a day.

In some embodiments, the method comprises administering a compound or formulation provided herein in an acute treatment scenario. In some embodiments, the method comprises treatment of a patient naive to treatment. In some embodiments, the method comprises administering a compound or formulation provided herein in a chronic treatment scenario. In some embodiments, the method comprises administering a compound or formulation provided herein in a maintenance therapy scenario. In an acute treatment scenario, the administered dosage of keratolytic conjugate may be higher than the administered dosage of keratolytic conjugate employed in a chronic treatment scenario or a maintenance therapy scenario. In an acute treatment scenario, the keratolytic conjugate may be different from the keratolytic conjugate employed in a chronic treatment scenario. In some embodiments, the course of therapy begins in the initial phase of therapy as an acute treatment scenario and later transitions into a chronic treatment scenario or a maintenance therapy scenario. In some embodiments, the meibomian gland opening pharmacological agent administered in the acute treatment scenario is a keratolytic agent and/or keratoplastic agent, and the pharmacological agent administered in the chronic treatment scenario or a maintenance therapy scenario is a keratolytic conjugate.

In some embodiments, an initial treatment is administered (e.g., by a physician or healthcare professional) to an individual to initially open a blockage of the meibomian gland, such as by placing a more highly concentrated formulation of one of the keratolytic conjugate provided herein. In the event the higher concentration formulations are required, the application thereof may require ocular shielding or other activity to minimize the impact of irritation or disruption of the ocular surface or surrounding tissues. Following such a procedure, a patient may be given a different formulation of keratolytic conjugate to take home to apply periodically to the lid margin to maintain the patency of the meibomian gland. Such application may occur twice daily, once a day, weekly or monthly, depending on the formulation activity and the therapeutic product profile of the formulation.

Provided in some embodiments of the methods of treatment described herein is the location of the topical administration of the composition. In some embodiments, the composition comprising a keratolytic conjugate is administered such that no irritation to eye occurs. In some embodiments, the composition comprising a keratolytic conjugate is administered to the eye lid margin.

In some embodiments of the methods of treatment provided herein is the use of a protective element provided to the eye to avoid irritation to the eye. Although the formulations described herein are generally non-irritating, in some embodiments (e.g., high concentration of agent or when used on a sensitive eye) a protective element provides an additional layer of safety and comfort for the patient. In some embodiments, the composition comprising a keratolytic conjugate is administered while an eye shield is placed on the eye to reduce contact of the pharmacological agent with the cornea and/or conjunctiva such that reduced irritation to eye occurs. In some embodiments, the eye shield is a contact lens or an eye covering. In some embodiments, the eye covering comprises a self-adhesive. In some embodiments, the composition comprising a keratolytic conjugate is administered while the lid is pulled away from the globe to reduce contact of the pharmacological agent with the cornea and/or conjunctiva such that reduced irritation to eye occurs.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### I. Chemical Synthesis

Solvents, reagents, and starting materials were purchased from commercial vendors and used as received unless otherwise described. All reactions were performed at room temperature unless otherwise stated. Starting materials were purchased from commercial sources or synthesized according to the methods described herein or using literature procedures.

### Abbreviations

The following abbreviations are used in the Examples and other parts of the examples:
CDCl₃: Deuterated chloroform
DBU: 1,8-Diazabicyclo[5,4,0]undec-7-ene
DCM: Dichloromethane
DIPEA: N,N-Diisopropylethylamine
DMAP: 4-Dimethylaminopyridine
DMF: N,N-Dimethylformamide
EDCI: 1-(3-dimethylaminopropyl)-3-ethyl-carbodiimide hydrochloride
Equiv: equivalent(s)
EtOAc: Ethyl acetate
h: Hour(s)
HATU: (1-[Bis(dimethylamino)methylene]-1H-1,2,3-triazolo[4,5-b]pyridinium 3-oxide hexafluorophosphate
HPLC: High Performance Liquid Chromatography
LCMS: Liquid chromatography-mass spectrometry
M: Molar
MeCN: Acetonitrile
MeOH: Methanol
Min(s): Minute(s)
o/n: overnight
r.t.: Room temperature
Rt: Retention time
sat.: Saturated
TEA: Triethylamine
TFA: Trifluoroacetic acid
THF: Tetrahydrofuran
vac: Vacuum

### Analytical Methods:

Method A: Waters Acquity UPLC BEH C18 1.7 m, 2.1×30 mm; A=water+0.1% formic acid; B=MeCN; 45° C.; % B: 0.0 min 5% 0.60 mL/min, 0.05 min 5% 0.60 mL/min, 1.6 min 95% 0.60 mL/min, 2.25 min 95% 0.75 mL/min, 2.26 min 5% 0.60 mL/min, 2.60 min 5% 0.60 mL/min.

Method B: Waters Acquity UPLC BEH C18 1.7 m, 2.1×30 mm; A=water+10 mM ammonium bicarbonate; B=MeCN; 45° C.; % B: 0.0 min 5% 0.60 mL/min, 0.05 min 5% 0.60 mL/min, 1.6 min 95% 0.60 mL/min, 2.25 min 95% 0.75 mL/min, 2.26 min 5% 0.60 mL/min, 2.60 min 5% 0.60 mL/min.

Method C: Waters Acquity UPLC BEH C18 1.7 m, 2.1×30 mm; A=water+0.1% formic acid; B=MeCN; 45° C.; % B: 0.0 min 5% 0.35 mL/min, 0.05 min 5% 0.35 mL/min, 5.0 min 95% 0.35 mL/min, 6.5 min 95% 0.35 mL/min, 6.6 min 5% 0.35 mL/min, 9.0 min 5% 0.35 mL/min

Method D: Waters Acquity UPLC BEH C18 1.7 m, 2.1×30 mm; A=water+0.1% ammonia; B=MeCN; 45° C.; % B: 0.0 min 5% 0.35 mL/min, 0.05 min 5% 0.35 mL/min, 5.0 min 95% 0.35 mL/min, 6.5 min 95% 0.35 mL/min, 6.6 min 5% 0.35 mL/min, 9.0 min 5% 0.35 mL/min.

Method E: Waters Acquity UPLC BEH C18 1.7 m, 2.1×30 mm; A=water+10 mM ammonium bicarbonate; B=MeCN; 45° C.; % B: 0.0 min 5% 0.35 mL/min, 0.05 min 5% 0.35 mL/min, 5.0 min 95% 0.35 mL/min, 6.5 min 95% 0.35 mL/min, 6.6 min 5% 0.35 mL/min, 9.0 min 5% 0.35 mL/min.

Method F: Phenomenex Gemini NX C18 5 m, 150×4.6 mm; A=water+0.1% formic acid; B=MeOH+0.1% formic acid; 40° C.; % B: 0.0 min 5% 1.5 mL/min, 0.5 min 5% 1.5 mL/min, 7.5 min 95% 1.5 mL/min, 10.0 min 95% 1.5 mL/min, 10.1 min 5% 1.5 mL/min, 13.0 min 5% 1.5 mL/min.

Method G: Phenomenex Gemini NX C18 5 m, 150×4.6 mm; A=water+0.1% formic acid; B=MeCN+0.1% formic acid; 40° C.; % B: 0.0 min 5% 1.5 mL/min, 0.5 min 5% 1.5 mL/min, 7.5 min 95% 1.5 mL/min, 10.0 min 95% 1.5 mL/min, 10.1 min 5% 1.5 mL/min, 13.0 min 5% 1.5 mL/min.

Method H: Waters Acquity UPLC BEH C18 1.7 m, 2.1×50 mm; A=water+0.1% formic acid; B=MeCN; 45° C.; % B: 0.0 min 5% 0.60 mL/min, 0.05 min 5% 0.60 mL/min, 1.6 min 95% 0.60 mL/min, 2.25 min 95% 0.75 mL/min, 2.26 min 5% 0.60 mL/min, 2.60 min 5% 0.60 mL/min.

Method I: Waters Acquity UPLC BEH C18 1.7 m, 2.1×50 mm; A=water+10 mM ammonium bicarbonate; B=MeCN; 45° C.; % B: 0.0 min 5% 0.60 mL/min, 0.05 min 5% 0.60 mL/min, 1.6 min 95% 0.60 mL/min, 2.25 min 95% 0.75 mL/min, 2.26 min 5% 0.60 mL/min, 2.60 min 5% 0.60 mL/min.

Method J: Waters Acquity CSH C18 1.7 m, 2.1×100 mm; A=water+0.1% formic acid; B=MeCN+0.1% formic acid; 40° C.; % B: 0.0 min 5% 0.5 min 5%, 5.0 min 95%, 6.5 min, 6.6 min 5%, 9.0 min 5% 0.35 mL/min.

Method K: Waters Acquity CSH C18 1.7 m, 2.1×100 mm; A=pH 9 ammonium bicarbonate 10 mM aqueous solution; B=MeCN; 40° C.; % B: 0.0 min 5% 0.5 min 5%, 5.0 min 95%, 6.5 min, 6.6 min 5%, 9.0 min 5% 0.35 mL/min.

Method L: Waters Sunfire C18 3.5 μm, 4.6 mm×50 mm; A=water+0.1% formic acid; B=MeCN; 45° C.; % B: 0.0 min 5% 2.25 mL/min 1.00 min 37.5% 2.20 mL/min 3.00 min 95% 2.2 mL/min 3.5 min 95% 2.3 mL/min 3.51 min 5% 2.3 mL/min 4.00 min 5% 2.25 mL/min.

### Chemical Synthesis Example 1

### General Procedure A: Chloroester Formation

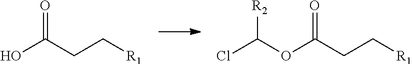

A mixture of carboxylic acid (1.0 equiv), sulfochoridate (1.4 equiv), NaHC₃ (4.0 equiv), tetrabutyl ammonium hydrogen sulphate (10 mol %), DCM and water (1:1) were stirred vigorously at r.t. for 16 hours. It was then passed through a phase separator, washed with sat. NaHCO₃ solution and sat. brine solution, dried (MgSO₄), filtered and the solvent concentrated in vacuo to afford the desired product without any further purification.

TABLE 10

| Chemical Synthesis | | |
|---|---|---|
| Example | Name and structure | Analytical data |
| 1 | tert-Butyl (chloromethyl) succinate 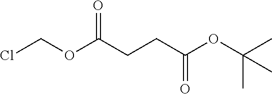 | Synthesised according to general procedure A, using 4- (tert-butoxy)-4-oxobutanoic acid (17.2 mmol) and chloromethyl chlorosulfate (24.1 mmol). ¹H-NMR (400 MHz, CDCl₃) δ 4.97 (s, 2H), 1.72-2.09 (m, 4H), 0.69 (s, 9H). |
| | Colourless oil, 3.70 g, 98% | |
| 2 | tert-Butyl (1-chloroethyl) succinate 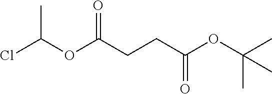 | Synthesised according to general procedure A, using 4- (tert-butoxy)-4-oxobutanoic acid (2.9 mmol) and 1- chloroethyl sulfochloridate (4.0 mmol). ¹H-NMR (400 MHz, CDCl₃) δ 6.55 (q, J = 6.0 Hz, 1H), 2.48- 2.70 (m, 4H), 1.78-1.86 (m, |
| | Colourless oil, 386 mg, 57% | 3H), 1.37-1.45 (m, 9H). |
| 3 | Chloromethyl (R)-5-(1,2- dithiolan-3-yl)pentanoate 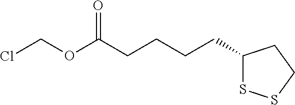 [SMILES: O=C(CCCC[C@@H]1CCSS1)OCCl] | Synthesised according to general procedure A, using lipoic acid (2.4 mmol) and chloromethyl chlorosulfate (3.4 mmol). The compound was used immediately in the next step without any further purification. |
| | Yellow oil, 240 mg, 37% | |
| 4 | 1-Chloroethyl 5-((R)-1,2- dithiolan-3-yl)pentanoate 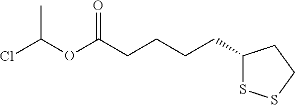 | Synthesised according to general procedure A, using lipoic acid (3.8 mmol) and 1- chloroethyl sulfurochloridate (5.3 mmol). ¹H-NMR (400 MHz, CDCl₃): δ 6.53 (q, J = 5.8 Hz, 1H), 3.48- 3.61 (m, 1H), 3.04-3.22 (m, 2H), 2.30-2.53 (m, 3H), 1.84- 1.96 (m, 1H), 1.77 (d, J = 6.0 |
| | Yellow oil, 617 mg, 34% | Hz, 3H), 1.59-1.74 (m, 4H), |
| | | 1.39-1.58 (m, 2H) |

### Chemical Synthesis Example 5

### (R)-((5-(1,2-Dithiolan-3 yl)pentanoyl)oxy)methyl tert-butyl succinate

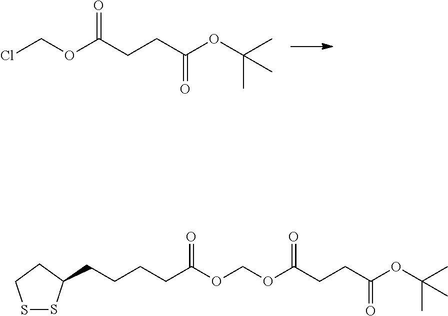 [SMILES: CC(C)(C)OC(=O)CCC(=O)OCCl ; CC(C)(C)OC(=O)CCC(=O)OCOC(=O)CCCC[C@@H]1CCSS1]

To a solution of lipoic acid (4.17 g, 20.2 mmol) and DIPEA (8.78 mL, 50.4 mmol) in DMF (30 mL), tert-butyl (chloromethyl) succinate (3.74 g, 16.8 mmol) was added and the reaction mixture stirred at 50° C. for 16 h. The reaction was diluted with EtOAc and washed with 1:1 H₂O-sat. brine solution, dried (MgSO₄), filtered and the solvent evaporated in vacuo. The crude product was purified by flash chromatography (Biotage Isolera Four; 100 g Sfar cartridge) eluting with isohexane→60% EtOAc-isohexane to afford (R)-((5-(1,2-dithiolan-3-yl)pentanoyl)oxy)methyl tert-butyl succinate as a yellow oil (3.15 g, 48%). LCMS (Method A): Rt=2.05 min; [M+Na]⁺=415.2. ¹H-NMR (400 MHz, CDCl₃) δ 5.73 (s, 2H), 3.48-3.59 (m, 1H), 3.03-3.22 (m, 2H), 2.56-2.67 (m, 2H), 2.49-2.56 (m, 2H), 2.38-2.49 (m, 1H), 2.34 (t, J=7.3 Hz, 2H), 1.82-1.94 (m, 1H), 1.53-1.74 (m, 4H), 1.36-1.52 (m, 11H).

### Chemical Synthesis Example 6

### 1-((5-((R)-1,2-Dithiolan-3-yl)pentanoyl)oxy)ethyl tert-butyl succinate

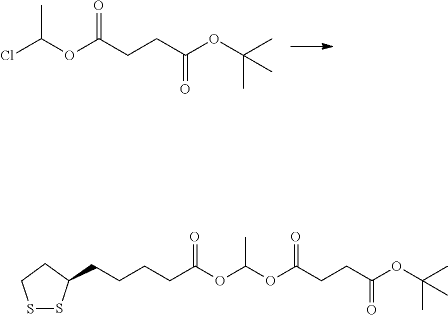 [SMILES: CC(Cl)OC(=O)CCC(=O)OC(C)(C)C ; CC(OC(=O)CCCC[C@@H]1CCSS1)OC(=O)CCC(=O)OC(C)(C)C]

To a solution of lipoic acid (270 mg, 1.31 mmol) and DIPEA (0.68 mL, 3.90 mmol) in DMF (2.0 mL), tert-butyl (1-chloroethyl) succinate (386 mg, 1.60 mmol) was added and the reaction mixture stirred at 50° C. for 16 h. The reaction mixture was diluted with EtOAc and washed with 1:1 H₂O-sat. brine solution, dried (MgSO₄), filtered and the solvent evaporated in vacuo. The crude product was purified by flash chromatography (Biotage Isolera Four; 100 g Sfar cartridge) eluting with 75% EtOAc-isohexane to afford 1-((5-((R)-1,2-dithiolan-3-yl)pentanoyl)oxy)ethyl tert-butyl succinate as a yellow oil (114 mg, 21%). LCMS (Method A): Rt=2.17 min; [M+H]⁺=407.2. ¹H NMR (400 MHz, CDCl₃) δ 6.84-6.89 (m, 1H), 3.55 (dt, J=14.7, 6.4 Hz, 1H), 3.09-3.20 (m, 1H), 2.44-2.63 (m, 6H), 2.29-2.33 (m, 1H), 1.86-1.94 (m, 1H), 1.60-1.71 (m, 2H), 1.51-1.59 (m, 3H), 1.46 (dd, J=5.5, 1.4 Hz, 3H), 1.43-1.44 (m, 10H).

### Chemical Synthesis Example 7

### (R)-4-(((5-(1,2-Dithiolan-3-yl)pentanoyl)oxy)methoxy)-4-oxobutanoic acid

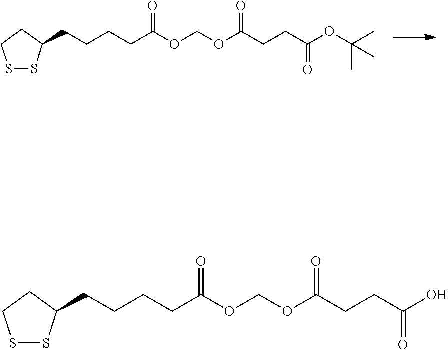 [SMILES: CC(C)(C)OC(=O)CCC(=O)OCOC(=O)CCCC[C@@H]1CCSS1 ; O=C(O)CCC(=O)OCOC(=O)CCCC[C@@H]1CCSS1]

To a solution of (R)-((5-(1,2-Dithiolan-3-yl)pentanoyl)oxy)methyl tert-butyl succinate (110 mg, 0.28 mmol) in DCM (3.0 mL), was added TFA (400 μL, 5.23 mmol) and the reaction mixture stirred at r.t. for 3 h. The pH of the reaction mixture was then adjusted to pH 4 with sat. NaHCO₃(aq) sol. and the reaction mixture passed through a phase separator. The filtrate was evaporated in vacuo to afford (R)-4-(((5-(1,2-dithiolan-3-yl)pentanoyl)oxy)methoxy)-4-oxobutanoic acid as a colorless oil (80.0 mg, 85%). This was used without further purification. LCMS (Method A): Rt=1.66 min; [M+NH₄]⁺=354.2.

### Chemical Synthesis Example 8

### 4-(1-((5-((R)-1,2-Dithiolan-3-yl)pentanoyl)oxy)ethoxy)-4-oxobutanoic acid

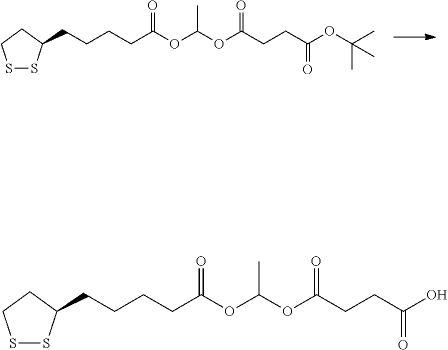 [SMILES: CC(OC(=O)CCCC[C@@H]1CCSS1)OC(=O)CCC(=O)OC(C)(C)C ; CC(OC(=O)CCCC[C@@H]1CCSS1)OC(=O)CCC(=O)O]

To a solution of 1-((5-((R)-1,2-dithiolan-3-yl)pentanoyl)oxy)ethyl tert-butyl succinate (114 mg, 0.280 mmol) in DCM (4.0 mL), was added TFA (400 μL, 5.96 mmol) and the reaction mixture stirred at r.t. for 3 h. The pH of the reaction mixture was then adjusted to pH 4 with sat. NaHCO_{3(aq)} and the reaction mixture passed through a phase separator. The filtrate was evaporated in vacuo to afford 4-(1-((5-((R)-1,2-dithiolan-3-yl)pentanoyl)oxy)ethoxy)-4-oxobutanoic acid as a colorless oil (27.0 mg, 27%). This was used without further purification.

LCMS (Method A): Rt=1.78 min; [M+NH₄]⁺=368.1; [M+Na]⁺=373.1.

### Chemical Synthesis Example 9

### Benzyl (2S,5R)-5-((7H-pyrrolo[2,3-d]pyrimidin-4-yl)amino)-2-methylpiperidine-1-carboxylate

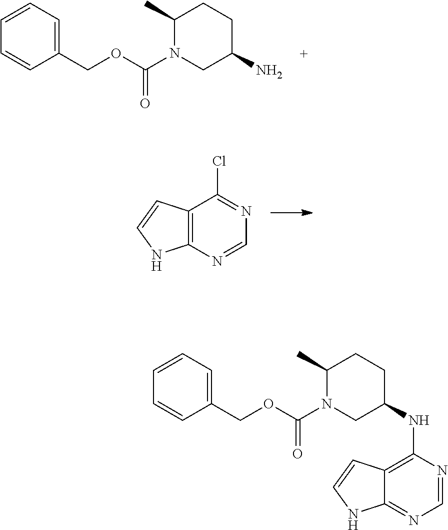 [SMILES: C[C@H]1CC[C@@H](N)CN1C(=O)OCc1ccccc1 ; Clc1ncnc2[nH]ccc12 ; C[C@H]1CC[C@@H](Nc2ncnc3[nH]ccc23)CN1C(=O)OCc1ccccc1]

A mixture of benzyl (2S,5R)-5-amino-2-methylpiperidine-1-carboxylate (2.00 g, 8.05 mmol), potassium carbonate (3.56 g, 25.8 mmol) and 4-chloro-7H-pyrrolo(2,3,d)pyrimidine (1.30 g, 8.48 mmol) in water (20 mL) was stirred at 90° C. for 4 days. The reaction mixture was diluted with EtOAc (20 mL) and the organic phase separated, washed with water (10 mL) and sat. brine solution (10 mL), dried (MgSO₄), filtered and evaporated in vacuo. The crude product was purified by flash chromatography (Biotage SP1; 50 g Sfar cartridge) eluting with isohexane→acetone to afford benzyl (2S,5R)-5-((7H-pyrrolo[2,3-d]pyrimidin-4-yl)amino)-2-methylpiperidine-1-carboxylate (2.87 g, 98%) as a light brown solid. LCMS (Method B): Rt=1.68 min; [M+H]⁺=366.2. ¹H-NMR (400 MHz, DMSO-D6) δ 11.50 (s, 1H), 8.09 (s, 1H), 7.26-7.46 (m, 5H), 7.20 (d, J=8.2 Hz, 1H), 7.08 (t, J=2.7 Hz, 1H), 6.47-6.61 (m, 1H), 4.91-5.24 (m, 2H), 3.94-4.49 (m, 3H), 2.71 (br s, 1H), 1.44-1.93 (m, 4H), 1.07-1.26 (m, 3H).

### Chemical Synthesis Example 10

### N-((3R,6S)-6-Methylpiperidin-3-yl)-7H-pyrrolo[2,3-d]pyrimidin-4-amine

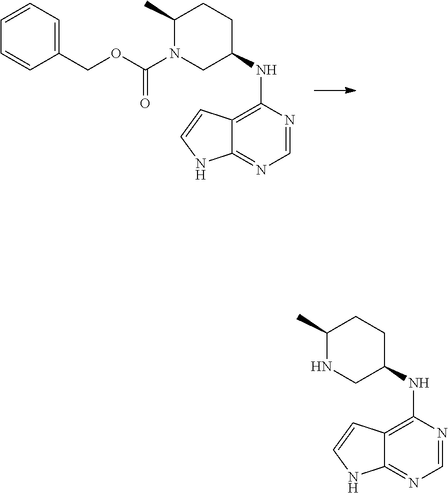 [SMILES: C[C@H]1CC[C@@H](Nc2ncnc3[nH]ccc23)CN1C(=O)OCc1ccccc1 ; C[C@H]1CC[C@@H](Nc2ncnc3[nH]ccc23)CN1]

Benzyl (2S,5R)-5-((7H-pyrrolo[2,3-d]pyrimidin-4-yl)amino)-2-methylpiperidine-1-carboxylate (7.08 g, 19.4 mmol) was dissolved in ethanol (40 mL) and palladium 10% wt on carbon (˜0.5 g) added. The solution was placed under N₂ in a large parr flask (500 mL). The resulting mixture was shaken on a parr apparatus (40 psi of H₂ at r.t. for 5 h). The reaction mixture was filtered through a pad of Celite, and the cake was washed with EtOAc. The filtrate was concentrated in vacuo to afford N-((3R,6S)-6-methylpiperidin-3-yl)-7H-pyrrolo[2,3-d]pyrimidin-4-amine (4.34 g, 97%) as an off-white solid. LCMS (Method B): Rt=1.14 min; [M+H]⁺=232.2. ¹H-NMR (400 MHz, DMSO-D6) δ 11.44 (s, 1H), 8.03 (s, 1H), 7.03 (d, J=3.6 Hz, 1H), 6.82 (d, J=7.3 Hz, 1H), 6.60 (d, J=2.3 Hz, 1H), 4.09-4.11 (m, 1H), 2.87-2.97 (m, 1H), 2.71-2.84 (m, 1H), 2.49-2.63 (m, 1H), 2.07 (br s, 1H), 1.80-1.93 (m, 1H), 1.58 (tt, J=12.7, 4.0 Hz, 1H), 1.34-1.45 (m, 1H), 1.27 (m, 1H), 0.96 (d, J=6.4 Hz, 3H).

### Chemical Synthesis Example 11

### 1-((2S,5R)-5-((7H-Pyrrolo[2,3-d]pyrimidin-4-yl)amino)-2-methylpiperidin-1-yl)prop-2-en-1-one

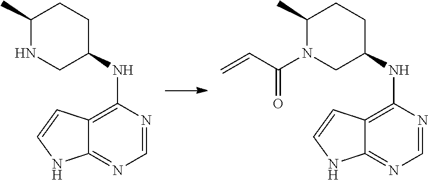 [SMILES: C[C@H]1CC[C@@H](Nc2ncnc3[nH]ccc23)CN1 ; C=CC(=O)N1C[C@H](Nc2ncnc3[nH]ccc23)CC[C@@H]1C]

To a stirred solution of N-((3R,6S)-6-methylpiperidin-3-yl)-7H-pyrrolo[2,3-d]pyrimidin-4-amine (4.34 g, 18.8 mmol) in THF (80 mL) and sat. NaHCO₃ solution (80 mL) was added acryloyl chloride (1.82 mL, 22.5 mmol) dropwise at 0° C. After addition, the resulting mixture was stirred at 0° C. for 2 h. The reaction mixture was diluted with water and extracted with EtOAc (3×50 mL). The combined organics were washed with sat. brine solution (30 mL), dried (MgSO₄), filtered and the solvent evaporated in vacuo. The crude product was purified by flash chromatography (Biotage SP1; 100 g Sfar cartridge) eluting with DCM→10% MeOH-DCM to afford 1-((2S,5R)-5-((7H-pyrrolo[2,3-d]pyrimidin-4-yl)amino)-2-methylpiperidin-1-yl)prop-2-en-1-one (2.24 g, 42%) as a white solid. LCMS (Method C): Rt=2.27 min; [M+H]⁺=286.2. LCMS (Method E): Rt=2.38 min; [M+H]⁺=286.2. ¹H-NMR (400 MHz, DMSO-D6) δ 11.53 (s, 1H), 8.07 (d, J=11.9 Hz, 1H), 7.24 (dd, J=18.3, 7.3 Hz, 1H), 7.04-7.05 (m, 1H), 6.69-6.80 (m, 1H), 6.46-6.57 (m, 1H), 6.05 (dd, J=19.2, 2.3 Hz, 1H), 5.63 (dd, J=12.8, 2.3 Hz, 1H), 4.76 (br s, 0.5H), 4.51 (br s, 0.5H), 4.32 (br s, 0.5H), 3.89-4.22 (m, 1.5H), 2.81-3.01 (m, 0.5H), 2.48-2.67 (m, 0.5H), 1.51-1.90 (m, 4H), 1.05-1.26 (m, 3H).

### Chemical Synthesis Examples 12, 13 & 14

### (4-(((3R,6S)-1-Acryloyl-6-methylpiperidin-3-yl)amino)-7H-pyrrolo[2,3-d]pyrimidin-7-yl)methyl 5-((R)-1,2-dithiolan-3-yl)pentanoate (12), (4-(((3R,6S)-1-acryloyl-6-methylpiperidin-3-yl)amino)-7H-pyrrolo[2,3-d]pyrimidin-7-yl)methyl 5-((3R)-2-oxido-1,2-dithiolan-3-yl)pentanoate (13), and (4-(((3R,6S)-1-acryloyl-6-methylpiperidin-3-yl)amino)-7H-pyrrolo[2,3-d]pyrimidin-7-yl)methyl 5-((3R)-1-oxido-1,2-dithiolan-3-yl)pentanoate (14)

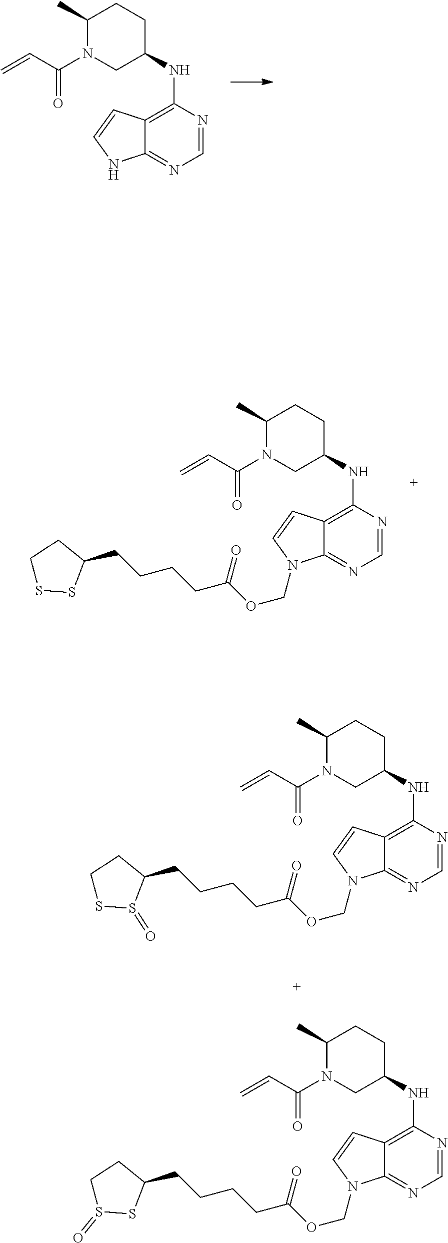 [SMILES: C=CC(=O)N1C[C@H](Nc2ncnc3[nH]ccc23)CC[C@@H]1C ; C=CC(=O)N1C[C@H](Nc2ncnc3c2ccn3COC(=O)CCCC[C@@H]2CCSS2)CC[C@@H]1C ; C=CC(=O)N1C[C@H](Nc2ncnc3c2ccn3COC(=O)CCCC[C@@H]2CCSS2=O)CC[C@@H]1C ; C=CC(=O)N1C[C@H](Nc2ncnc3c2ccn3COC(=O)CCCC[C@@H]2CCS(=O)S2)CC[C@@H]1C]

To a solution of 1-((2S,5R)-5-((7H-pyrrolo[2,3-d]pyrimidin-4-yl)amino)-2-methylpiperidin-1-yl)prop-2-en-1-one (200 mg, 0.70 mmol) in DMF (2 mL) at 0° C., sodium hydride (60% dispersion in oil, 33.6 mg, 0.84 mmol) was added and the mixture stirred at 0° C. for 15 min. Chloromethyl 5-[(3R)-dithiolan-3-yl]pentanoate (267 mg, 1.05 mmol) was added and the reaction stirred at 0° C. for 1 h. The reaction was diluted with EtOAc (10 mL) and washed with 1:1 water-sat. brine solution (5×15 mL), dried (MgSO₄), filtered and concentrated in vacuo. The crude product was purified by flash chromatography (Biotage SP1; 25 g Sfar cartridge) eluting with isohexane→acetone followed by preparative reversed-phase HPLC to afford (4-(((3R,6S)-1-Acryloyl-6-methylpiperidin-3-yl)amino)-7H-pyrrolo[2,3-d]pyrimidin-7-yl)methyl 5-((R)-1,2-dithiolan-3-yl)pentanoate, (4-(((3R,6S)-1-acryloyl-6-methylpiperidin-3-yl)amino)-7H-pyrrolo[2,3-d]pyrimidin-7-yl)methyl 5-((3R)-2-oxido-1,2-dithiolan-3-yl)pentanoate, and (4-(((3R,6S)-1-acryloyl-6-methylpiperidin-3-yl)amino)-7H-pyrrolo[2,3-d]pyrimidin-7-yl)methyl 5-((3R)-1-oxido-1,2-dithiolan-3-yl)pentanoate.

(4-(((3R,6S)-1-acryloyl-6-methylpiperidin-3-yl)amino)-7H-pyrrolo[2,3-d]pyrimidin-7-yl)methyl 5-((R)-1,2-dithiolan-3-yl)pentanoate (21.0 mg, 6%) as a light yellow oil. LCMS (Method C): Rt=4.27 min; [M+H]⁺=504.1. LCMS (Method E): Rt=4.93 min; [M+H]⁺=504.1. ¹H-NMR (400 MHz, DMSO-D6) δ 8.16 (d, J=12.4 Hz, 1H), 7.47 (dd, J=15.8, 7.1 Hz, 1H), 7.22 (d, J=3.7 Hz, 1H), 6.65-6.88 (m, 1H), 6.61 (d, J=3.7 Hz, 1H), 5.97-6.14 (m, 3H), 5.63 (dd, J=10.3, 2.1 Hz, 1H), 4.76 (br s, 0.5H), 4.51 (d, J=9.2 Hz, 0.5H), 4.33 (br s, 0.5H), 3.81-4.22 (m, 1.5H), 3.39-3.59 (m, 1H), 2.74-3.19 (m, 2.5H), 2.49-2.68 (m, 0.5H), 2.15-2.37 (m, 3H), 1.35-1.91 (m, 9H), 1.00-1.35 (m, 5H).

(4-(((3R,6S)-1-acryloyl-6-methylpiperidin-3-yl)amino)-7H-pyrrolo[2,3-d]pyrimidin-7-yl)methyl 5-((3R)-2-oxido-1,2-dithiolan-3-yl)pentanoate and (4-(((3R,6S)-1-acryloyl-6-methylpiperidin-3-yl)amino)-7H-pyrrolo[2,3-d]pyrimidin-7-yl)methyl 5-((3R)-1-oxido-1,2-dithiolan-3-yl)pentanoate (2.8 mg, 1%) as a white solid. LCMS (Method C): Rt=3.35 min; [M+H]⁺=520.1. LCMS (Method E): Rt=3.99 min; [M+H]⁺=520.2.

### Chemical Synthesis Example 15

### 1-(4-(((3R,6S)-1-Acryloyl-6-methylpiperidin-3-yl)amino)-7H-pyrrolo[2,3-d]pyrimidin-7-yl)-5-((R)-1,2-dithiolan-3-yl)pentan-1-one

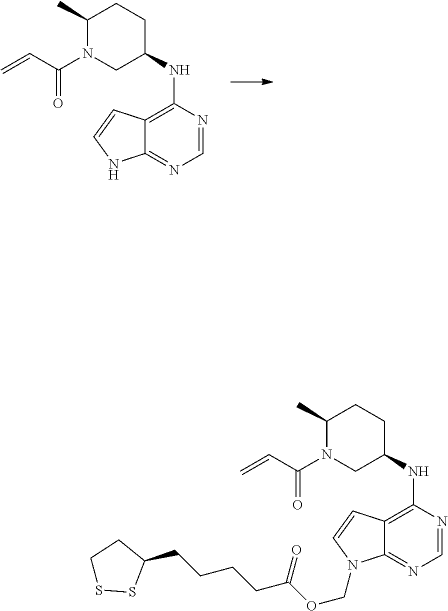 [SMILES: C=CC(=O)N1C[C@H](Nc2ncnc3[nH]ccc23)CC[C@@H]1C ; C=CC(=O)N1C[C@H](Nc2ncnc3c2ccn3COC(=O)CCCC[C@@H]2CCSS2)CC[C@@H]1C]

Lipoic acid (75.9 mg, 0.37 mmol) and 1-(3-dimethylaminopropyl)-3-ethyl-carbodiimide hydrochloride (94.0 mg, 0.49 mmol) were dissolved in DMF (1.0 mL) and stirred at r.t. for 1 h. 4-(Dimethylamino)pyridine (29.9 mg, 0.25 mmol) and 1-((2S,5R)-5-((7H-pyrrolo[2,3-d]pyrimidin-4-yl)amino)-2-methylpiperidin-1-yl)prop-2-en-1-one (70.0 mg, 0.25 mmol) were added and the reaction mixture stirred at r.t. for 16 h. The reaction mixture was diluted with EtOAc (10 mL) and washed with 1:1 water-sat. brine solution (5×15 mL), dried (MgSO₄), filtered and the solvent evaporated in vacuo. The crude product was purified by flash chromatography (Biotage SP1; 10 g Sfar cartridge) eluting with isohexane→80% acetone-isohexane to afford 1-(4-(((3R,6S)-1-acryloyl-6-methylpiperidin-3-yl)amino)-7H-pyrrolo[2,3-d]pyrimidin-7-yl)-5-((R)-1,2-dithiolan-3-yl)pentan-1-one (52 mg, 45%) as a light yellow solid. LCMS (Method C): Rt=5.19 min; [M+H]⁺=474.2. LCMS (Method E): Rt=5.45 min; [M+H]⁺=474.2. ¹H-NMR (400 MHz, DMSO-D6) δ 8.29 (d, J=8.2 Hz, 1H), 7.57-7.66 (m, 2H), 6.61-6.90 (m, 2H), 6.05 (dd, J=16.7, 2.5 Hz, 1H), 6.63 (dd, J=10.3, 2.5 Hz, 1H), 4.76 (br s, 0.5H), 4.41-4.61 (m, 0.5H), 4.34 (br s, 0.5H), 3.86-4.20 (m, 1.5H), 3.51-3.74 (m, 1H), 3.34-3.51 (m, 2H), 2.99-3.21 (m, 2H), 2.92 (t, J=11.4 Hz, 0.5H), 2.50-2.70 (m, 0.5H), 2.31-2.43 (m, 1H), 1.36-1.94 (m, 11H), 1.06-1.30 (m, 3H).

### Chemical Synthesis Examples 16 & 17

### 1-(4-(((3R,6S)-1-Acryloyl-6-methylpiperidin-3-yl)amino)-7H-pyrrolo[2,3-d]pyrimidin-7-yl)-5-((3R)-2-oxido-1,2-dithiolan-3-yl)pentan-1-one (16) and 1-(4-(((3R,6S)-1-Acryloyl-6-methylpiperidin-3-yl)amino)-7H-pyrrolo[2,3-d]pyrimidin-7-yl)-5-((3R)-1-oxido-1,2-dithiolan-3-yl)pentan-1-one (17)

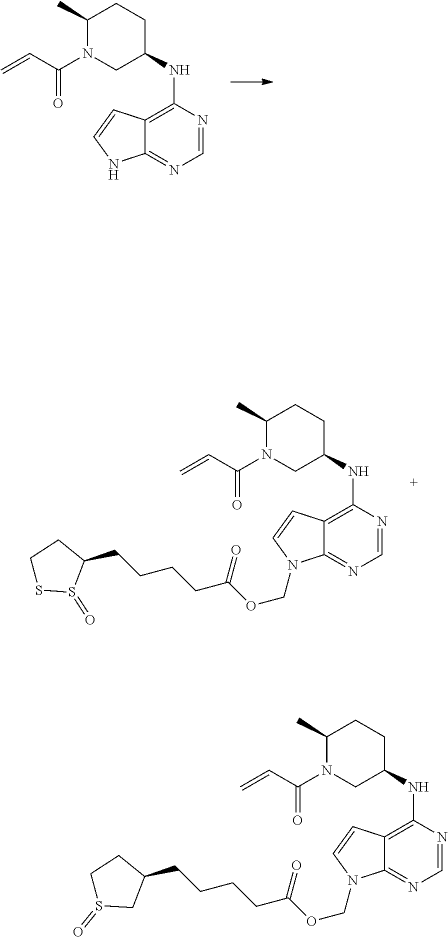 [SMILES: C=CC(=O)N1C[C@H](Nc2ncnc3[nH]ccc23)CC[C@@H]1C ; C=CC(=O)N1C[C@H](Nc2ncnc3c2ccn3COC(=O)CCCC[C@@H]2CCSS2=O)CC[C@@H]1C ; C=CC(=O)N1C[C@H](Nc2ncnc3c2ccn3COC(=O)CCCC[C@@H]2CCS(=O)C2)CC[C@@H]1C]

5-[(3R)-2-Oxodithiolan-3-yl]pentanoic acid (105 mg, 0.47 mmol) and 1-(3-dimethylaminopropyl)-3-ethyl-carbodiimide hydrochloride (121 mg, 0.63 mmol) were dissolved in DMF (1.0 mL) and stirred at r.t. for 1 h. 4-(Dimethylamino)pyridine (38.5 mg, 0.32 mmol) and 1-((2S,5R)-5-((7H-pyrrolo[2,3-d]pyrimidin-4-yl)amino)-2-methylpiperidin-1-yl)prop-2-en-1-one (90.0 mg, 0.32 mmol) were added and the mixture stirred at r.t for 16 hours. The reaction mixture was diluted with EtOAc (10 mL) and washed with 1:1 water-sat. brine solution (5×15 mL), dried (MgSO₄), filtered and the solvent evaporated in vacuo. The crude product was purified by flash chromatography (Biotage SP1; 10 g Sfar cartridge) eluting with isohexane→acetone to afford 1-(4-(((3R,6S)-1-acryloyl-6-methylpiperidin-3-yl)amino)-7H-pyrrolo[2,3-d]pyrimidin-7-yl)-5-((3R)-2-oxido-1,2-dithiolan-3-yl)pentan-1-one and 1-(4-(((3R,6S)-1-acryloyl-6-methylpiperidin-3-yl)amino)-7H-pyrrolo[2,3-d]pyrimidin-7-yl)-5-((3R)-1-oxido-1,2-dithiolan-3-yl)pentan-1-one (30 mg, 19%) as a white solid. LCMS (Method C): Rt=4.01 min; [M+H]⁺=490.2. LCMS (Method E): Rt=4.37 mins; [M+H]^{+=490.2.}

### Chemical Synthesis Example 18

### Methyl 4-(4-(((3R,6S)-1-acryloyl-6-methylpiperidin-3-yl)amino)-7H-pyrrolo[2,3-d]pyrimidin-7-yl)-4-oxobutanoate

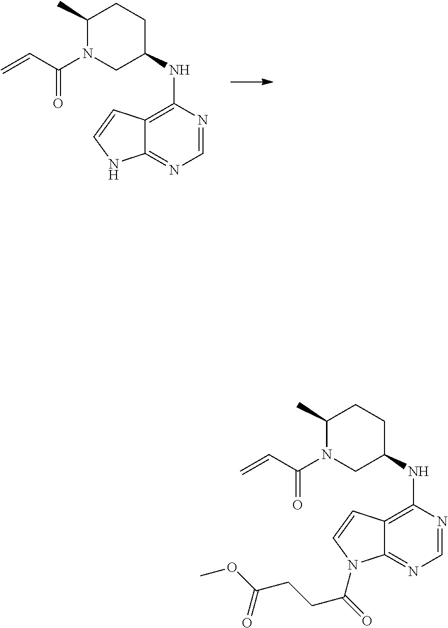 [SMILES: C=CC(=O)N1C[C@H](Nc2ncnc3[nH]ccc23)CC[C@@H]1C ; C=CC(=O)N1C[C@H](Nc2ncnc3c2ccn3C(=O)CCC(=O)OC)CC[C@@H]1C]

Mono-methyl hydrogen succinate (69.4 mg, 0.53 mmol) and 1-(3-dimethylaminopropyl)-3-ethyl-carbodiimide hydrochloride (134 mg, 0.70 mmol) were dissolved in DMF (1.0 mL) and stirred at r.t. for 1 h. 1-((2S,5R)-5-((7H-pyrrolo[2,3-d]pyrimidin-4-yl)amino)-2-methylpiperidin-1-yl)prop-2-en-1-one (100 mg, 0.35 mmol) and 4-(dimethylamino)pyridine (43.0 mg, 0.35 mmol) were added and the mixture was stirred at r.t for 16 hours. The reaction mixture was diluted with EtOAc (5 mL) and washed with 1:1 water-sat. brine solution (5×10 mL), dried (MgSO₄), filtered and the solvent evaporated in vacuo. The crude product was purified by flash chromatography (Biotage SP1; 10 g Sfar cartridge) eluting with isohexane→acetone to afford methyl 4-(4-(((3R,6S)-1-acryloyl-6-methylpiperidin-3-yl)amino)-7H-pyrrolo[2,3-d]pyrimidin-7-yl)-4-oxobutanoate (87.0 mg, 62%) as a white solid. LCMS (Method C): Rt=3.85 min; [M+H]⁺=400.3. LCMS (Method E): Rt=4.21 min; [M+H]⁺=400.2. ¹H-NMR (400 MHz, DMSO-D6) δ 8.30 (d, J=8.2 Hz, 1H), 7.54-7.84 (m, 2H), 6.58-6.99 (m, 2H), 6.05 (dd, J=16.4, 2.3 Hz, 1H), 5.63 (dd, J=10.4, 2.3 Hz, 1H), 4.76 (br s, 0.5H), 4.44-4.66 (m, 0.5H), 4.34 (br s, 0.5H), 3.90-4.24 (m, 1.5H), 3.65 (t, J=12.8 Hz, 2H), 3.57 (s, 3H), 2.80-3.06 (m, 0.5H), 2.72 (t, J=6.4 Hz, 2H), 2.50-2.65 (m, 0.5H), 1.47-1.94 (m, 4H), 1.01-1.34 (m, 3H).

### Chemical Synthesis Example 19

### ((4-(4-(((3R,6S)-1-Acryloyl-6-methylpiperidin-3-yl)amino)-7H-pyrrolo[2,3-d]pyrimidin-7-yl)-4-oxobutanoyl)oxy)methyl 5-((R)-1,2-dithiolan-3-yl)pentanoate

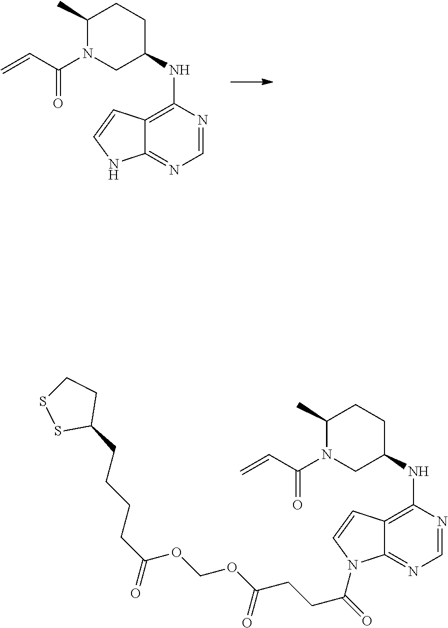 [SMILES: C=CC(=O)N1C[C@H](Nc2ncnc3[nH]ccc23)CC[C@@H]1C ; C=CC(=O)N1C[C@H](Nc2ncnc3c2ccn3C(=O)CCC(=O)OCOC(=O)CCCC[C@@H]2CCSS2)CC[C@@H]1C]

1-(3-Dimethylaminopropyl)-3-ethyl-carbodiimide hydrochloride (68.3 mg, 0.36 mmol) was suspended in anhydrous DCM (2.0 mL) and DMF (0.5 mL), then 4-[5-[(3R)-dithiolan-3-yl]pentanoyloxymethoxy]-4-oxo-butanoic acid (48.0 mg, 0.14 mmol) was added and the mixture stirred at r.t. for 1 h. 4-(Dimethylamino)pyridine (17.4 mg, 0.14 mmol) and 1-((2S,5R)-5-((7H-pyrrolo[2,3-d]pyrimidin-4-yl)amino)-2-methylpiperidin-1-yl)prop-2-en-1-one (40.6 mg, 0.14 mmol) were added and the mixture stirred at r.t. for 16 hours. The reaction mixture was evaporated in vacuo and the residue re-dissolved in DMSO and purified by preparative reversed-phase HPLC to afford ((4-(4-(((3R,6S)-1-acryloyl-6-methylpiperidin-3-yl)amino)-7H-pyrrolo[2,3-d]pyrimidin-7-yl)-4-oxobutanoyl)oxy)methyl 5-((R)-1,2-dithiolan-3-yl)pentanoate (6.3 mg, 7%) as a white solid. LCMS (Method C): Rt=5.21 min; [M+H]⁺=604.2. LCMS (Method E): Rt=5.35 min; [M+H]⁺=604.2.

### Chemical Synthesis Example 20

### Chloromethyl 4-(((3R,6S)-1-acryloyl-6-methylpiperidin-3-yl)amino)-7H-pyrrolo[2,3-d]pyrimidine-7-carboxylate

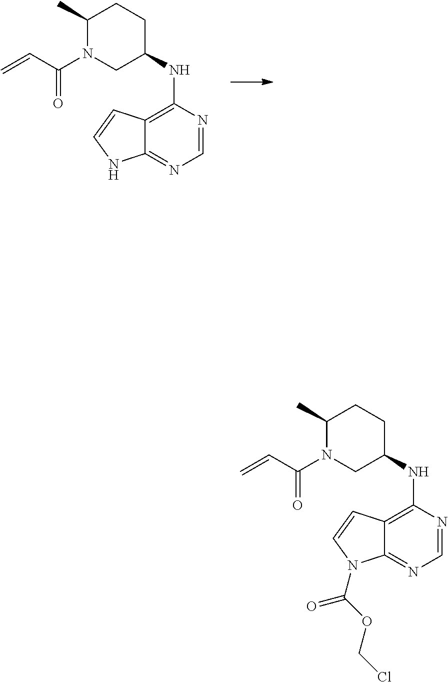 [SMILES: C=CC(=O)N1C[C@H](Nc2ncnc3[nH]ccc23)CC[C@@H]1C ; C=CC(=O)N1C[C@H](Nc2ncnc3c2ccn3C(=O)OCCl)CC[C@@H]1C]

To a solution of 1-((2S,5R)-5-((7H-pyrrolo[2,3-d]pyrimidin-4-yl)amino)-2-methylpiperidin-1-yl)prop-2-en-1-one (300 mg, 1.05 mmol) in THF (3.0 mL) at 0° C., DIPEA (0.22 mL, 1.26 mmol) was added, and the reaction mixture stirred for 15 min at 0° C. Chloromethyl chloroformate (0.12 mL, 1.26 mmol) was added and the reaction mixture stirred at 0° C. for 1 h and then at r.t. for 1 h. The solvent was evaporated in vacuo and the crude product purified by flash chromatography (Biotage SP1; 25 g Sfar cartridge) eluting with isohexane→acetone to afford chloromethyl 4-(((3R,6S)-1-acryloyl-6-methylpiperidin-3-yl)amino)-7H-pyrrolo[2,3-d]pyrimidine-7-carboxylate (180 mg, 39%) as a white solid. LCMS (Method B): Rt=1.48 min; [M+H]⁺=378.2. ¹H-NMR (400 MHz, DMSO-D6) δ 8.30 (br s, 1H), 7.70 (br s, 1H), 7.52 (d, J=3.7 Hz, 1H), 6.87 (d, J=4.1 Hz, 1H), 6.64-6.84 (m, 1H), 6.14 (s, 2H), 6.05 (dd, J=16.9, 2.3 Hz, 1H), 5.64 (dd, J=10.5, 2.3 Hz, 1H), 4.76 (br s, 0.5H), 4.53 (br s, 0.5H), 4.33 (br s, 0.5H), 3.83-4.21 (m, 1.5H), 2.93 (t, J=11.7 Hz, 0.5H), 2.51-2.75 (m, 0.5H), 1.55-1.87 (m, 4H), 1.15 (dd, J=21.8, 6.6 Hz, 3H).

### Chemical Synthesis Example 21

### ((5-((R)-1,2-Dithiolan-3-yl)pentanoyl)oxy)methyl 4-(((3R,6S)-1-acryloyl-6-methylpiperidin-3-yl)amino)-7H-pyrrolo[2,3-d]pyrimidine-7-carboxylate

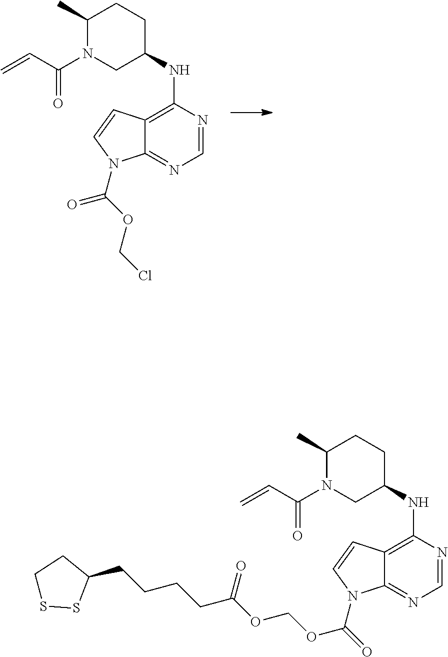 [SMILES: C=CC(=O)N1C[C@H](Nc2ncnc3c2ccn3C(=O)OCCl)CC[C@@H]1C ; C=CC(=O)N1C[C@H](Nc2ncnc3c2ccn3C(=O)OCOC(=O)CCCC[C@@H]2CCSS2)CC[C@@H]1C]

To a solution of chloromethyl 4-(((3R,6S)-1-acryloyl-6-methylpiperidin-3-yl)amino)-7H-pyrrolo[2,3-d]pyrimidine-7-carboxylate (90.0 mg, 0.16 mmol) in DMF (1.0 mL), (R)-5-(1,2-dithiolan-3-yl)pentanoic acid (66 mg, 0.31 mmol) and DIPEA (97 μL, 0.55 mmol) were added and the reaction mixture was stirred at r.t. for 16 hours. The reaction mixture was diluted with EtOAc (5 mL) and washed with 1:1 water-sat. brine solution (5×10 mL), dried (MgSO₄), filtered and the solvent evaporated in vacuo. The residue was re-dissolved in DMSO and purified by preparative reversed-phase HPLC to afford ((5-((R)-1,2-Dithiolan-3-yl)pentanoyl)oxy)methyl 4-(((3R,6S)-1-acryloyl-6-methylpiperidin-3-yl)amino)-7H-pyrrolo[2,3-d]pyrimidine-7-carboxylate (7.2 mg, 8%) as a white solid. LCMS (Method C): Rt=4.73 min; [M+H]⁺=548.1. LCMS (Method E): Rt=4.91 min; [M+H]⁺=548.1. ¹H-NMR (400 MHz, DMSO-D6) δ 8.18-8.38 (m, 1H), 7.58-7.74 (m, 1H), 7.46 (d, J=3.7 Hz, 1H), 6.57-6.89 (m, 2H), 6.05 (dd, J=16.7, 2.5 Hz, 1H), 5.95 (s, 2H), 5.64 (dd, J=10.5, 2.3 Hz, 1H), 4.76 (br s, 0.5H), 4.43-4.62 (m, 0.5H), 4.26-4.41 (m, 0.5H), 3.90-4.20 (m, 1.5H), 3.40-3.64 (m, 0.5H), 2.83-3.16 (m, 2H), 2.50-2.67 (m, 0.5H), 2.39 (t, J=7.3 Hz, 2H), 2.29 (m, 1H), 1.00-1.93 (m, 15H).

### Chemical Synthesis Examples 22 & 23

### 1-(4-(((3R,6S)-1-Acryloyl-6-methylpiperidin-3-yl)amino)-7H-pyrrolo[2,3-d]pyrimidin-7-yl)ethyl isopropyl carbonate (22) and isopropyl 4-(((3R,6S)-1-acryloyl-6-methylpiperidin-3-yl)amino)-7H-pyrrolo[2,3-d]pyrimidine-7-carboxylate (23)

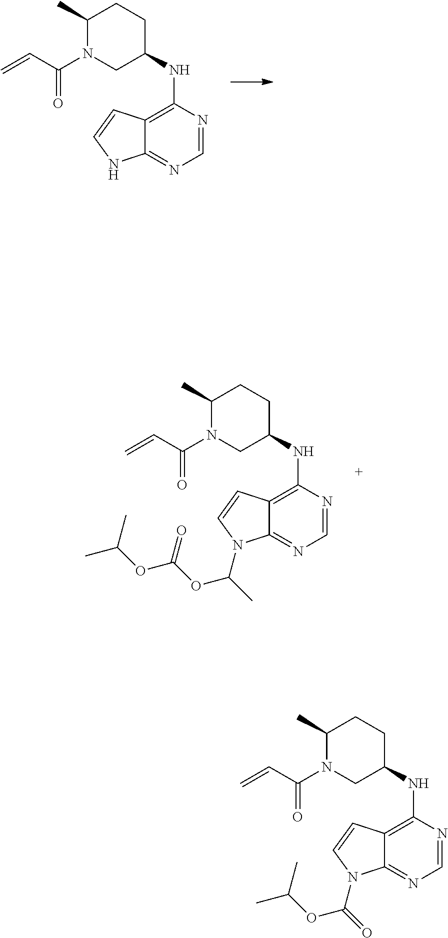 [SMILES: C=CC(=O)N1C[C@H](Nc2ncnc3[nH]ccc23)CC[C@@H]1C ; C=CC(=O)N1C[C@H](Nc2ncnc3c2ccn3C(C)OC(=O)OC(C)C)CC[C@@H]1C ; C=CC(=O)N1C[C@H](Nc2ncnc3c2ccn3C(=O)OC(C)C)CC[C@@H]1C]

To a solution of 1-((2S,5R)-5-((7H-pyrrolo[2,3-d]pyrimidin-4-yl)amino)-2-methylpiperidin-1-yl)prop-2-en-1-one (60.0 mg, 0.21 mmol) in DMF (2.0 mL) at 0° C., sodium hydride (10.0 mg, 0.25 mmol, 60% dispersion in oil) was added and the mixture was left stirring at r.t. for 15 min. 1-chloroethyl isopropyl carbonate (48 μL, 0.32 mmol) was added and the reaction mixture stirred at r.t. for 30 min. The reaction mixture was diluted with EtOAc (5 mL) and washed with 1:1 water-sat. brine solution (5×10 mL), dried (MgSO₄), filtered and concentrated in vacuo. The crude product was purified by flash chromatography (Biotage SP1; 10 g Sfar cartridge) eluting with isohexane→acetone to afford 1-(4-(((3R,6S)-1-acryloyl-6-methylpiperidin-3-yl)amino)-7H-pyrrolo[2,3-d]pyrimidin-7-yl)ethyl isopropyl carbonate and isopropyl 4-(((3R,6S)-1-acryloyl-6-methylpiperidin-3-yl)amino)-7H-pyrrolo[2,3-d]pyrimidine-7-carboxylate.

1-(4-(((3R,6S)-1-acryloyl-6-methylpiperidin-3-yl)amino)-7H-pyrrolo[2,3-d]pyrimidin-7-yl)ethyl isopropyl carbonate (15.0 mg, 17%), as an off-white solid. LCMS (Method C): Rt=3.61 min; [M+H]⁺=416.3. LCMS (Method E): Rt=4.46 min; [M+H]⁺=416.2.

Isopropyl 4-(((3R,6S)-1-acryloyl-6-methylpiperidin-3-yl)amino)-7H-pyrrolo[2,3-d]pyrimidine-7-carboxylate (26.0 mg, 33%) was obtained as white gum. LCMS (Method C): Rt=3.60 min; [M+H]⁺=372.3. LCMS (Method E): Rt=4.14 min; [M+H]⁺=372.2. ¹H-NMR (400 MHz, DMSO-D6) δ 8.26 (d, J=11.0 Hz, 1H), 7.58-7.71 (m, 1H), 7.47 (d, J=3.9 Hz, 1H), 6.67-6.87 (m, 2H), 6.05 (dd, J=16.9, 2.3 Hz, 1H), 5.64 (dd, J=10.5, 1.8 Hz, 1H), 5.10 (sep, J=6.4 Hz, 1H), 4.75 (br s, 0.5H), 4.56 (br s, 0.5H), 4.33 (br s, 0.5H), 3.89-4.16 (m, 1.5H), 2.81-3.02 (m, 0.5H), 2.49-2.70 (m, 0.5H), 1.55-1.88 (m, 4H), 1.29-1.40 (m, 6H), 1.11-1.23 (m, 3H).

### Chemical Synthesis Example 24

### (4-(((3R,4R)-1-(2-Cyanoacetyl)-4-methylpiperidin-3-yl)(methyl)amino)-7H-pyrrolo[2,3-d]pyrimidin-7-yl)methyl 5-((R)-1,2-dithiolan-3-yl)pentanoate

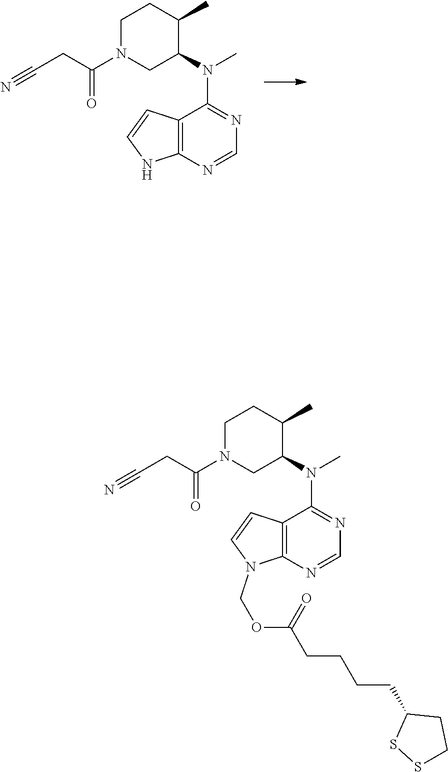 [SMILES: C[C@@H]1CCN(C(=O)CC#N)C[C@@H]1N(C)c1ncnc2[nH]ccc12 ; C[C@@H]1CCN(C(=O)CC#N)C[C@@H]1N(C)c1ncnc2c1ccn2COC(=O)CCCC[C@@H]1CCSS1]

To a solution of Tofacitinib (50 mg, 0.16 mmol) in DMF (2.0 mL), sodium hydride (60% dispersion in oil, 7.0 mg, 0.18 mmol) was added and the reaction mixture stirred at r.t. for 15 min. Chloromethyl-(R)-5-(1,2-dithiolan-3-yl)pentanoate (75 mg, 2.9 mmol) was then added and the reaction mixture stirred at 0° C. for 15 min. The reaction mixture was partitioned between water (5 mL) and EtOAc (5 mL) and the layers were separated. The organic phase was washed successively with sat. NaHCO_{3(aq)} and sat. brine solution, dried (MgSO₄), filtered and the solvent evaporated in vacuo. The crude product was purified by preparative reversed-phase HPLC to afford (4-(((3R,4R)-1-(2-cyanoacetyl)-4-methylpiperidin-3-yl)(methyl)amino)-7H-pyrrolo[2,3-d]pyrimidin-7-yl)methyl 5-((R)-1,2-dithiolan-3-yl)pentanoate as a white solid (3.8 mg, 5%). LCMS (Method C): Rt=5.12 min; [M+H]⁺=531.2, LCMS (Method E): Rt=5.39 min; [M+H]⁺=531.2.

### Chemical Synthesis Example 25

### 3-((3R,4R)-3-((7-(5-((R)-1,2-Dithiolan-3-yl)pentanoyl)-7H-pyrrolo[2,3-d]pyrimidin-4-yl)(methyl)amino)-4-methylpiperidin-1-yl)-3-oxopropanenitrile

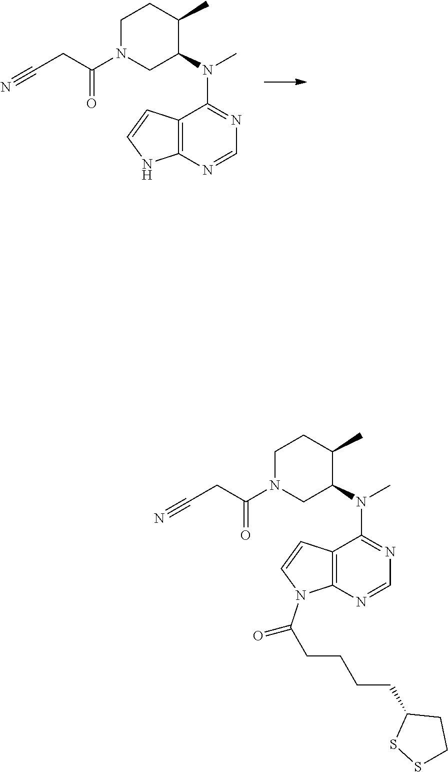 [SMILES: C[C@@H]1CCN(C(=O)CC#N)C[C@@H]1N(C)c1ncnc2[nH]ccc12 ; C[C@@H]1CCN(C(=O)CC#N)C[C@@H]1N(C)c1ncnc2c1ccn2C(=O)CCCC[C@@H]1CCSS1]

1-(3-Dimethylaminopropyl)-3-ethyl-carbodiimide hydrochloride (61 mg, 0.32 mmol) was dissolved in anhydrous DCM (2.0 mL), lipoic acid (26 mg, 0.13 mmol) was added and the mixture stirred at r.t. for 1 h. Separately, DMAP (16 mg, 0.13 mmol) was dissolved in anhydrous DCM (2.0 mL), Tofacitinib (40 mg, 0.13 mmol) was added and the mixture stirred at r.t. for 10 min. The two solutions were combined and the reaction mixture stirred at r.t. for 16 h. The solvent was evaporated in vacuo and the crude product purified by flash chromatography (Biotage Isolera four; 10 g Sfar cartridge) eluting with 70% methyl acetate in pentane. The solvent was evaporated by flow of N₂ to afford 3-((3R,4R)-3-((7-(5-((R)-1,2-dithiolan-3-yl)pentanoyl)-7H-pyrrolo[2,3-d]pyrimidin-4-yl)(methyl)amino)-4-methylpiperidin-1-yl)-3-oxopropanenitrile as a yellow solid (20.3 mg, 32%). LCMS (Method C): Rt=5.30 min; [M+H]⁺=501.2, LCMS (Method E): Rt=5.55 min; [M+H]⁺=501.2.

### Chemical Synthesis Example 26

### ((4-(4-(((3R,4R)-1-(2-Cyanoacetyl)-4-methylpiperidin-3-yl)(methyl)amino)-7H-pyrrolo[2,3-d]pyrimidin-7-yl)-4-oxobutanoyl)oxy)methyl 5-((R)-1,2-dithiolan-3-yl)pentanoate

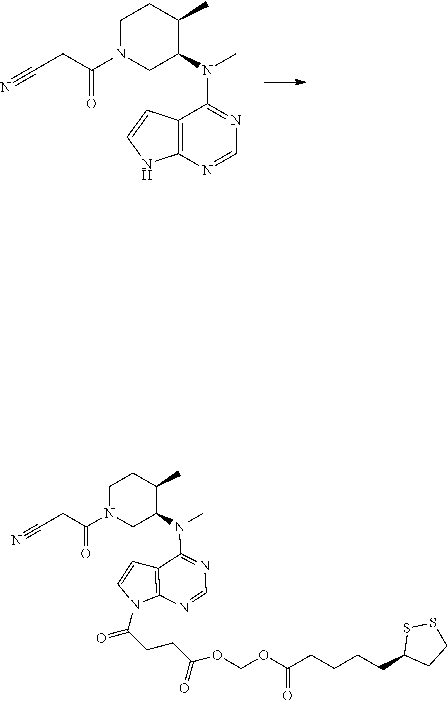 [SMILES: C[C@@H]1CCN(C(=O)CC#N)C[C@@H]1N(C)c1ncnc2[nH]ccc12 ; C[C@@H]1CCN(C(=O)CC#N)C[C@@H]1N(C)c1ncnc2c1ccn2C(=O)CCC(=O)OCOC(=O)CCCC[C@@H]1CCSS1]

1-(3-Dimethylaminopropyl)-3-ethyl-carbodiimide hydrochloride (132 mg, 0.69 mmol) was dissolved in anhydrous DCM (2.0 mL), (R)-4-(((5-(1,2-dithiolan-3-yl)pentanoyl)oxy)methoxy)-4-oxobutanoic acid (93 mg, 0.28 mmol) was then added and the mixture was stirred at r.t. for 1 h. Separately, DMAP (33 mg, 0.28 mmol) was dissolved in anhydrous DCM (2.0 mL), Tofacitinib (86 mg, 0.28 mmol) was added, the mixture was stirred at r.t. for 10 min. The two solutions were combined and the reaction mixture stirred at r.t. for 16 h. The solvent was evaporated in vacuo and the crude product purified by preparative reversed-phase HPLC to afford ((4-(4-(((3R,4R)-1-(2-cyanoacetyl)-4-methylpiperidin-3-yl)(methyl)amino)-7H-pyrrolo[2,3-d]pyrimidin-7-yl)-4-oxobutanoyl)oxy)methyl 5-((R)-1,2-dithiolan-3-yl)pentanoate as a white solid (2.0 mg, 2%). LCMS (Method C): Rt=5.30 min; [M+H]⁺=631.2, LCMS (Method E): Rt=5.40 min; [M+H]⁺=631.2.

### Chemical Synthesis Example 27

### 1-((4-(4-(((3R,4R)-1-(2-Cyanoacetyl)-4-methylpiperidin-3-yl)(methyl)amino)-7H-pyrrolo [2,3-d]pyrimidin-7-yl)-4-oxobutanoyl)oxy)ethyl 5-((R)-1,2-dithiolan-3-yl)pentanoate

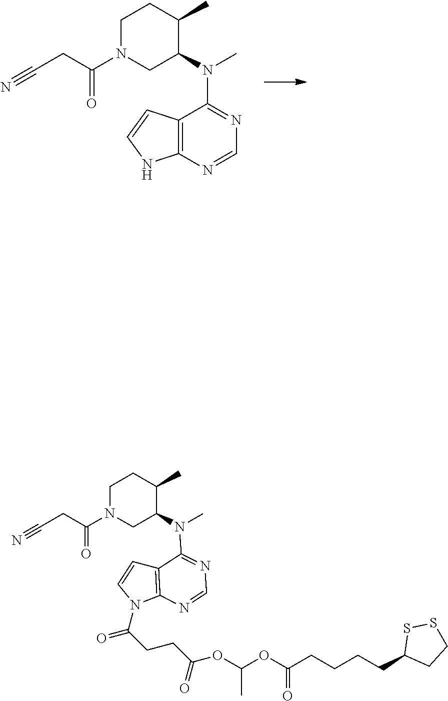 [SMILES: C[C@@H]1CCN(C(=O)CC#N)C[C@@H]1N(C)c1ncnc2[nH]ccc12 ; CC(OC(=O)CCCC[C@@H]1CCSS1)OC(=O)CCC(=O)n1ccc2c(N(C)[C@H]3CN(C(=O)CC#N)CC[C@H]3C)ncnc21]

1-(3-Dimethylaminopropyl)-3-ethyl-carbodiimide hydrochloride (37 mg 0.19 mmol) was dissolved in anhydrous DCM (2.0 mL), 4-(1-((5-((R)-1,2-dithiolan-3-yl)pentanoyl)oxy)ethoxy)-4-oxobutanoic acid (27 mg, 77 μmol) was added and the reaction mixture stirred at r.t. for 1 h. Separately, DMAP (9.4 mg, 77 μmol) was dissolved in anhydrous DCM (2.0 mL), Tofacitinib (24 mg, 77 μmol) was added, the mixture was stirred at r.t. for 10 min. The two solutions were combined and the reaction mixture stirred at r.t. for 16 h. The solvent was evaporated in vacuo and the crude product purified by preparative reversed-phase HPLC to afford 1-((4-(4-(((3R,4R)-1-(2-cyanoacetyl)-4-methylpiperidin-3-yl)(methyl)amino)-7H-pyrrolo [2,3-d]pyrimidin-7-yl)-4-oxobutanoyl)oxy)ethyl 5-((R)-1,2-dithiolan-3-yl)pentanoate as a white solid (1.0 mg, 2%). LCMS (Method C): Rt=5.52 min; [M+H]⁺=645.3, LCMS (Method E): Rt=5.64 min; [M+H]⁺=645.3.

### Chemical Synthesis Example 28

### Methyl 4-(4-(((3R,4R)-1-(2-cyanoacetyl)-4-methylpiperidin-3-yl)(methyl)amino)-7H-pyrrolo[2,3-d]pyrimidin-7-yl)-4-oxobutanoate

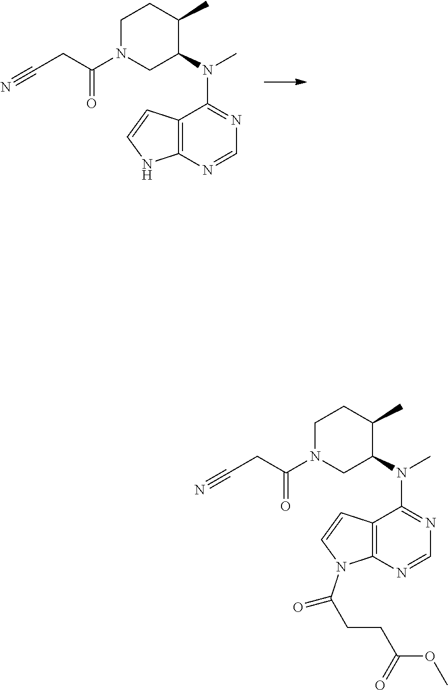 [SMILES: C[C@@H]1CCN(C(=O)CC#N)C[C@@H]1N(C)c1ncnc2[nH]ccc12 ; COC(=O)CCC(=O)n1ccc2c(N(C)[C@H]3CN(C(=O)CC#N)CC[C@H]3C)ncnc21]

1-(3-Dimethylaminopropyl)-3-ethyl-carbodiimide hydrochloride (46 mg, 0.24 mmol) was dissolved in anhydrous DCM (2.0 mL), mono-methyl hydrogen succinate (13 mg, 96 μmol) was added and the mixture stirred at r.t. for 1 h. Separately, DMAP (12 mg, 96 μmol) was dissolved in anhydrous DCM (2.0 mL), Tofacitinib (30 mg, 96 μmol) was added, the mixture was stirred at r.t. for 10 min. The two solutions were combined and the reaction mixture stirred at r.t. for 16h. The solvent was evaporated in vacuo and the crude product was purified by flash chromatography (Biotage Isolera four; 10 g Sfar cartridge) eluting with 100% EtOAc, to afford methyl 4-(4-(((3R,4R)-1-(2-cyanoacetyl)-4-methylpiperidin-3-yl)(methyl)amino)-7H-pyrrolo[2,3-d]pyrimidin-7-yl)-4-oxobutanoate as a beige oil (19.8 mg, 48%). LCMS (Method C): Rt=3.98 min; [M+H]⁺=427.3, LCMS (Method D): Rt=3.29 min; [M+H]⁺=427.3.

### Chemical Synthesis Example 29

### Ethyl 4-(4-(((3R,4R)-1-(2-cyanoacetyl)-4-methylpiperidin-3-yl)(methyl)amino)-7H-pyrrolo[2,3-d]pyrimidin-7-yl)-4-oxobutanoate

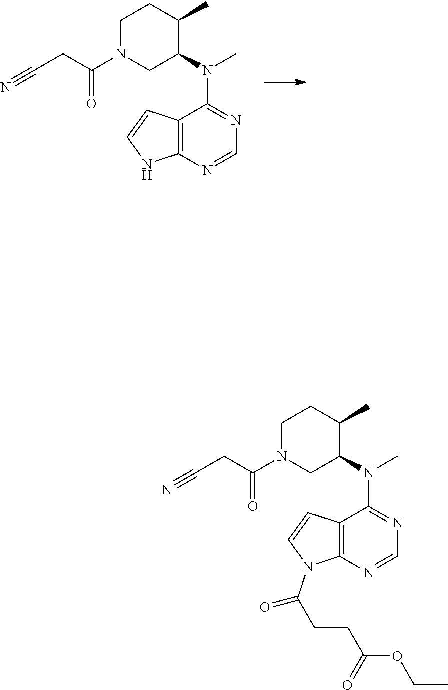 [SMILES: C[C@@H]1CCN(C(=O)CC#N)C[C@@H]1N(C)c1ncnc2[nH]ccc12 ; CCOC(=O)CCC(=O)n1ccc2c(N(C)[C@H]3CN(C(=O)CC#N)CC[C@H]3C)ncnc21]

1-(3-Dimethylaminopropyl)-3-ethyl-carbodiimide hydrochloride (46 mg, 0.24 mmol) was dissolved in anhydrous DCM (2.0 mL), mono-ethyl succinate (14 mg, 96 μmol) was added and the reaction mixture stirred at r.t. for 1 h. Separately, DMAP (12 mg, 96 μmol) was dissolved in anhydrous DCM (2.0 mL), Tofacitinib (30 mg, 96 μmol) was added and the reaction mixture was stirred at r.t. for 10 min. The two solutions were combined, and the reaction mixture stirred at r.t. for 16 h. The solvent was evaporated in vacuo and the crude product purified by flash chromatography (Biotage Isolera four; 10 g Sfar cartridge) eluting with 100% EtOAc, to afford ethyl 4-(4-(((3R,4R)-1-(2-cyanoacetyl)-4-methylpiperidin-3-yl)(methyl)amino)-7H-pyrrolo[2,3-d]pyrimidin-7-yl)-4-oxobutanoate as a colorless oil (11.5 mg, 27%). LCMS (Method C): Rt=4.33 min; [M+H]⁺=441.2, LCMS (Method D): Rt=4.64 min; [M+H]⁺=441.2.

### Chemical Synthesis Example 30

### 1-(4-(Methyl((1r,4r)-4-((N-methylsulfamoyl)methyl)cyclohexyl)amino)-7H-pyrrolo[2,3-d]pyrimidin-7-yl)ethyl 5-((R)-1,2-dithiolan-3-yl)pentanoate

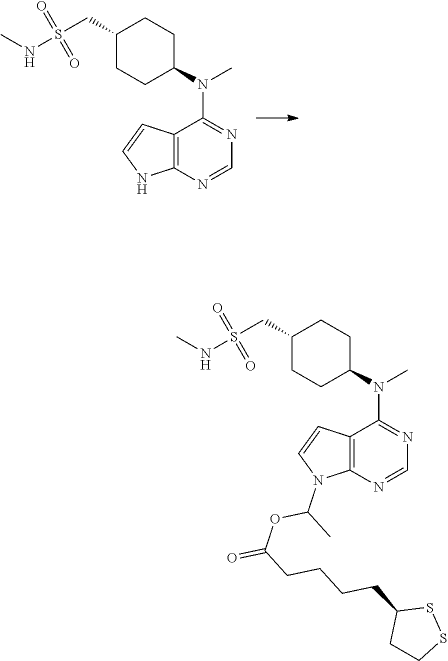

To a solution of Oclacitinib (40 mg, 0.12 mmol) in DMF (1.5 mL), sodium hydride (60% dispersion in oil, 4.7 mg, 0.12 mmol) was added and the reaction mixture stirred at r.t. for 15 min. 1-Chloroethyl 5-[(R)-1,2-dithiolan-3-yl]pentanoate (38 mg, 0.14 mmol) and NaI (18 mg, 0.12 mmol) were added and the reaction mixture stirred at 50° C. for 16 hours. The reaction mixture was partitioned between water (10 mL) and EtOAc (10 mL). The layers were separated and the organic phase washed successively with sat. NaHCO_{3(aq)} and sat. brine solution, dried (MgSO₄), filtered and the solvent evaporated in vacuo. The crude product was purified by preparative reversed-phase HPLC to afford 1-(4-(methyl((1r,4r)-4-((N-methylsulfamoyl)methyl)cyclohexyl)amino)-7H-pyrrolo[2,3-d]pyrimidin-7-yl)ethyl 5-((R)-1,2-dithiolan-3-yl)pentanoate as a white solid (1.9 mg, 3%). LCMS (Method C): Rt=4.16 min; [M+H]⁺=570.1, LCMS (Method E): Rt=5.26 min; [M+H]⁺=570.1

### Chemical Synthesis Examples 31, 32, & 33

### (4-(methyl((1r,4r)-4-((N-methylsulfamoyl)methyl)cyclohexyl)amino)-7H-pyrrolo[2,3-d]pyrimidin-7-yl)methyl 5-((R)-1,2-dithiolan-3-yl)pentanoate (31), (4-(methyl((1r,4r)-4-((N-methylsulfamoyl)methyl)cyclohexyl)amino)-7H-pyrrolo[2,3-d]pyrimidin-7-yl)methyl 5-((3R)-1-oxido-1,2-dithiolan-3-yl)pentanoate (32), and (4-(methyl((1r,4r)-4-((N-methylsulfamoyl)methyl)cyclohexyl)amino)-7H-pyrrolo[2,3-d]pyrimidin-7-yl)methyl 5-((3R)-2-oxido-1,2-dithiolan-3-yl)pentanoate (33)

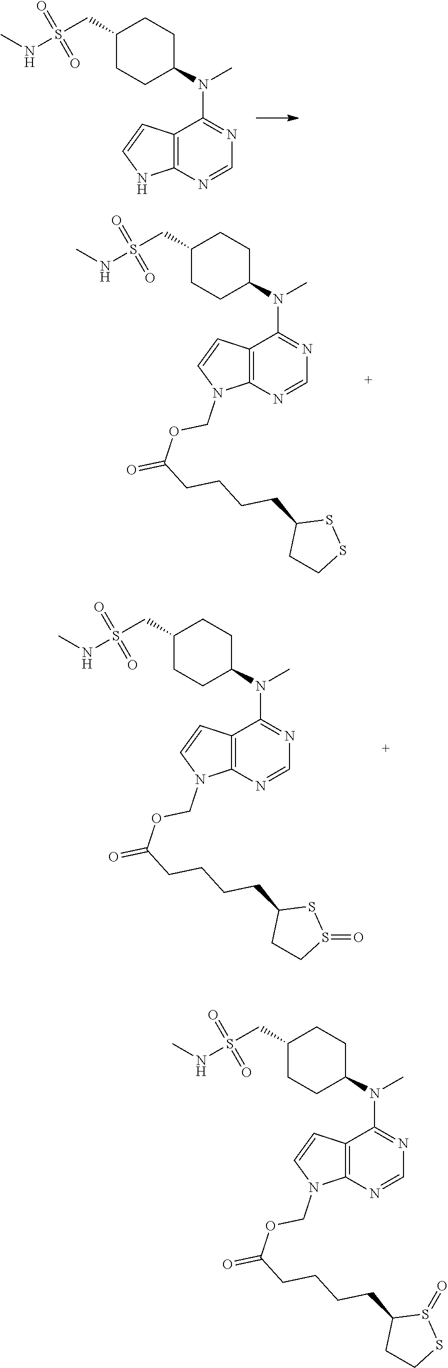

To a solution of Oclacitinib (20 mg, 59 μmol) in DMF (1.5 mL), sodium hydride (60% dispersion in oil, 2.4 mg, 59 μmol) was added and the reaction mixture stirred at r.t. for 15 min. Chloromethyl-(R)-5-(1,2-dithiolan-3-yl)pentanoate (33 mg, 0.13 mmol) was added and the reaction mixture stirred at r.t for 2 h. The reaction mixture was partitioned between water (5 mL) and EtOAc (5 mL). The layers were separated and the organic phase washed successively with sat. NaHCO_{3(aq)} and sat. brine solution, dried (MgSO₄), filtered and the solvent evaporated in vacuo. The crude product was purified by preparative reversed-phase HPLC to afford (4-(methyl((1r,4r)-4-((N-methylsulfamoyl)methyl)cyclohexyl)amino)-7H-pyrrolo[2,3-d]pyrimidin-7-yl)methyl 5-((R)-1,2-dithiolan-3-yl)pentanoate, (4-(methyl((1r,4r)-4-((N-methylsulfamoyl)methyl)cyclohexyl)amino)-7H-pyrrolo[2,3-d]pyrimidin-7-yl)methyl 5-((3R)-1-oxido-1,2-dithiolan-3-yl)pentanoate, and (4-(methyl((1r,4r)-4-((N-methylsulfamoyl)methyl)cyclohexyl)amino)-7H-pyrrolo[2,3-d]pyrimidin-7-yl)methyl 5-((3R)-2-oxido-1,2-dithiolan-3-yl)pentanoate.

(4-(Methyl((1r,4r)-4-((N-methylsulfamoyl)methyl)cyclohexyl)amino)-7H-pyrrolo[2,3-d]pyri-midin-7-yl)methyl 5-((R)-1,2-dithiolan-3-yl)pentanoate as a white solid (5.6 mg, 27%). LCMS (Method C): Rt=4.08 min; [M+H]⁺=556.1. LCMS (Method D): Rt=5.04 min; [M+H]⁺=556.1. ¹H-NMR (400 MHz, DMSO-D6) δ 8.08-8.24 (m, 1H), 7.29 (d, J=3.7 Hz, 1H), 6.87 (q, J =5.0 Hz, 1H), 6.64 (d, J=3.2 Hz, 1H), 6.09-6.15 (m, 2H), 4.64 (d, J=9.6 Hz, 1H), 3.46-3.57 (m, 2H), 3.04-3.20 (m, 4H), 2.94-3.00 (m, 2H), 2.54-2.59 (m, 3H), 2.28-2.35 (m, 2H), 2.03-2.05 (m, 2H), 1.24-1.88 (m, 14H).

(4-(Methyl((1r,4r)-4-((N-methylsulfamoyl)methyl)cyclohexyl)amino)-7H-pyrrolo[2,3-d]pyrimidin-7-yl)methyl 5-((3R)-1-oxido-1,2-dithiolan-3-yl)pentanoate and (4-(methyl((1r,4r)-4-((N-methylsulfamoyl)methyl)cyclohexyl)amino)-7H-pyrrolo[2,3-d]pyrimidin-7-yl)methyl 5-((3R)-2-oxido-1,2-dithiolan-3-yl)pentanoate as a white solid (1.1 mg, 3%). LCMS (Method C): Rt=3.23 min; [M+H]⁺=572.1. LCMS (Method D): Rt=4.12 min; [M+H]⁺=572.1.

### Chemical Synthesis Example 34

### 1-((1r,4r)-4-((7-(5-((R)-1,2-Dithiolan-3-yl)pentanoyl)-7H-pyrrolo[2,3-d]pyrimidin-4-yl)(methyl)amino)cyclohexyl)-N-methylmethanesulfonamide

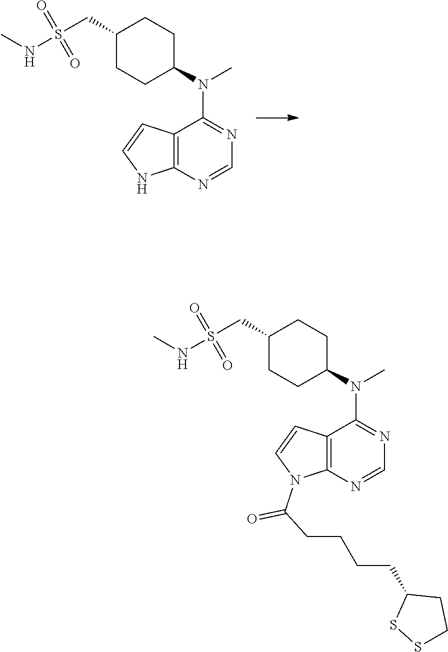

1-(3-Dimethylaminopropyl)-3-ethyl-carbodiimide hydrochloride (57 mg, 0.30 mmol) was dissolved in anhydrous DCM (2.0 mL), lipoic acid (25 mg, 0.12 mmol) was added and the reaction mixture stirred at r.t. for 1 h. Separately, DMAP (15 mg, 0.12 mmol) was dissolved in anhydrous DCM (2.0 mL), Oclacitinib (40 mg, 0.12 mmol) was added, and the reaction mixture stirred at r.t. for 10 min. The two solutions were combined, and the reaction mixture stirred at r.t. for 16 h. The solvent was evaporated in vacuo and the crude product purified by flash chromatography (Biotage Isolera four; 10 g Sfar cartridge) eluting with 70% methyl acetate in pentane, followed by evaporation by N₂ to afford 1-((1R,4r)-4-((7-(5-((R)-1,2-dithiolan-3-yl)pentanoyl)-7H-pyrrolo[2,3-d]pyrimidin-4-yl)(methyl)amino)cyclohexyl)-N-methylmethanesulfonamide as a yellow solid (26.7 mg, 43%). LCMS (Method C): Rt=5.11 min; [M+H]⁺=526.2. LCMS (Method E): Rt=5.64 min; [M+H]⁺=526.2.

### Chemical Synthesis Examples 35 & 36

### N-methyl-1-((1R,4r)-4-(methyl(7-(5-((3R)-2-oxido-1,2-dithiolan-3-yl)pentanoyl)-7H-pyrrolo[2,3-d]pyrimidin-4-yl)amino)cyclohexyl)methanesulfonamide (35) and N-methyl-1-((1R,4r)-4-(methyl(7-(5-((3R)-1-oxido-1,2-dithiolan-3-yl)pentanoyl)-7H-pyrrolo[2,3-d]pyrimidin-4-yl)amino)cyclohexyl)methanesulfonamide (36)

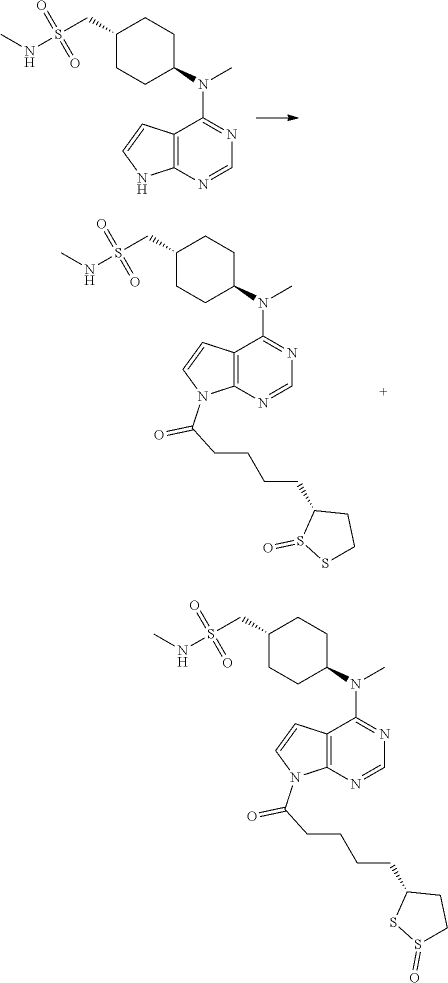

1-(3-Dimethylaminopropyl)-3-ethyl-carbodiimide hydrochloride (71 mg, 0.37 mmol) was dissolved in anhydrous DCM (2.0 mL), 5-[(3R)-2-oxido-1,2-dithiolan-3-yl]pentanoic acid (33 mg, 0.15 mmol) was added and the reaction mixture stirred at r.t. for 1 h. Separately, DMAP (18 mg, 0.15 mmol) was dissolved in anhydrous DCM (2.0 mL), Oclacitinib (50 mg, 0.15 mmol) was added and the reaction mixture stirred at r.t. for 10 min. The two solutions were combined and the reaction mixture stirred at r.t. for 16h. The solvent was evaporated in vacuo and the crude product purified by flash chromatography (Biotage Isolera four; 10 g Sfar cartridge) eluting with 100% EtOAc to afford N-methyl-1-((1R,4r)-4-(methyl(7-(5-((3R)-2-oxido-1,2-dithiolan-3-yl)pentanoyl)-7H-pyrrolo[2,3-d]pyrimidin-4-yl)amino)cyclohexyl)methanesulfonamide and N-methyl-1-((1R,4r)-4-(methyl(7-(5-((3R)-1-oxido-1,2-dithiolan-3-yl)pentanoyl)-7H-pyrrolo[2,3-d]pyrimidin-4-yl)amino)cyclohexyl)methanesulfonamide as a colorless oil (13.3 mg, 17%). LCMS (Method C): Rt=3.85 min; [M+H]⁺=542.2. LCMS (Method E): Rt=4.55 min; [M+H]⁺=542.2.

### Chemical Synthesis Example 37

### Ethyl 4-(4-(methyl((1r,4r)-4-((N-methylsulfamoyl)methyl)cyclohexyl)amino)-7H-pyrrolo [2,3-d]pyrimidin-7-yl)-4-oxobutanoate

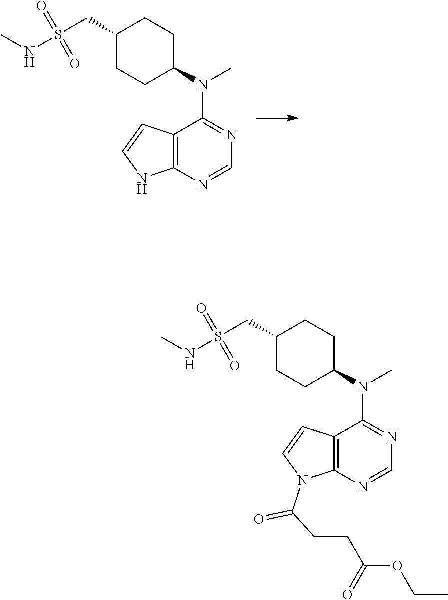

1-(3-Dimethylaminopropyl)-3-ethyl-carbodiimide hydrochloride (71 mg, 0.37 mmol) was dissolved in anhydrous DCM (2.0 mL), mono-ethyl succinate (22 mg, 0.15 mmol) was then added and the reaction mixture stirred at r.t. for 1 h. Separately, DMAP (18 mg, 0.15 mmol) was dissolved in anhydrous DCM (2.0 mL), Oclacitinib (50 mg, 0.15 mmol) was added and the reaction mixture stirred at r.t. for 10 min. The two solutions were combined and the reaction mixture stirred at r.t. for 16 h. The solvent was evaporated in vacuo and the crude product purified by flash chromatography (Biotage Isolera four; 10 g Sfar cartridge) eluting with 80% EtOAc in isohexane, to afford ethyl 4-(4-(methyl((1r,4r)-4-((N-methylsulfamoyl)methyl)cyclohexyl)amino)-7H-pyrrolo [2,3-d]pyrimidin-7-yl)-4-oxobutanoate as a white solid (14.3 mg, 21%). LCMS (Method C): Rt=4.12 min; [M+H]⁺=466.2. LCMS (Method E): Rt=4.72 min; [M+H]⁺=466.2. ¹H-NMR (400 MHz, DMSO-D6) δ 8.30-8.34 (m, 1H), 7.67 (d, J=4.1 Hz, 1H), 6.87 (q, J=4.9 Hz, 2H), 4.02-4.09 (m, 2H), 3.70 (t, J=6.4 Hz, 2H), 3.17 (d, J=7.8 Hz, 3H), 2.95 (d, J=6.0 Hz, 2H), 2.74 (t, J=6.4 Hz, 2H), 2.58-2.61 (m, 3H), 1.99-2.06 (m, 2H), 1.82-1.91 (m, 1H), 1.68-1.75 (m, 4H), 1.23-1.34 (m, 2H), 1.12-1.21 (m, 4H).

### Chemical Synthesis Examples 38 & 39

### 1-(4-(1-((R)-2-Cyano-1-cyclopentylethyl)-1H-pyrazol-4-yl)-7H-pyrrolo[2,3-d]pyrimidin-7-yl)ethyl 5-((R)-1,2-dithiolan-3-yl)pentanoate (38) and (S)-3-(4-(7-(5-((R)-1,2-Dithiolan-3-yl)pentanoyl)-7H-pyrrolo[2,3-d]pyrimidin-4-yl)-1H-pyrazol-1-yl)-3-cyclopentylpropanenitrile (39)

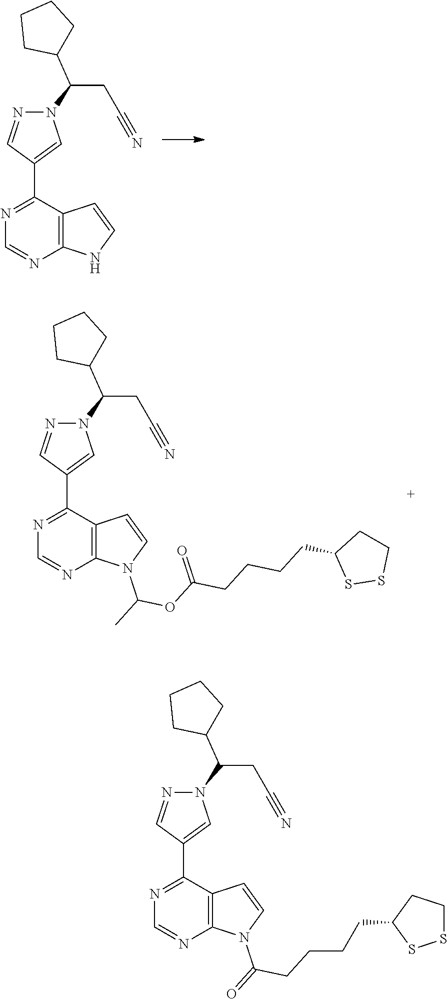

To a solution of Ruxolitinib (50 mg, 0.16 mmol) in DMF (2.0 mL), sodium hydride (60% dispersion in oil, 7.5 mg, 0.19 mmol) was added and the reaction mixture stirred at r.t. for 15 min. 1-Chloroethyl 5-[-1,2-dithiolan-3-yl]pentanoate (110 mg, 0.41 mmol) and NaI (25 mg, 0.16 mmol) were then added and the reaction mixture stirred at 50° C. for 16 h. The reaction mixture was partitioned between water (10 mL) and EtOAc (10 mL). The layers were separated and the organic phase washed successively with sat. NaHCO_{3(aq)} and sat. brine solution, dried (MgSO₄), filtered and the solvent evaporated in vacuo. The crude product was purified by preparative reversed-phase HPLC to afford 1-(4-(1-((R)-2-cyano-1-cyclopentylethyl)-1H-pyrazol-4-yl)-7H-pyrrolo[2,3-d]pyrimidin-7-yl)ethyl 5-((R)-1,2-dithiolan-3-yl)pentanoate and (S)-3-(4-(7-(5-((R)-1,2-dithiolan-3-yl)pentanoyl)-7H-pyrrolo[2,3-d]pyrimidin-4-yl)-1H-pyrazol-1-yl)-3-cyclopentylpropanenitrile.

1-(4-(1-((R)-2-Cyano-1-cyclopentylethyl)-1H-pyrazol-4-yl)-7H-pyrrolo[2,3-d]pyrimidin-7-yl)ethyl-5-((R)-1,2-dithiolan-3-yl)pentanoate as an off-white solid (2.2 mg, 3%). LCMS (Method C): Rt=5.59 min; [M+H]⁺=539.2. LCMS (Method D): Rt=5.71 min; [M+H]⁺=539.2.

(S)-3-(4-(7-(5-((R)-1,2-Dithiolan-3-yl)pentanoyl)-7H-pyrrolo[2,3-d]pyrimidin-4-yl)-1H-pyra-zol-1-yl)-3-cyclopentylpropanenitrile as a white solid (7.0 mg, 9%). LCMS (Method C): Rt =6.04 min; [M+H]⁺=495.2. LCMS (Method E): Rt=6.03 min; [M+H]⁺=495.2. ¹H-NMR (400 MHz, DMSO-D6) δ 8.89-8.94 (m, 2H), 8.40 (d, J=22.0 Hz, 1H), 8.12 (d, J=4.1 Hz, 1H), 7.32 (d, J=4.1 Hz, 1H), 4.55 (td, J=9.6, 4.5 Hz, 1H), 3.64-3.73 (m, 1H), 3.48-3.56 (m, 2H), 3.10-3.29 (m, 4H), 2.33-2.47 (m, 2H), 1.88-1.95 (m, 1H), 1.71-1.87 (m, 4H), 1.47-1.69 (m, 6H), 1.12-1.45 (m, 4H).

### Chemical Synthesis Examples 40, 41, & 42

### [4-[(1S)-1-(2-Cyano-1-cyclopentyl-ethyl)pyrazol-4-yl]pyrrolo[2,3-d]pyrimidin-7-yl]methyl 5-[(3R)-dithiolan-3-yl]pentanoate (40), (4-(1-((R)-2-cyano-1-cyclopentylethyl)-1H-pyrazol-4-yl)-7H-pyrrolo[2,3-d]pyrimidin-7-yl)methyl 5-((3R)-2-oxido-1,2-dithiolan-3-yl) pentanoate (41), and (4-(1-((R)-2-cyano-1-cyclopentylethyl)-1H-pyrazol-4-yl)-7H-pyrrolo[2,3-d]pyrimidin-7-yl)methyl 5-((3R)-1-oxido-1,2-dithiolan-3-yl)pentanoate (42)

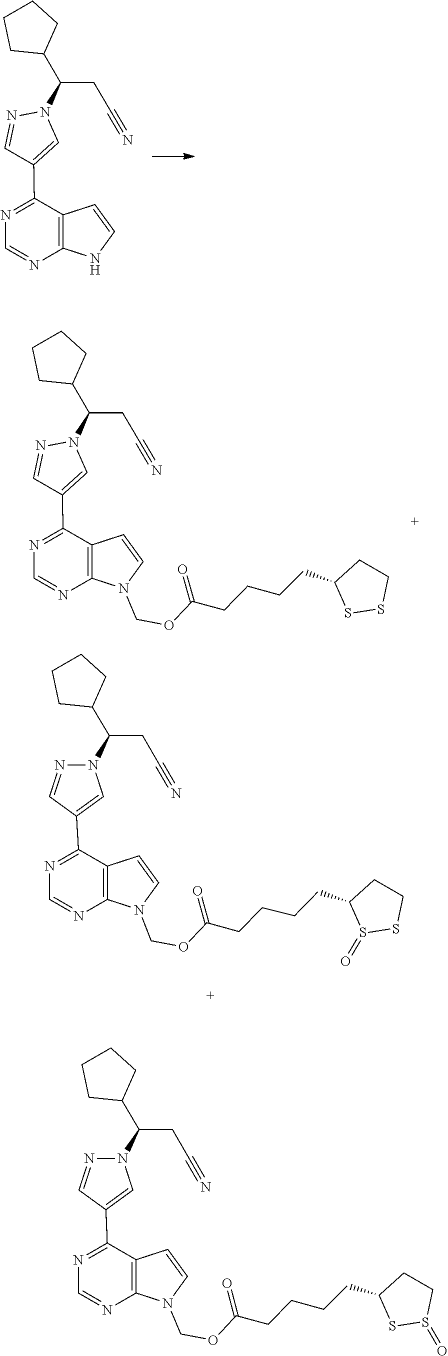 [SMILES: N#CC[C@H](C1CCCC1)n1cc(-c2ncnc3[nH]ccc23)cn1 ; N#CC[C@H](C1CCCC1)n1cc(-c2ncnc3c2ccn3COC(=O)CCCC[C@@H]2CCSS2)cn1 ; N#CC[C@H](C1CCCC1)n1cc(-c2ncnc3c2ccn3COC(=O)CCCC[C@@H]2CCSS2=O)cn1 ; N#CC[C@H](C1CCCC1)n1cc(-c2ncnc3c2ccn3COC(=O)CCCC[C@@H]2CCS(=O)S2)cn1]

To a solution of Ruxolitinib (60 mg, 0.20 mmol) in DMF (2.0 mL), sodium hydride (60% dispersion in oil, 12 mg, 0.30 mmol) was added and the reaction mixture stirred at r.t. for 15 min. Chloromethyl-(R)-5-(1,2-dithiolan-3-yl)pentanoate (100 mg, 0.39 mmol) was then added and the reaction mixture stirred at r.t for 2 h. The reaction mixture was partitioned between water and EtOAc. The layers were separated and the organic phase washed successively with sat. NaHCO_{3(aq)} and sat. brine solution, dried (MgSO₄), filtered and the solvent evaporated in vacuo. The crude product was purified by preparative reversed-phase HPLC to afford [4-[(1S)-1-(2-cyano-1-cyclopentyl-ethyl)pyrazol-4-yl]pyrrolo[2,3-d]pyrimidin-7-yl]methyl 5-[(3R)-dithiolan-3-yl]pentanoate, (4-(1-((R)-2-cyano-1-cyclopentylethyl)-1H-pyrazol-4-yl)-7H-pyrrolo[2,3-d]pyrimidin-7-yl)methyl 5-((3R)-2-oxido-1,2-dithiolan-3-yl) pentanoate, and (4-(1-((R)-2-cyano-1-cyclopentylethyl)-1H-pyrazol-4-yl)-7H-pyrrolo[2,3-d]pyrimidin-7-yl)methyl 5-((3R)-1-oxido-1,2-dithiolan-3-yl)pentanoate.

[4-[(1S)-1-(2-Cyano-1-cyclopentyl-ethyl)pyrazol-4-yl]pyrrolo[2,3-d]pyrimidin-7-yl]methyl 5-[(3R)-dithiolan-3-yl]pentanoate as a beige solid (11.7 mg, 11%). LCMS (Method C): Rt=5.13 min; [M+H]⁺=525.1. LCMS (Method D): Rt=5.17 min; [M+H]⁺=525.1.

(4-(1-((R)-2-Cyano-1-cyclopentylethyl)-1H-pyrazol-4-yl)-7H-pyrrolo[2,3-d]pyrimidin-7-yl)methyl 5-((3R)-2-oxido-1,2-dithiolan-3-yl) pentanoate and (4-(1-((R)-2-cyano-1-cyclopentylethyl)-1H-pyrazol-4-yl)-7H-pyrrolo[2,3-d]pyrimidin-7-yl)methyl 5-((3R)-1-oxido-1,2-dithiolan-3-yl)pentanoate as a white solid (5.2 mg, 5%). LCMS (Method C): Rt=5.75 min; [M+H]⁺=541.1. LCMS (Method D): Rt=6.22 min; [M+H]⁺=541.1.

### Chemical Synthesis Examples 43 & 44

### (3R)-3-Cyclopentyl-3-(4-(7-(5-((3R)-2-oxido-1,2-dithiolan-3-yl)pentanoyl)-7H-pyrrolo[2,3-d]pyrimidin-4-yl)-1H-pyrazol-1-yl)propanenitrile (43) and (3R)-3-Cyclopentyl-3-(4-(7-(5-((3R)-1-oxido-1,2-dithiolan-3-yl)pentanoyl)-7H-pyrrolo[2,3-d]pyrimidin-4-yl)-1H-pyrazol-1-yl)propanenitrile (44)

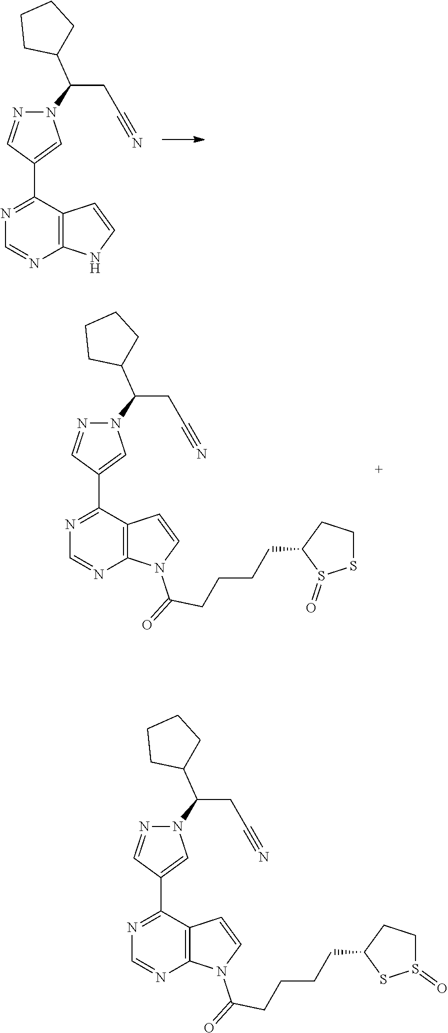 [SMILES: N#CC[C@H](C1CCCC1)n1cc(-c2ncnc3[nH]ccc23)cn1 ; N#CC[C@H](C1CCCC1)n1cc(-c2ncnc3c2ccn3C(=O)CCCC[C@@H]2CCSS2=O)cn1 ; N#CC[C@H](C1CCCC1)n1cc(-c2ncnc3c2ccn3C(=O)CCCC[C@@H]2CCS(=O)S2)cn1]

1-(3-Dimethylaminopropyl)-3-ethyl-carbodiimide hydrochloride (63 mg, 0.33 mmol) was dissolved in anhydrous DCM (4.0 mL), 5-[(3R)-2-oxido-1,2-dithiolan-3-yl]pentanoic acid (29.1 mg, 0.13 mmol) was added and the reaction mixture stirred at r.t. for 1 h. Separately, DMAP (16 mg, 0.13 mmol) was dissolved in anhydrous DCM (4.0 mL), Ruxolitinib (40 mg, 0.13 mmol) was added and the reaction mixture stirred at r.t. for 10 min. The two solutions were combined, and the reaction mixture stirred at r.t. for 16 h. The solvent was evaporated in vacuo and the crude product purified by preparative reversed-phase HPLC to afford (3R)-3-cyclopentyl-3-(4-(7-(5-((3R)-2-oxido-1,2-dithiolan-3-yl)pentanoyl)-7H-pyrrolo[2,3-d]pyrimidin-4-yl)-1H-pyrazol-1-yl)propanenitrile and (3R)-3-cyclopentyl-3-(4-(7-(5-((3R)-1-oxido-1,2-dithiolan-3-yl)pentanoyl)-7H-pyrrolo[2,3-d]pyrimidin-4-yl)-1H-pyrazol-1-yl)propanenitrile as a white solid (3.6 mg, 6%). LCMS (Method C): Rt=4.99 min; [M+H]⁺=511.2. LCMS (Method D): Rt=5.02 min; [M+H]⁺=511.2.

### Chemical Synthesis Example 45

### Methyl (R)-4-(4-(1-(2-cyano-1-cyclopentylethyl)-1H-pyrazol-4-yl)-7H-pyrrolo[2,3-d]pyrimidin-7-yl)-4-oxobutanoate

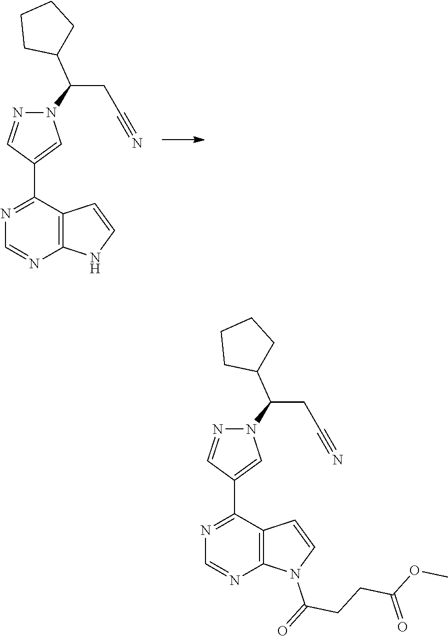 [SMILES: N#CC[C@H](C1CCCC1)n1cc(-c2ncnc3[nH]ccc23)cn1 ; COC(=O)CCC(=O)n1ccc2c(-c3cnn([C@H](CC#N)C4CCCC4)c3)ncnc21]

1-(3-Dimethylaminopropyl)-3-ethyl-carbodiimide hydrochloride (63 mg, 0.33 mmol) was dissolved in anhydrous DCM (2 mL), mono-methyl hydrogen succinate (17 mg, 0.13 mmol) was added and the reaction mixture stirred at r.t. for 1 h. Separately, DMAP (16 mg, 0.13 mmol) was dissolved in anhydrous DCM (2 mL), Ruxolitinib (40 mg, 0.13 mmol) was added and the reaction mixture was stirred at r.t. for 10 min. The two solutions were combined, and the reaction mixture stirred at r.t. for 16 h. The solvent was evaporated in vacuo and the crude product purified by flash chromatography (Biotage Isolera four; 10 g Sfar cartridge) eluting with 70% EtOAc in isohexane, to afford methyl (R)-4-(4-(1-(2-cyano-1-cyclopentylethyl)-1H-pyrazol-4-yl)-7H-pyrrolo[2,3-d]pyrimidin-7-yl)-4-oxobutanoate as a white solid (43.0 mg, 78%). LCMS (Method C): Rt=4.89 min; [M+H]⁺=421.3. LCMS (Method D): Rt=4.90 min; [M+H]⁺=421.3.

### Chemical Synthesis Example 46

### Ethyl (R)-4-(4-(1-(2-cyano-1-cyclopentylethyl)-1H-pyrazol-4-yl)-7H-pyrrolo[2,3-d]pyrimi-din-7-yl)-4-oxobutanoate

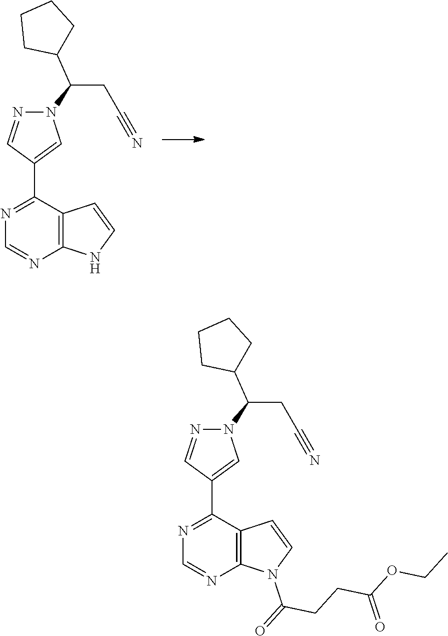 [SMILES: N#CC[C@H](C1CCCC1)n1cc(-c2ncnc3[nH]ccc23)cn1 ; CCOC(=O)CCC(=O)n1ccc2c(-c3cnn([C@H](CC#N)C4CCCC4)c3)ncnc21]

1-(3-Dimethylaminopropyl)-3-ethyl-carbodiimide hydrochloride (63 mg, 0.33 mmol) was dissolved in anhydrous DCM (2.0 mL), mono-ethyl succinate (19 mg, 0.13 mmol) was then added and the mixture was stirred at r.t. for 1 h. Separately, DMAP (16 mg, 0.13 mmol) was dissolved in anhydrous DCM (2 mL), Ruxolitinib (40 mg, 0.13 mmol) was added and the reaction mixture was stirred at r.t. for 10 min. The two solutions were combined, and the reaction mixture stirred at r.t. for 16 h. The solvent was evaporated in vacuo and the crude product purified by flash chromatography (Biotage Isolera four; 10 g Sfar cartridge) eluting with 70% EtOAc in isohexane to afford ethyl (R)-4-(4-(1-(2-cyano-1-cyclopentylethyl)-1H-pyrazol-4-yl)-7H-pyrrolo[2,3-d]pyrimi-din-7-yl)-4-oxobutanoate as a white solid (26.7 mg, 47%). LCMS (Method C): Rt=5.14 min; [M+H]⁺=435.2. LCMS (Method D): Rt=5.17 min; [M+H]⁺=435.2.

### Chemical Synthesis Example 47

### tert-Butyl (R)-4-(4-(1-(2-cyano-1-cyclopentylethyl)-1H-pyrazol-4-yl)-7H-pyrrolo[2,3-d]pyrimidin-7-yl)-4-oxobutanoate

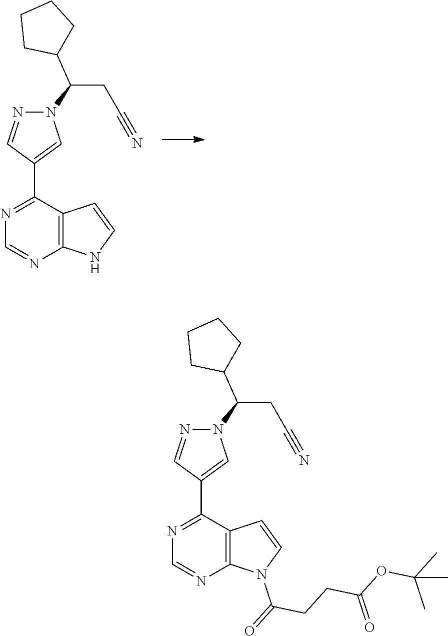 [SMILES: N#CC[C@H](C1CCCC1)n1cc(-c2ncnc3[nH]ccc23)cn1 ; CC(C)(C)OC(=O)CCC(=O)n1ccc2c(-c3cnn([C@H](CC#N)C4CCCC4)c3)ncnc21]

1-(3-Dimethylaminopropyl)-3-ethyl-carbodiimide hydrochloride (125 mg, 0.65 mmol) was dissolved in anhydrous DCM (3.0 mL), 4-(tert-butoxy)-4-oxobutanoic acid (46 mg, 0.26 mmol) was added and the mixture was stirred at r.t. for 1 h. Separately, DMAP (32 mg, 0.26 mmol) was dissolved in anhydrous DCM (3.0 mL), Ruxolitinib (80 mg, 0.26 mmol) was added and the mixture stirred at r.t. for 10 min. The two solutions were combined, and the reaction mixture stirred at r.t. for 16 h. The solvent was evaporated in vacuo and the crude product was purified by flash chromatography (Biotage Isolera four; 10 g Sfar cartridge) eluting with 70% EtOAc in isohexane, to afford tert-butyl (R)-4-(4-(1-(2-cyano-1-cyclopentylethyl)-1H-pyrazol-4-yl)-7H-pyrrolo[2,3-d]pyrimidin-7-yl)-4-oxobutanoate as a white solid (84.0 mg, 70%). LCMS (Method C): Rt=5.59 min; [M+H]⁺=483.3. LCMS (Method D): Rt=5.62 min; [M+H]⁺=483.3

### Chemical Synthesis Examples 48 & 49

### 1-(4-(1-((R)-2-Cyano-1-cyclopentylethyl)-1H-pyrazol-4-yl)-7H-pyrrolo[2,3-d]pyrimidin-7-yl)ethyl isobutyrate (48) and (R)-3-cyclopentyl-3-(4-(7-isobutyryl-7H-pyrrolo[2,3-d]pyrimidin-4-yl)-1H-pyrazol-1-yl)propanenitrile (49)

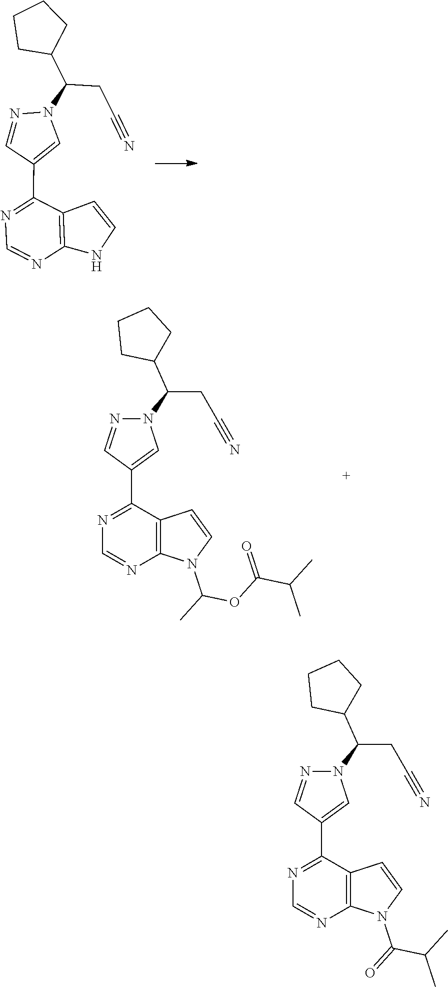 [SMILES: N#CC[C@H](C1CCCC1)n1cc(-c2ncnc3[nH]ccc23)cn1 ; CC(C)C(=O)OC(C)n1ccc2c(-c3cnn([C@H](CC#N)C4CCCC4)c3)ncnc21 ; CC(C)C(=O)n1ccc2c(-c3cnn([C@H](CC#N)C4CCCC4)c3)ncnc21]

To a solution of Ruxolitinib (75 mg, 0.23 mmol) in DMF (2.5 mL), sodium hydride (60% dispersion in oil, 10 mg, 0.42 mmol) was added and the reaction mixture stirred at r.t. for 15 min. 1-Chloroethyl 2-methylpropanoate (120 mg, 0.80 mmol) and NaI (10 mg, 0.29 mmol) were added and the reaction mixture stirred at r.t for 3 h. The reaction mixture was partitioned between water and EtOAc. The layers were separated and the organic phase washed successively with sat. NaHCO_{3(aq)} and sat. brine solution, dried (MgSO₄), filtered and the solvent evaporated in vacuo. The crude product was purified by preparative reversed-phase HPLC to afford 1-(4-(1-((R)-2-cyano-1-cyclopentylethyl)-1H-pyrazol-4-yl)-7H-pyrrolo[2,3-d]pyrimidin-7-yl)ethyl isobutyrate and (R)-3-cyclopentyl-3-(4-(7-isobutyryl-7H-pyrrolo[2,3-d]pyrimidin-4-yl)-1H-pyrazol-1-yl)propanenitrile.

1-(4-(1-((R)-2-Cyano-1-cyclopentylethyl)-1H-pyrazol-4-yl)-7H-pyrrolo[2,3-d]pyrimidin-7-yl)ethyl isobutyrate as a colorless gum (17.8 mg, 19%). LCMS (Method C): Rt=4.69 min; [M+H]⁺=421.3. LCMS (Method D): Rt=4.79 min; [M+H]⁺=421.3. ¹H-NMR (400 MHz, DMSO-D6) δ 8.79 (s, 1H), 8.71 (s, 1H), 8.35 (s, 1H), 7.89 (d, J=3.7 Hz, 1H), 7.25 (q, J=6.3 Hz, 1H), 7.09 (d, J=3.7 Hz, 1H), 4.50 (td, J=9.6, 4.1 Hz, 1H), 3.10-3.26 (m, 2H), 2.31-2.44 (m, 1H), 1.72-1.86 (m, 4H), 1.08-1.67 (m, 7H), 1.01 (d, J=6.9 Hz, 3H), 0.95 (d, J=7.3 Hz, 3H).

(R)-3-Cyclopentyl-3-(4-(7-isobutyryl-7H-pyrrolo[2,3-d]pyrimidin-4-yl)-1H-pyrazol-1-yl)propanenitrile as a white solid (20.6 mg, 24%). LCMS (Method C): Rt=5.13 min; [M+H]⁺=377.2. LCMS (Method D): Rt=5.02 min; [M+H]⁺=377.2.

### Chemical Synthesis Examples 50 & 51

### 1-(4-(1-((R)-2-Cyano-1-cyclopentylethyl)-1H-pyrazol-4-yl)-7H-pyrrolo[2,3-d]pyrimidin-7-yl)ethyl isopropyl carbonate (50) and isopropyl (R)-4-(1-(2-cyano-1-cyclopentylethyl)-1H-pyrazol-4-yl)-7H-pyrrolo[2,3-d]pyrimidine-7-carboxylate (51)

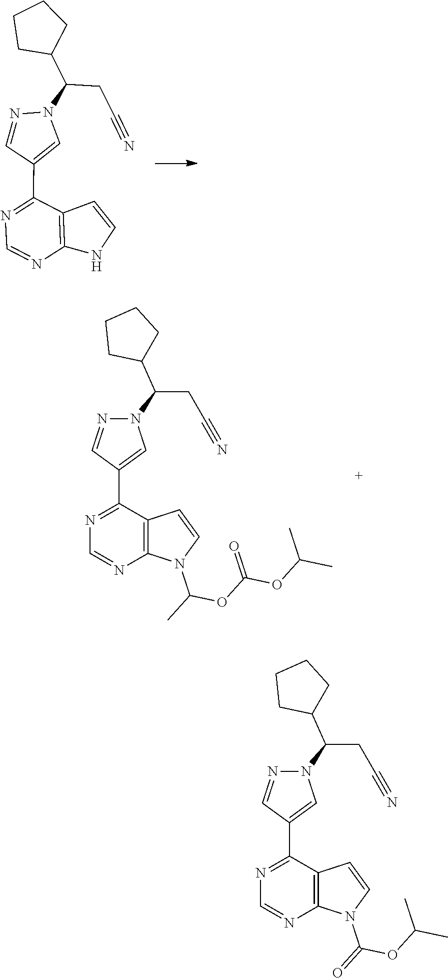 [SMILES: N#CC[C@H](C1CCCC1)n1cc(-c2ncnc3[nH]ccc23)cn1 ; CC(C)OC(=O)OC(C)n1ccc2c(-c3cnn([C@H](CC#N)C4CCCC4)c3)ncnc21 ; CC(C)OC(=O)n1ccc2c(-c3cnn([C@H](CC#N)C4CCCC4)c3)ncnc21]

To a solution of Ruxolitinib (50 mg, 0.16 mmol) in DMF (1.5 mL), sodium hydride (60% dispersion in oil, 7 mg, 0.29 mmol) was added and the reaction mixture stirred at r.t. for 15 min. 1-Chloroethyl isopropyl carbonate (60 mg, 0.36 mmol) and NaI (6 mg, 40 μmol) were added and the reaction mixture stirred at r.t. for 4 h. The reaction mixture was partitioned between water and EtOAc. The layers were separated, and the organic phase washed successively with sat. NaHCO_{3(aq)} and sat. brine solution, dried (MgSO₄), filtered and the solvent evaporated in vacuo. The crude product was purified by preparative reversed-phase HPLC to afford 1-(4-(1-((R)-2-cyano-1-cyclopentylethyl)-1H-pyrazol-4-yl)-7H-pyrrolo[2,3-d]pyrimidin-7-yl)ethyl isopropyl carbonate and isopropyl (R)-4-(1-(2-cyano-1-cyclopentylethyl)-1H-pyrazol-4-yl)-7H-pyrrolo[2,3-d]pyrimidine-7-carboxylate.

1-(4-(1-((R)-2-Cyano-1-cyclopentylethyl)-1H-pyrazol-4-yl)-7H-pyrrolo[2,3-d]pyrimidin-7-yl)ethyl isopropyl carbonate was afforded as a white solid (15.9 mg, 22%). LCMS (Method G): Rt=7.24 min; [M+H]⁺=437.3. LCMS (Method D): Rt=5.71 min; [M+H]⁺=437.3.

Isopropyl (R)-4-(1-(2-cyano-1-cyclopentylethyl)-1H-pyrazol-4-yl)-7H-pyrrolo[2,3-d]pyrimi-dine-7-carboxylate was afforded as a white solid (10.5 mg, 16%). LCMS (Method G): Rt=7.04 min; [M+H]⁺=393.3. LCMS (Method D): Rt=6.48 min; [M+H]⁺=393.3.

### Chemical Synthesis Example 52

### 1-((5-((R)-1,2-Dithiolan-3-yl)pentanoyl)oxy)ethyl (1r,4R)-4-cyano-4-(3-(cyclopentyloxy)-4-methoxyphenyl)cyclohexane-1-carboxylate

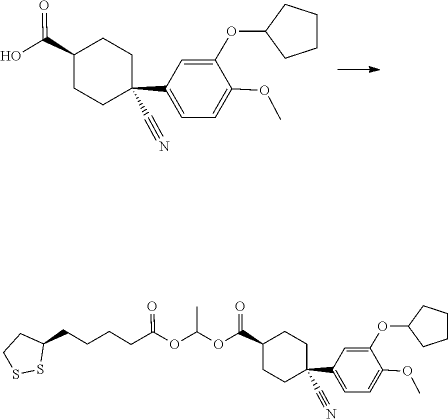 [SMILES: COc1ccc([C@]2(C#N)CC[C@H](C(=O)O)CC2)cc1OC1CCCC1 ; COc1ccc([C@]2(C#N)CC[C@H](C(=O)OC(C)OC(=O)CCCC[C@@H]3CCSS3)CC2)cc1OC1CCCC1]

To a solution of Cilomilast (25.0 mg, 70.0 μmol) in DMF (1.5 mL) was added DIPEA (44 μL, 0.255 mmol) and 1-chloroethyl 5-[(3R)-dithiolan-3-yl]pentanoate (125 mg, 0.47 mmol) and the reaction mixture stirred at 50° C. for 48 h. To the reaction mixture was added water (3 mL) and EtOAc (3 mL) and the layers were separated. The aqueous phase was acidified and ethyl acetate (5 mL)added. The combined organic phases were dried (MgSO₄) and the solvent evaporated in vacuo. The resulting residue was dissolved in DMSO and purified by preparative HPLC to afford 1-((5-((R)-1,2-dithiolan-3-yl)pentanoyl)oxy)ethyl (1r,4R)-4-cyano-4-(3-(cyclopentyloxy)-4-methoxyphenyl)cyclohexane-1-carboxylate (3.1 mg, 3%) as a yellow oil. LCMS (Method C): Rt =6.13 min; [M+NH₄]⁺=593.2.

### Chemical Synthesis Examples 53 & 54

1-((5-((3S)-1-Oxido-1,2-dithiolan-3-yl)pentanoyl)oxy)ethyl (1r,4R)-4-cyano-4-(3-(cyclopentyloxy)-4-methoxyphenyl)cyclohexane-1-carboxylate (53) and 1-((5-((3S)-2-oxido-1,2-dithiolan-3-yl)pentanoyl)oxy)ethyl (1r,4R)-4-cyano-4-(3-(cyclopentyloxy)-4-methoxyphenyl)cyclohexane-1-carboxylate (54)

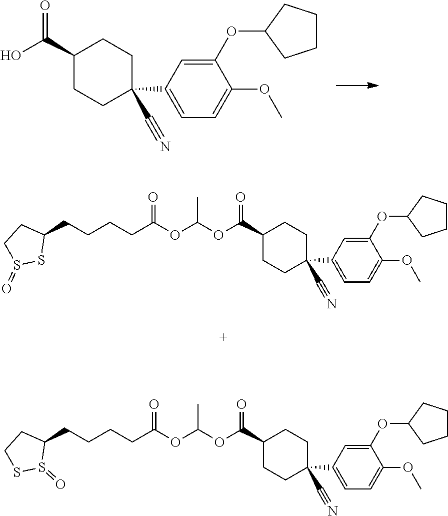 [SMILES: COc1ccc([C@]2(C#N)CC[C@@H](C(=O)O)CC2)cc1OC1CCCC1 ; COc1ccc([C@]2(C#N)CC[C@@H](C(=O)OC(C)OC(=O)CCCC[C@@H]3CCS(=O)S3)CC2)cc1OC1CCCC1 ; COc1ccc([C@]2(C#N)CC[C@@H](C(=O)OC(C)OC(=O)CCCC[C@@H]3CCSS3=O)CC2)cc1OC1CCCC1]

To a solution of Cilomilast (25.0 mg, 70.0 μmol) in DMF (1.5 mL) was added DIPEA (44.0 μL, 0.255 mmol) and 1-chloroethyl 5-[(3R)-dithiolan-3-yl]pentanoate (125 mg, 0.470 mmol). The reaction mixture was stirred at 50° C. for 48 h. To the reaction mixture was added water (3 mL) and ethyl acetate (3 mL). The layers were separated and the organic phase washed with sat. NaHCO_{3(aq)}, dried (MgSO₄), filtered and the solvent evaporated in vacuo. The crude product was purified by flash chromatography eluting with 20% EtOAc-isohexane→80% EtOAc-isohexane. The combined fractions were concentrated in vacuo to afford an orange oil. The oil was left to stand at r.t. under a blanket flow of air for 24 h to afford 1-((5-((3S)-1-oxido-1,2-dithiolan-3-yl)pentanoyl)oxy)ethyl (1r,4S)-4-cyano-4-(3-(cyclopentyloxy)-4-methoxyphenyl)cyclohexane-1-carboxylate and 1-((5-((3S)-2-oxido-1,2-dithiolan-3-yl)pentanoyl)oxy)ethyl (1r,4S)-4-cyano-4-(3-(cyclopentyloxy)-4-methoxyphenyl)cyclohexane-1-carboxylate (3.7 mg, 3%) as a yellow oil. LCMS (Method C): Rt=5.40 min; [M+H]⁺=592.2.

### Chemical Synthesis Example 55

### 1-(Isobutyryloxy)ethyl (1r,4r)-4-cyano-4-(3-(cyclopentyloxy)-4-methoxyphenyl)cyclohexane-1-carboxylate

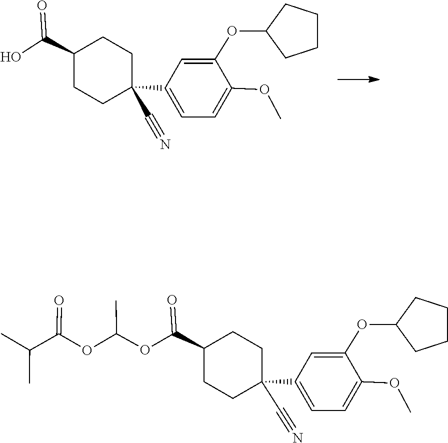 [SMILES: COc1ccc([C@]2(C#N)CC[C@@H](C(=O)O)CC2)cc1OC1CCCC1 ; COc1ccc([C@]2(C#N)CC[C@@H](C(=O)OC(C)OC(=O)C(C)C)CC2)cc1OC1CCCC1]

To a solution of Cilomilast (25.0 mg, 70.0 μmol) in DMF (1.5 mL) was added DIPEA (44.0 μL, 0.255 mmol) and 1-chloroethyl isobutyrate (33.0 mg, 0.219 mmol) and the reaction mixture stirred at 50° C. for 24 h. To the reaction mixture was added water (3 mL) and ethyl acetate (3 mL). The layers were separated and the organic phase washed with NaHCO_{3(aq)} (3 mL), dried (MgSO₄), filtered and the solvent evaporated in vacuo. The resulting residue was dissolved in DMSO and purified by preparative reversed-phase HPLC to afford 1-(isobutyryloxy)ethyl (1r,4r)-4-cyano-4-(3-(cyclopentyloxy)-4-methoxyphenyl)cyclohexane-1-carboxylate (4.5 mg, 14%) as an orange oil. LCMS (Method F): Rt=8.60 min; [M+NH₄]⁺=475.3.

### Chemical Synthesis Example 56

### (8S,9S,10R,11S,13S,14S,17R)-11,17-Dihydroxy-10,13-dimethyl-3-oxo-6,7,8,9,10,11,12,13,14,15,16,17-dodecahydro-3H-cyclopenta[a]phenanthrene-17-carboxylic acid

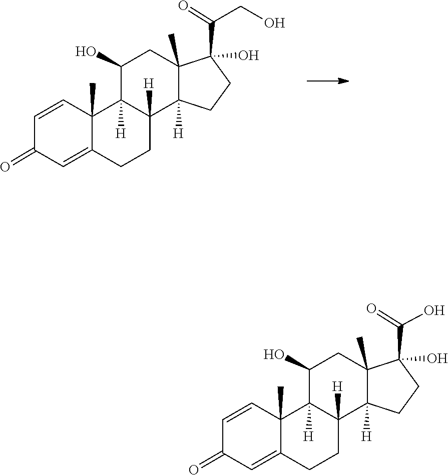

To a solution of prednisolone (0.50 g, 1.40 mmol) in THF (4.0 mL) and MeOH (1.0 mL) was added a warm solution (50° C.) of sodium periodate (0.89 g, 4.2 mmol) in water (3.3 mL). The resulting suspension was stirred at r.t. for 18 h. The reaction mixture was then concentrated in vacuo, diluted with water (5 mL) and the solid precipitate collected by filtration. The solid was further washed with water (2×5 mL) then dried in a vac oven at 40° C. for 16 h to afford (8S,9S,1R,11S,13S,14S,17R)-11,17-dihydroxy-10,13-dimethyl-3-oxo-6,7,8,9,10,11,12,13,14,15,16,17-dodecahydro-3H-cyclopenta[a]phenanthrene-17-carboxylic acid as a white solid (0.44 g, 92%). LCMS (Method C): Rt=3.71 min; [M+H]⁺=347.3. LCMS (Method E): Rt=2.71 min; [M+H]⁺=347.2.

### Chemical Synthesis Example 57

### (8S,9S,10R,11S,13S,14S,17R)-17-((Ethoxycarbonyl)oxy)-11-hydroxy-10,13-dimethyl-3-oxo-6,7,8,9,10,11,12,13,14,15,16,17-dodecahydro-3H-cyclopenta[a]phenanthrene-17-carboxylic acid

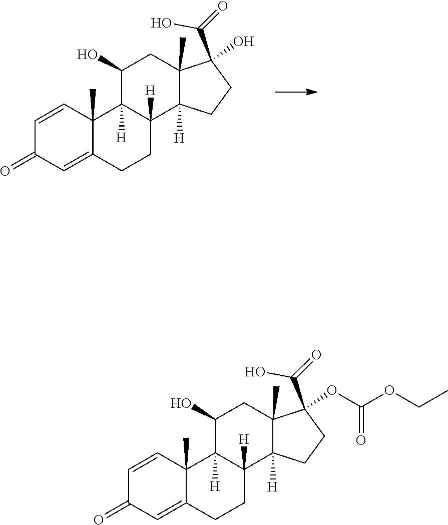

To a vigorously stirred mixture of (8S,9S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthrene-17-carboxylic acid (40 mg, 0.115 mmol), DCM (3.0 mL) and water (3.0 mL) was added ethyl chloroformate (33 μL, 0.35 mmol) as a solution in DCM (1.0 mL) and the mixture stirred at r.t. for 1 h. The reaction mixture was passed through a phase separator. Diethylamine (24 μL, 0.23 mmol) was added and the mixture stirred at r.t. for 5 days. Additional diethylamine (24 μL, 0.23 mmol) was added and the mixture stirred at r.t. for a further 4 h. The solvent was evaporated in vacuo and the crude product purified by preparative reversed-phase HPLC to afford (8S,9S,10R,11S,13S,14S,17R)-17-((ethoxycarbonyl)oxy)-11-hydroxy-10,13-dimethyl-3-oxo-6,7,8,9,10,11,12,13,14,15,16,17-dodecahydro-3H-cyclopenta[a]phenanthrene-17-carboxylic acid as a white solid (30.6 mg, 63%). LCMS (Method C): Rt=4.38 min; [M+H]⁺=419.3. LCMS (Method E): Rt=3.05 min; [M+H]⁺=419.3.

### Chemical Synthesis Example 58

### (8S,9S,10R,11S,13S,14S,17R)-17-((5-((R)-1,2-Dithiolan-3-yl)pentanoyl)oxy)-11-hydroxy-10,13-dimethyl-3-oxo-6,7,8,9,10,11,12,13,14,15,16,17-dodecahydro-3H-cyclopenta[a]phenanthrene-17-carboxylic acid

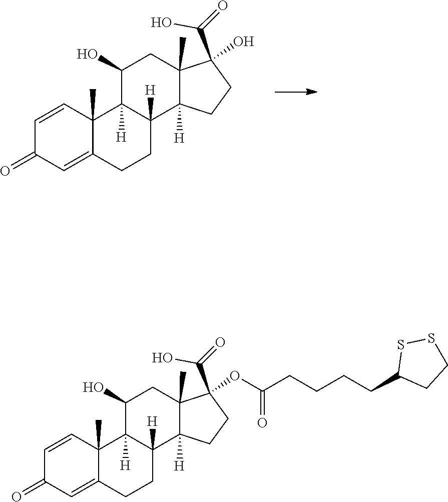

To a solution of Lipoic acid (30 mg, 0.14 mmol) in DMF (2.0 mL) was added DIPEA (60 μL, 0.35 mmol) and HATU (60 mg, 0.16 mmol). The resulting solution was stirred at r.t. for 1 h. (8S,9S,10R,11S,13S,14S,17R)-11,17-Dihydroxy-10,13-dimethyl-3-oxo-6,7,8,9,10,11,12,13,14,15,16,17-dodecahydro-3H-cyclopenta[a]phenanthrene-17-carboxylic acid (50 mg, 0.14 mmol) was added and the reaction mixture stirred at r.t. for 7 days. The reaction mixture was diluted with EtOAc (30 mL), washed with H₂O (3×15 mL) then sat. brine solution (15 mL). The organic phase was dried (MgSO₄), filtered and the solvent evaporated to afford the crude material as a yellow gum. Purification by preparative reversed-phase HPLC afforded (8S,9S,10R,11S,13S,14S,17R)-17-((5-((R)-1,2-dithiolan-3-yl)pentanoyl)oxy)-11-hydroxy-10,13-dimethyl-3-oxo-6,7,8,9,10,11,12,13,14,15,16,17-dodecahydro-3H-cyclopenta[a]phenanthrene-17-carboxylic acid as a white solid (39.6 mg, 51%). LCMS (Method C): Rt=5.13 min; [M+H]⁺=535.2. LCMS (Method E): Rt=3.63 min; [M+H]⁺=535.2.

### Chemical Synthesis Examples 59, 60, & 61

### Chloromethyl (8S,9S,10R,11S,13S,14S,17R)-17-((5-((R)-1,2-dithiolan-3-yl)pentanoyl)oxy)-11-hydroxy-10,13-dimethyl-3-oxo-6,7,8,9,10,11,12,13,14,15,16,17-dodecahydro-3H-cyclopenta[a]phenanthrene-17-carboxylate (59), chloromethyl (8S,9S,10R,11S,13S,14S,17R)-11-hydroxy-10,13-dimethyl-17-((5-((3R)-2-oxido-1,2-dithiolan-3-yl)pentanoyl)oxy)-3-oxo-6,7,8,9,10,11,12,13,14,15,16,17-dodecahydro-3H-cyclopenta[a]phenanthrene-17-carboxylate (60), and chloromethyl (8S,9S,10R,11S,13S,14S,17R)-11-hydroxy-10,13-dimethyl-17-((5-((3R)-1-oxido-1,2-dithiolan-3-yl)pentanoyl)oxy)-3-oxo-6,7,8,9,10,11,12,13,14,15,16,17-dodecahydro-3H-cyclopenta[a]phenanthrene-17-carboxylate (61)

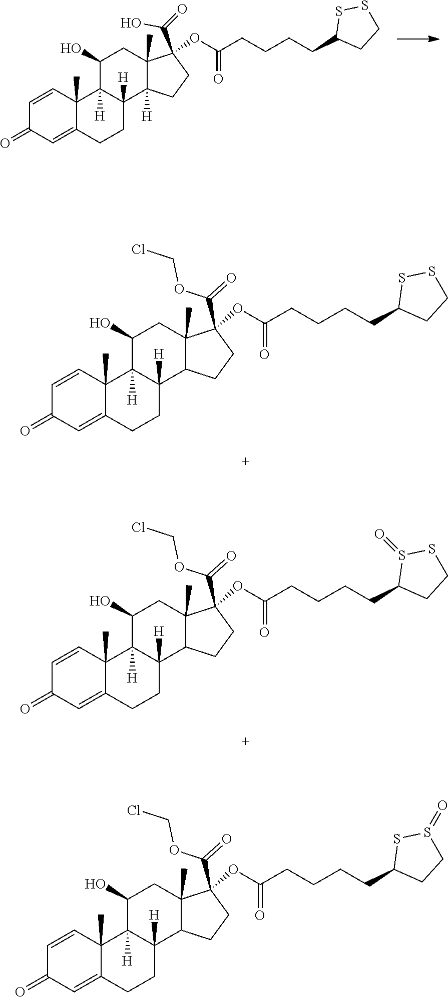

To a mixture of (8S,9S,10R,11S,13S,14S,17R)-17-((5-((R)-1,2-dithiolan-3-yl)pentanoyl)oxy)-11-hydroxy-10,13-dimethyl-3-oxo-6,7,8,9,10,11,12,13,14,15,16,17-dodecahydro-3H-cyclopenta[a]phenanthrene-17-carboxylic acid (60 mg, 0.112 mmol), tetrabutylammonium hydrogen sulfate (3.8 mg, 11 μmol) and sodium bicarbonate (75 mg, 0.90 mmol) in 1:1 water-DCM (6 mL) was added chloromethyl chlorosulfate (34 μL, 0.337 mmol). The reaction mixture was stirred vigorously at r.t. for 18 h. The reaction mixture was passed through a phase separator and the filtrate evaporated in vacuo. Purification by preparative reversed-phase HPLC afforded chloromethyl (8S,9S,10R,11S,13S,14S,17R)-17-((5-((R)-1,2-dithiolan-3-yl)pentanoyl)oxy)-11-hydroxy-10,13-dimethyl-3-oxo-6,7,8,9,10,11,12,13,14,15,16,17-dodecahydro-3H-cyclopenta[a]phenanthrene-17-carboxylate, chloromethyl (8S,9S,10R,11S,13S,14S,17R)-11-hydroxy-10,13-dimethyl-17-((5-((3R)-2-oxido-1,2-dithiolan-3-yl)pentanoyl)oxy)-3-oxo-6,7,8,9,10,11,12,13,14,15,16,17-dodecahydro-3H-cyclopenta[a]phenanthrene-17-carboxylate, and chloromethyl (8S,9S,10R,11S,13S,14S,17R)-11-hydroxy-10,13-dimethyl-17-((5-((3R)-1-oxido-1,2-dithiolan-3-yl)pentanoyl)oxy)-3-oxo-6,7,8,9,10,11,12,13,14,15,16,17-dodecahydro-3H-cyclopenta[a]phenanthrene-17-carboxylate.

Chloromethyl (8S,9S,10R,11S,13S,14S,17R)-17-((5-((R)-1,2-dithiolan-3-yl)pentanoyl)oxy)-11-hydroxy-10,13-dimethyl-3-oxo-6,7,8,9,10,11,12,13,14,15,16,17-dodecahydro-3H-cyclopenta[a]phenanthrene-17-carboxylate as an off-white solid (25.6 mg, 39%). LCMS (Method C): Rt=5.83 min; [M+H]⁺=583.2. LCMS (Method E): Rt=5.81 min; [M+H]⁺=583.2.

Chloromethyl (8S,9S,10R,11S,13S,14S,17R)-11-hydroxy-10,13-dimethyl-17-((5-((3R)-2-oxido-1,2-dithiolan-3-yl)pentanoyl)oxy)-3-oxo-6,7,8,9,10,11,12,13,14,15,16,17-dodecahydro-3H-cyclopenta[a]phenanthrene-17-carboxylate and chloromethyl (8S,9S,10R,11S,13S,14S,17R)-11-hydroxy-10,13-dimethyl-17-((5-((3R)-1-oxido-1,2-dithiolan-3-yl)pentanoyl)oxy)-3-oxo-6,7,8,9,10,11,12,13,14,15,16,17-dodecahydro-3H-cyclopenta[a]phenanthrene-17-carboxylate as a white solid (9.5 mg, 14%). LCMS (Method C): Rt=4.98 min; [M+H]⁺=599.2. LCMS (Method E): Rt=4.98 min; [M+H]⁺=599.2.

### Chemical Synthesis Example 62

### (R)-2,2-Dimethylthiazolidine-4-carboxylic acid

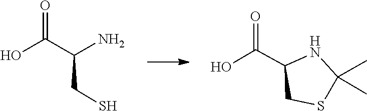

A suspension of L-cysteine (5.00 g, 40.0 mmol) in anhydrous acetone (125 mL) was heated under reflux (80° C.) under an atmosphere of nitrogen for 40 h. The reaction mixture was cooled to r.t., filtered through a Celite cartridge, concentrated to 50% of its original volume then left to stand at r.t for 24 h. The solid was collected, washed with acetone (10 mL) then dried to afford (R)-2,2-dimethylthiazolidine-4-carboxylic acid as a white solid (5.29 g, 82%). ¹H-NMR (400 MHz, D₂O) δ 4.41-4.45 (m, 1H), 3.46 (dd, J=12.4, 8.2 Hz, 1H), 3.31 (dd, J=12.4, 7.3 Hz, 1H), 1.66 (s, 3H), 1.64 (s, 3H).

### Chemical Synthesis Example 63

### (R)-3-Acetyl-2,2-dimethylthiazolidine-4-carboxylic acid

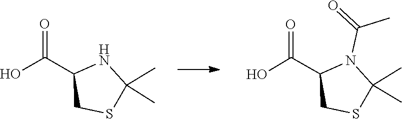

To a mixture of (R)-2,2-dimethylthiazolidine-4-carboxylic acid (2.00 g, 12.4 mmol) in acetone (100 mL) under an atmosphere of N₂ was added acetic anhydride (2.30 mL, 24.8 mmol) followed by DBU (3.70 mL, 24.8 mmol). The resultant solution was stirred at r.t. for 18 h. Sat. aqueous NH₄Cl (30 mL) was added to the reaction mixture and then stirred for 10 min. The mixture was extracted into EtOAc (2×50 mL), washed with water (50 mL) then sat. brine solution (50 mL), dried (MgSO₄), filtered and the solvent evaporated in vacuo to afford a first batch of (R)-3-acetyl-2,2-dimethylthiazolidine-4-carboxylic acid as a brown solid (0.82 g, 33%). LCMS (Method A): Rt=1.15 min; [M−H]⁻=202.1. LCMS (Method B): Rt=0.51 min; [M−H]⁻=202.1.

2M HCl_{(aq)} was added to the combined aqueous washings until pH 1 was reached. The product was extracted into EtOAc (2×50 mL). The combined organics were washed with sat. brine solution (50 mL), dried (MgSO₄), filtered and the solvent evaporated in vacuo to afford a second batch of (R)-3-acetyl-2,2-dimethylthiazolidine-4-carboxylic acid as a yellow solid (1.56 g, 62%). LCMS (Method A): Rt=1.12 min; [M−H]⁻=202.1. LCMS (Method B): Rt=0.29 min; [M−H]⁻=202.1. ¹H-NMR (400 MHz, ACETONE-D6) δ 11.17 (br s, 1H), 5.09 (dd, J=6.0, 1.4 Hz, 1H), 3.41 (dd, J=11.9, 6.0 Hz, 1H), 3.30 (dd, J=11.9, 0.9 Hz, 1H), 2.03 (s, 3H), 1.83 (s, 3H), 1.79 (s, 3H).

### Chemical Synthesis Example 64

### (8S,9S,10R,11S,13S,14S,17R)-17-(((R)-3-Acetyl-2,2-dimethylthiazolidine-4-carbonyl)oxy)-11-hydroxy-10,13-dimethyl-3-oxo-6,7,8,9,10,11,12,13,14,15,16,17-dodecahydro-3H-cyclopenta[a]phenanthrene-17-carboxylic acid

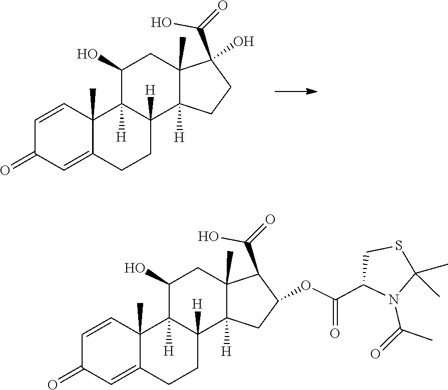

To a solution of (R)-3-acetyl-2,2-dimethylthiazolidine-4-carboxylic acid (29 mg, 0.144 mmol) in DMF (2.0 mL) was added DIPEA (0.060 mL, 0.346 mmol) and HATU (60 mg, 0.159 mmol). The resulting solution was stirred at r.t. for 1 h. (8S,9S,10R,11S,13S,14S,17R)-11,17-Dihydroxy-10,13-dimethyl-3-oxo-6,7,8,9,10,11,12,13,14,15,16,17-dodecahydro-3H-cyclopenta[a]phenanthrene-17-carboxylic acid (50 mg, 0.144 mmol) was added to the reaction mixture and stirring continued at r.t. for 16 h. The reaction mixture was diluted with EtOAc (30 mL), washed with water (3×15 mL) then sat. brine solution (15 mL), dried (MgSO₄), filtered and the solvent evaporated in vacuo. The crude product was purified by preparative reversed-phase HPLC to afford (8S,9S,10R,11S,13S,14S,17R)-17-(((R)-3-acetyl-2,2-dimethylthiazolidine-4-carbonyl)oxy)-11-hydroxy-10,13-dimethyl-3-oxo-6,7,8,9,10,11,12,13,14,15,16,17-dodecahydro-3H-cyclopenta[a]phenanthrene-17-carboxylic acid as a white solid (27.2 mg, 35%). LCMS (Method A): Rt=1.64 min; [M+H]⁺=532.2.

### Chemical Synthesis Example 65

### (8S,9S,10R,11S,13S,14S,17R)-17-((Acetyl-L-cysteinyl)oxy)-11-hydroxy-10,13-dimethyl-3-oxo-6,7,8,9,10,11,12,13,14,15,16,17-dodecahydro-3H-cyclopenta[a]phenanthrene-17-carboxylic acid

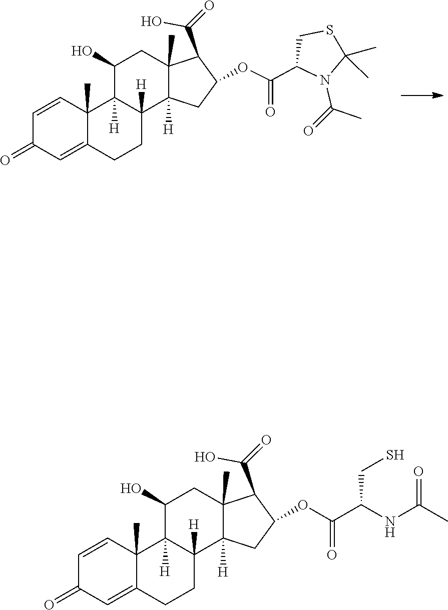

A solution of (8S,9S,10R,11S,13S,14S,17R)-17-(((R)-3-acetyl-2,2-dimethylthiazolidine-4-carbonyl)oxy)-11-hydroxy-10,13-dimethyl-3-oxo-6,7,8,9,10,11,12,13,14,15,16,17-dodecahydro-3H-cyclopenta[a]phenanthrene-17-carboxylic acid (58 mg, 0.11 mmol) in trifluoroacetic acid (2.0 mL) was heated at 40° C. for 3 h. The reaction mixture was evaporated to dryness in vacuo then purified by preparative reversed-phase HPLC to afford (8S,9S,10R,11S,13S,14S,17R)-17-((acetyl-L-cysteinyl)oxy)-11-hydroxy-10,13-dimethyl-3-oxo-6,7,8,9,10,11,12,13,14,15,16,17-dodecahydro-3H-cyclopenta[a]phenanthrene-17-carboxylic acid (34.0 mg, 63%) as a white solid. LCMS (Method C): Rt=3.90 min; [M+Na]=514.2. LCMS (Method E): Rt=2.91 min; [M+H]⁺=492.1.

### Chemical Synthesis Example 66

### (8S,9S,10R,11S,13S,14S,17R)-17-((Chloromethoxy)carbonyl)-11-hydroxy-10,13-dimethyl-3-oxo-6,7,8,9,10,11,12,13,14,15,16,17-dodecahydro-3H-cyclopenta[a]phenanthren-17-yl (R)-3-acetyl-2,2-dimethylthiazolidine-4-carboxylate

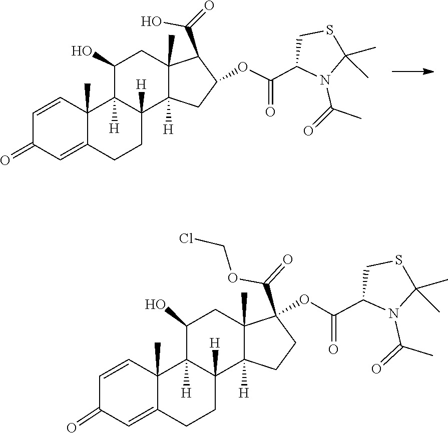

To a mixture of (8S,9S,10R,11S,13S,14S,17R)-17-(((R)-3-acetyl-2,2-dimethylthiazolidine-4-carbonyl)oxy)-11-hydroxy-10,13-dimethyl-3-oxo-6,7,8,9,10,11,12,13,14,15,16,17-dodecahydro-3H-cyclopenta[a]phenanthrene-17-carboxylic acid (50 mg, 0.094 mmol), tetrabutylammonium hydrogen sulfate (3.2 mg, 0.009 mmol) and sodium bicarbonate (63 mg, 0.75 mmol) in 1:1 DCM-H₂O (6 mL) was added chloromethyl chlorosulfate (29 μL, 0.28 mmol). The reaction mixture was stirred vigorously at r.t. for 18 h. The reaction mixture was passed through a phase separator with additional washing with DCM (3 mL) then the combined filtrate evaporated in vacuo to afford (8S,9S,10R,11S,13S,14S,17R)-17-((chloromethoxy)carbonyl)-11-hydroxy-10,13-dimethyl-3-oxo-6,7,8,9,10,11,12,13,14,15,16,17-dodecahydro-3H-cyclopenta[a]phenanthren-17-yl (R)-3-acetyl-2,2-dimethylthiazolidine-4-carboxylate as a yellow gum (53.4 mg, 98%). This was used without further purification. LCMS (Method A): Rt=1.84 min; [M+H]⁺=580.2.

### Chemical Synthesis Example 67

### Chloromethyl (8S,9S,10R,11S,13S,14S,17R)-17-((acetyl-L-cysteinyl)oxy)-11-hydroxy-10,13-dimethyl-3-oxo-6,7,8,9,10,11,12,13,14,15,16,17-dodecahydro-3H-cyclopenta[a]phenanthrene-17-carboxylate

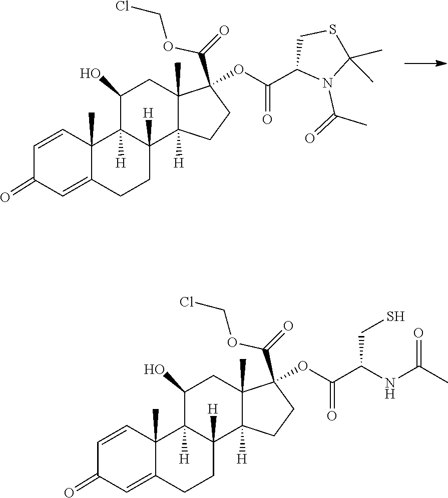

A solution of (8S,9S,10R,11S,13S,14S,17R)-17-((chloromethoxy)carbonyl)-11-hydroxy-10,13-dimethyl-3-oxo-6,7,8,9,10,11,12,13,14,15,16,17-dodecahydro-3H-cyclopenta[a]phenanthren-17-yl (R)-3-acetyl-2,2-dimethylthiazolidine-4-carboxylate (53.4 mg, 90 μmol) in trifluoroacetic acid (2.0 mL) was heated at 40° C. for 3 hours. The reaction mixture was evaporated to dryness in vacuo, redissolved in DMSO then purified by preparative reversed-phase HPLC to afford chloromethyl (8S,9S,10R,11S,13S,14S,17R)-17-((acetyl-L-cysteinyl)oxy)-11-hydroxy-10,13-dimethyl-3-oxo-6,7,8,9,10,11,12,13,14,15,16,17-dodecahydro-3H-cyclopenta[a]phenanthrene-17-carboxylate (23.6 mg, 47%) as a white solid. LCMS (Method C): Rt=4.58 min; [M+Na]=562.1. LCMS (Method E): Rt=4.58 min; [M+H]⁺=540.2.

### Chemical Synthesis Example 68

### (6S,8S,9R,10S,11S,13S,14S,16R,17R)-17-((5-((R)-1,2-Dithiolan-3-yl)pentanoyl)oxy)-6,9-difluoro-11-hydroxy-10,13,16-trimethyl-3-oxo-6,7,8,9,10,11,12,13,14,15,16,17-dodecahydro-3H-cyclopenta[a]phenanthrene-17-carboxylic acid

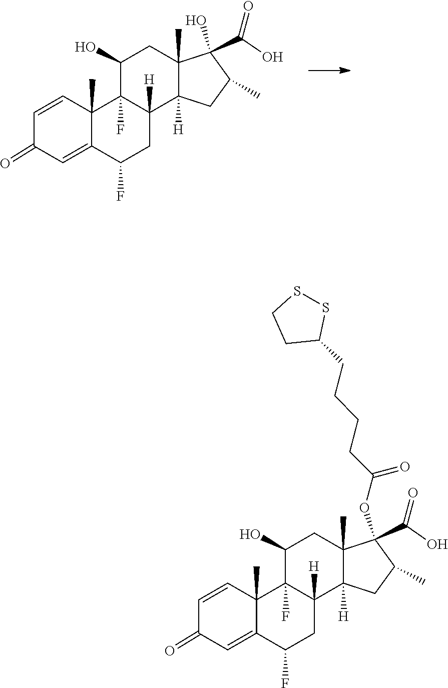

To a solution of (6S,8S,9R,10S,11S,13S,14S,16R,17R)-6,9-difluoro-11,17-dihydroxy-10,13,16-trimethyl-oxo-6,7,8,9,10,11,12,13,14,15,16,17-dodecahydro-3H-cyclopenta[a]phenanthrene-17-carboxylic acid (100 mg, 0.252 mmol) in DMF (2.0 mL) was added (R)-(+)-Lipoic acid (52 mg, 0.252 mmol) and HATU (115 mg, 0.303 mmol) followed by DIPEA (0.13 mL, 0.757 mmol). The reaction was stirred for 2 hours at r.t. The product was purified directly by prep HPLC to give (6S,8S,9R,10S,11S,13S,14S,16R,17R)-17-((5-((R)-1,2-dithiolan-3-yl)pentanoyl)oxy)-6,9-difluoro-11-hydroxy-10,13,16-trimethyl-3-oxo-6,7,8,9,10,11,12,13,14,15,16,17-dodecahydro-3H-cyclopenta[a]phenanthrene-17-carboxylic acid (116 mg, 0.198 mmol, 78%) as a yellow foam. LC-MS (Method H): Rt=1.88, m/z [M+H]⁺=585.3; [M+Na]⁺=607.4, 100%.

### Chemical Synthesis Example 69

### Cyanomethyl (6S,8S,9R,10S,11S,13S,14S,16R,17R)-17-((5-((R)-1,2-dithiolan-3-yl)pentanoyl)oxy)-6,9-difluoro-11-hydroxy-10,13,16-trimethyl-3-oxo-6,7,8,9,10,11,12,13,14,15,16,17-dodecahydro-3H-cyclopenta[a]phenanthrene-17-carboxylate (73A)

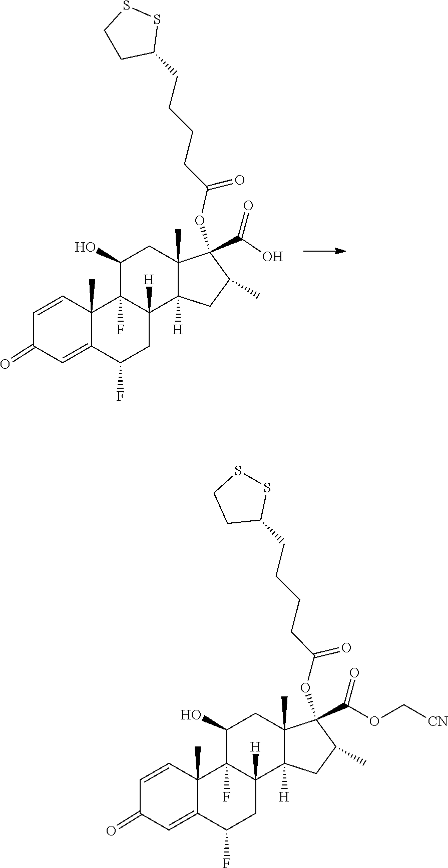

To a solution of (6S,8S,9R,10S,11S,13S,14S,16R,17R)-17-((5-((R)-1,2-Dithiolan-3-yl)pentanoyl)oxy)-6,9-difluoro-11-hydroxy-10,13,16-trimethyl-3-oxo-6,7,8,9,10,11,12,13,14,15,16,17-dodecahydro-3H-cyclopenta[a]phenanthrene-17-carboxylic acid (116 mg, 0.198 mmol) in DMF (2.0 mL) was added chloroacetonitrile (13 μL, 0.198 mmol) and TEA (55 μL, 0.395 mmol) and the reaction was stirred for 72 hours. The reaction was concentrated in vacuo and the residue was purified by column chromatography (silica, sfar 10 g, 0-10% MeOH in DCM) to give cyanomethyl (6S,8S,9R,10S,11S,13S,14S,16R,17R)-17-((5-((R)-1,2-dithiolan-3-yl)pentanoyl)oxy)-6,9-difluoro-11-hydroxy-10,13,16-trimethyl-3-oxo-6,7,8,9,10,11,12,13,14,15,16,17-dodecahydro-3H-cyclopenta[a]phenanthrene-17-carboxylate (101 mg, 0.161 mmol, 81%) as an off-white solid. LC-MS (Method J): Rt=5.55, [M+H]^{+=624.4, 99}%, LC-MS (Method K): Rt=5.53, [M+H]⁺=624.3, 99%. ¹H NMR (400 MHz, DMSO-d6) δ 7.22-7.26 (m, 2H), 7.15-7.17 (m, 1H), 6.29 (dd, J=10.1, 1.8 Hz, 1H), 6.10 (s, 1H), 5.53-5.70 (m, 2H), 4.98 (s, 2H), 4.18 (t, J=4.1 Hz, 1H), 3.53-3.59 (m, 1H), 3.05-3.21 (m, 2H), 2.31-2.40 (m, 3H), 2.21-2.29 (m, 2H), 2.02-2.12 (m, 2H), 1.78-1.87 (m, 2H), 1.59-1.66 (m, 2H), 1.48-1.56 (m, 6.5H), 1.32-1.40 (m, 1.5H), 1.19-1.25 (m, 1H), 0.98 (s, 3H), 0.85 (d, J=7.3 Hz, 3H).

### Chemical Synthesis Example 70

### (6S,8S,9R,10S,11S,13S,14S,16R,17R)-17-(((R)-3-Acetyl-2,2-dimethylthiazolidine-4-carbonyl)oxy)-6,9-difluoro-11-hydroxy-10,13,16-trimethyl-3-oxo-6,7,8,9,10,11,12,13,14,15,16,17-dodecahydro-3H-cyclopenta[a]phenanthrene-17-carboxylic acid

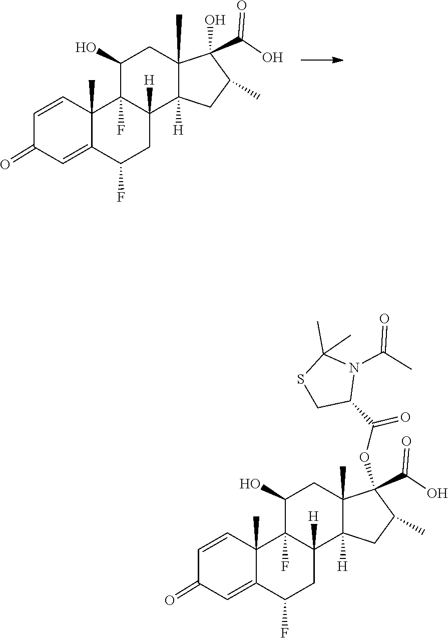

To a solution of (4R)-3-acetyl-2,2-dimethyl-thiazolidine-4-carboxylic acid (77 mg, 0.378 mmol) in DCM (5.0 mL) was added (6S,8S,9R,10S,11S,13S,14S,16R,17R)-6,9-difluoro-11,17-dihydroxy-10,13,16-trimethyl-oxo-6,7,8,9,10,11,12,13,14,15,16,17-dodecahydro-3H-cyclopenta[a]phenanthrene-17-carboxylic acid (150 mg, 0.378 mmol), HATU (173 mg, 0.454 mmol) and DIPEA (0.20 mL, 1.14 mmol). The solution was left to stir at r.t. for 3 hours. The reaction was concentrated in vacuo and purified by prep HPLC to give (6S,8S,9R,10S,11S,13S,14S,16R,17R)-17-(((R)-3-acetyl-2,2-dimethylthiazolidine-4-carbonyl)oxy)-6,9-difluoro-11-hydroxy-10,13,16-trimethyl-3-oxo-6,7,8,9,10,11,12,13,14,15,16,17-dodecahydro-3H-cyclopenta[a]phenanthrene-17-carboxylic acid (168 mg, 0.289 mmol, 76%) as a white solid. LC-MS (Method H): Rt=1.77, m/z [M+H]⁺=582.2; 95%.

### Chemical Synthesis Example 71

### (6S,8S,9R,10S,11S,13S,14S,16R,17R)-17-((Cyanomethoxycarbonyl)-6,9-difluoro-11-hydroxy-10,13,16-trimethyl-3-oxo-6,7,8,9,10,11,12,13,14,15,16,17-dodecahydro-3H-cyclopenta[a]phenanthrene-17-yl (R)-3-acetyl-2,2-dimethylthiazolidine-4-carboxylate

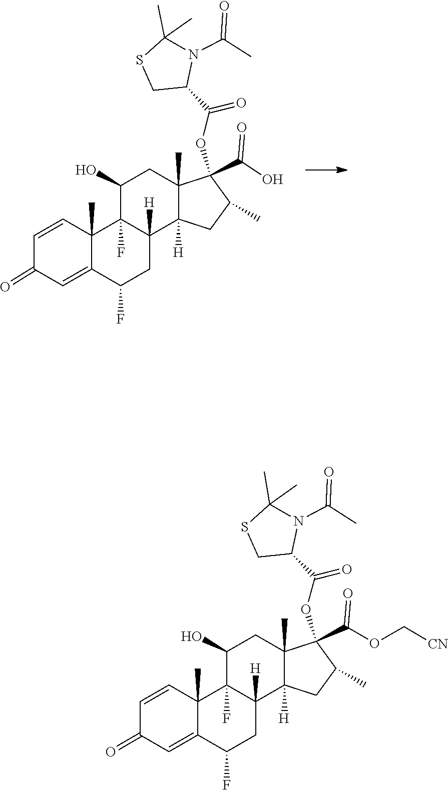

To a solution of (6S,8S,9R,10S,11S,13S,14S,16R,17R)-17-(((R)-3-acetyl-2,2-dimethylthiazolidine-4-carbonyl)oxy)-6,9-difluoro-11-hydroxy-10,13,16-trimethyl-3-oxo-6,7,8,9,10,11,12,13,14,15,16,17-dodecahydro-3H-cyclopenta[a]phenanthrene-17-carboxylic acid (168 mg, 0.289 mmol) in DMF (1.0 mL) was added chloroacetonitrile (22 uL, 0.347 mmol) and TEA (81 uL, 0.578 mmol). The reaction was left to stir at r.t. for 16 hours. The solvent was removed in vacuo and redissolved in DCM (10 mL) before being washed with sat. NaHCO₃ (10 mL) followed by sat. NH₄Cl (10 mL) and brine (10 mL) before being passed through a phase separator and concentrated in vacuo. The crude was purified by flash column chromatography (silica, sfar 10 g, 0-10% MeOH in DCM) to give (6S,8S,9R,10S,11S,13S,14S,16R,17R)-17-((cyanomethoxycarbonyl)-6,9-difluoro-11-hydroxy-10,13,16-trimethyl-3-oxo-6,7,8,9,10,11,12,13,14,15,16,17-dodecahydro-3H-cyclopenta[a]phenanthrene-17-yl (R)-3-acetyl-2,2-dimethylthiazolidine-4-carboxylate (37 mg, 0.0596 mmol, 20%,) as an off-white solid. LC-MS (Method L): Rt=2.73, m/z 621.3 μM+H]⁺, 84%.

### Chemical Synthesis Example 72

### Cyanomethyl (6S,8S,9R,10S,11S,13S,14S,16R,17R)-17-((acetyl-L-cysteinyl)oxy)-6,9-difluoro-11-hydroxy-10,13,16-trimethyl-3-oxo-6,7,8,9,10,11,12,13,14,15,16,17dodecahydro-3H-cyclopenta[a]phenanthrene-17-carboxylate (73B)

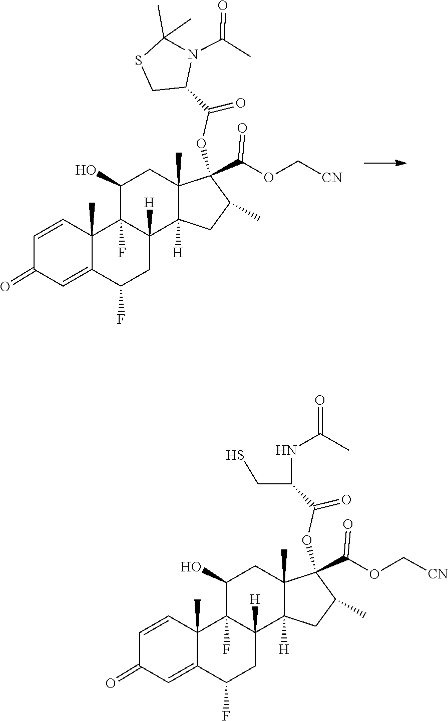

To a solution of (6S,8S,9R,10S,115,13S,14S,16R,17R)-17-((cyanomethoxycarbonyl)-6,9-difluoro-11-hydroxy-10,13,16-trimethyl-3-oxo-6,7,8,9,10,11,12,13,14,15,16,17-dodecahydro-3H-cyclopenta[a]phenanthrene-17-yl (R)-3-acetyl-2,2-dimethylthiazolidine-4-carboxylate (37 mg, 0.0596 mmol) in DCM (0.20 mL) in a sealed vial was added TFA (0.80 mL). The reaction was heated to 40° C. and left to stir for 5 hours. The reaction was concentrated in vacuo and purified by prep HPLC to give cyanomethyl (6S,8S,9R,10S,11S,13S,14S,16R,17R)-17-((acetyl-L-cysteinyl)oxy)-6,9-difluoro-11-hydroxy--10,13,16-trimethyl-3-oxo-6,7,8,9,10,11,12,13,14,15,16,17dodecahydro-3H-cyclopenta[a]phenanthrene-17-carboxylate (20 mg, 0.0344 mmol, 58%) as a white solid. LC-MS (Method J): Rt=4.38 min; [M+Na]⁺=603.4, 92%, LC-MS (Method K): Rt=4.36 min; [M+H]⁺=581.4, 84%. ¹H NMR (400 MHz, DMSO-d6) δ 8.27 (d, J=7.8 Hz, 1H), 7.26 (dd, J=10.1, 1.4 Hz, 1H), 6.30 (dd, J=10.5, 1.8 Hz, 1H), 6.11 (s, 1H), 5.71-5.54 (m, 2H), 4.95-4.97 (m, 2H), 4.40 (td, J=7.9, 4.7 Hz, 1H), 4.18 (td, J=4.6, 2.0 Hz, 1H), 3.13-3.18 (m, 1H), 2.78-2.85 (m, 1H), 2.65-2.75 (m, 1H), 2.59 (t, J=8.7 Hz, 1H), 2.24 (dd, J=6.6, 4.4 Hz, 1H), 2.00-2.12 (m, 2H), 1.76-1.85 (m, 4H), 1.60 (d, J=13.3 Hz, 1H), 1.43-1.52 (m, 4H), 1.14-1.20 (m, 1H), 0.98 (s, 3H), 0.87 (d, J=7.3 Hz, 3H).

Other compounds provided herein (e.g., in Table 1-9) are prepared according to a similar process as provided for the examples provided hereinabove (e.g., Chemical Synthesis Examples 1-72).

### II. Biological Evaluation

### Example 1: Rabbit Cornea Homogenate Stability Assay

Determining Rabbit Cornea Homogenate stability of the test compounds was performed using UPLC-MS. The assay was performed at two concentrations of Rabbit Cornea Homogenate (0.15 mg/mL and 0.45 mg/mL total protein) so that any hydrolysis observed can be assigned as esterase dependent or not.

### Rabbit Cornea Homogenisation

Three to four rabbit corneas (e.g., New Zealand Whites (NZW) or Dutch Belted (DB)) of approx. 50 mg each were sliced and scraped with a scalpel and tweezers until reduced to small (1-3 mm), thin pieces. These were transferred into a glass vial containing approximately 2 mL of cold DPBS pH 7.4 buffer.

Sample was cooled intermittently on ice and shear homogenized for 3 minutes, then centrifuged for 3 min at 13,000 g. The supernatant was pipetted off into a vial, and total protein concentration determined at 280 nm. Sample was stored at −78° C.

### Rabbit Cornea Esterase Assay

### Preparation of Stock Solutions:

10 mM Compound DMSO stocks were diluted to 10 μM in a glass vial: 10 μL of 10 mM Compound stock was added to 9,990 μl 50 mM DPBS, pH 7.4 buffer. Esterase homogenate was diluted to 300 ng/μl and 900 ng/μL in DPBS.

### Assay Conditions:

A heater shaker was set to 37° C. Into a suitable 96 well plate (Run Plate), 70 μL of 300 or 900 ng/μl esterase homogenate was pipetted into two rows as compounds were analyzed in duplicate (2 min, 5 min, 10 min, 20 min and 45 min). The plate was sealed and then warmed at 37° C. for 5 min.

Two 96 deep-well plates were put on ice (Kill Plates). To these, 990 μL of 50:50 MeCN—H₂O were added to required rows, labelled 0 min 2 min, 5 min, 10 min, 20 min and 45 min. The plates were covered to minimize evaporation.

To both rows of the Run Plate, 70 μL of 10 μM compound solution was added. At the appropriate time point, 10 μL of the assay mixture was added to the matching kill plate well containing 990 μL of 50:50 cold MeCN—H₂O. Samples were analyzed as soon as practicable by UPLC-MS (Waters Xevo TQ-S).

### Assay Conditions for Lipoic Acid Analysis:

A heater shaker was set to 37° C. Into a suitable 96 well plate (Run Plate), 80 μL of 300 or 900 ng/μL esterase homogenate was pipetted into two rows as compounds were analyzed in duplicate (2 min, 5 min, 10 min, 20 min and 45 min). The plate was sealed and then warmed at 37° C. for 5 min.

Two 96 shallow-well plates were placed on ice (Kill Plates). To these, 180 μL of 60:40 MeCN-H₂O+0.1% acetic acid are added to required rows. The plates were sealed to minimize evaporation.

To both rows of the Run Plate, 80 μL of 10 μM compound solution was added. At the appropriate time point, 20 μL of the assay mixture was added to the matching kill plate well containing 180 μL of 60:40 cold MeCN-H₂O+0.1% acetic acid. For lipoic acid analysis, samples were analyzed as soon as practicable by LCMS (Waters Xevo TQ-S). For parent conjugate and parent analysis the samples are diluted further 1 in 10: 20 μL supernatant is added to 180 μL of 50:50 MeCN—H₂O.

Parent conjugate, parent and keratolytic concentrations were determined against appropriate standard response curves and the half-life (T1/2) of the parent conjugate was calculated using the measured concentration of the parent conjugate at each time point in the linear region of the log —linear plot.

### Example 2: Aqueous Hydrolysis Stability Assay

Determination of aqueous stability of the test compounds was performed using UPLC-MS. A test compound 10 mM stock solution was prepared in DMSO. 10 μL of the DMSO stock solution was dissolved in 990 μL of DPBS pH 7.4 buffer to prepare a 100 μM stock. A further dilution was made by dissolving 75 μl of 100 μM stock into 225 μL of DPBS. Final DMSO concentration is 0.25%. The solution is kept at 37° C. and injected without delay into the LCMS (Waters Xevo TQ-). Additional injections were performed at appropriate time points.

Half-life (T_{1/2}) of the parent conjugate was calculated using the peak area or measured concentration of the parent conjugate at each time point in the linear region of the log—linear plot.

Aqueous Stability and Hydrolysis Rates of Example Compounds

TABLE 11

| | Aqueous Stability | Esterase | Esterase | Esterase |
|---|---|---|---|---|
| Chemical | (DPBS) | (0.15 mg/mL) | (0.45 mg/mL) | (0.90 mg/mL) |
| Synthesis | % parent released | % parent released | % parent released | % parent released |
| Example(s) | after t min | after 45 min | after 45 min | after 45 min |
| 12 | A (t = 44) | B (NZW) | B (NZW) | n.t. |
| 13 & 14 | A (t = 44) | A (NZW) | A (NZW) | n.t. |
| 15 | A (t = 44) | D (NZW) | D (NZW) | n.t. |
| 16 & 17 | A (t = 44) | A (NZW) | B (NZW) | n.t. |
| 18 | A (t = 44) | B (NZW) | B (NZW) | n.t |
| 19 | A (t = 44) | B (NZW) | B (NZW) | n.t. |
| 21 | A (t = 45) | C (NZW) | D (NZW) | n.t. |
| 22 | n.t. | D (NZW) | D (NZW) | n.t. |
| 23 | n.t | A (NZW) | A (NZW) | n.t. |
| 24 | n.t. | A (NZW) | A (NZW) | n.t. |
| 25 | A (t = 44) | D (NZW) | D (NZW) | n.t. |
| 26 | A (t = 44) | B (NZW) | B (NZW) | n.t. |
| 27 | A (t = 44) | B (NZW) | C (NZW) | n.t. |
| 28 | n.t. | n.t. | D (DB) | D (DB) |
| 29 | n.t. | B (NZW) | C (NZW) | D (DB) |
| | | | D (DB) | |
| 30 | B (t = 43) | B (NZW) | B (NZW) | n.t. |
| 31 | A (t = 44) | C (NZW) | C (NZW) | n.t. |
| 32 & 33 | n.t. | B (NZW) | C (NZW) | n.t. |
| 34 | n.t. | C (NZW) | C (NZW) | n.t. |
| 35 & 36 | A (t = 43) | B (NZW) | B (NZW) | n.t. |
| 37 | A (t = 42) | D (NZW) * | D (NZW) * | n.t. |
| 38 | A (t = 48) | B (NZW) | C (NZW) | n.t. |
| 39 | B (t = 45) | B (NZW) | C (NZW) | n.t. |
| 40 | A (t = 45) | D (NZW) | D (NZW) | n.t. |
| 41 & 42 | n.t. | n.t. | D (DB) | D (DB) |
| 43 & 44 | n.t. | B (NZW) | B (NZW) | n.t. |
| 45 | A (t = 44.2) | C (NZW) | D (NZW) | n.t. |
| 46 | n.t. | C (NZW) | D (NZW) | n.t |
| 47 | A (t = 45) | B (NZW) | B (NZW) | n.t. |
| 48 | n.t. | A (NZW) | A (NZW) | n.t. |
| 49 | n.t. | D NZW) | D (NZW) | n.t. |
| 50 | B (t = 44) | D (DB) | D (DB) | n.t. |
| 51 | A (t = 45) | A (DB) | B (DB) | n.t. |
| 52 | n.t. | D (DB) | D (DB) | n.t. |
| 53 & 54 | A (t = 45) | C (NZW) | C (NZW) | n.t. |
| 55 | A (t = 45) | D (DB) | D (DB) | n.t. |

A: <25%; B: 25% to 50%; C: 51% to 75%; D: >75%; n.t. = not tested.
* n = 2

TABLE 12

| | Aqueous | | | | | | | |
|---|---|---|---|---|---|---|---|---|
| Chemical | Stability | | Esterase | | Esterase | | Esterase | Esterase |
| Synthesis | (DPBS) T_{1/2} | | (0.15 mg/mL) | | (0.45 mg/mL) | | (0.9 mg/mL) | (2.0 mg/mL) |
| Example(s) | after t min | | T_{1/2} (45 min) | | T_{1/2} (45 min) | | T_{1/2} (45 min) | T_{1/2} (45 min) |
| 58 | >120 | (t = 44 min) | >120 | (NZW) | >120 | (NZW) | n.t. | n.t. |
| 59 | 31.6 | (t = 45) | n.t. | | n.t. | | 22.6 (NZW) | 21.3 (NZW) |
| 60 & 61 | n.t. | | 117 | (NZW) | 98 | (NZW) | n.t. | n.t. |
| 65 | 33.7 | (t = 42) | >120 | (NZW) | 119 | (NZW) | n.t. | n.t. |

| | | | | | | |
|---|---|---|---|---|---|---|
| 67 | >120 | (t = 44) | n.t. | n.t. | 47.7 (NZW) | 33.7 (NZW) |

a: <40 min; b: 40 min to 80 min; c: >80 min; n.t. = not tested.

TABLE 13

| | Aqueous Stability | Esterase | Esterase | Esterase |
|---|---|---|---|---|
| Chemical | (DPBS) | (0.15 mg/mL) | (0.45 mg/mL) | (0.90 mg/mL) |
| Synthesis | % parent released | % parent released | % parent released | % parent released |
| Example(s) | after t min | after 45 min | after 45 min | after 45 min |
| 73A | A (t = 45) | A | A | n.t. |
| 73B | A (t = 45) | A | A | n.t. |

A: <25%; B: 25% to 50%; C: 51% to 75%; D: >75%; n.t. = not tested.

### Example 3: Mouse Model of Experimental Dry Eye Disease

Female C57BL/6 mice (6-8 weeks old) or female HEL BCR Tg mice (6-8 weeks old) are commercially obtained. Experimental dry eye is induced as described by Niederkorn, et al. (J. Immunol. 2006,176:3950-3957) and Dursun et al. (Invest. Ophthalmol. Vis. Sci. 2002, 43:632-638). In brief, mice are exposed to desiccating stress in perforated cages with constant airflow from fans positioned on both sides and room humidity maintained at 30% to 35%. Injection of scopolamine hydrobromide (0.5 mg/0.2 mL; Sigma-Aldrich, St. Louis, MO) is administered subcutaneously, three times a day (8:00 AM, 12:00 noon, and 5:00 PM), on alternating hind-flanks to augment disease. Mice are exposed to desiccating stress for 3 weeks. Untreated control mice are maintained in a nonstressed environment at 50% to 75% relative humidity without exposure to forced air. Test animals are exposed to test compound and subsequently tear samples are obtained to determine stability of test compounds, and tissue samples are taken to determine presence of pro-inflammatory biomarkers.

### Example 4: Thiol Assay

### Stratum Corneum Preparation

Epidermis pieces are transferred and incubated overnight from 25-37° C. in a container containing 100 mL of 0.0005% trypsin (diluted in PBS). The stratum corneum pieces are removed and washed twice with HPLC grade water in a petri dish (145 mm), removing intact cells. The dish and/or pieces are shaken, producing nearly transparent layers. The stratum corneum is then transferred to a petri dish (145 mm), washed with hexane, and shaken to remove fats. Each piece is gently mounted on an absorbent paper. Each piece is transferred to an Eppendorf tube, allowing residual solvents to evaporate for a few minutes.

### Thiol Assay

Compounds (e.g., 50 μL; 1 μM to 800 μM) are applied to the isolated stratum corneum at room temperature for a period of 1-24 hours. The pieces are gently mixed with the compounds by pipetting. Following incubation, about 200 μL of 10 M sodium hydroxide is added, incubating for 1 hour at room temperature with continuous blending (e.g., vortexing) until the stratum corneum disintegrates. About 200 μL of 10 M hydrochloric acid is added to normalize pH, vortexing the samples. The samples are centrifuged (e.g., for 20 min at 16,000×g) at room temperature. The supernatant (middle layer) is transferred to an Eppendorf tube. The free thiols are isolated by adding tricholoracetic acid (e.g., 400 μL) and vortexing. The tubes are centrifuged (e.g., for 10 min at 16,000×g) at room temperature. The supernatant is removed, and Ellman's reagent solution (e.g., 220 μL) is added to the remaining pellet. After mixing, 100 μL for each tube is transferred to a 96 well plate in the dark. The plate is incubated for about 5 minutes at room temperature while shaking. The optical absorbance at 412 nm is detected and recorded.

### Example 5: Glucocorticoid Binding Assay

GR (h) (agonist radioligand) assay performed by Eurofins Cerep, France using a method similar to the one described hereinbelow.

The human lymphoblast cell line IM9 is used as a source of the soluble glucocorticoid receptor (GR). The cells are grown to densities of 1 to 10×10⁵ cells per milliliter in RPMI 1640 media containing 10% fetal bovine serum, penicillin (100 U/ml), streptomycin (100 μg/ml), and 2 mM L-glutamine at 370 and 7% CO2 in a humidified incubator. The IM9 cells are harvested from the media by centrifugation for 10 minutes at 1500 g. Cells are washed in 12 volumes of Dulbecco's phosphate-buffered saline (PBS) and re-pelleted. Washed cells are resuspended in five to six volumes (per volume of packed cells) of homogenization buffer (10 mM TES, 10 mM sodium molybdate, 1 mM EDTA, pH 7.4, 20 mM 2-mercaptoethanol, and 10% glycerol), and the cells are broken by nitrogen cavitation using 2×15 minutes at 600 to 750 psi nitrogen in the N₂ cavitator at 0° C. Cell disruption is confirmed by Hoffman contrast microscopy using a Nikon Diaphot. The broken cell preparation is then centrifuged at 27,000 g for 15 minutes, and the resultant supernatant was centrifuged at 103,000 g for 60 minutes at 4° C. The amount of protein in the supernatant fraction is determined using a BCA assay kit with a bovine serum albumin standard. Aliquots of the supernatant fraction are snap frozen in a dry ice-acetone bath and stored at −70° C. Competitive binding assays are done in duplicate in homogenization buffer (total volume of 200 IA) by mixing 1 mg of IM9 cytosol, 0.05/ρCi (3 nM) of ³H-dexamethasone, and Compounds described herein (10⁻⁵ to 10⁻¹¹ M). After incubation at 0° C. for 16 to 18 hours, the assay is stopped by the addition of 100 μg of a charcoal-dextran mixture (2% activated charcoal, 0.5% dextran in 10 mM Tris, 1 mM EDTA, pH 7.4). The assay mixture is further incubated at 0° C. for 10 minutes before being centrifuged for 5 minutes at 8200 g. A 100-μl sample of the supernatant (protein-bound steroid fraction) is assayed for radioactivity by liquid scintillation spectrometry, and the IC₅₀ values were determined graphically.

In some instances, the glucocorticoid binding of a compound described herein is provided in Table 14. In some instances, a free-acid is inactive in the glucocorticoid binding assay. In some instances, an ester is active in the glucocorticoid binding assay. In some instances, an ester retains activity, such as when attached to a keratolytic agent (or a radical thereof), in the glucocorticoid binding assay.

TABLE 14

| Chemical Synthesis | Glucocorticoid | |
|---|---|---|
| Example(s) | EC₅₀ | |
| 65 | 0.35 | nM |
| 69 | >100 | nM |
| 68 | 21 | nM |
| 70 | >100 | nM |

### III. Preparation of Pharmaceutical Dosage Forms

### Example 1: Solution for Topical Ophthalmic Use

The active ingredient is a compound of any one of Table 1, Table 2, Table 3, Table 4, Table 5, Table 6, Table 7, Table 8, or Table 9, or a pharmaceutically acceptable salt thereof, and is formulated as a solution with a concentration of from 0.1-1.5% w/v.

1-111. (canceled)

112. A compound having a structure represented by Formula (A-II): 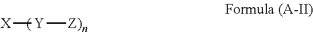
wherein:
X is an immunomodulator radical;
each Y is independently a linker;
each Z is independently a radical of a keratolytic agent; and
n is 1-3;
or a pharmaceutically acceptable salt or solvate thereof.

113. The compound of claim 112, or a pharmaceutically acceptable salt or solvate thereof, wherein:
n is 1 or 2; and
X is selected from the group consisting of a radical of a phosphodiesterase (PDE) inhibitor, a radical of a Janus kinase (JAK) inhibitor, a radical of a folate reductase inhibitor, a radical of a steroid; and a radical of a calcineurin inhibitor.

114. The compound of claim 113, or a pharmaceutically acceptable salt or solvate thereof, wherein X is a radical of a JAK inhibitor.

115. The compound of claim 114, or a pharmaceutically acceptable salt or solvate thereof, wherein X is a radical of a JAK1, JAK2, and/or JAK3 inhibitor.

116. The compound of claim 112, or a pharmaceutically acceptable salt or solvate thereof, wherein X has a structure represented by Formula (I-B): 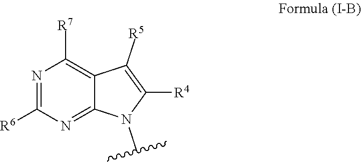
wherein:
R⁴ is hydrogen, halogen, or alkyl;
R⁵ is hydrogen, halogen, or alkyl;
R⁶ is hydrogen, halogen, or alkyl;
R⁷ is —NR^{b}R^{c} or optionally substituted heterocyclyl; and
R^{b} and R^{c} are each independently hydrogen, alkyl, optionally substituted cycloalkyl, or optionally substituted heterocyclyl.

117. The compound of claim 112, or a pharmaceutically acceptable salt or solvate thereof, wherein each linker is the same, and is a bond, substituted or unsubstituted alkyl, or substituted or unsubstituted heteroalkyl.

118. The compound of claim 112, or a pharmaceutically acceptable salt or solvate thereof, wherein: each linker is different, and is independently a bond, substituted or unsubstituted alkyl, or substituted or unsubstituted heteroalkyl.

119. The compound of claim 112, or a pharmaceutically acceptable salt or solvate thereof, wherein each radical of a keratolytic agent is a radical of the same keratolytic agent, and comprises one or more groups, each group being independently selected from the group consisting of —O—, oxo, substituted or unsubstituted alkyl, substituted or unsubstituted heteroalkyl, substituted or unsubstituted alkoxyl, substituted or unsubstituted cycloalkyl, and substituted or unsubstituted heterocyclyl.

120. The compound of claim 112, or a pharmaceutically acceptable salt or solvate thereof, wherein each radical of a keratolytic agent is a radical of a different keratolytic agent, and comprises one or more groups, each group being independently selected from the group consisting of —O—, oxo, substituted or unsubstituted alkyl, substituted or unsubstituted heteroalkyl, substituted or unsubstituted alkoxyl, substituted or unsubstituted cycloalkyl, and substituted or unsubstituted heterocyclyl.

121. The compound of claim 112, or a pharmaceutically acceptable salt or solvate thereof, wherein each radical of a keratolytic agent is alkyl substituted with one or more substituent, each substituent being independently selected from the group consisting of oxo, hydroxy, alkyl, alkoxy, and substituted or unsubstituted heterocyclyl.

122. The compound of claim 112, or a pharmaceutically acceptable salt or solvate thereof, wherein: each radical of a keratolytic agent is heteroalkyl substituted with one or more substituent, each substituent being independently selected from the group consisting of oxo, alkyl, thioalkyl, and substituted or unsubstituted heterocyclyl.

123. The compound of claim 112, or a pharmaceutically acceptable salt or solvate thereof, wherein each radical of a keratolytic agent has a structure represented by: 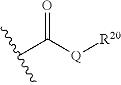
wherein:
Q is —O— or —(CR¹⁸R¹⁹)ₘ—;
m is 1-6;
each R¹⁸ and R¹⁹ is independently H, halo, alkyl, alkoxy, haloalkyl, or thioalkyl;
or an adjacent R¹⁸ and R¹⁹ combine to the atoms to which they are attached to form an oxo; and
R²⁰ is alkyl, heteroalkyl, heterocyclyl, alkoxy, or hydroxy, the alkyl, heteroalkyl, heterocycloalkyl, or alkoxy each independently being optionally substituted.

124. A compound having the structure: 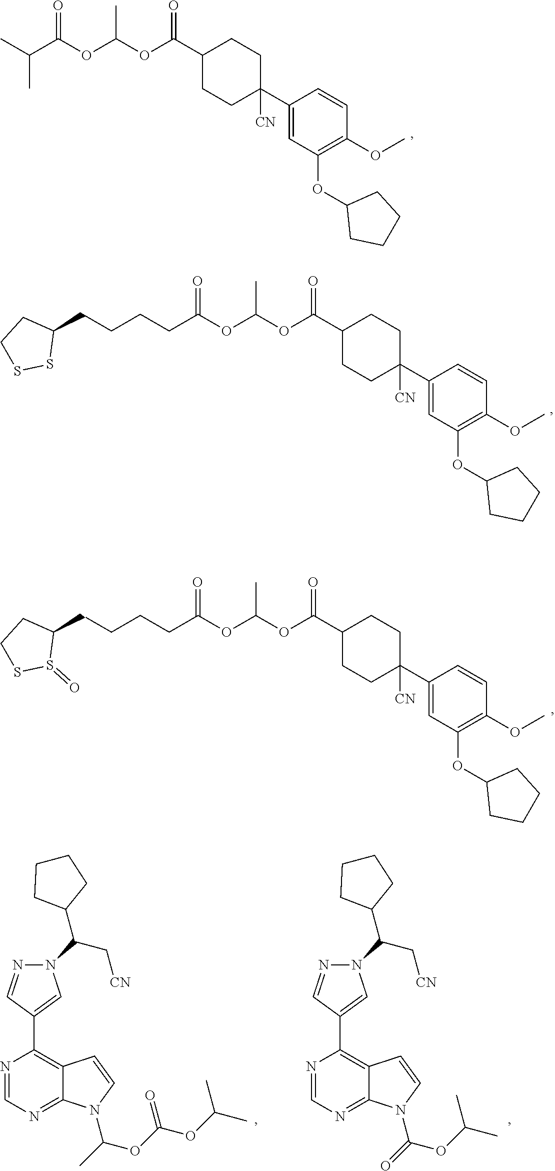 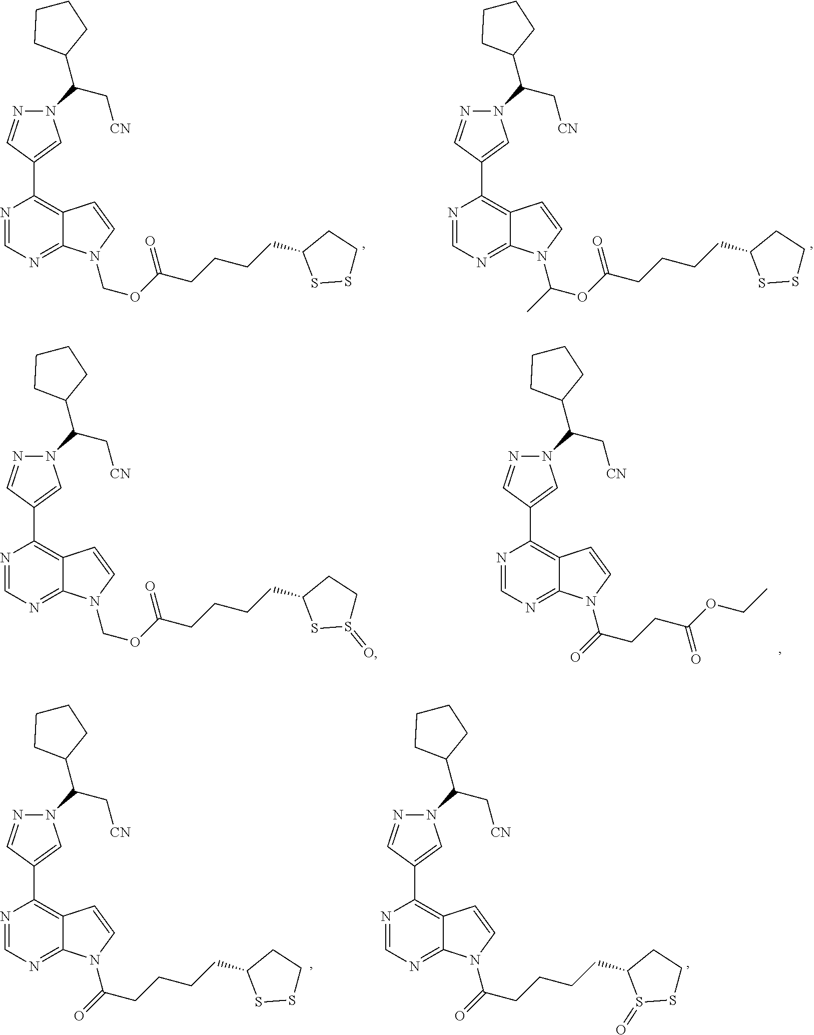 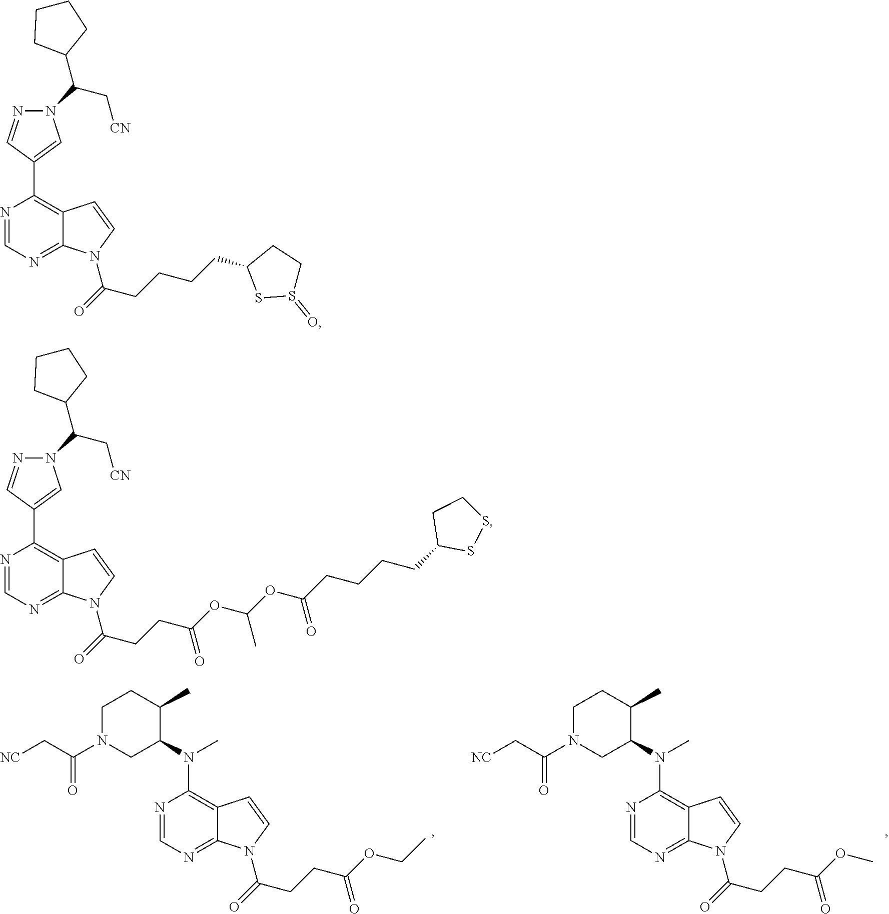 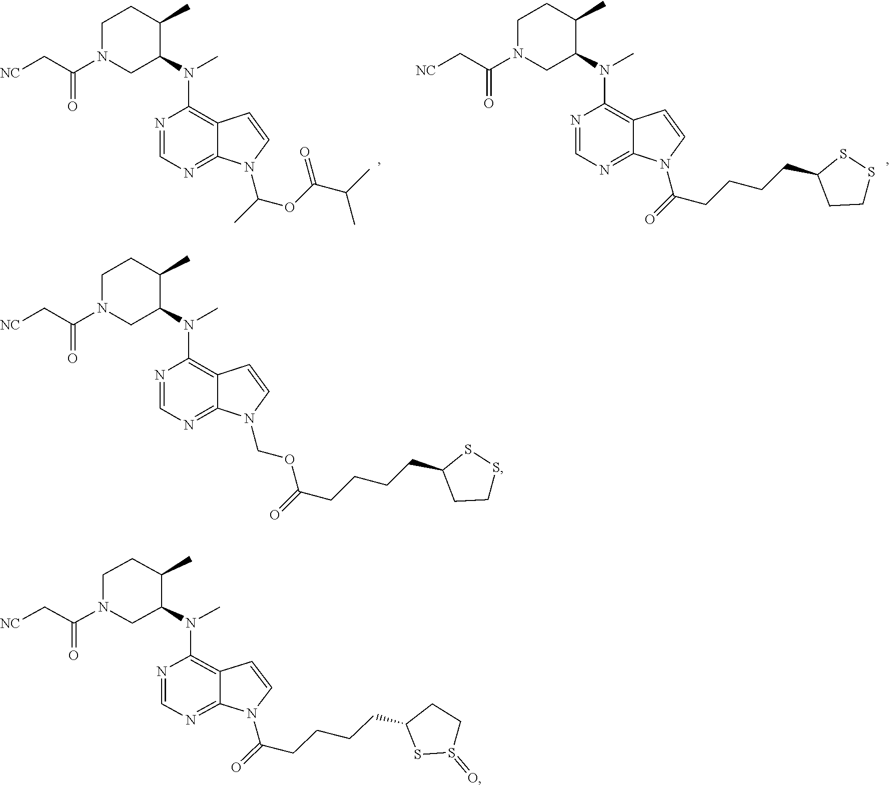 [SMILES: CC(C)C(=O)OC(C)n1ccc2c(N(C)[C@H]3CN(C(=O)CC#N)CC[C@H]3C)ncnc21 ; C[C@@H]1CCN(C(=O)CC#N)C[C@@H]1N(C)c1ncnc2c1ccn2C(=O)CCCC[C@@H]1CCSS1 ; C[C@@H]1CCN(C(=O)CC#N)C[C@@H]1N(C)c1ncnc2c1ccn2COC(=O)CCCC[C@@H]1CCSS1 ; C[C@@H]1CCN(C(=O)CC#N)C[C@@H]1N(C)c1ncnc2c1ccn2C(=O)CCCC[C@@H]1CCS(=O)S1] 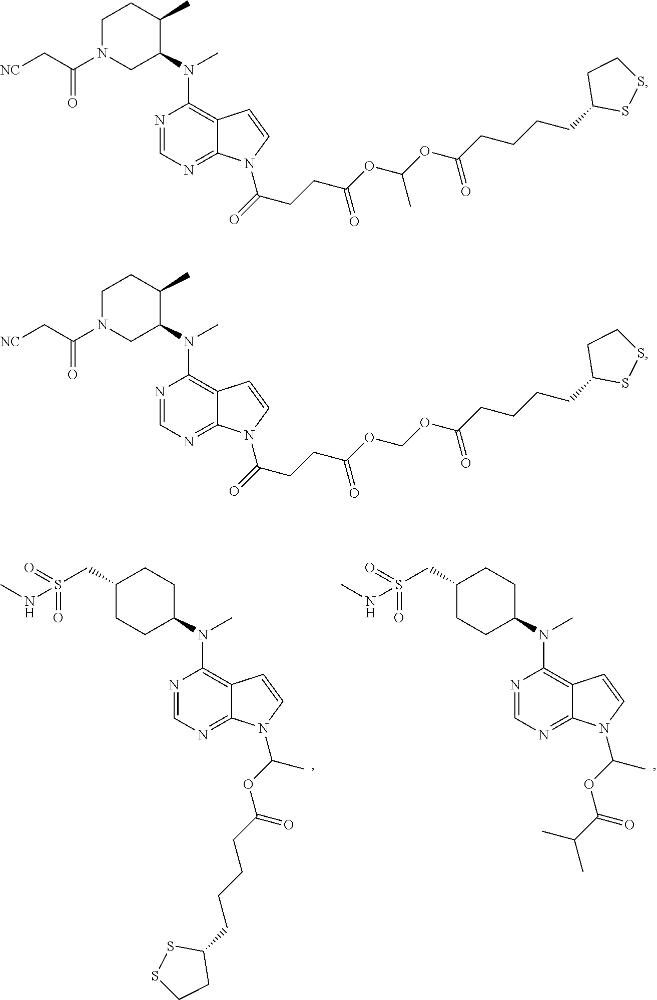 [SMILES: CC(OC(=O)CCCC[C@@H]1CCSS1)OC(=O)CCC(=O)n1ccc2c(N(C)[C@H]3CN(C(=O)CC#N)CC[C@H]3C)ncnc21 ; C[C@@H]1CCN(C(=O)CC#N)C[C@@H]1N(C)c1ncnc2c1ccn2C(=O)CCC(=O)OCOC(=O)CCCC[C@@H]1CCSS1 ; CNS(=O)(=O)C[C@H]1CC[C@H](N(C)c2ncnc3c2ccn3C(C)OC(=O)CCCC[C@@H]2CCSS2)CC1 ; CNS(=O)(=O)C[C@H]1CC[C@H](N(C)c2ncnc3c2ccn3C(C)OC(=O)C(C)C)CC1] 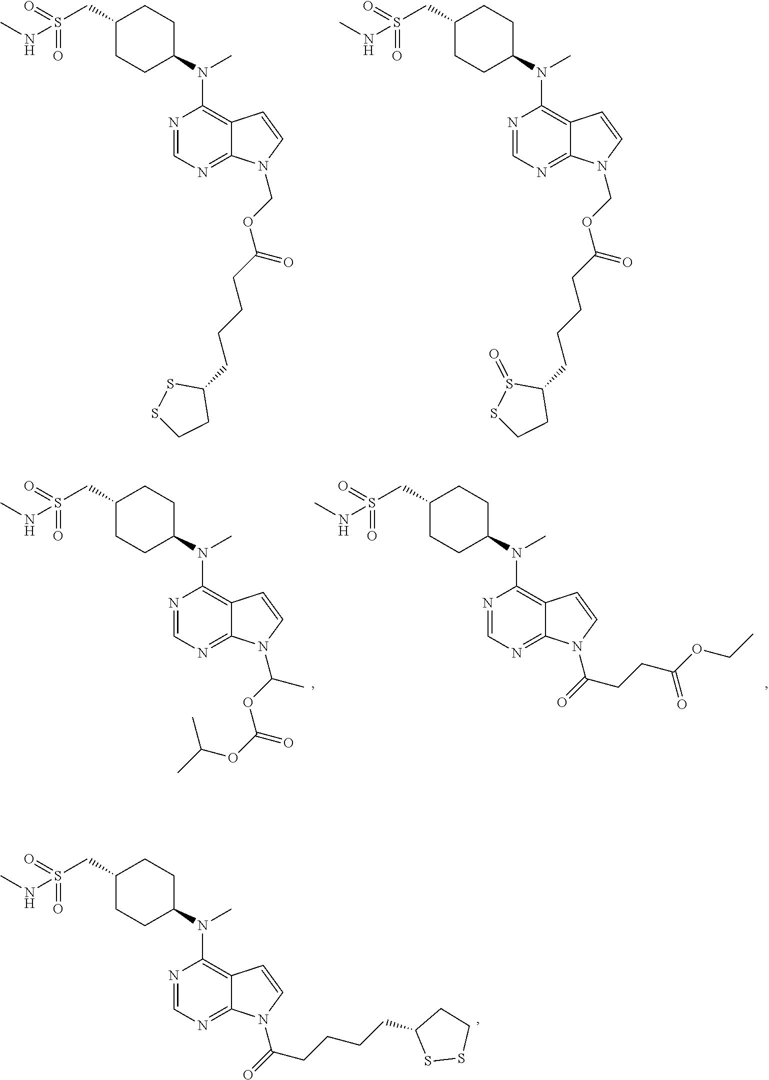 [SMILES: CNS(=O)(=O)C[C@H]1CC[C@H](N(C)c2ncnc3c2ccn3COC(=O)CCCC[C@@H]2CCSS2)CC1 ; CNS(=O)(=O)C[C@H]1CC[C@H](N(C)c2ncnc3c2ccn3COC(=O)CCCC[C@@H]2CCSS2=O)CC1 ; CNS(=O)(=O)C[C@H]1CC[C@H](N(C)c2ncnc3c2ccn3C(C)OC(=O)OC(C)C)CC1 ; CCOC(=O)CCC(=O)n1ccc2c(N(C)[C@H]3CC[C@H](CS(=O)(=O)NC)CC3)ncnc21 ; CNS(=O)(=O)C[C@H]1CC[C@H](N(C)c2ncnc3c2ccn3C(=O)CCCC[C@@H]2CCSS2)CC1] 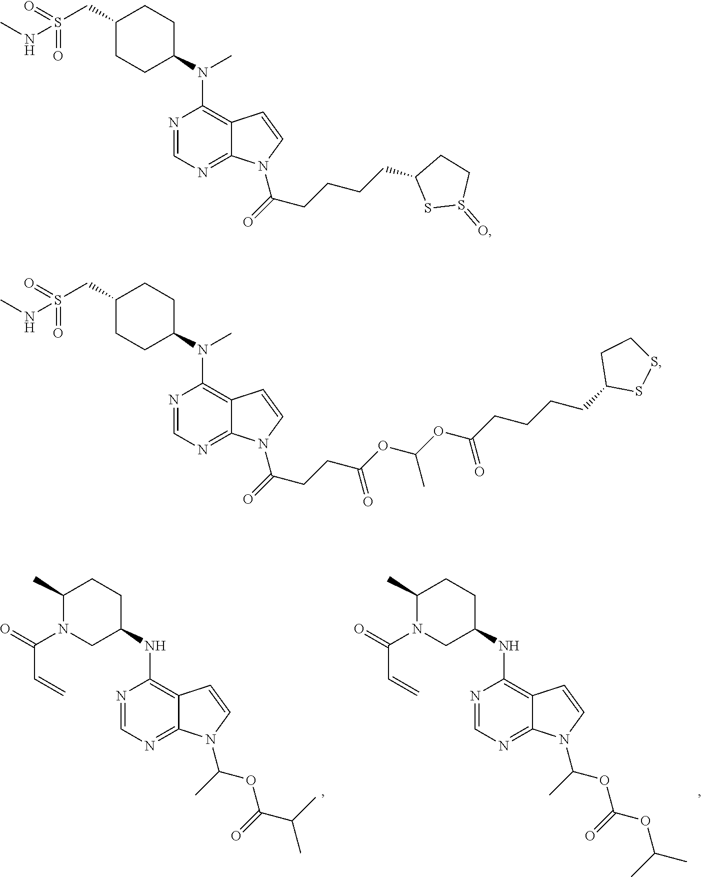 [SMILES: CNS(=O)(=O)C[C@H]1CC[C@H](N(C)c2ncnc3c2ccn3C(=O)CCCC[C@@H]2CCS(=O)S2)CC1 ; CNS(=O)(=O)C[C@H]1CC[C@H](N(C)c2ncnc3c2ccn3C(=O)CCC(=O)OC(C)OC(=O)CCCC[C@@H]2CCSS2)CC1 ; C=CC(=O)N1C[C@H](Nc2ncnc3c2ccn3C(C)OC(=O)C(C)C)CC[C@@H]1C ; C=CC(=O)N1C[C@H](Nc2ncnc3c2ccn3C(C)OC(=O)OC(C)C)CC[C@@H]1C] 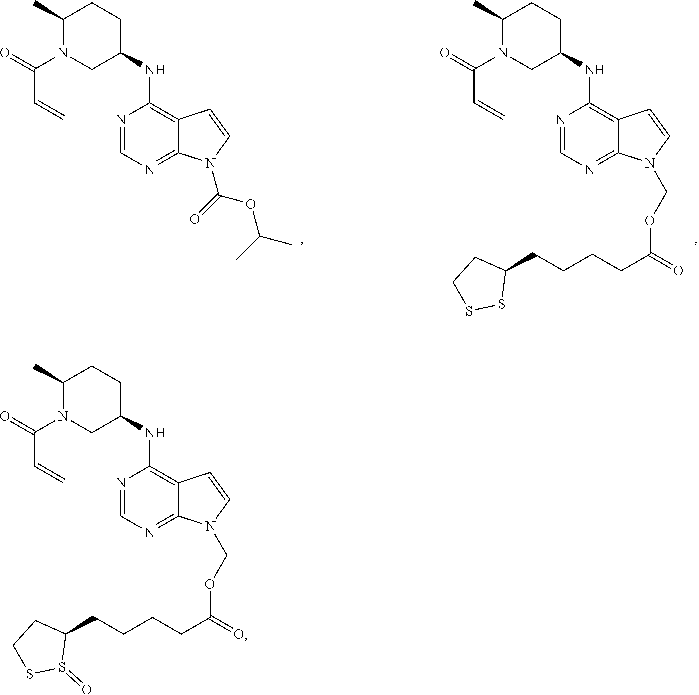 [SMILES: C=CC(=O)N1C[C@H](Nc2ncnc3c2ccn3C(=O)OC(C)C)CC[C@@H]1C ; C=CC(=O)N1C[C@H](Nc2ncnc3c2ccn3COC(=O)CCCC[C@@H]2CCSS2)CC[C@@H]1C ; C=CC(=O)N1C[C@H](Nc2ncnc3c2ccn3COC(=O)CCCC[C@@H]2CCSS2=O)CC[C@@H]1C] 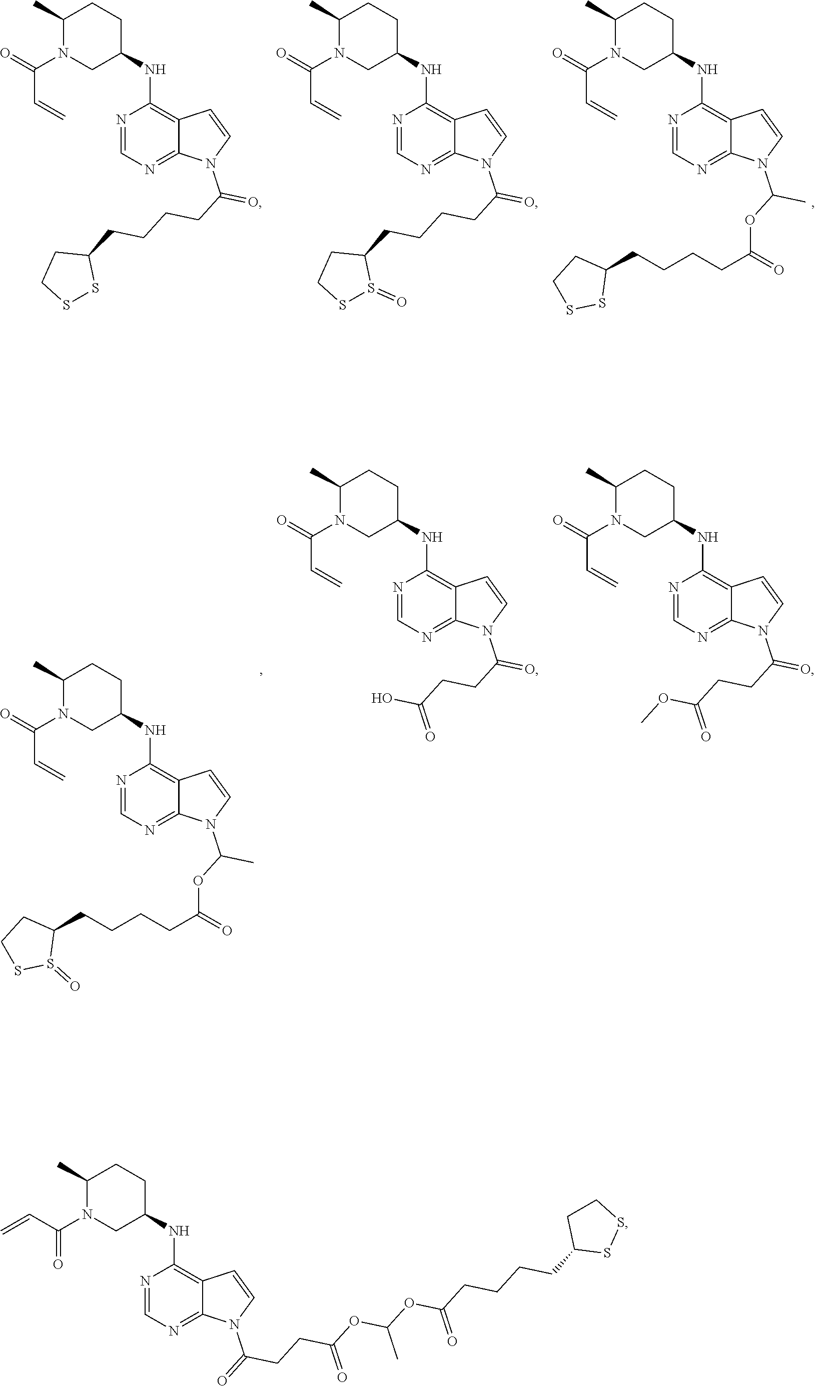 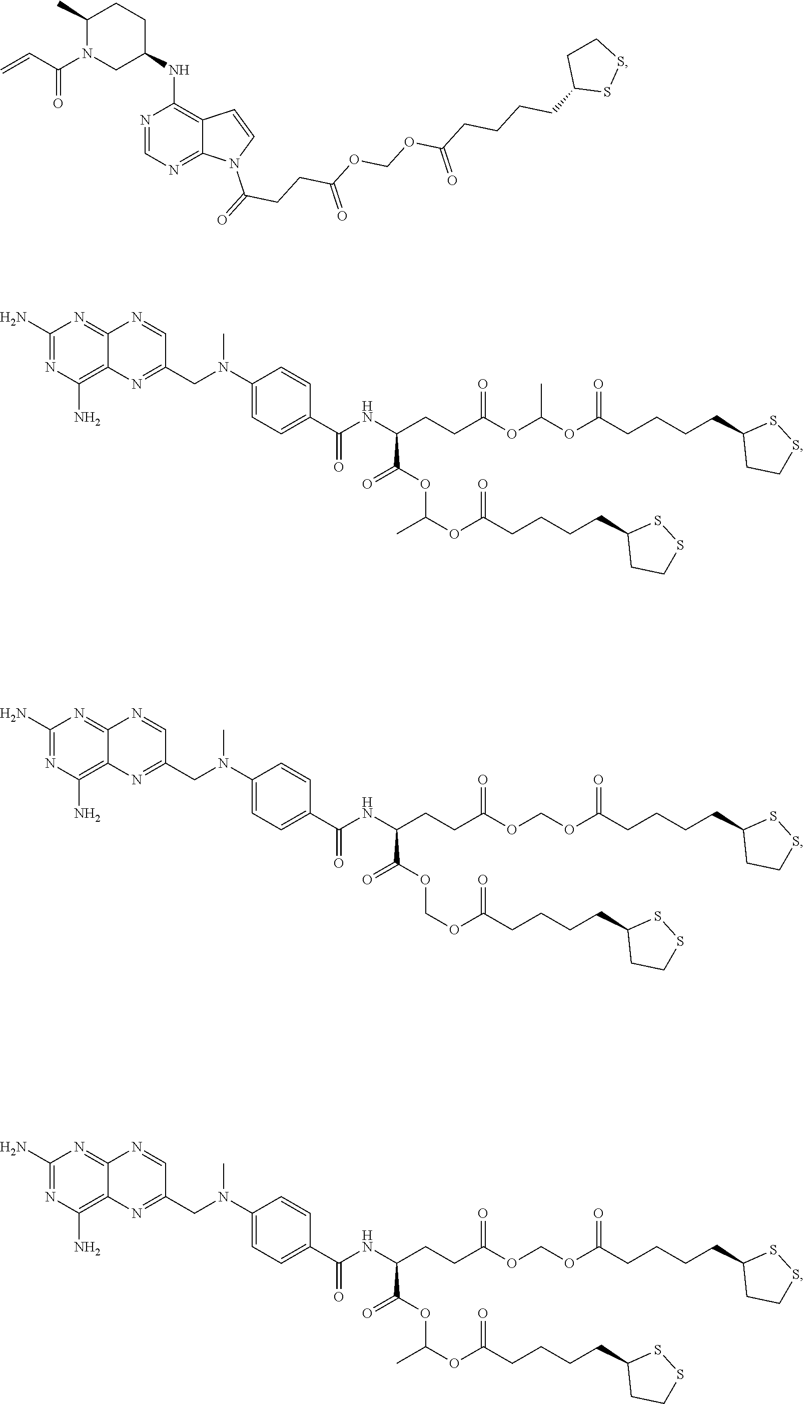 [SMILES: C=CC(=O)N1C[C@H](Nc2ncnc3c2ccn3C(=O)CCC(=O)OCOC(=O)CCCC[C@@H]2CCSS2)CC[C@@H]1C ; CC(OC(=O)CCCC[C@@H]1CCSS1)OC(=O)CC[C@H](NC(=O)c1ccc(N(C)Cc2cnc3nc(N)nc(N)c3n2)cc1)C(=O)OC(C)OC(=O)CCCC[C@@H]1CCSS1 ; CN(Cc1cnc2nc(N)nc(N)c2n1)c1ccc(C(=O)N[C@@H](CCC(=O)OCOC(=O)CCCC[C@@H]2CCSS2)C(=O)OCOC(=O)CCCC[C@@H]2CCSS2)cc1 ; CC(OC(=O)CCCC[C@@H]1CCSS1)OC(=O)[C@H](CCC(=O)OCOC(=O)CCCC[C@@H]1CCSS1)NC(=O)c1ccc(N(C)Cc2cnc3nc(N)nc(N)c3n2)cc1] 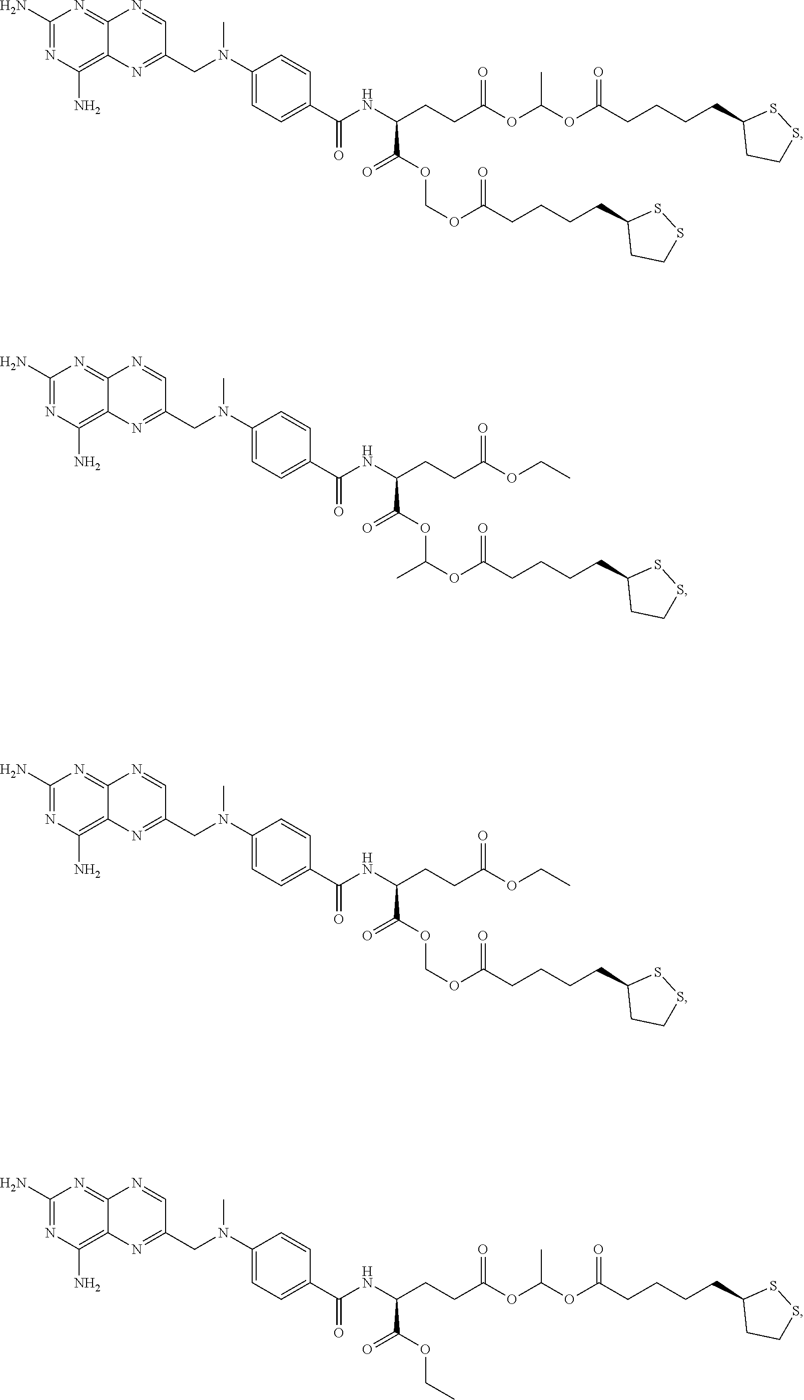 [SMILES: CC(OC(=O)CCCC[C@@H]1CCSS1)OC(=O)CC[C@H](NC(=O)c1ccc(N(C)Cc2cnc3nc(N)nc(N)c3n2)cc1)C(=O)OCOC(=O)CCCC[C@@H]1CCSS1 ; CCOC(=O)CC[C@H](NC(=O)c1ccc(N(C)Cc2cnc3nc(N)nc(N)c3n2)cc1)C(=O)OC(C)OC(=O)CCCC[C@@H]1CCSS1 ; CCOC(=O)CC[C@H](NC(=O)c1ccc(N(C)Cc2cnc3nc(N)nc(N)c3n2)cc1)C(=O)OCOC(=O)CCCC[C@@H]1CCSS1 ; CCOC(=O)[C@H](CCC(=O)OC(C)OC(=O)CCCC[C@@H]1CCSS1)NC(=O)c1ccc(N(C)Cc2cnc3nc(N)nc(N)c3n2)cc1] 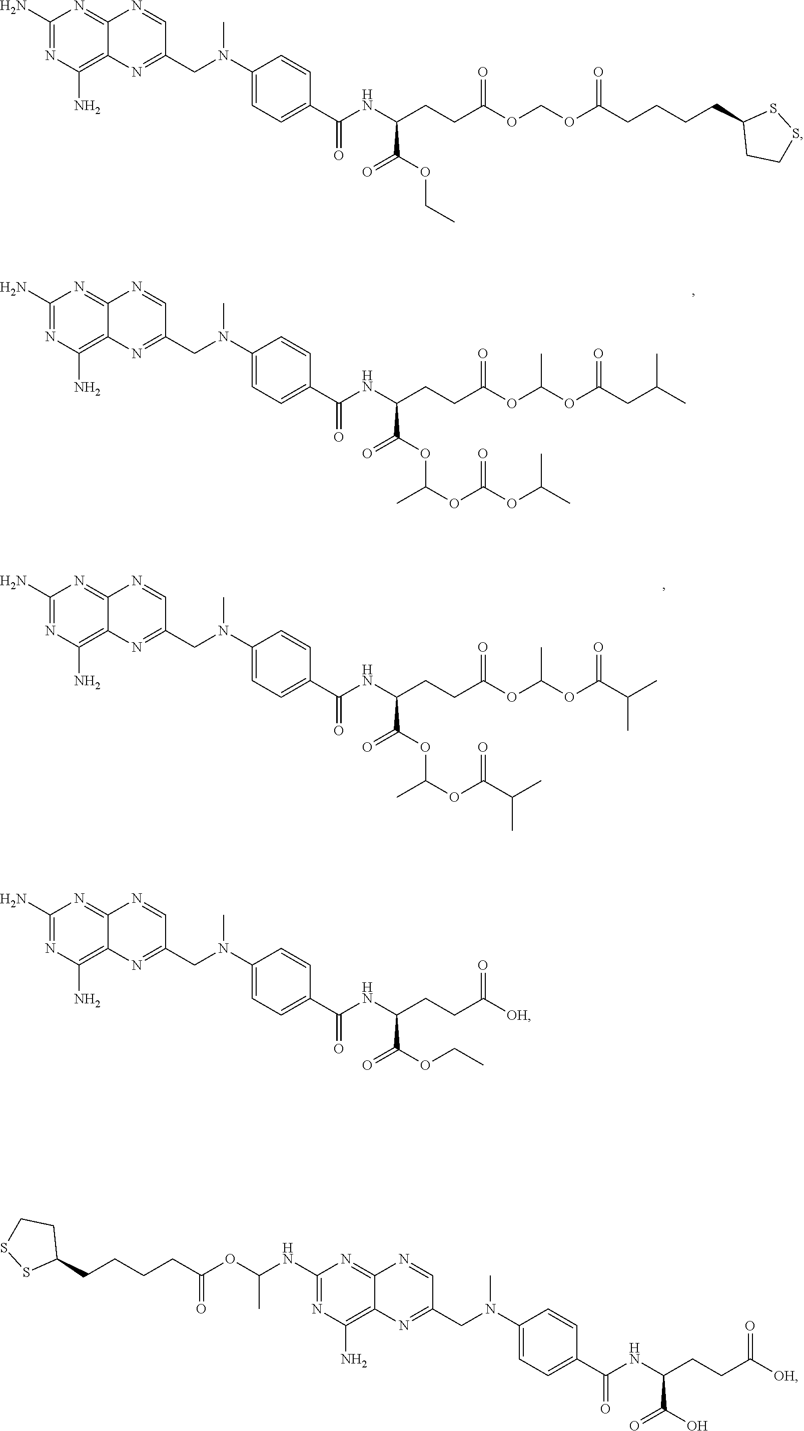 [SMILES: CCOC(=O)[C@H](CCC(=O)OCOC(=O)CCCC[C@@H]1CCSS1)NC(=O)c1ccc(N(C)Cc2cnc3nc(N)nc(N)c3n2)cc1 ; CC(C)CC(=O)OC(C)OC(=O)CC[C@H](NC(=O)c1ccc(N(C)Cc2cnc3nc(N)nc(N)c3n2)cc1)C(=O)OC(C)OC(=O)OC(C)C ; CC(OC(=O)CC[C@H](NC(=O)c1ccc(N(C)Cc2cnc3nc(N)nc(N)c3n2)cc1)C(=O)OC(C)OC(=O)C(C)C)OC(=O)C(C)C ; CCOC(=O)[C@H](CCC(=O)O)NC(=O)c1ccc(N(C)Cc2cnc3nc(N)nc(N)c3n2)cc1 ; CC(Nc1nc(N)c2nc(CN(C)c3ccc(C(=O)N[C@@H](CCC(=O)O)C(=O)O)cc3)cnc2n1)OC(=O)CCCC[C@@H]1CCSS1] 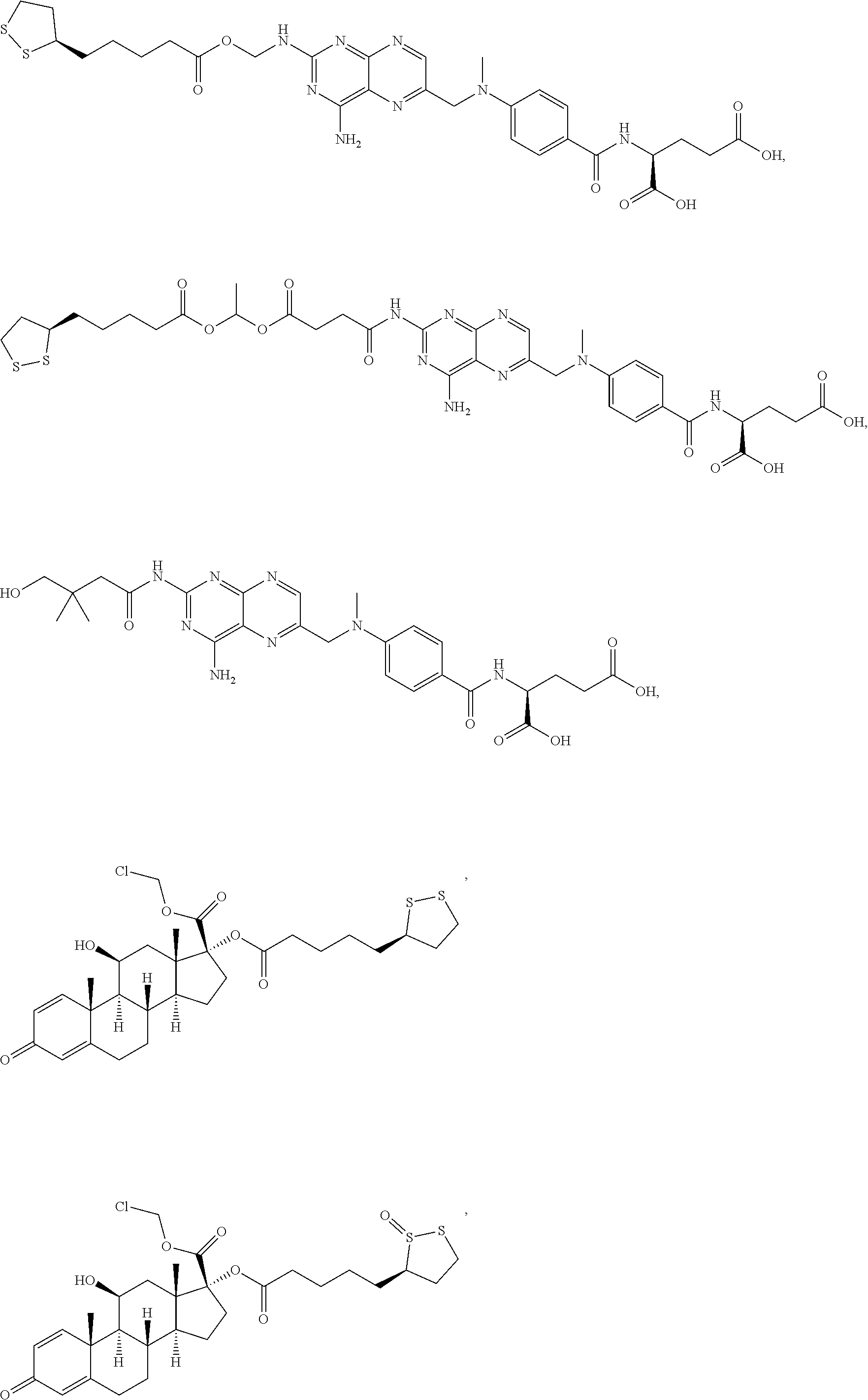 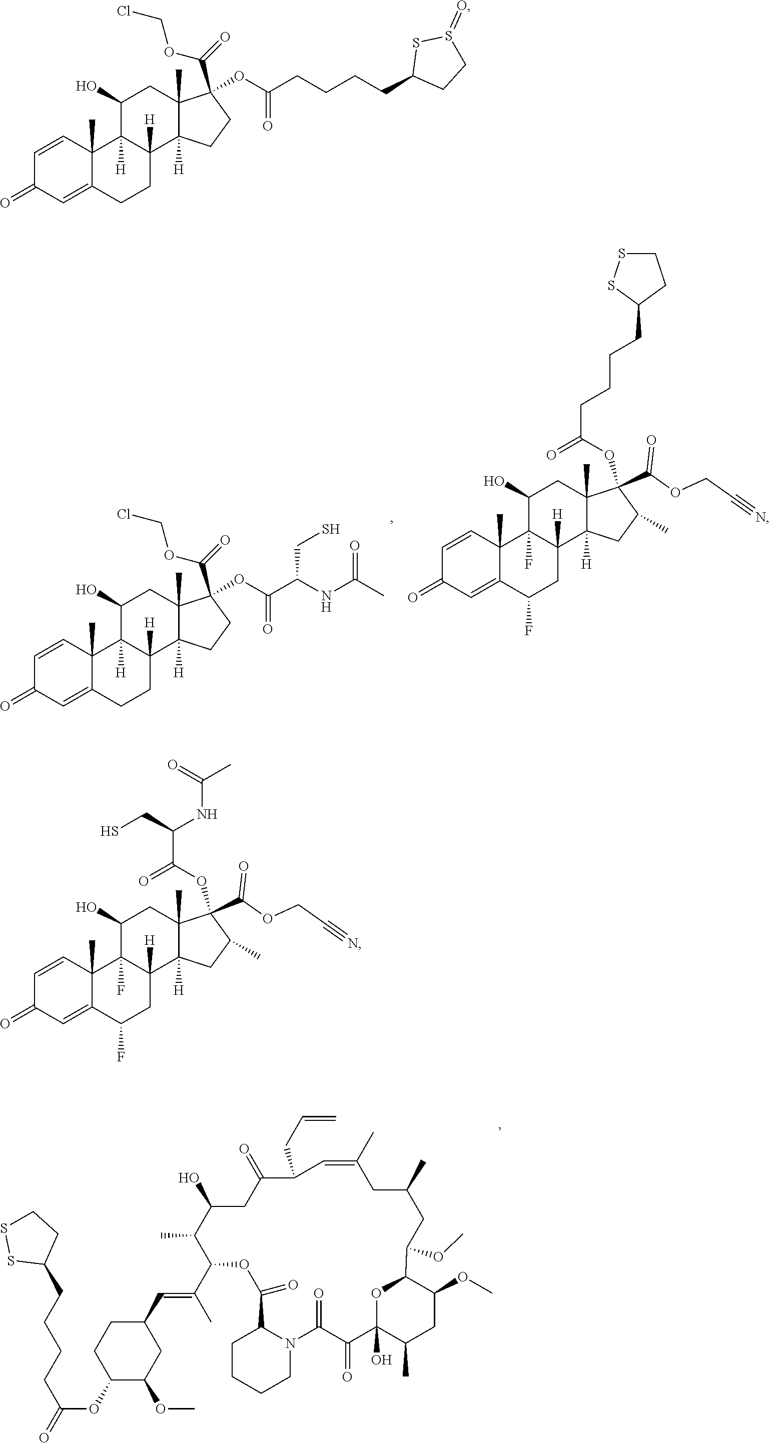 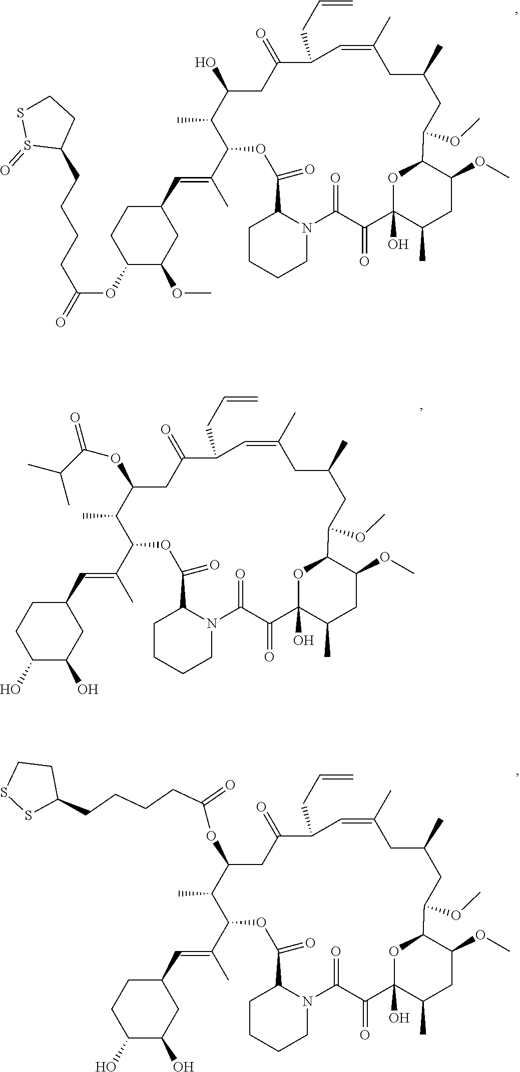 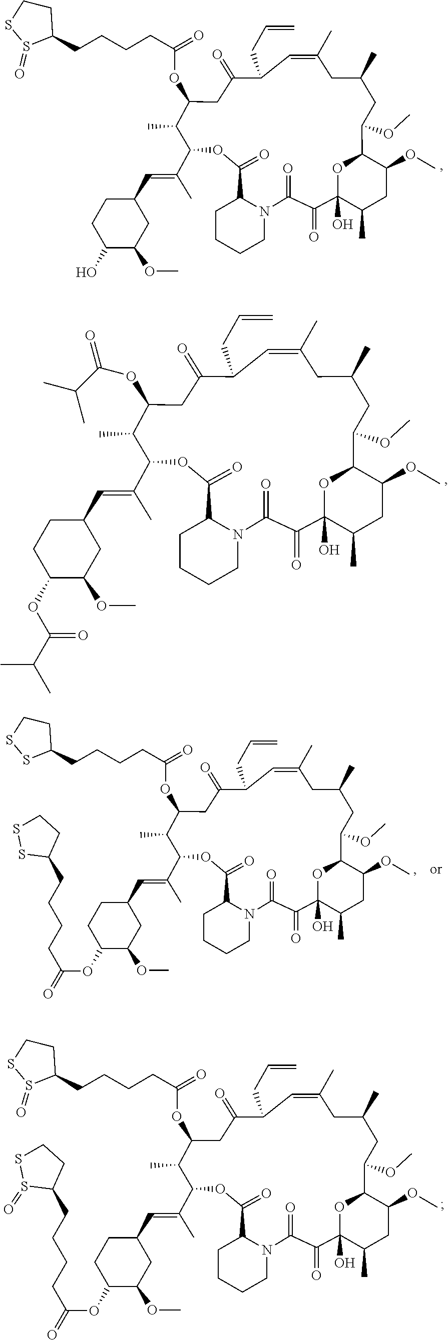
or a pharmaceutically acceptable salt or solvate thereof.

125. A compound having the structure: 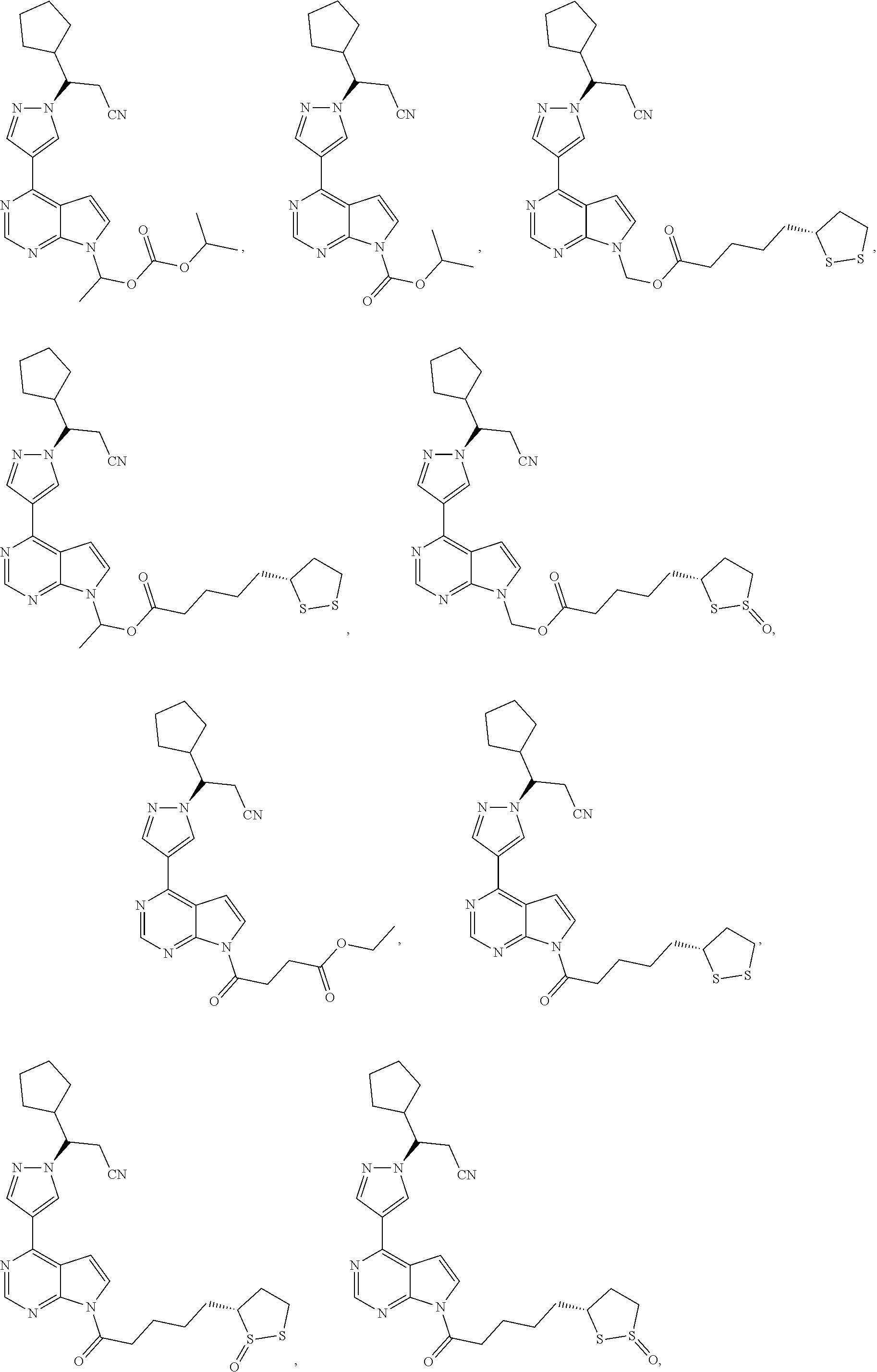 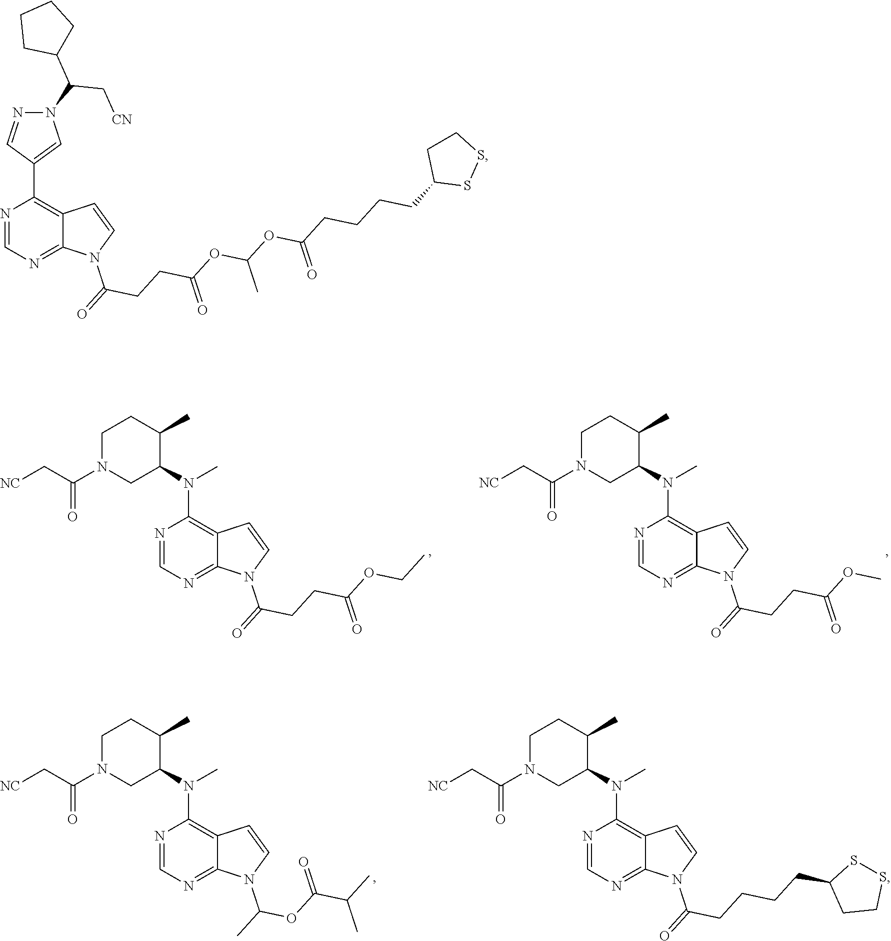 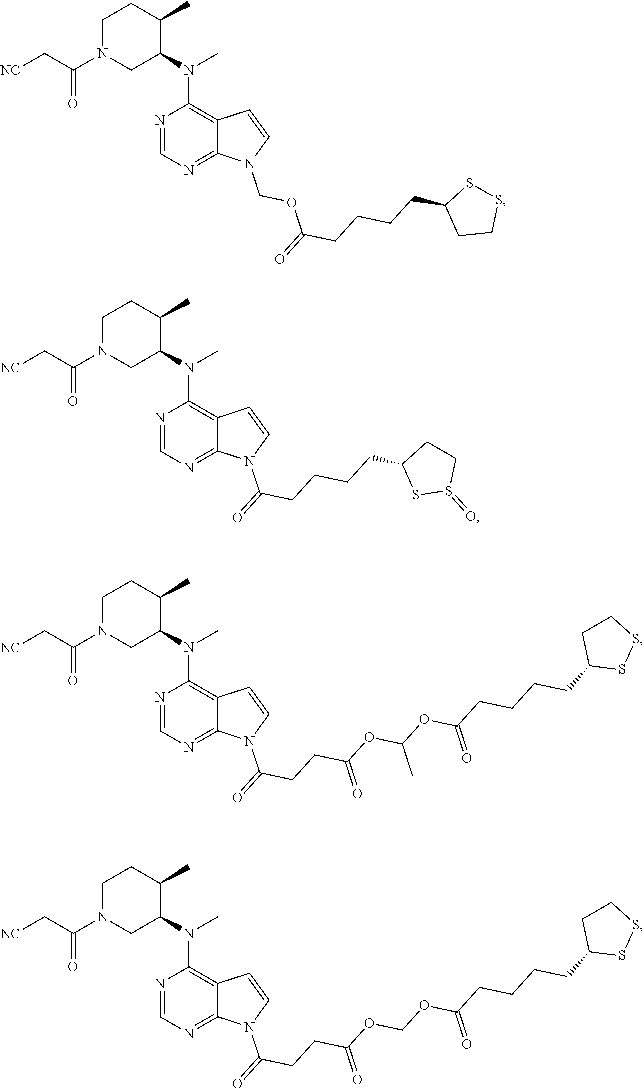 [SMILES: C[C@@H]1CCN(C(=O)CC#N)C[C@@H]1N(C)c1ncnc2c1ccn2COC(=O)CCCC[C@@H]1CCSS1 ; C[C@@H]1CCN(C(=O)CC#N)C[C@@H]1N(C)c1ncnc2c1ccn2C(=O)CCCC[C@@H]1CCS(=O)S1 ; CC(OC(=O)CCCC[C@@H]1CCSS1)OC(=O)CCC(=O)n1ccc2c(N(C)[C@H]3CN(C(=O)CC#N)CC[C@H]3C)ncnc21 ; C[C@@H]1CCN(C(=O)CC#N)C[C@@H]1N(C)c1ncnc2c1ccn2C(=O)CCC(=O)OCOC(=O)CCCC[C@@H]1CCSS1] 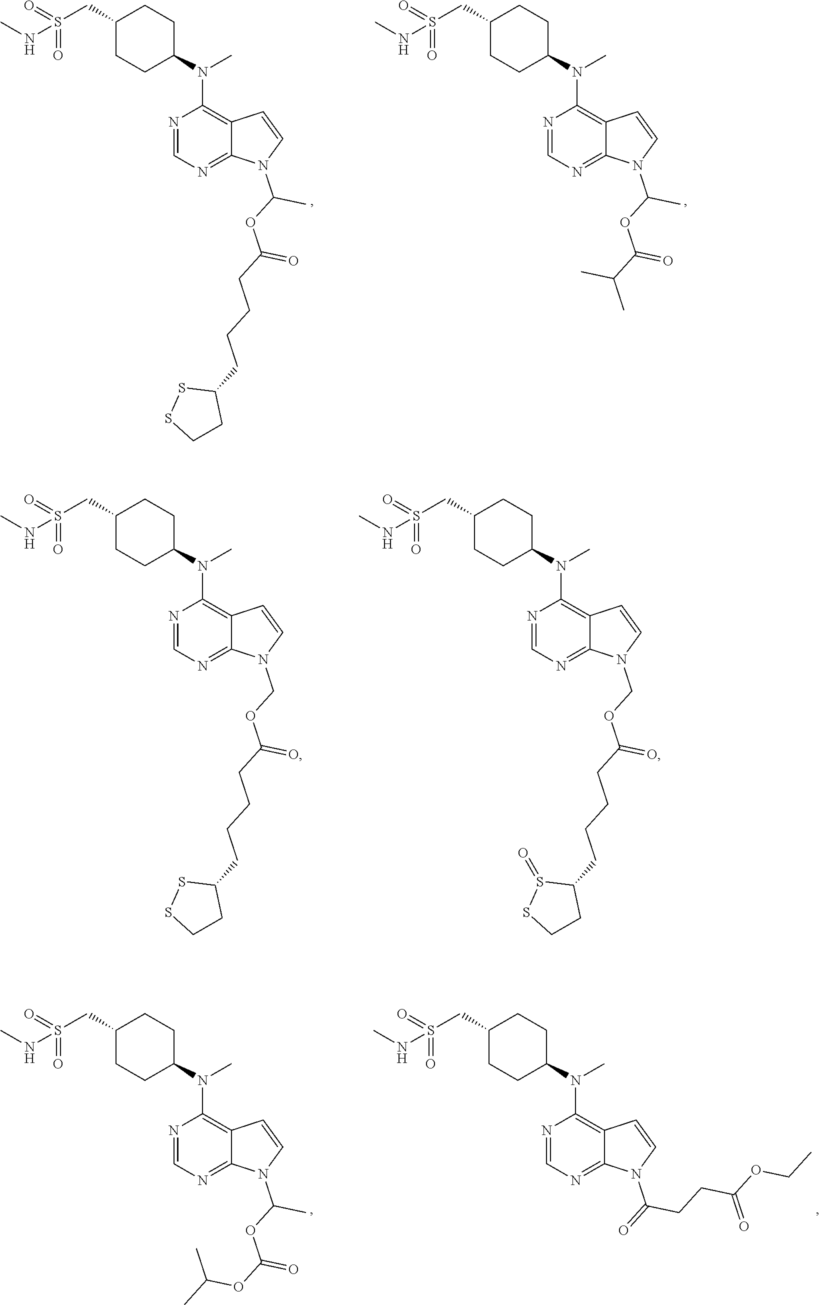 [SMILES: CNS(=O)(=O)C[C@H]1CC[C@H](N(C)c2ncnc3c2ccn3C(C)OC(=O)CCCC[C@@H]2CCSS2)CC1 ; CNS(=O)(=O)C[C@H]1CC[C@H](N(C)c2ncnc3c2ccn3C(C)OC(=O)C(C)C)CC1 ; CNS(=O)(=O)C[C@H]1CC[C@H](N(C)c2ncnc3c2ccn3COC(=O)CCCC[C@@H]2CCSS2)CC1 ; CNS(=O)(=O)C[C@H]1CC[C@H](N(C)c2ncnc3c2ccn3COC(=O)CCCC[C@@H]2CCSS2=O)CC1 ; CNS(=O)(=O)C[C@H]1CC[C@H](N(C)c2ncnc3c2ccn3C(C)OC(=O)OC(C)C)CC1 ; CCOC(=O)CCC(=O)n1ccc2c(N(C)[C@H]3CC[C@H](CS(=O)(=O)NC)CC3)ncnc21] 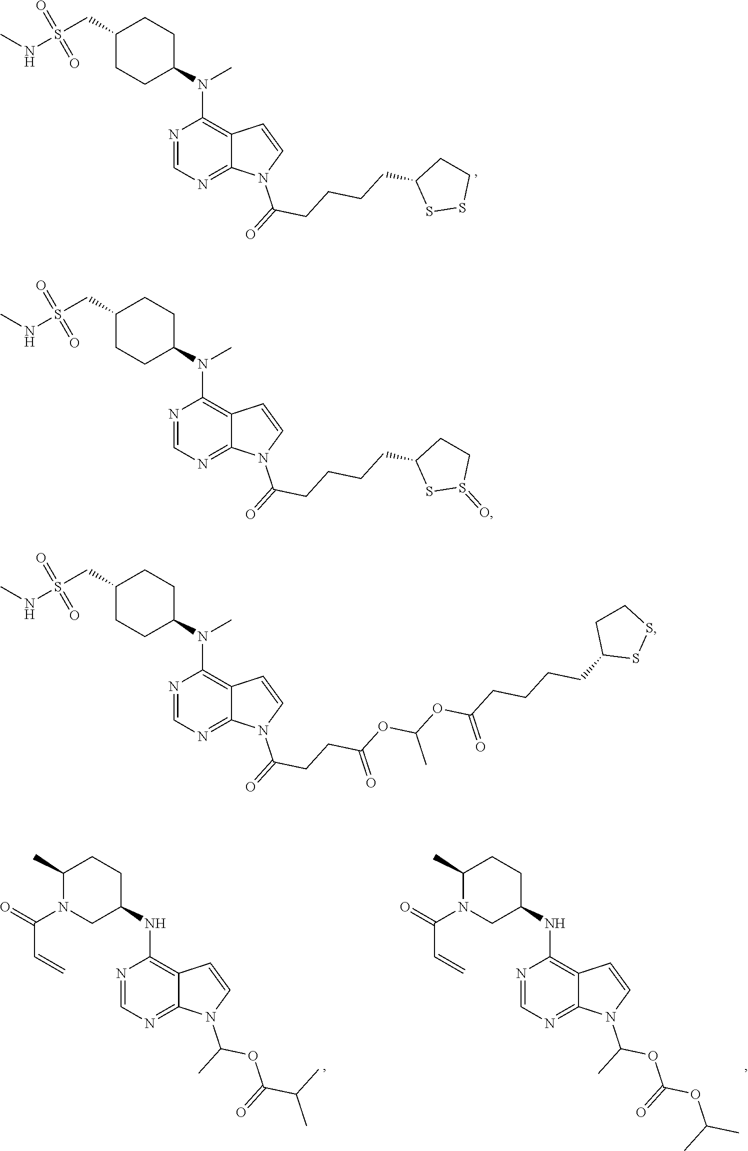 [SMILES: CNS(=O)(=O)C[C@H]1CC[C@H](N(C)c2ncnc3c2ccn3C(=O)CCCC[C@@H]2CCSS2)CC1 ; CNS(=O)(=O)C[C@H]1CC[C@H](N(C)c2ncnc3c2ccn3C(=O)CCCC[C@@H]2CCS(=O)S2)CC1 ; CNS(=O)(=O)C[C@H]1CC[C@H](N(C)c2ncnc3c2ccn3C(=O)CCC(=O)OC(C)OC(=O)CCCC[C@@H]2CCSS2)CC1 ; C=CC(=O)N1C[C@H](Nc2ncnc3c2ccn3C(C)OC(=O)C(C)C)CC[C@@H]1C ; C=CC(=O)N1C[C@H](Nc2ncnc3c2ccn3C(C)OC(=O)OC(C)C)CC[C@@H]1C] 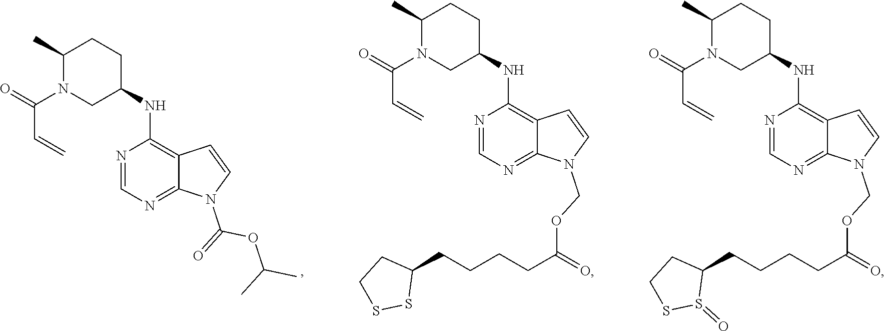 [SMILES: C=CC(=O)N1C[C@H](Nc2ncnc3c2ccn3C(=O)OC(C)C)CC[C@@H]1C ; C=CC(=O)N1C[C@H](Nc2ncnc3c2ccn3COC(=O)CCCC[C@@H]2CCSS2)CC[C@@H]1C ; C=CC(=O)N1C[C@H](Nc2ncnc3c2ccn3COC(=O)CCCC[C@@H]2CCSS2=O)CC[C@@H]1C] 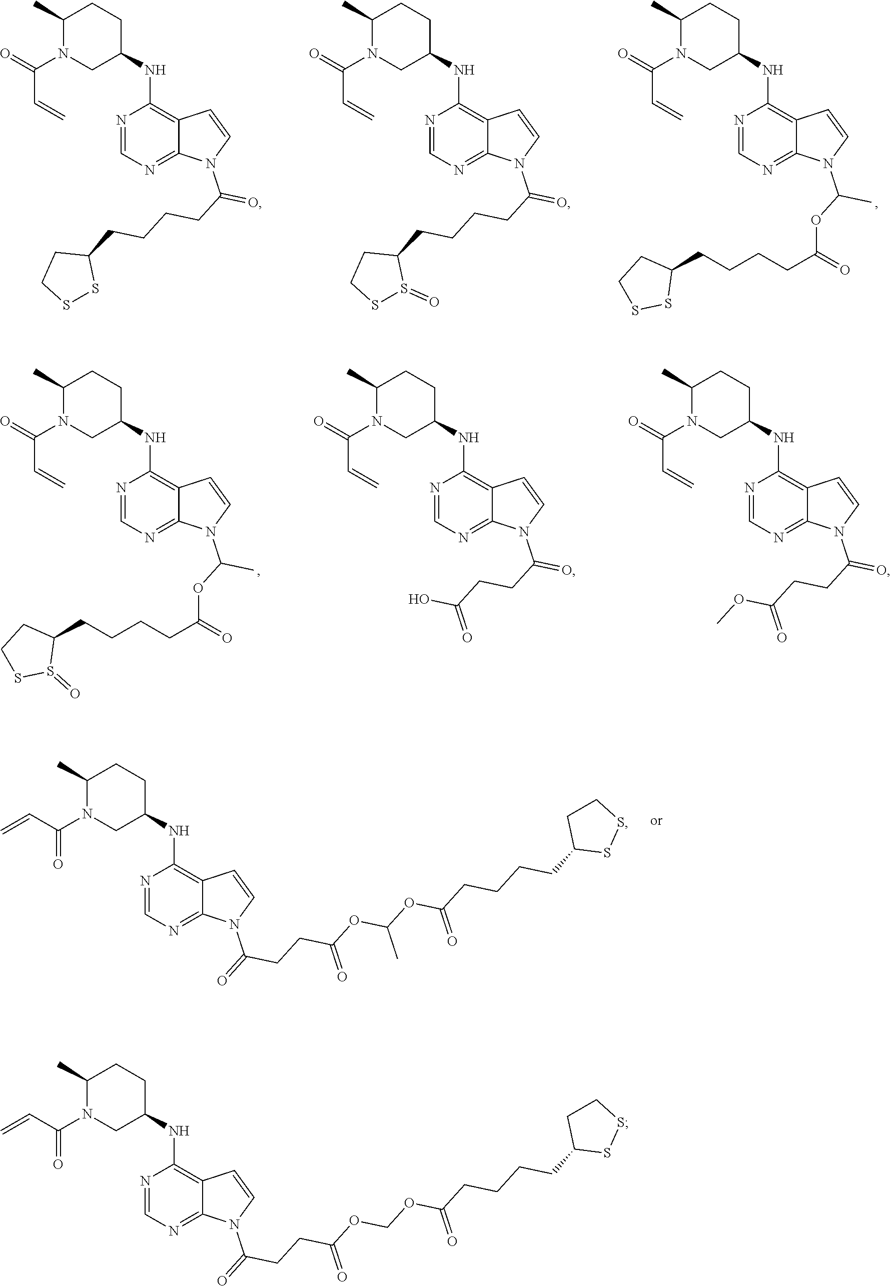
or a pharmaceutically acceptable salt or solvate thereof.

126. A pharmaceutical composition comprising a compound of claim 112, or a pharmaceutically acceptable salt or solvate thereof, and at least one pharmaceutically acceptable excipient.

127. A method of treating a dermal or an ocular disease or disorder in an individual,
the method comprising administering a compound of claim 112 to the individual.

128. The method of claim 127, wherein the dermal or the ocular disease or disorder is associated with keratosis, microbial infiltration, microbial infection, inflammation, or any combination thereof.
